(19) United States
(12) Patent Application Publication
(10) Pub. No.: US 2023/0322722 A1
(43) Pub. Date: Oct. 12, 2023

(54) MODULATORS OF MYC FAMILY PROTO-ONCOGENE PROTEIN

(71) Applicant: Nalo Therapeutics, Belmont, CA (US)

(72) Inventors: William Greenlee, Teaneck, NJ (US); Stephen J. Shuttleworth, Palo Alto, CA (US); Keith Wilson, Belmont, CA (US)

(21) Appl. No.: 18/023,298

(22) PCT Filed: Aug. 25, 2021
(86) PCT No.: PCT/US2021/047489

§ 371 Date: Feb. 24, 2023

Related U.S. Application Data
(60) Provisional application No. 63/070,753, filed on Aug. 26, 2020.
(60) Provisional application No. 63/070,762, filed on Aug. 26, 2020.

(51) Int. Cl. C07D 401/14; C07D 403/14; C07D 471/04; C07D 405/14; C07D 487/10; C07D 413/14; C07D 487/04; A61K 45/06

(52) U.S. Cl. CPC C07D 401/14; C07D 403/14; C07D 471/04; A61K 45/06; C07D 487/10; C07D 413/14; C07D 487/04; C07D 405/14

(57) ABSTRACT

Disclosed herein are compounds and compositions having potency in the modulation of Myc family proteins. Such compounds and compositions can be used in the treatment of proliferative diseases, such as cancer, or in the treatment of disease where modulation of Myc family proteins is desired. Also disclosed herein are methods of using said compounds and compositions.

### CROSS REFERENCE TO RELATED APPLICATIONS

This application claims priority to and the benefit of U.S. Provisional Patent Application Nos. 63/070,753 filed Aug. 26, 2020, and 63/070,762 filed Aug. 26, 2020, each of which is incorporated herein by reference in its entirety.

### BACKGROUND

The MYC proto-oncogene family comprises three members: C-MYC, MYCN, and MYCL. These oncogenes encode c-Myc, N-Myc, and L-Myc oncoproteins, respectively, which belong to a family of “super-transcription factors” that regulate the transcription of more than 15% of the entire genome, Recent studies in mouse models have suggested that the regulation of oncogenic Myc proteins could potentially lead to the development of cancer therapeutics, as it has been demonstrated that even transient inactivation of Myc causes tumor regression. However, the development of drugs and therapeutics that directly targets Myc proteins has met with two major challenges. First, Myc proteins lack a well-defined active site for the binding of small molecules, thus providing challenges for the functional modulation or inhibition of their activities. Second, Myc proteins are predominantly located in cell nuclei, and targeting nuclear Myc proteins with antibodies can be technically challenging. These challenges have spawned strategies for indirect regulation of Myc proteins.

For example, amplification and overexpression of N-Myc can lead to tumorigenesis. Excess N-My is associated with a variety of tumors, e.g., neuroblastomas. MYCN can also be activated in tumors through somatic mutation.

C-Myc can also be constitutively expressed in various cancers such as cervix, colon, breast, lung and stomach cancers. Such constitutive expression can lead to increased expression of other genes that are involved in cell proliferation.

Amplification of the, e.g., N-Myc gene in patients frequently results in poor health outcomes. However, strategies for direct modulation of Myc proteins remain elusive, as the Myc proteins are not easily targeted.

Therefore, an ongoing need exists for small-molecule therapeutic modulators of Myc proteins for the treatment of various ailments, diseases and disorders, e.g., cancer.

### SUMMARY

The present disclosure provides compounds and compositions that are useful as Myc protein modulators, and methods of using the same. Furthermore, the present disclosure contemplates using disclosed compounds and compositions as direct modulators of Myc proteins in the treatment of proliferative disease, such as cancer, or in the treatment of diseases where modulation of Myc family proteins is desired.

For example, the present disclosure provides a compound of Formula I:

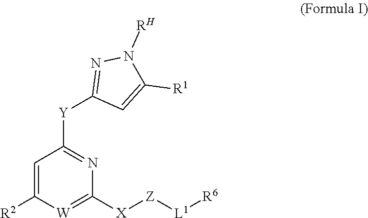

or a pharmaceutically acceptable salt, stereoisomer, and/or N-oxide thereof, wherein:
W is selected from the group consisting of N, C—H, and C—F;
X is selected from the group consisting of NR^{A}, O, S, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —S(O)_{w}—, —NR^{A}—S(O)_{w}—, —CHR^{L}—NR^{A}—S(O)_{w}—, —NR^{A}—S(O)_{w}—CHR^{L}—, —S(O)_{w}—NR^{A}—, —CH₂—S(O)_{w}—NR^{A}—, —S(O)_{w}—NR^{A}—CHR^{L}—, —CHR^{L}—C(O)—, —C(O)—, and bond, where w is 0, 1 or 2;
Z is 4-10 membered heterocyclic having at least one nitrogen, wherein the nitrogen is bound to L1, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-phenyl, C₁₋₃alkylene-heteroaryl, —C(O)-heteroaryl, phenoxy, and H; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of a bond, H and methyl (optionally subsisted by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be substituted by methyl, halo, cyano, oxo, or hydroxyl

Also disclosed herein are compounds of Formula Iaa:

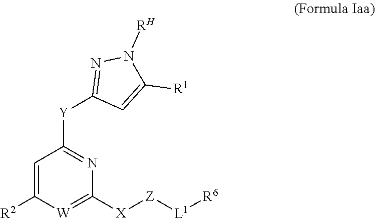

or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂C(CH₂)₂, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —S(O)_{w}—, —NR^{A}—S(O)_{w}—, —CHR^{L}—NR^{A}—S(O)_{w}—, —NR^{A}—S(O)_{w}—CHR^{L}—, —S(O)_{w}—NR^{A}—, —CH₂—S(O)_{w}—NR^{A}—, —S(O)_{w}—NR^{A}—CHR^{L}—, —CHR^{L}—C(O)—, —C(O)—, and bond, where w is 0, 1 or 2;
Z is selected from a 6-10 membered spiroheterocycle, a 6-10 membered fused bicyclic heterocyclic, and a 6-10 membered bridged cycloheteroalkyl each having at least one nitrogen, wherein the nitrogen is bound to L1, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-phenyl, C₁₋₃alkylene-heteroaryl, —C(O)-heteroaryl, phenoxy, and H; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of a bond, H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

In another aspect, provided herein is a compound of Formula III:

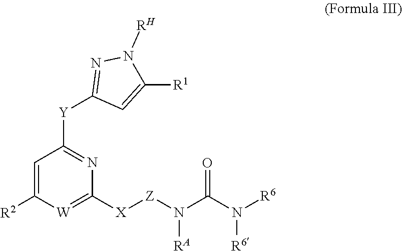

or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂, C(CH₂)₂, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ and R^{6′}, together with the nitrogen attached to R⁶ and R^{6′}, form a 4-8 membered monocyclic heterocyclyl or a 8-10 membered bicyclic heterocyclyl; wherein the monocyclic heterocyclyl or bicyclic heterocyclyl may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

Pharmaceutical compositions comprising a disclosed compound or a pharmaceutically acceptable salt, stereoisomer, and/or N-oxide thereof, as described herein, for example a disclosed pharmaceutical composition may include least one or more pharmaceutically acceptable carriers, diluents, stabilizers, excipients, dispersing agents, suspending agents, and/or thickening agents. The present disclosure also provides a method of manufacturing of the compounds described herein, or a pharmaceutically acceptable salt, stereoisomer, and/or N-oxide thereof.

A method of modulating the amount and activity of a Myc family protein (i.e., C-Myc, N-Myc, L-Myc, or human Myc) is also provided, for example, an activity of a Myc family protein may be modulated in a cell by contacting a cell with an effective amount of a compound as described herein, or a pharmaceutically acceptable salt, stereoisomer, and/or N-oxide thereof.

The present disclosure also provides a method of treating a Myc family protein associated disease in a subject in need thereof, the method comprising administering a therapeutically effective amount of a compound described herein, or a pharmaceutically acceptable salt, stereoisomer, and/or N-oxide thereof, including embodiments in any examples, tables, or figures. In some embodiments, the subject is a human subject and the disease is a proliferative disease, such as cancer.

### DETAILED DESCRIPTION

### Definitions

Unless defined otherwise, all technical and scientific terms used herein have the same meaning as commonly understood by one of ordinary skill in the art to which this disclosure pertains.

It is understood that the definitions provided herein are not intended to be mutually exclusive. Accordingly, some chemical moieties may fall within the definition of more than one term.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, n-butoxy, tert-butoxy, sec-butoxy, n-pentoxy, n-hexoxy, 1,2-dimethylbutoxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

As used herein, the term “alkylene” refers to a di-radical alkyl group. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

As used herein, the terms “alkenylene,” “alkynylene,” “arylene,” “arylalkylene,” and “alkylarylene” refer to di-radical alkenyl, alkynyl, aryl, arylalkyl, and alkylaryl groups, respectively.

As used herein, the term “azido” refers to group —N₃.

As used herein, the term “carboxyl,” “carboxy” or “carboxylate” refers to —CO₂H or salts thereof.

As used herein, the term “carbamoyl” refers to the group NH₂CO—.

The terms “cycloalkyl” or a “carbocyclic group” as used herein refers to a saturated or partially unsaturated hydrocarbon group of, for example, 3-10, 3-6, or 4-6 carbons, referred to herein as C₃₋₁₀cycloalkyl, or C₄₋₆cycloalkyl, respectively, and which may be monocyclic or bicyclic ring structures, e.g. 4-9 or 4-6 membered saturated ring structures, including bridged, fused or spirocyclic rings. Exemplary cycloalkyl groups include, but are not limited to, adamantanyl, cyclohexyl, cyclopentyl, cyclopentenyl, cyclobutyl, cyclopropyl, and indanyl.

As used herein, the groups

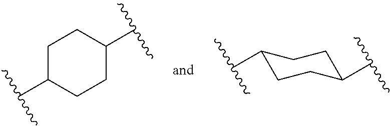 [SMILES: *C1CCC(*)CC1 ; *C1CCC(*)CC1]

and are used interchangeably and refer to a cyclohexyl group.

As used herein, the term “cyano” and “carbonitrile” refer to the group —CN.

As used herein, the term “formyl” refers to the group —C(O)H.

As used herein, the term “guanidino” refers to the group —NHC(═NH)NH₂.

As used herein, the terms “halo” and “halogen” are used in the conventional sense to refer to a chloro, bromo, fluoro or iodo substituent.

As used herein, the terms “hydroxy” and “hydroxyl” refer to the group —OH.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl” or “heterocyclic group” are art-recognized and refer to e.g. saturated or partially unsaturated, 4-10 membered monocyclic or bicyclic ring structures, or e.g. 4-9 or 4-6 membered saturated ring structures, including bridged, fused or spirocyclic rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

As used herein, the term “nitro” refers to the group —NO₂.

As used herein, the term “oxo” refers to the group (═O) or (O).

As used herein, the term “isomers” refers to compounds comprising the same numbers and types of atoms or components, but with different structural arrangement and connectivity of the atoms.

As used herein, the term “tautomer” refers to one of two or more structural isomers which readily convert from one isomeric form to another and which exist in equilibrium.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol ═ denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. Substituents around a carbocyclic or heterocyclic ring may be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the present disclosure embrace both solvated and unsolvated forms. In one embodiment, a disclosed compound is amorphous. In one embodiment, a disclosed compound is a single polymorph. In another embodiment, a disclosed compound is a mixture of polymorphs. In another embodiment, a disclosed compound is in a crystalline form.

The present disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent

As used herein, singular articles such as “a,” “an” and “the” and similar referents in the context of describing the elements are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (i.e., “such as”) provided herein, is intended merely to better illuminate the embodiments and does not pose a limitation on the scope of the claims unless otherwise stated.

In some embodiments, where the use of the term “about” is before a quantitative value, the present disclosure also includes the specific quantitative value itself, unless specifically stated otherwise. As used herein, the term “about” refers to a 10% variation from the nominal value unless otherwise indicated or inferred. Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remains operable. Moreover, two or more steps or actions can be conducted simultaneously.

As used herein, a dash (“-”) that is not between two letters or symbols refers to a point of bonding or attachment for a substituent. For example, —NH₂ is attached through the nitrogen atom.

As used herein, the terms “active agent,” “drug,” “pharmacologically active agent” and “active pharmaceutical ingredient” are used interchangeably to refer to a compound or composition which, when administered to a subject, induces a desired pharmacologic or physiologic effect by local or systemic action or both.

As used herein, the term “prodrug” refers to compounds that are transformed in vivo to provide a compound or pharmaceutically acceptable salt, hydrate or solvate of the compound described herein. The transformation can occur by various mechanisms (i.e., esterase, amidase, phosphatase, oxidative and/or reductive metabolism) in various locations (i.e., in the intestinal lumen or upon transit into the intestine, blood, or liver).

As used herein, the term “modulator” refers to a compound or composition that increases or decreases the level of a target or the function of a target, which may be, but is not limited to, a Myc family protein, such as c-Myc, N-Myc, L-Myc and human Myc.

As used herein, the term “degrader” refers to a compound or composition that decreases the amount of a target or the activity of a target. In some embodiments, the target may be, but is not limited to, a Myc family protein comprising c-Myc, N-Myc, L-Myc and human Myc.

As used herein, the term “degrading” refers to a method or process that decreases the amount of a target or the activity of a target. In some embodiments, the target may be, but is not limited to, a Myc family protein comprising c-Myc, N-Myc, L-Myc and human Myc.

As used herein, the term “Myc family protein” refers to any one of the proteins c-Myc, N-Myc, or L-Myc as described herein. In some embodiments, a Myc protein is a c-Myc protein. In some embodiments, a Myc protein is a N-Myc protein. In some embodiments, a Myc protein is a L-Myc protein. In some embodiments, a Myc protein is a human c-Myc protein. In some embodiments, a Myc protein is a human N-Myc protein. In some embodiments, a Myc protein is a human L-Myc protein. In some embodiments, a Myc family protein is a human Myc family protein.

As used herein, the terms “N-Myc” and “MycN” can be used interchangeably and refer to the protein “V-Myc myelocytomatosis viral related oncogene, neuroblastoma derived” and include the wildtype and mutant forms of the protein. In some embodiments, MycN refers to the protein associated with one or more of database entries of Entrez Gene 4613, OMIM 164840, UniProt P04198, and RegSeq NP_005369.

As used herein, the term “c-Myc” refers to the protein “V-Myc myelocytomatosis viral oncogene” and include the wildtype and mutant forms of the protein. In some embodiments, c-Myc refers to the protein associated with one or more of database entries of Entrez Gene 4609, OMIM 190080, UniProt P01106, and RegSeq NP_002458.

As used herein, the term “L-Myc” refers to the protein “V-Myc myelocytomatosis viral oncogene homolog, lung carcinoma derived” and include the wildtype and mutant forms of the protein. In some embodiments, L-Myc refers to the protein associated with one or more of database entries of Entrez Gene 4610, OMIM 164850, UniProt P12524, and RegSeq NP_001028253.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines, felines, equines, bovines, ovines, rodentia, etc. and primates, i.e., non-human primates, and humans. Non-human animal models, i.e., mammals, non-human primates, murines, lagomorpha, etc. may be used for experimental investigations.

As used herein, the terms “treating,” “treatment,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human and includes: (a) preventing the disease or a symptom of a disease from occurring in a subject which may be predisposed to the disease but has not yet been diagnosed as having it (i.e., including diseases that may be associated with or caused by a primary disease); (b) inhibiting the disease, i.e., arresting its development; and (c) relieving the disease, i.e., causing regression of the disease (i.e., reduction in of tumor burden). In some embodiments, certain methods described herein treat cancer associated with the signaling pathway of a Myc family protein, such as c-Myc, N-Myc, L-Myc or human Myc.

As used herein, the term “pharmaceutically acceptable salt” refers to a salt which is acceptable for administration to a subject. It is understood that such salts, with counter ions, will have acceptable mammalian safety for a given dosage regime. Such salts can also be derived from pharmaceutically acceptable inorganic or organic bases and from pharmaceutically acceptable inorganic or organic acids, and may comprise organic and inorganic counter ions. The neutral forms of the compounds described herein may be converted to the corresponding salt forms by contacting the compound with a base or acid and isolating the resulting salts.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like.

Other examples of salts include anions of the compounds of the present disclosure compounded with a suitable cation such as N⁺, NH₄⁺, and NW₄⁺ (where W can be a C₁-C₈ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

As used herein, the terms “determining,” “measuring,” “assessing,” and “assaying” are used interchangeably and include both quantitative and qualitative determinations.

As used herein, the phrase “signaling pathway” refers to a series of interactions between cellular components, both intracellular and extracellular, that conveys a change to one or more other components in a living organism, which may cause a subsequent change to additional component. Optionally, the changes conveyed by one signaling pathway may propagate to other signaling pathway components. Examples of cellular components include, but are not limited to, proteins, nucleic acids, peptides, lipids and small molecules.

As used herein, the terms “effective amount” and “therapeutically effective amount” are used interchangeably and refer to the amount of a compound that, when administered to a mammal or other subject for treating a disease, condition, or disorder, is sufficient to affect such treatment for the disease, condition, or disorder. The “effective amount” or “therapeutically effective amount” will vary depending on the compound, the disease and its severity and the age, weight, etc., of the subject to be treated.

As used herein, the terms “pharmaceutically acceptable excipient,” “pharmaceutically acceptable diluent,” “pharmaceutically acceptable carrier,” and “pharmaceutically acceptable adjuvant” refer to an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “a pharmaceutically acceptable excipient, diluent, carrier and adjuvant” as used in the specification and claims includes both one and more than one such excipient, diluent, carrier, and adjuvant.

As used herein, the term “pharmaceutical composition” is meant to encompass a composition suitable for administration to a subject, such as a mammal, especially a human. In general a “pharmaceutical composition” is sterile, and free of contaminants that are capable of eliciting an undesirable response within the subject (i.e., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of (R)-enantiomer and (S)-enantiomer, and enantio-enriched stereomeric mixtures comprising of (R)- and (S)-enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### Compounds

The disclosure is generally directed to compounds that modulate (e.g., degrade) MycN and/or MycC, and may therefore have significant antineoplastic properties. The disclosed compounds and pharmaceutical compositions thereof find use in a variety of applications in which the modulation of the amount and activity of a Myc protein is desired, including use as potent antineoplastic agents.

Thus provided herein, in part, is a compound of Formula I:

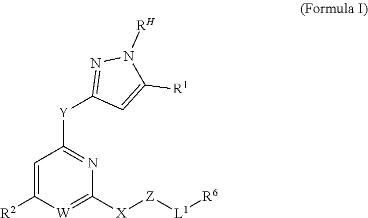

or a pharmaceutically acceptable salt, stereoisomer, and/or N-oxide thereof, wherein:
W is selected from the group consisting of N, C—H, and C—F;
X is selected from the group consisting of NR^{A}, O, S, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —S(O)_{w}—, —NR^{A}—S(O)_{w}—, —CHR^{L}—NR^{A}—S(O)_{w}—, —NR^{A}—S(O)_{w}—CHR^{L}, —S(O)_{w}—NR^{A}—, —CH₂—S(O)_{w}—NR^{A}—, —S(O)_{w}—NR^{A}—CHR^{L}—, —CHR^{L}—C(O)—, —C(O)—, and bond, where w is 0, 1 or 2;
Z is 4-10 membered heterocyclic having at least one nitrogen, wherein the nitrogen is bound to L1, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-phenyl, C₁₋₃alkylene-heteroaryl, —C(O)-heteroaryl, phenoxy, and H; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of a bond, H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

Exemplary disclosed compounds may be represented by the Formula I-A:

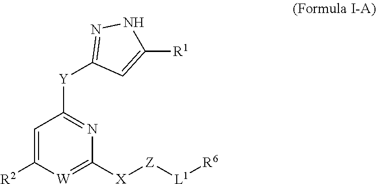

or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂ and C(CH₂)₂;
Y is selected from the group consisting of NH, N—CH₃, O, S, CH₂, CF₂, C(CH₃)₂, and C(CH₂CH₂);
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —NR^{A}—S(O)_{w}—, —CHR^{L}—NR^{A}—S(O)_{w}—, —NR^{A}—S(O)_{w}—CHR^{L}—, —S(O)_{w}—NR^{A}—, —CH₂—S(O)_{w}—NR^{A}—, and —S(O)_{w}—NR^{A}—CHR^{L}—, where w is 0, 1 or 2;
Z is 4-10 membered heterocyclic having at least one nitrogen, wherein the nitrogen is bound to L1, wherein Z may optionally be substituted by one or two substituents each independently selected from the group consisting of halo, hydroxyl, C₁-C₄ alkyl (optionally substituted by one, two or three halogens), —C(O)OH, and —C(O)—O—C₁₋₄alkyl;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-heteroaryl, —C(O)-heteroaryl, and phenoxy; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of a bond, H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

In some embodiments, for example, W is N, and a compound of the disclosure has the

### Formula Ia:

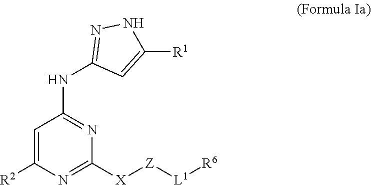

or a pharmaceutically acceptable salt, stereoisomer, and/or N-oxide thereof.

In some embodiments, R¹ is a 5-6 membered heterocyclyl or C₃₋₆cycloalkyl. For example, R¹ is selected from the group consisting of: 2-tetrahydrofuranyl, 3-tetrahydrofuranyl, 2-oxetanyl, cyclohexyl, cyclopropyl, cyclobutyl and cyclopentyl. In some embodiments, R¹ is cyclopropyl. In some embodiments, wherein R¹ is cyclopentyl.

In other embodiments, R¹ is selected from the group consisting of methyl and ethyl.

In some embodiments, X is NR^{A}.

In some embodiments, Z is selected from the group consisting of 4-6 membered monocyclic heterocycle, a 6-10 membered spiroheterocycle, a 6-10 membered fused bicyclic heterocyclic, and a 6-10 membered bridged cycloheteroalkyl.

Exemplary disclosed compounds are compounds of Formula Iaa:

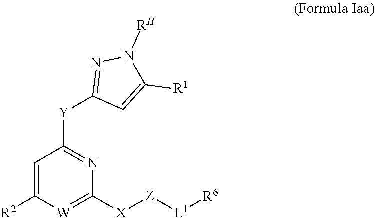

or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂C(CH₂)₂, NR^{A}CH₂, NR^{A}C(O), and C(O);
Y is selected from the group consisting of NH, N—CH₃, O, S, CH₂, CF₂, CH(CH₃), C(CH₃)₂, and C(CH₂C₂);
R^{H} is selected from the group consisting of H, C₁₋₃alkyl, —C(O)—C₁₋₃alkyl and C(O)—O—C₁₋₃alkyl;
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —S(O)_{w}—, —NR^{A}—S(O)_{w}—, —CHR^{L}—NR^{A}—S(O)_{w}—, —NR^{A}—S(O)_{w}—CHR^{L}, —S(O)_{w}—NR^{A}—, —CH₂—S(O)_{w}—NR^{A}—, —S(O)_{w}—NR^{A}—CHR^{L}—, —CHR^{L}—C(O)—, —C(O)—, and bond, where w is 0, 1 or 2;
Z is selected from a 6-10 membered spiroheterocycle, a 6-10 membered fused bicyclic heterocyclic, and a 6-10 membered bridged cycloheteroalkyl each having at least one nitrogen, wherein the nitrogen is bound to L1, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-phenyl, C₁₋₃alkylene-heteroaryl, —C(O)-heteroaryl, phenoxy, and H; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of a bond, H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

Exemplary disclosed compounds may be represented by the Formula II:

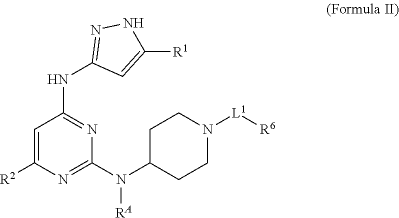

or a pharmaceutically acceptable salt, stereoisomer, and/or N-oxide thereof.

In some embodiments, contemplated compounds are represented by Formula IIa:

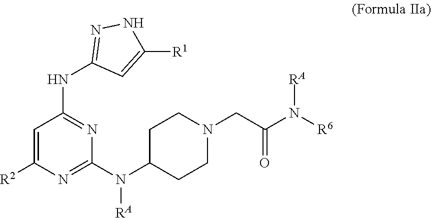

or a pharmaceutically acceptable salt, stereoisomer, and/or N-oxide thereof.

In some embodiments, contemplated compounds are represented by Formula IIb:

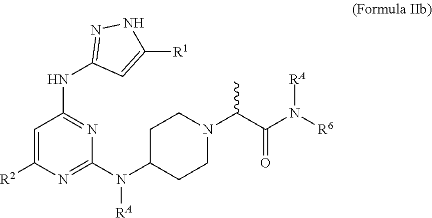

or a pharmaceutically acceptable salt, stereoisomer, and/or N-oxide thereof.

In some embodiments, contemplated compounds are represented by Formula IIc:

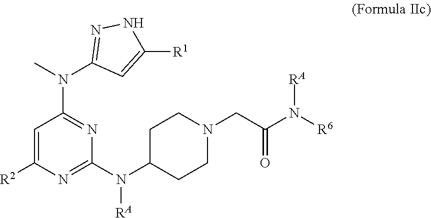

or a pharmaceutically acceptable salt, stereoisomer, and/or N-oxide thereof.

In other embodiments, the compounds disclosed herein are represented by Formula IId:

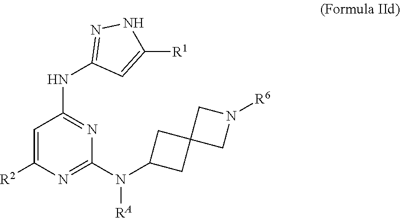

or a pharmaceutically acceptable salt, stereoisomer, and/or N-oxide thereof.

In some embodiments, the compounds disclosed herein are represented by Formula IIe:

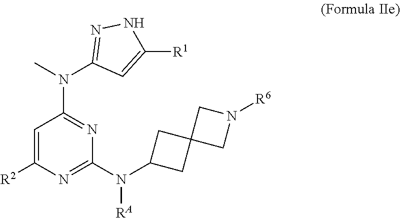

or a pharmaceutically acceptable salt, stereoisomer, and/or N-oxide thereof.

In other embodiments, the compounds disclosed herein are represented by Formula IIf:

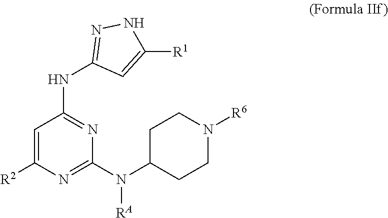

or a pharmaceutically acceptable salt, stereoisomer, and/or N-oxide thereof.

In some embodiments, R^{A} is selected from H and methyl.

In some embodiments, R⁶ is selected from the group consisting of a 8-10 membered bicyclic cycloalkyl and a 8-10 membered bicyclic heterocyclyl, wherein R⁶ is optionally substituted by one or two substituents each selected from the group consisting of cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O-heterocyclyl, heterocyclyl and heteroaryl.

In some embodiments, R⁶ is selected from the group consisting of a monocyclic or bridged C₃₋₆cycloalkyl, a monocyclic or bridged heterocyclyl, a bicyclic or fused heterocyclyl, and a heteroaryl, wherein R⁶ is optionally substituted by one or two substituents each selected from the group consisting of cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O— heterocyclyl, heterocyclyl and heteroaryl.

In some embodiments, R⁶ is selected from the group consisting of: indanyl, cyclohexyl, cyclobutyl, and cyclopentyl, wherein R⁶ is optionally substituted by one or two substituents each selected from the group consisting of cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O-heterocyclyl, heterocyclyl and heteroaryl. For example, R⁶ is indanyl.

In some embodiments, R⁶ is selected from the group consisting of heterocyclyl, phenyl, and heteroaryl.

In other embodiments, R⁶ is represented by:

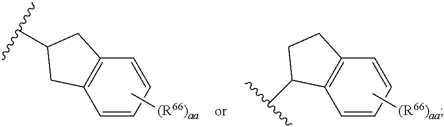

wherein R⁶⁶ is selected from the group consisting of H, halo, and cyano; and aa is 0, 1, or 2. In some embodiments, R⁶ is selected from the group consisting of:

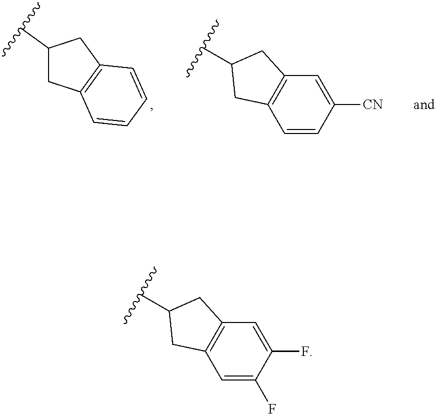 [SMILES: *C1Cc2ccccc2C1 ; *C1Cc2ccc(C#N)cc2C1 ; *C1Cc2cc(F)c(F)cc2C1]

For example, R⁶ is selected from the group consisting of:

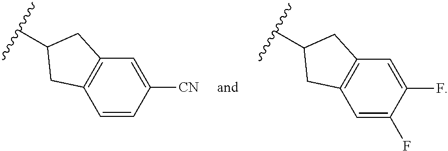 [SMILES: *C1Cc2ccc(C#N)cc2C1 ; *C1Cc2cc(F)c(F)cc2C1]

In some embodiments, R⁶ is selected from the group consisting of:

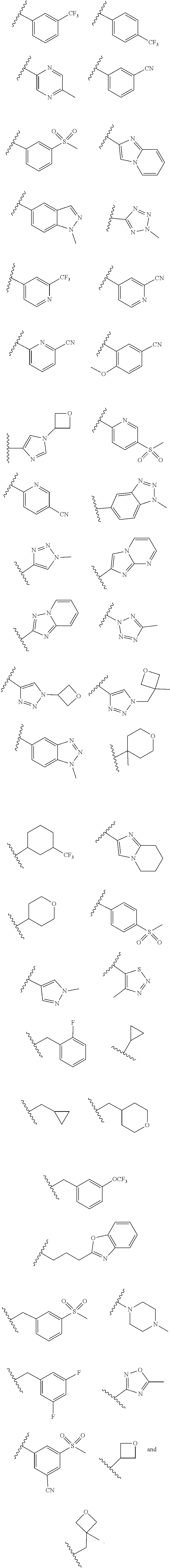

In some embodiments, R⁶ is selected from the group consisting of:

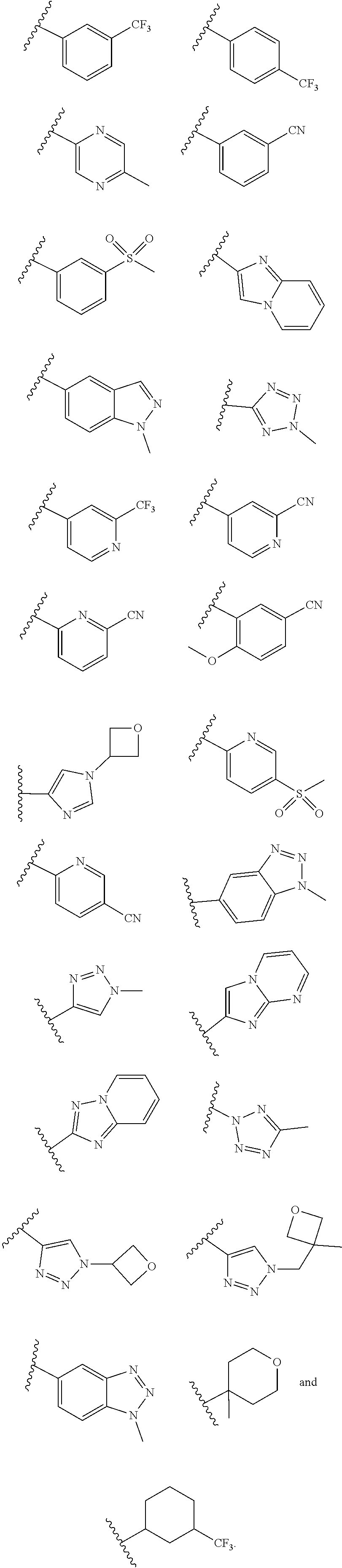

For example, R⁶ is selected from the group consisting of:

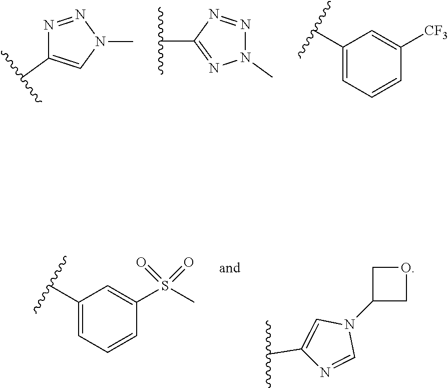

In other embodiments, R⁶ is methyl.

In other embodiments, R⁶ is methyl.

In some embodiments, R² is H.

Also disclosed herein is a compound represented by Formula III:

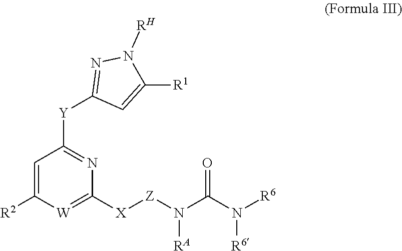

or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂, C(CH₂)₂, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ and R^{6′}, together with the nitrogen attached to R⁶ and R^{6′}, form a 4-8 membered monocyclic heterocyclyl or a 8-10 membered bicyclic heterocyclyl; wherein the monocyclic heterocyclyl or bicyclic heterocyclyl may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

Exemplary disclosed compounds are represented by Formula III-A:

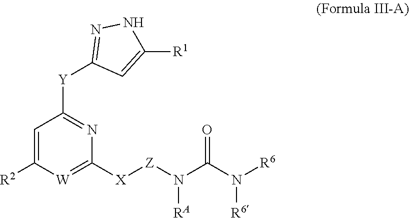

or a pharmaceutically acceptable salt, stereoisomer, and/or N-oxide thereof,

wherein:
W is selected from the group consisting of N, C—H, and C—F;
X is selected from the group consisting of NR^{A}, O, S, CH₂, C(CH₃)₂, CF₂ and C(CH₂)₂;
Y is selected from the group consisting of NH, N—CH₃, O, S, CH₂, CF₂, C(CH₃)₂, and C(CH₂CH₂);
Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, and —C(O)—O—C₁₋₄alkyl;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ and R^{6′}, together with the nitrogen attached to R⁶ and R^{6′}, form a 4-8 membered monocyclic heterocyclyl or a 8-10 membered bicyclic heterocyclyl; wherein the monocyclic heterocyclyl or bicyclic heterocyclyl may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

In some embodiments, for example, W is N, and a compound of the disclosure has the Formula IIa:

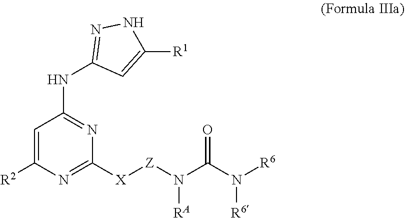

or a pharmaceutically acceptable salt, stereoisomer, and/or N-oxide thereof.

In some embodiments, R¹ is a 5-6 membered heterocyclyl or C₃₋₆cycloalkyl. For example, R¹ is selected from the group consisting of: 2-tetrahydrofuranyl, 3-tetrahydrofuranyl, 2-oxetanyl, cyclohexyl, cyclopropyl, cyclobutyl and cyclopentyl. In some embodiments, R¹ is cyclopropyl.

In other embodiments, R¹ is selected from the group consisting of methyl and ethyl.

In some embodiments, X is NR^{A}.

In some embodiments, Z is selected from the group consisting of cyclohexyl, cyclopentyl and cyclobutyl.

In some embodiments, Z is a C₅-C₉ bridged cycloalkyl.

In some embodiments, Z is a spiro C₅-C₁₀ bicycloalkyl.

In some embodiments, Z is a fused bicycloalkyl.

In some embodiments, Z is selected from the group consisting of:

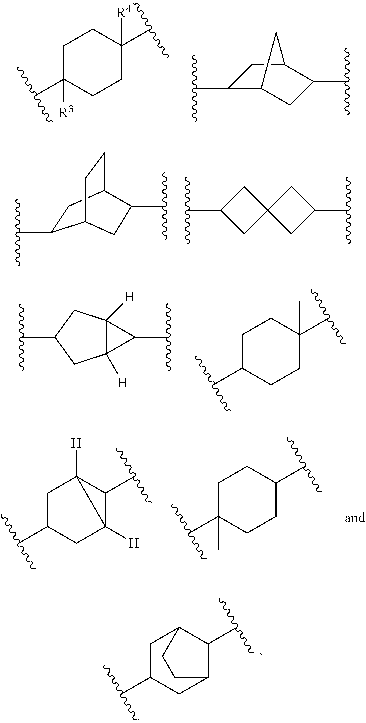

or a pharmaceutically acceptable salt, stereoisomer, and/or N-oxide thereof, wherein:
R³ is selected from the group consisting of H, C₁-C₄-alkyl, CO₂H and —C(O)—O—C₁₋₄alkyl;
R⁴ is H or C₁-C₄-alkyl; or R³ and R⁴ together form —CH₂— or —CH₂CH₂—.

Exemplary disclosed compounds may be represented by Formula IV:

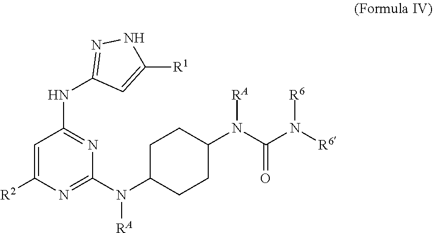

or a pharmaceutically acceptable salt, stereoisomer, and/or N-oxide thereof.

In some embodiments, the compounds disclosed herein are represented by Formula IVa:

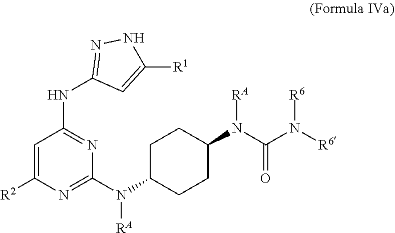

or a pharmaceutically acceptable salt, stereoisomer, and/or N-oxide thereof.

In some embodiments, R⁶ and R^{6′}, together with the nitrogen attached to R⁶ and R^{6′}, form an optionally substituted heterocycyl selected from the group consisting of:

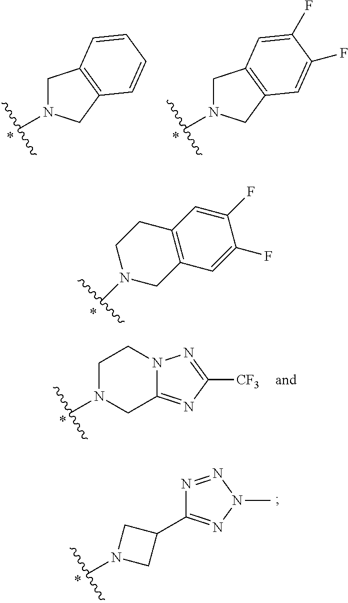 [SMILES: *N1Cc2ccccc2C1 ; *N1Cc2cc(F)c(F)cc2C1 ; *N1CCc2cc(F)c(F)cc2C1 ; *N1CCn2nc(C(F)(F)F)nc2C1 ; *N1CC(c2nnn(C)n2)C1]

wherein * denotes bonding to —C(O)—.

In some embodiments, R² is H.

A contemplated compound, for example, may be selected from the group consisting of:

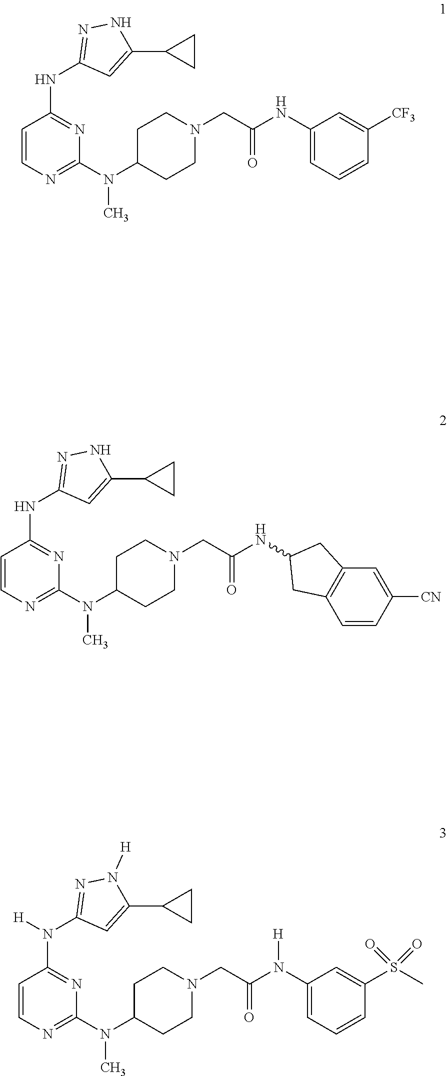 [SMILES: CN(c1nccc(Nc2cc(C3CC3)[nH]n2)n1)C1CCN(CC(=O)Nc2cccc(C(F)(F)F)c2)CC1 ; CN(c1nccc(Nc2cc(C3CC3)[nH]n2)n1)C1CCN(CC(=O)NC2Cc3ccc(C#N)cc3C2)CC1 ; CN(c1nccc(Nc2cc(C3CC3)[nH]n2)n1)C1CCN(CC(=O)Nc2cccc(S(C)(=O)=O)c2)CC1]

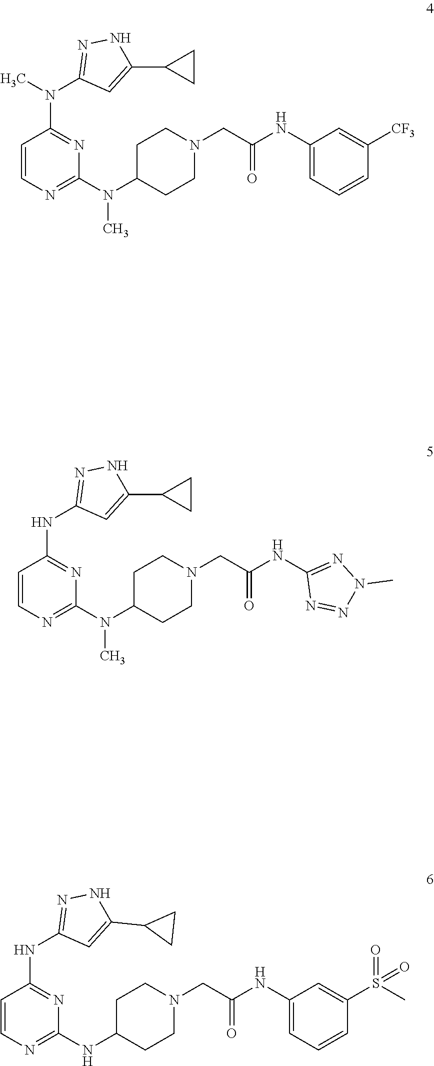 [SMILES: CN(c1cc(C2CC2)[nH]n1)c1ccnc(N(C)C2CCN(CC(=O)Nc3cccc(C(F)(F)F)c3)CC2)n1 ; CN(c1nccc(Nc2cc(C3CC3)[nH]n2)n1)C1CCN(CC(=O)Nc2nnn(C)n2)CC1 ; CS(=O)(=O)c1cccc(NC(=O)CN2CCC(Nc3nccc(Nc4cc(C5CC5)[nH]n4)n3)CC2)c1]

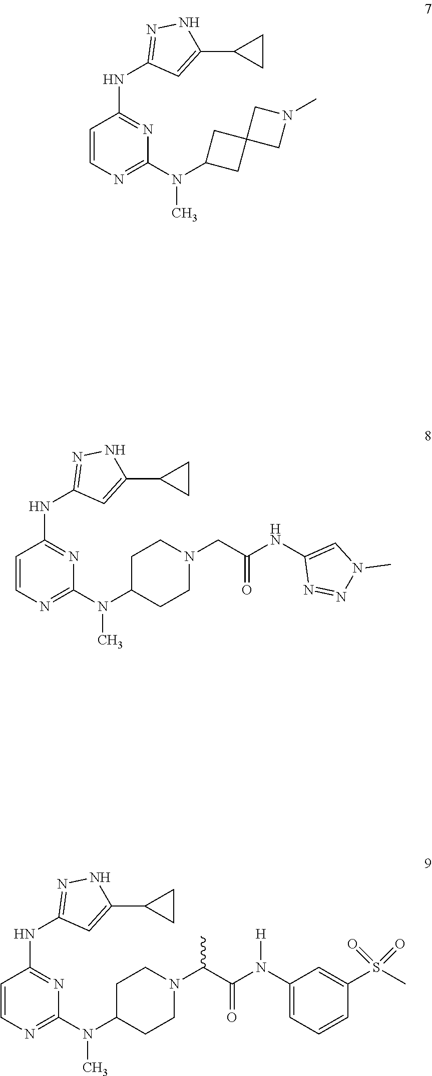 [SMILES: CN1CC2(CC(N(C)c3nccc(Nc4cc(C5CC5)[nH]n4)n3)C2)C1 ; CN(c1nccc(Nc2cc(C3CC3)[nH]n2)n1)C1CCN(CC(=O)Nc2cn(C)nn2)CC1 ; CC(C(=O)Nc1cccc(S(C)(=O)=O)c1)N1CCC(N(C)c2nccc(Nc3cc(C4CC4)[nH]n3)n2)CC1]

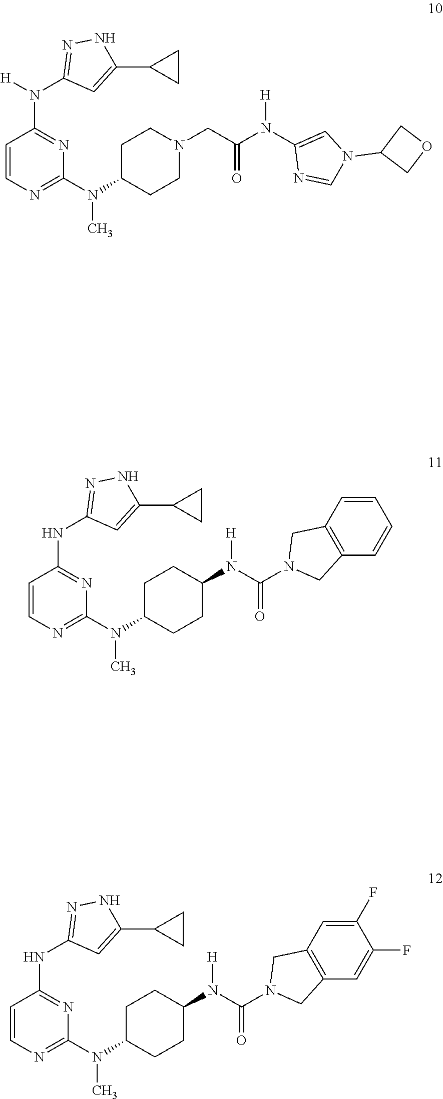

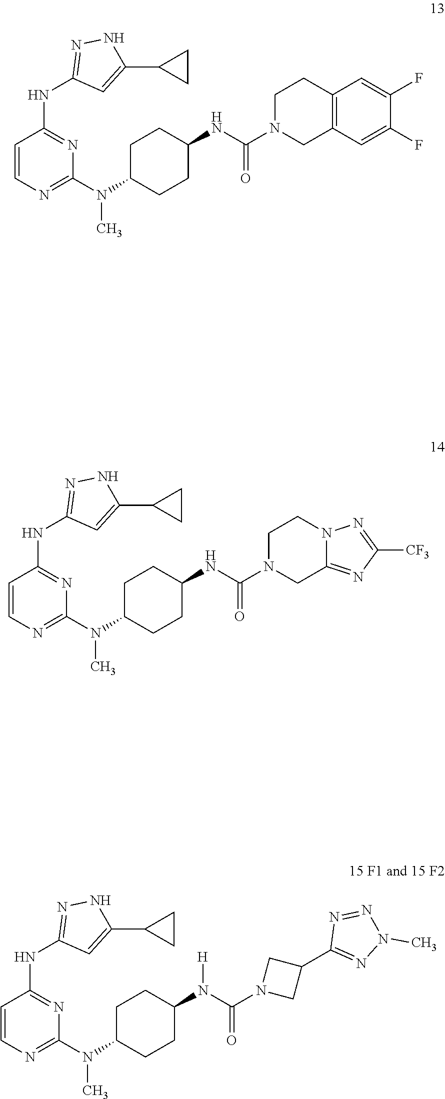 [SMILES: CN(c1nccc(Nc2cc(C3CC3)[nH]n2)n1)[C@H]1CC[C@H](NC(=O)N2CCc3cc(F)c(F)cc3C2)CC1 ; CN(c1nccc(Nc2cc(C3CC3)[nH]n2)n1)[C@H]1CC[C@H](NC(=O)N2CCn3nc(C(F)(F)F)nc3C2)CC1 ; Cn1nnc(C2CN(C(=O)N[C@H]3CC[C@H](N(C)c4nccc(Nc5cc(C6CC6)[nH]n5)n4)CC3)C2)n1]

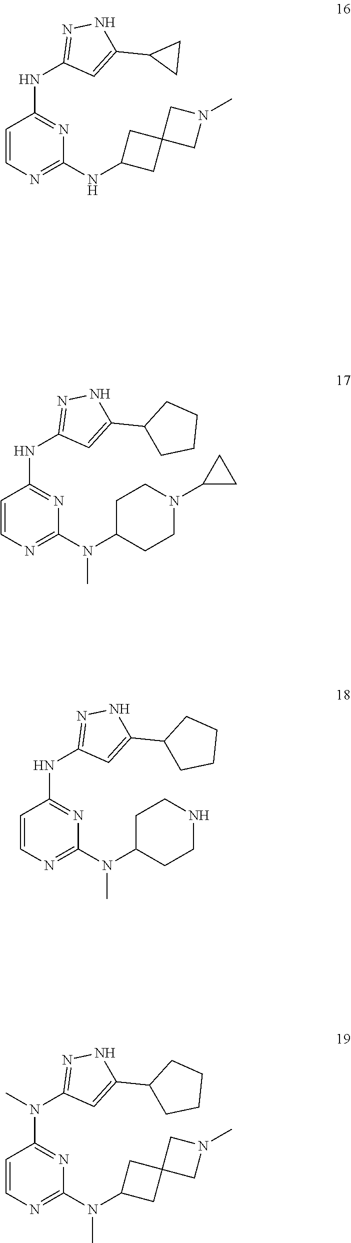 [SMILES: CN1CC2(CC(Nc3nccc(Nc4cc(C5CC5)[nH]n4)n3)C2)C1 ; CN(c1nccc(Nc2cc(C3CCCC3)[nH]n2)n1)C1CCN(C2CC2)CC1 ; CN(c1nccc(Nc2cc(C3CCCC3)[nH]n2)n1)C1CCNCC1 ; CN1CC2(CC(N(C)c3nccc(N(C)c4cc(C5CCCC5)[nH]n4)n3)C2)C1]

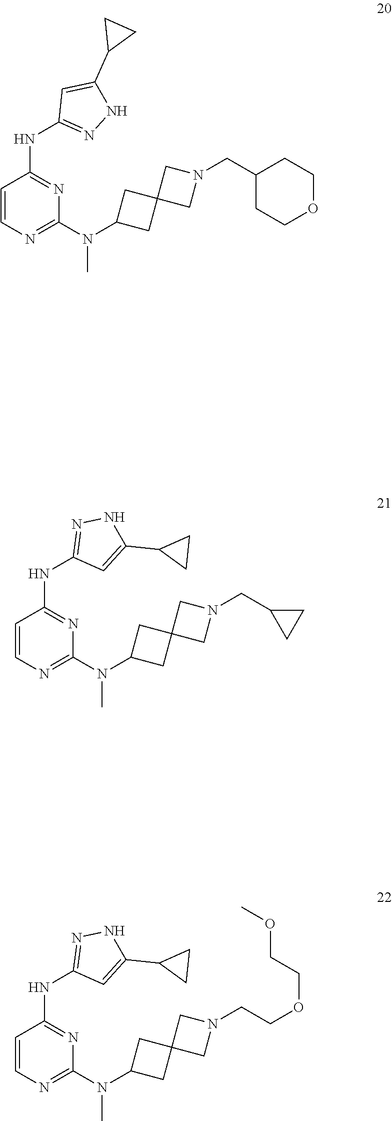 [SMILES: CN(c1nccc(Nc2cc(C3CC3)[nH]n2)n1)C1CC2(C1)CN(CC1CCOCC1)C2 ; CN(c1nccc(Nc2cc(C3CC3)[nH]n2)n1)C1CC2(C1)CN(CC1CC1)C2 ; COCCOCCN1CC2(CC(N(C)c3nccc(Nc4cc(C5CC5)[nH]n4)n3)C2)C1]

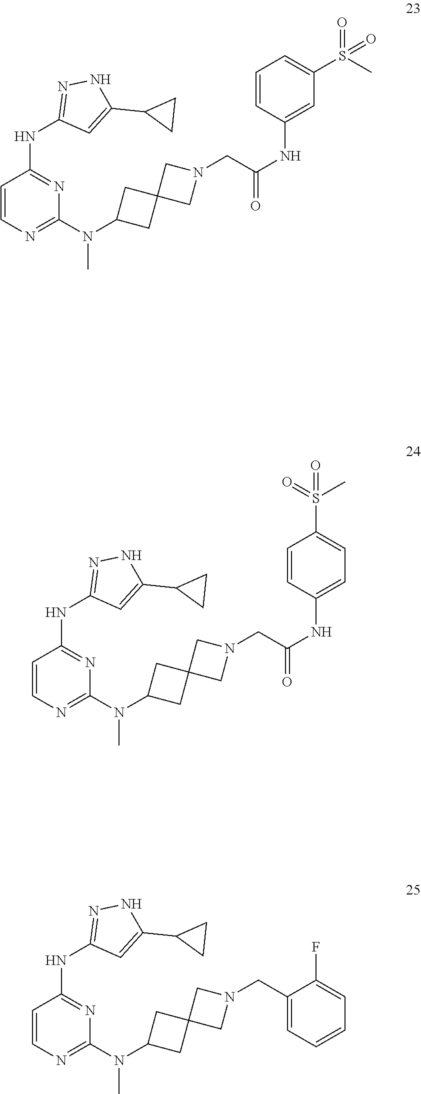 [SMILES: CN(c1nccc(Nc2cc(C3CC3)[nH]n2)n1)C1CC2(C1)CN(CC(=O)Nc1cccc(S(C)(=O)=O)c1)C2 ; CN(c1nccc(Nc2cc(C3CC3)[nH]n2)n1)C1CC2(C1)CN(CC(=O)Nc1ccc(S(C)(=O)=O)cc1)C2 ; CN(c1nccc(Nc2cc(C3CC3)[nH]n2)n1)C1CC2(C1)CN(Cc1ccccc1F)C2]

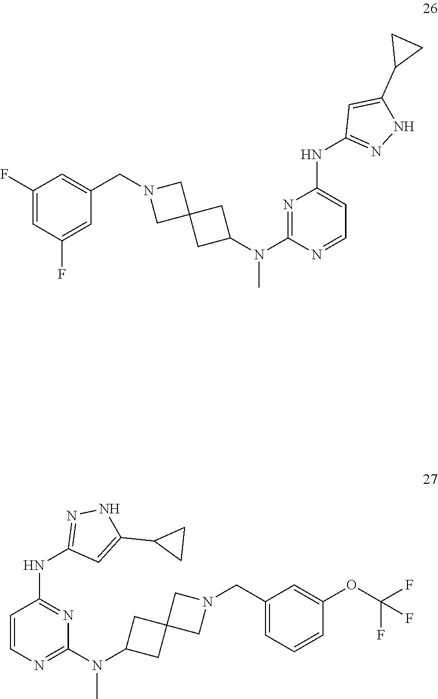 [SMILES: CN(c1nccc(Nc2cc(C3CC3)[nH]n2)n1)C1CC2(C1)CN(Cc1cc(F)cc(F)c1)C2 ; CN(c1nccc(Nc2cc(C3CC3)[nH]n2)n1)C1CC2(C1)CN(Cc1cccc(OC(F)(F)F)c1)C2]

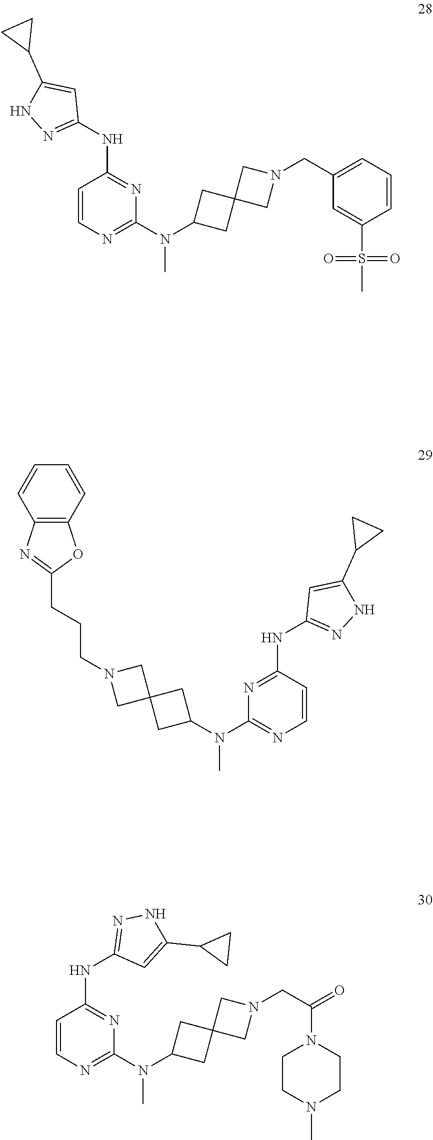 [SMILES: CN(c1nccc(Nc2cc(C3CC3)[nH]n2)n1)C1CC2(C1)CN(Cc1cccc(S(C)(=O)=O)c1)C2 ; CN(c1nccc(Nc2cc(C3CC3)[nH]n2)n1)C1CC2(C1)CN(CCCc1nc3ccccc3o1)C2 ; CN1CCN(C(=O)CN2CC3(CC(N(C)c4nccc(Nc5cc(C6CC6)[nH]n5)n4)C3)C2)CC1]

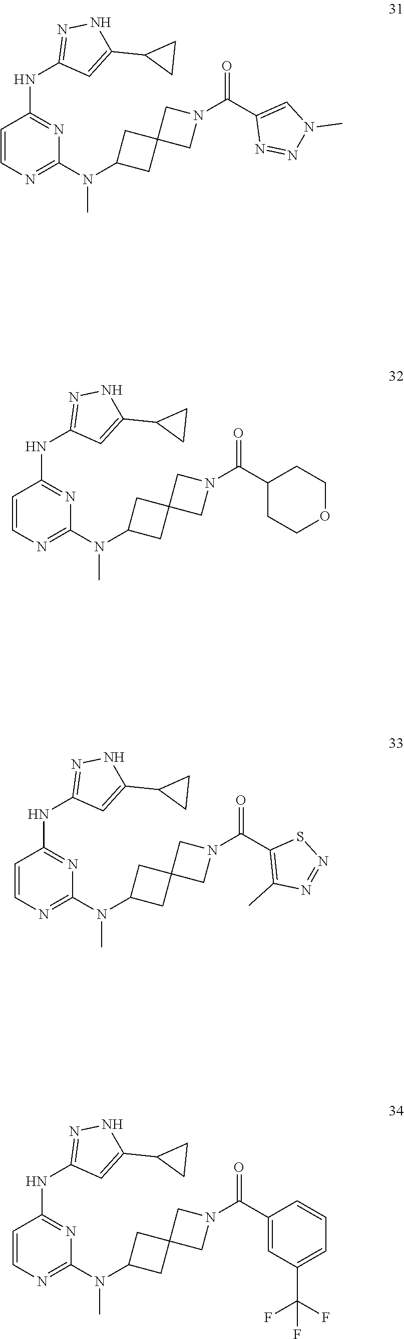 [SMILES: CN(c1nccc(Nc2cc(C3CC3)[nH]n2)n1)C1CC2(C1)CN(C(=O)c1cn(C)nn1)C2 ; CN(c1nccc(Nc2cc(C3CC3)[nH]n2)n1)C1CC2(C1)CN(C(=O)C1CCOCC1)C2 ; Cc1nnsc1C(=O)N1CC2(CC(N(C)c3nccc(Nc4cc(C5CC5)[nH]n4)n3)C2)C1 ; CN(c1nccc(Nc2cc(C3CC3)[nH]n2)n1)C1CC2(C1)CN(C(=O)c1cccc(C(F)(F)F)c1)C2]

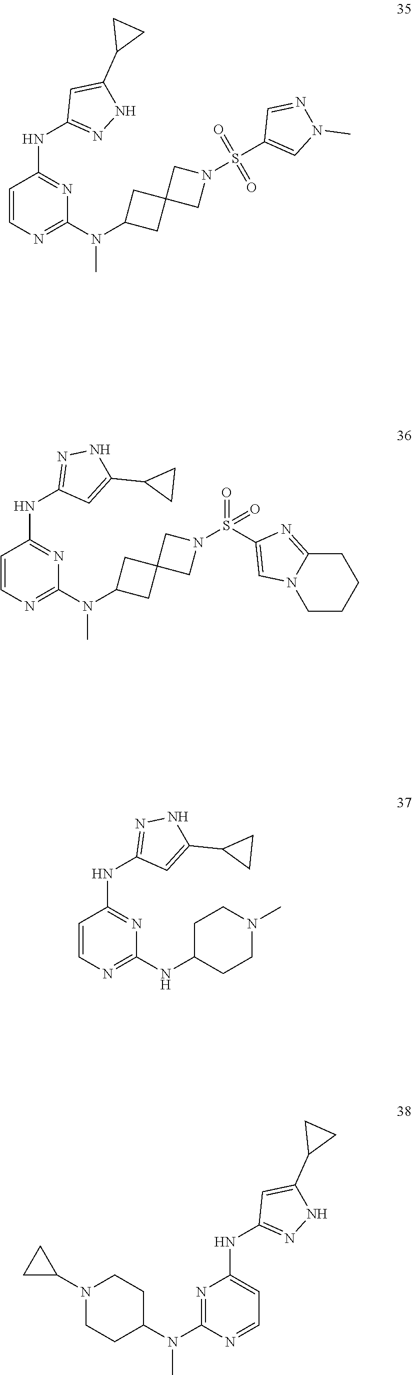 [SMILES: CN(c1nccc(Nc2cc(C3CC3)[nH]n2)n1)C1CC2(C1)CN(S(=O)(=O)c1cnn(C)c1)C2 ; CN(c1nccc(Nc2cc(C3CC3)[nH]n2)n1)C1CC2(C1)CN(S(=O)(=O)c1cn3c(n1)CCCC3)C2 ; CN1CCC(Nc2nccc(Nc3cc(C4CC4)[nH]n3)n2)CC1 ; CN(c1nccc(Nc2cc(C3CC3)[nH]n2)n1)C1CCN(C2CC2)CC1]

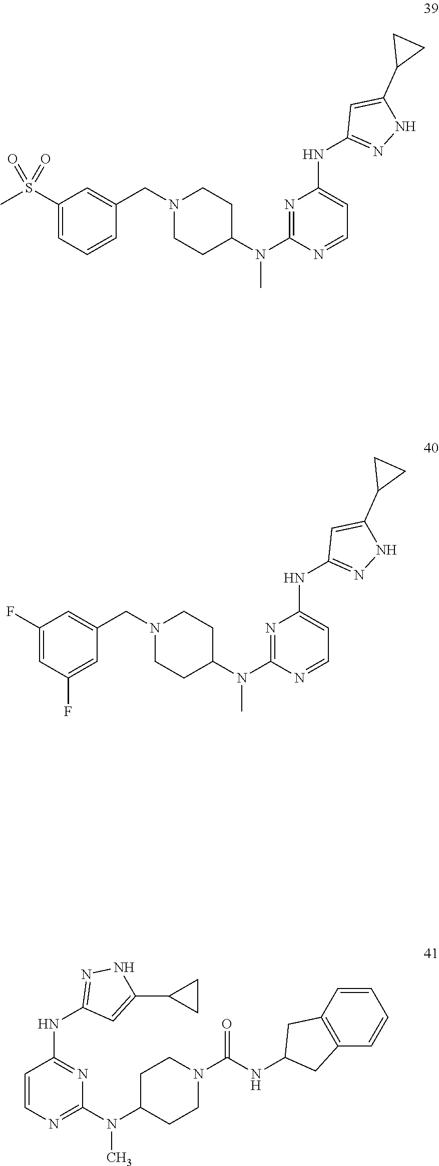 [SMILES: CN(c1nccc(Nc2cc(C3CC3)[nH]n2)n1)C1CCN(Cc2cccc(S(C)(=O)=O)c2)CC1 ; CN(c1nccc(Nc2cc(C3CC3)[nH]n2)n1)C1CCN(Cc2cc(F)cc(F)c2)CC1 ; CN(c1nccc(Nc2cc(C3CC3)[nH]n2)n1)C1CCN(C(=O)NC2Cc3ccccc3C2)CC1]

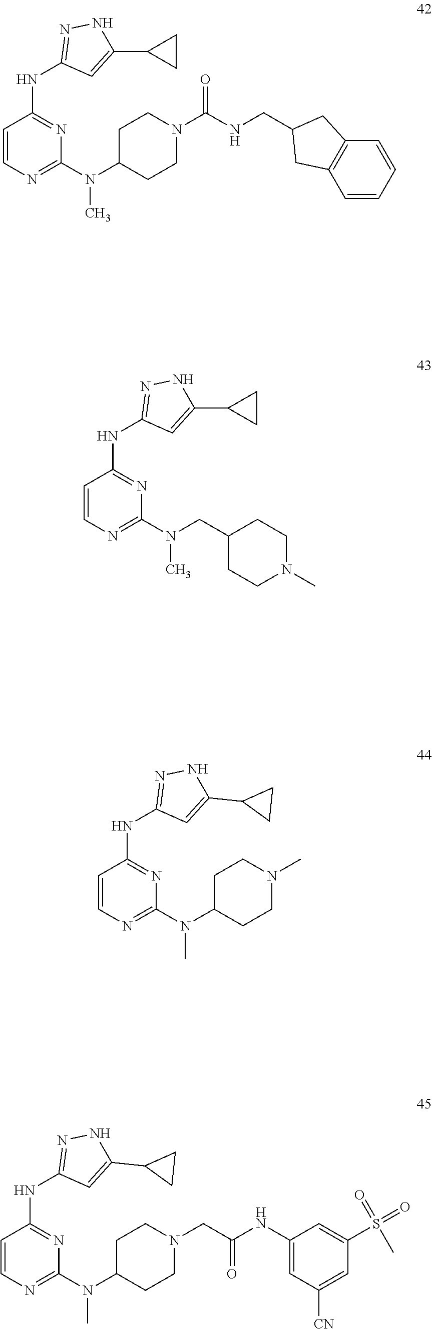 [SMILES: CN(c1nccc(Nc2cc(C3CC3)[nH]n2)n1)C1CCN(C(=O)NCC2Cc3ccccc3C2)CC1 ; CN1CCC(CN(C)c2nccc(Nc3cc(C4CC4)[nH]n3)n2)CC1 ; CN1CCC(N(C)c2nccc(Nc3cc(C4CC4)[nH]n3)n2)CC1 ; CN(c1nccc(Nc2cc(C3CC3)[nH]n2)n1)C1CCN(CC(=O)Nc2cc(C#N)cc(S(C)(=O)=O)c2)CC1]

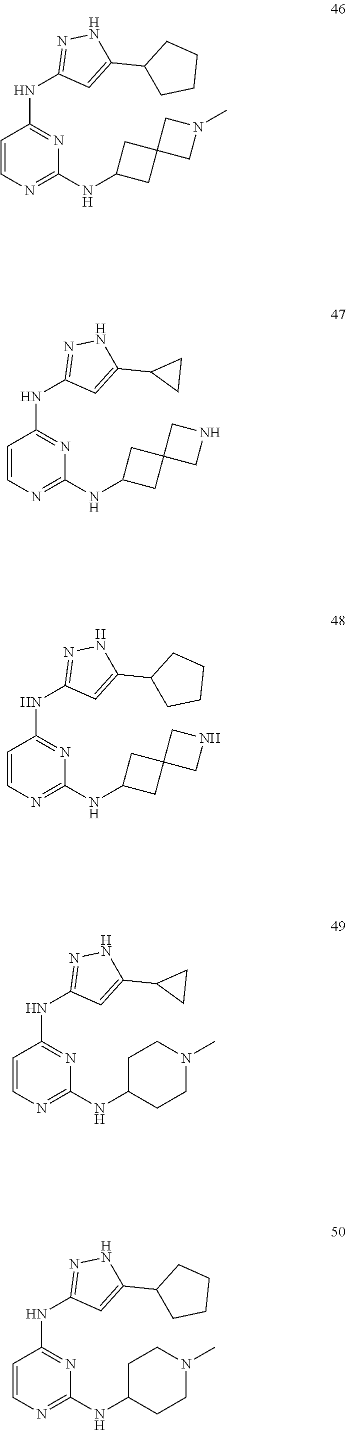 [SMILES: CN1CC2(CC(Nc3nccc(Nc4cc(C5CCCC5)[nH]n4)n3)C2)C1 ; c1cc(Nc2cc(C3CC3)[nH]n2)nc(NC2CC3(CNC3)C2)n1 ; c1cc(Nc2cc(C3CCCC3)[nH]n2)nc(NC2CC3(CNC3)C2)n1 ; CN1CCC(Nc2nccc(Nc3cc(C4CC4)[nH]n3)n2)CC1 ; CN1CCC(Nc2nccc(Nc3cc(C4CCCC4)[nH]n3)n2)CC1]

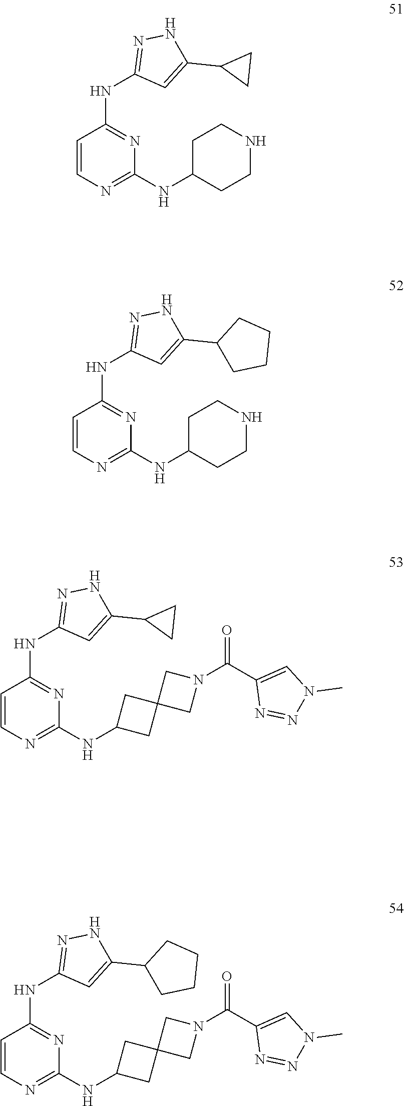 [SMILES: c1cc(Nc2cc(C3CC3)[nH]n2)nc(NC2CCNCC2)n1 ; c1cc(Nc2cc(C3CCCC3)[nH]n2)nc(NC2CCNCC2)n1 ; Cn1cc(C(=O)N2CC3(CC(Nc4nccc(Nc5cc(C6CC6)[nH]n5)n4)C3)C2)nn1 ; Cn1cc(C(=O)N2CC3(CC(Nc4nccc(Nc5cc(C6CCCC6)[nH]n5)n4)C3)C2)nn1]

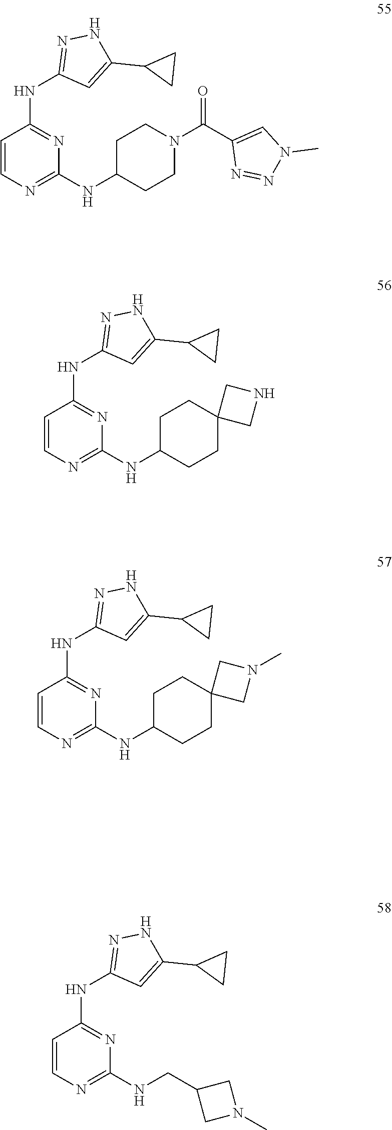 [SMILES: Cn1cc(C(=O)N2CCC(Nc3nccc(Nc4cc(C5CC5)[nH]n4)n3)CC2)nn1 ; c1cc(Nc2cc(C3CC3)[nH]n2)nc(NC2CCC3(CC2)CNC3)n1 ; CN1CC2(CCC(Nc3nccc(Nc4cc(C5CC5)[nH]n4)n3)CC2)C1 ; CN1CC(CNc2nccc(Nc3cc(C4CC4)[nH]n3)n2)C1]

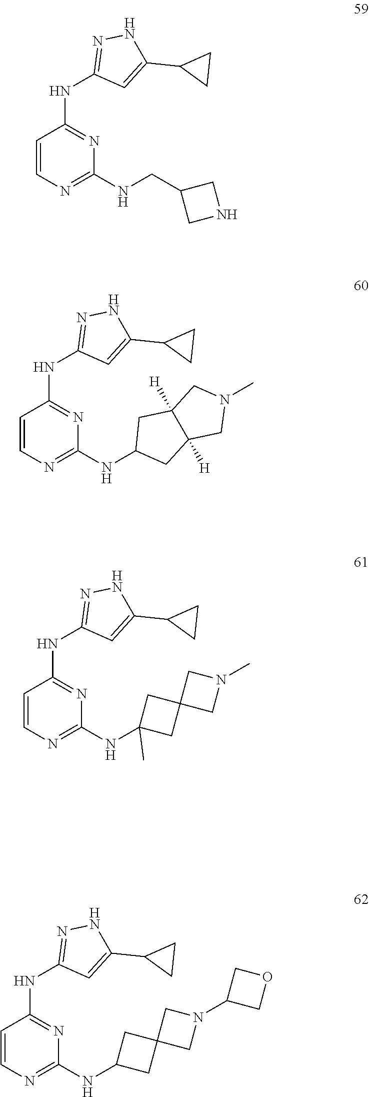 [SMILES: c1cc(Nc2cc(C3CC3)[nH]n2)nc(NCC2CNC2)n1 ; CN1C[C@H]2CC(Nc3nccc(Nc4cc(C5CC5)[nH]n4)n3)C[C@H]2C1 ; CN1CC2(C1)CC(C)(Nc1nccc(Nc3cc(C4CC4)[nH]n3)n1)C2 ; c1cc(Nc2cc(C3CC3)[nH]n2)nc(NC2CC3(C2)CN(C2COC2)C3)n1]

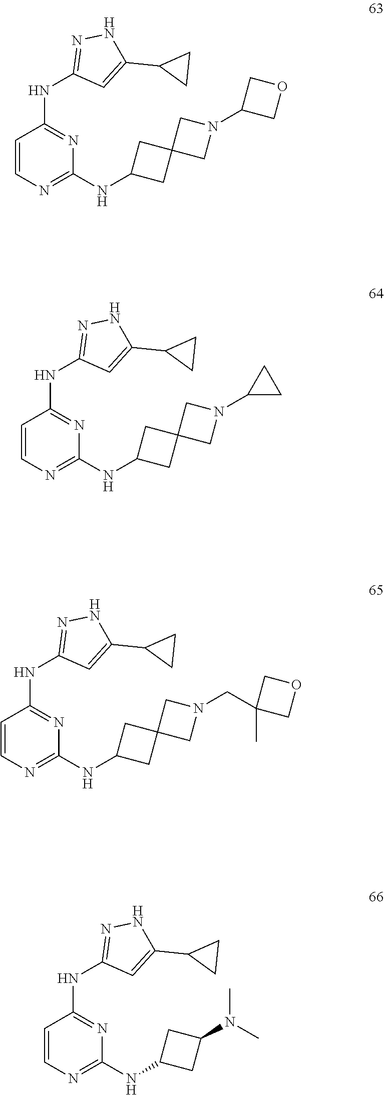

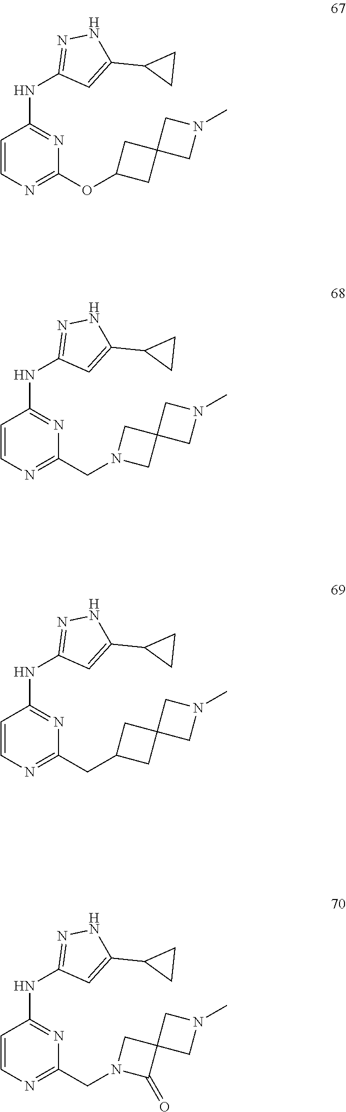 [SMILES: CN1CC2(CC(Oc3nccc(Nc4cc(C5CC5)[nH]n4)n3)C2)C1 ; CN1CC2(C1)CN(Cc1nccc(Nc3cc(C4CC4)[nH]n3)n1)C2 ; CN1CC2(CC(Cc3nccc(Nc4cc(C5CC5)[nH]n4)n3)C2)C1 ; CN1CC2(C1)CN(Cc1nccc(Nc3cc(C4CC4)[nH]n3)n1)C2=O]

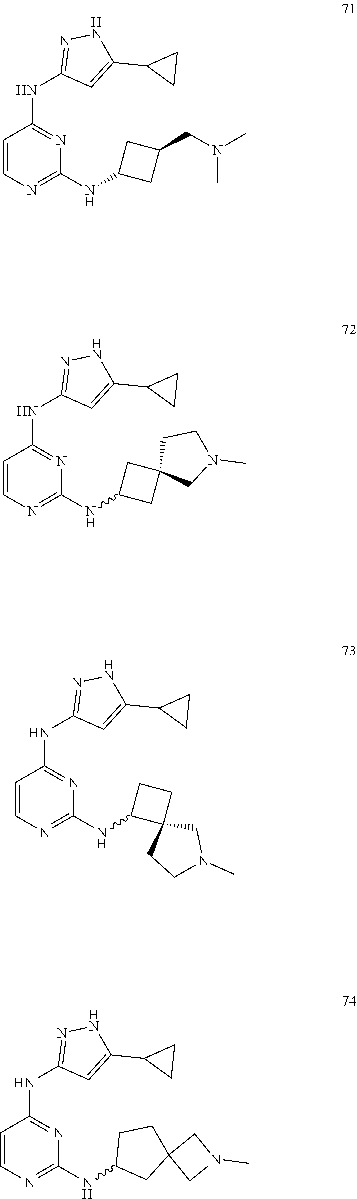

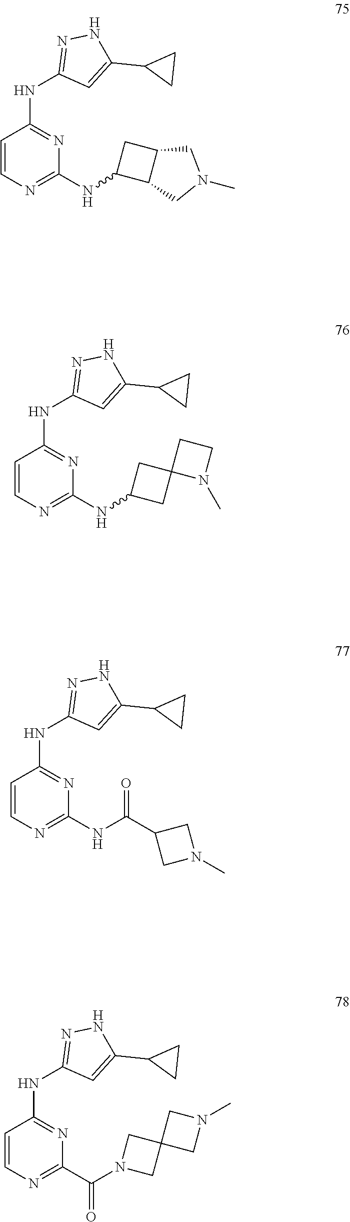 [SMILES: CN1C[C@H]2CC(Nc3nccc(Nc4cc(C5CC5)[nH]n4)n3)[C@H]2C1 ; CN1CCC12CC(Nc1nccc(Nc3cc(C4CC4)[nH]n3)n1)C2 ; CN1CC(C(=O)Nc2nccc(Nc3cc(C4CC4)[nH]n3)n2)C1 ; CN1CC2(C1)CN(C(=O)c1nccc(Nc3cc(C4CC4)[nH]n3)n1)C2]

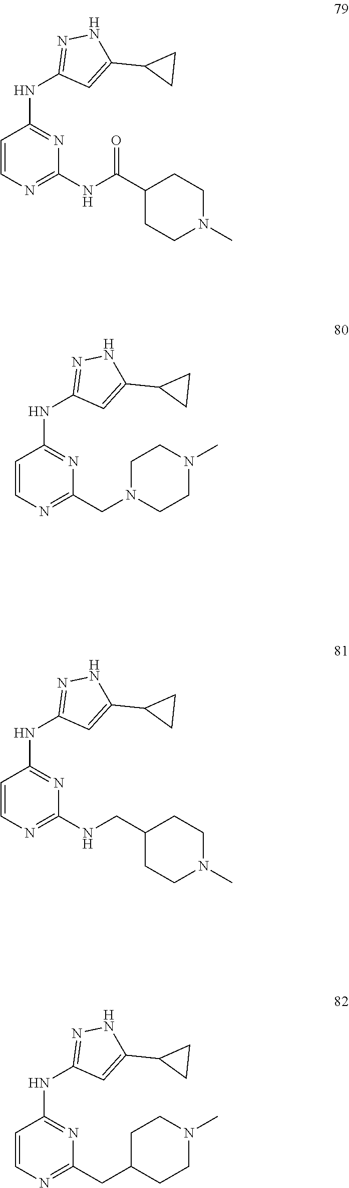 [SMILES: CN1CCC(C(=O)Nc2nccc(Nc3cc(C4CC4)[nH]n3)n2)CC1 ; CN1CCN(Cc2nccc(Nc3cc(C4CC4)[nH]n3)n2)CC1 ; CN1CCC(CNc2nccc(Nc3cc(C4CC4)[nH]n3)n2)CC1 ; CN1CCC(Cc2nccc(Nc3cc(C4CC4)[nH]n3)n2)CC1]

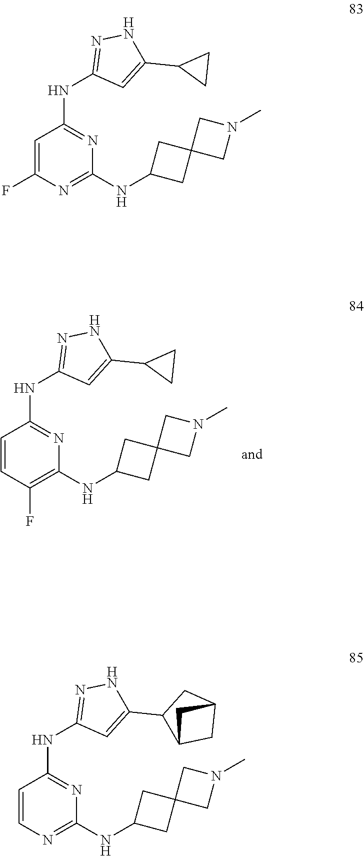 [SMILES: CN1CC2(CC(Nc3nc(F)cc(Nc4cc(C5CC5)[nH]n4)n3)C2)C1 ; CN1CC2(CC(Nc3nc(Nc4cc(C5CC5)[nH]n4)ccc3F)C2)C1 ; CN1CC2(CC(Nc3nccc(Nc4cc(C5C[C@H]6C[C@@H]5C6)[nH]n4)n3)C2)C1]

and a pharmaceutically acceptable salt, stereoisomer, and/or N-oxide thereof.

Also disclosed herein are compounds represented by Formula V:

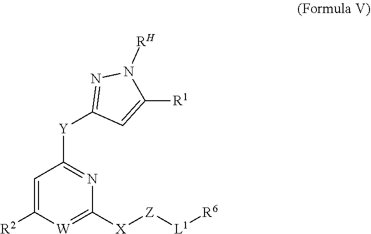

or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂, C(CH₂)₂, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —S(O)_{w}—, —NR^{A}—S(O)_{w}—, —CHR^{L}—NR^{A}—S(O)_{w}—, —NR^{A}—S(O)_{w}—CHR^{L}—, —S(O)_{w}—NR^{A}—, —CH₂—S(O)_{w}—NR^{A}—, —S(O)_{w}—NR^{A}—CHR^{L}—, —CHR^{L}—C(O)—, —C(O)—, and bond, where w is 0, 1 or 2;
Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-phenyl, C₁₋₃alkylene-heteroaryl, —C(O)-heteroaryl, phenoxy, and H; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl

Exemplary disclosed compounds may be represented by the Formula V-A:

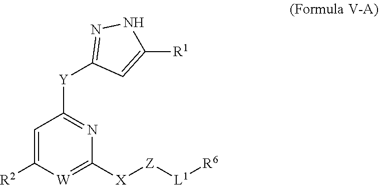

or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂ and C(CH₂)₂;
Y is selected from the group consisting of NH, N—CH₃, O, S, CH₂, CF₂, C(CH₃)₂, and C(CH₂CH₂);
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —NR^{A}—S(O)_{w}—, —CHR^{L}—NR^{A}—S(O)_{w}—, —NR^{A}—S(O)_{w}—CHR^{L}—, —S(O)_{w}—NR^{A}—, —CH₂—S(O)_{w}—NR^{A}—, and —S(O)_{w}—NR^{A}—CHR^{L}—, where w is 0, 1 or 2;
Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, and —C(O)—O—C₁₋₄alkyl;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-heteroaryl, —C(O)-heteroaryl, and phenoxy; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

In some embodiments, for example, W is N, and a compound of the disclosure has the Formula Va:

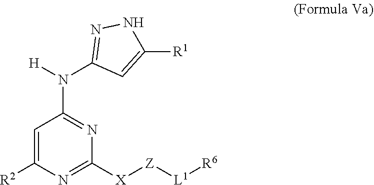

or a pharmaceutically acceptable salt, stereoisomer, and/or N-oxide thereof.

In some embodiments, R¹ is a 5-6 membered heterocyclyl or C₃₋₆cycloalkyl. For example, R¹ is selected from the group consisting of: 2-tetrahydrofuranyl, 3-tetrahydrofuranyl, 2-oxetanyl, cyclohexyl, cyclopropyl, cyclobutyl and cyclopentyl. In some embodiments, R¹ is cyclopropyl. In some embodiments, R¹ is cyclopentyl.

In other embodiments, R¹ is selected from the group consisting of methyl and ethyl.

In an exemplary embodiment, Z is selected from the group consisting of cyclohexyl, cyclopentyl and cyclobutyl.

In some embodiments, Z is a C₅-C₉ bridged cycloalkyl.

In some embodiments, Z is a spiro C₅-C₁₀ bicycloalkyl.

In some embodiments, Z is a fused bicycloalkyl.

In some embodiments, Z is selected from the group consisting of:

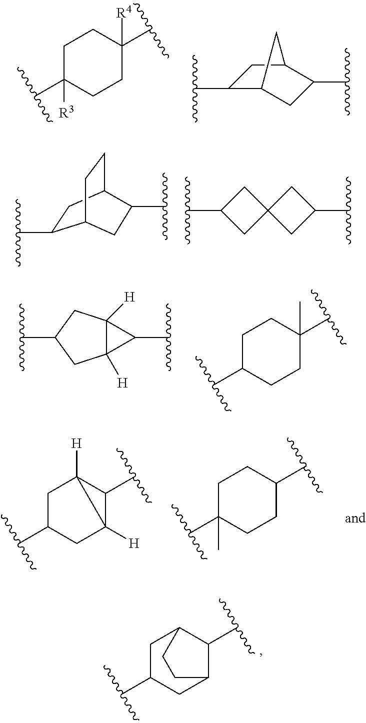

or a pharmaceutically acceptable salt, stereoisomer, and/or N-oxide thereof, wherein:
R³ is selected from the group consisting of H, C₁-C₄-alkyl, CO₂H and —C(O)—O—C₁₋₄alkyl;
R⁴ is H or C₁-C₄-alkyl; or
R³ and R⁴ together form —CH₂— or —CH₂CH₂—.

Also disclosed herein are compounds represented by Formula VI:

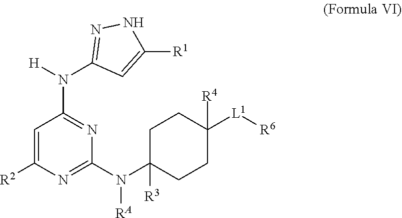

or a pharmaceutically acceptable salt, stereoisomer, and/or N-oxide thereof, wherein:
R³ is selected from the group consisting of H, C₁-C₄alkyl, CO₂H and —C(O)—O—C₁₋₄alkyl; and
R⁴ is selected from H or C₁-C₄alkyl.

Exemplary disclosed compounds may be represented by Formula VIa:

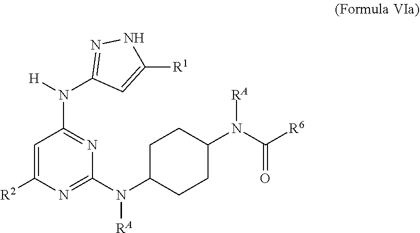

or a pharmaceutically acceptable salt, stereoisomer, and/or N-oxide thereof.

In other embodiments, the compounds may be represented by Formula VIb:

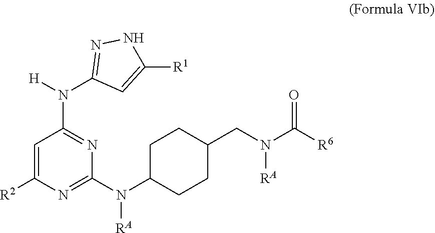

or a pharmaceutically acceptable salt, stereoisomer, and/or N-oxide thereof.

In some embodiments, the compounds may be represented by Formula VIc:

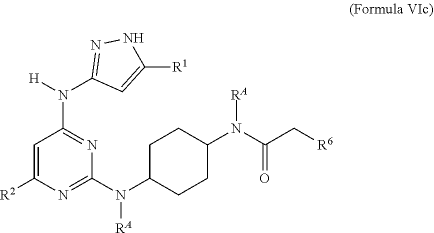

or a pharmaceutically acceptable salt, stereoisomer, and/or N-oxide thereof.

In some embodiments, the compounds may be represented by Formula VId:

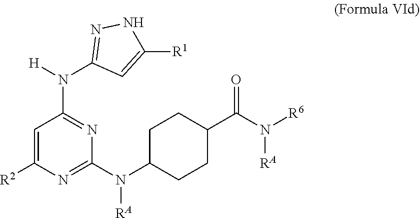

or a pharmaceutically acceptable salt, stereoisomer, and/or N-oxide thereof.

In some other embodiments, the compounds may be represented by Formula VIe:

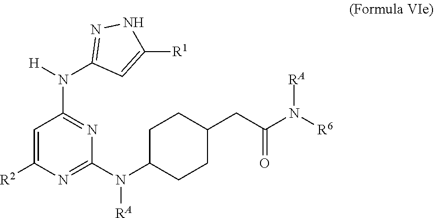

or a pharmaceutically acceptable salt, stereoisomer, and/or N-oxide thereof.

In other embodiments, the compounds may be represented by Formula VIf:

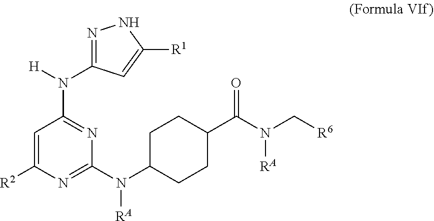

or a pharmaceutically acceptable salt, stereoisomer, and/or N-oxide thereof.

In some embodiments, the compounds may be represented by Formula VIg:

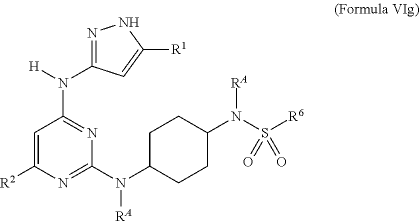

or a pharmaceutically acceptable salt, stereoisomer, and/or N-oxide thereof.

In some embodiments, the compounds may be represented by Formula VIh:

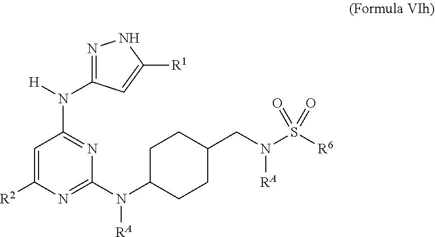

or a pharmaceutically acceptable salt, stereoisomer, and/or N-oxide thereof.

In some embodiments, the compounds may be represented by Formula VIi:

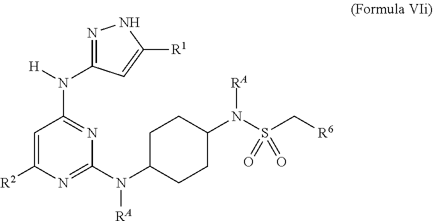

or a pharmaceutically acceptable salt, stereoisomer, and/or N-oxide thereof.

In other embodiments, the compounds may be represented by Formula VIj:

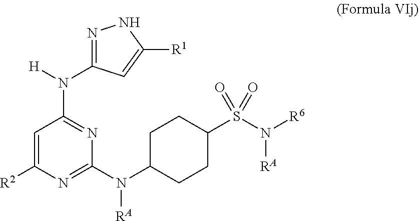

or a pharmaceutically acceptable salt, stereoisomer, and/or N-oxide thereof.

Also disclosed herein are compounds represented by Formula VIk:

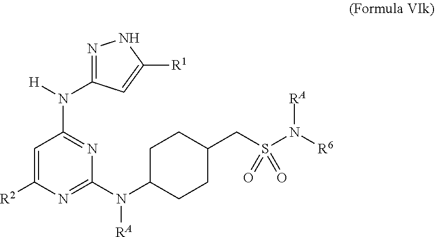

or a pharmaceutically acceptable salt, stereoisomer, and/or N-oxide thereof.

In some embodiments, the compounds may be represented by Formula VII:

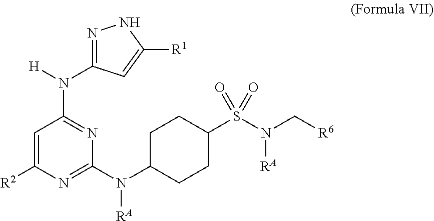

or a pharmaceutically acceptable salt, stereoisomer, and/or N-oxide thereof.

In some embodiments, R⁶ is selected from the group consisting of a 8-10 membered bicyclic cycloalkyl and a 8-10 membered bicyclic heterocyclyl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O-heterocyclyl, heterocyclyl and heteroaryl.

In some embodiments, R⁶ is selected from the group consisting of a monocyclic or bridged C₃₋₆cycloalkyl, a monocyclic or bridged heterocyclyl, a bicyclic or fused heterocyclyl, and a heteroaryl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O-heterocyclyl, heterocyclyl and heteroaryl.

In some embodiments, R⁶ is selected from the group consisting of: indanyl, cyclohexyl, cyclobutyl, and cyclopentyl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O-heterocyclyl, heterocyclyl and heteroaryl. For example, R⁶ is indanyl.

In some embodiments, R⁶ is selected from the group consisting of heterocyclyl, phenyl, and heteroaryl.

In some embodiments, R⁶ is represented by:

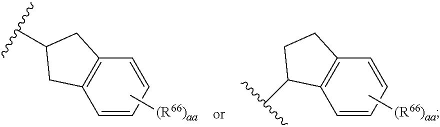

wherein R⁶⁶ is selected from the group consisting of H, halo, and cyano; and aa is 0, 1, or 2. For example, R⁶ is selected from the group consisting of:

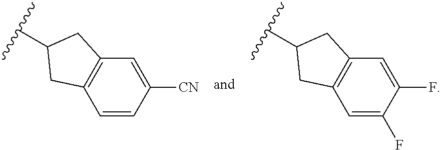 [SMILES: *C1Cc2ccc(C#N)cc2C1 ; *C1Cc2cc(F)c(F)cc2C1]

In further embodiments, R⁶ is selected from the group consisting of:

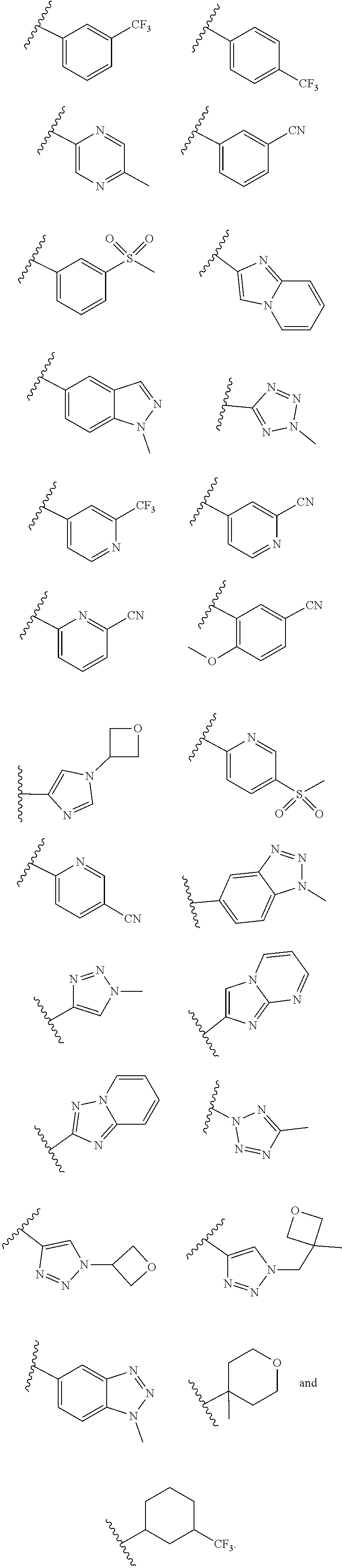

In some embodiments, R² is selected from the group consisting of H, —C(O)—O— methyl, and C(O)OH.

A contemplated compound, for example, may selected from the group consisting of:

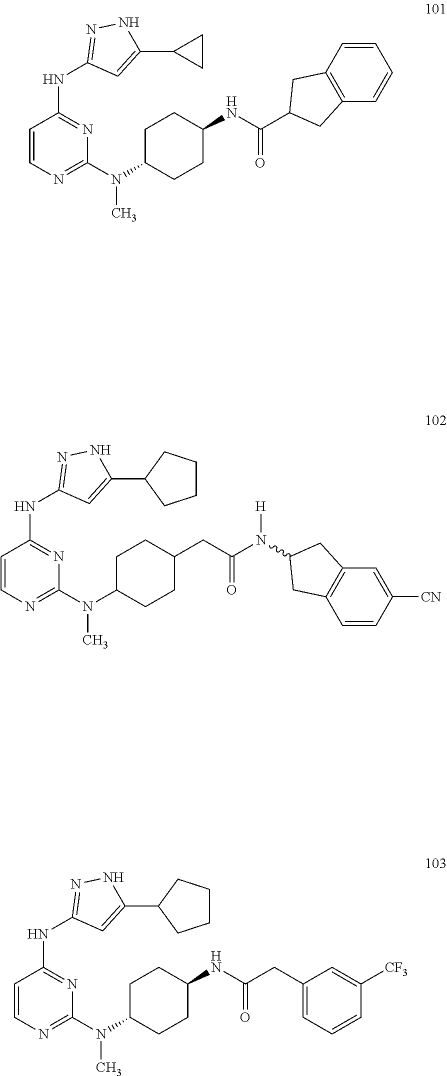 [SMILES: CN(c1nccc(Nc2cc(C3CC3)[nH]n2)n1)[C@H]1CC[C@H](NC(=O)C2Cc3ccccc3C2)CC1 ; CN(c1nccc(Nc2cc(C3CCCC3)[nH]n2)n1)C1CCC(CC(=O)NC2Cc3ccc(C#N)cc3C2)CC1 ; CN(c1nccc(Nc2cc(C3CCCC3)[nH]n2)n1)[C@H]1CC[C@H](NC(=O)Cc2cccc(C(F)(F)F)c2)CC1]

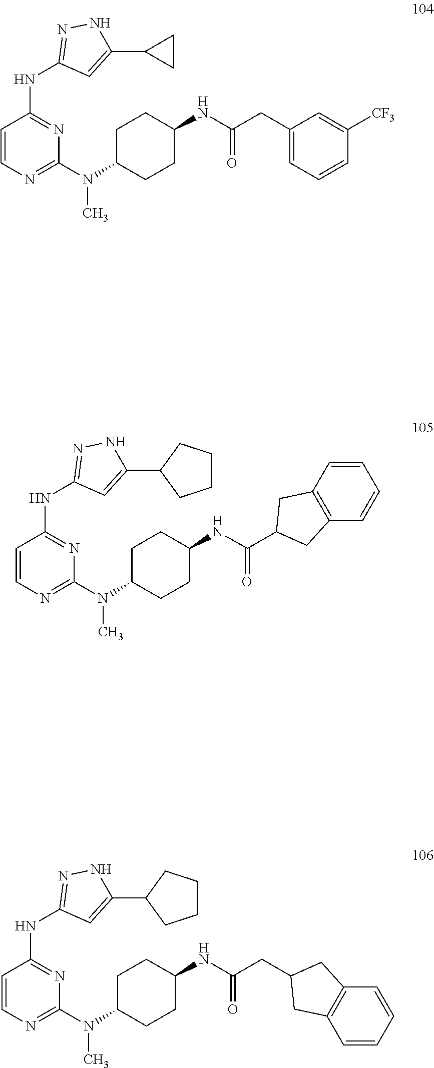 [SMILES: CN(c1nccc(Nc2cc(C3CC3)[nH]n2)n1)[C@H]1CC[C@H](NC(=O)Cc2cccc(C(F)(F)F)c2)CC1 ; CN(c1nccc(Nc2cc(C3CCCC3)[nH]n2)n1)[C@H]1CC[C@H](NC(=O)C2Cc3ccccc3C2)CC1 ; CN(c1nccc(Nc2cc(C3CCCC3)[nH]n2)n1)[C@H]1CC[C@H](NC(=O)CC2Cc3ccccc3C2)CC1]

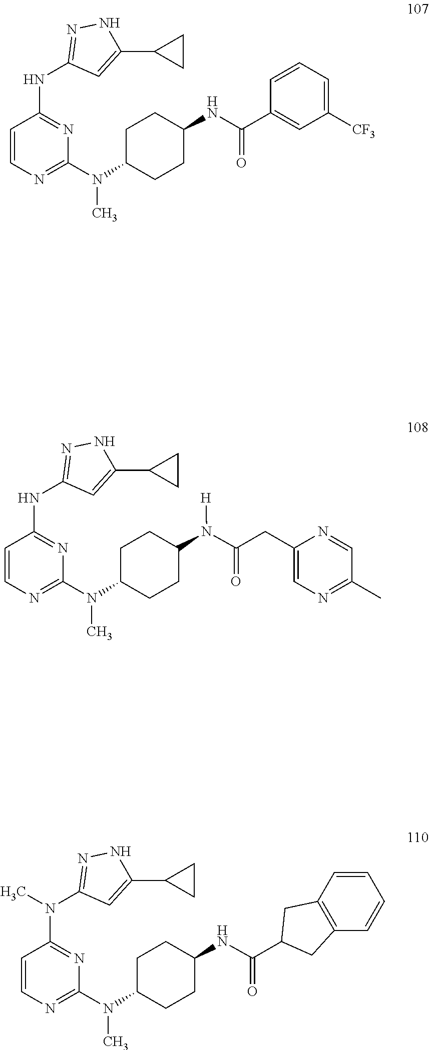

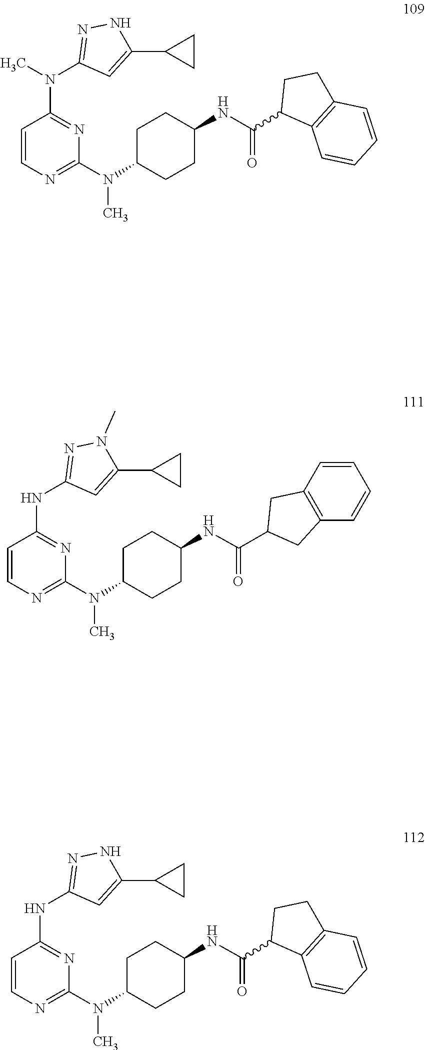 [SMILES: CN(c1cc(C2CC2)[nH]n1)c1ccnc(N(C)[C@H]2CC[C@H](NC(=O)C3CCc4ccccc43)CC2)n1 ; Cn1nc(Nc2ccnc(N(C)[C@H]3CC[C@H](NC(=O)C4Cc5ccccc5C4)CC3)n2)cc1C1CC1 ; CN(c1nccc(Nc2cc(C3CC3)[nH]n2)n1)[C@H]1CC[C@H](NC(=O)C2CCc3ccccc32)CC1]

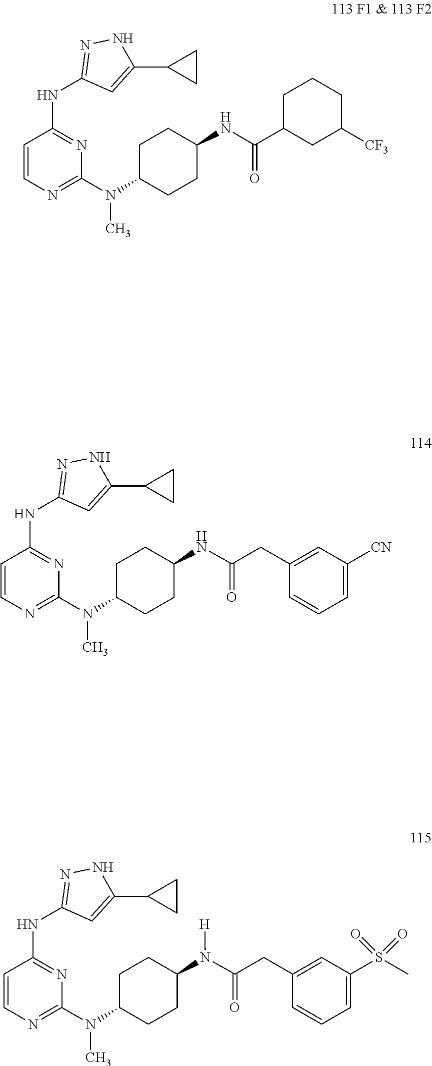 [SMILES: CN(c1nccc(Nc2cc(C3CC3)[nH]n2)n1)[C@H]1CC[C@H](NC(=O)C2CCCC(C(F)(F)F)C2)CC1 ; CN(c1nccc(Nc2cc(C3CC3)[nH]n2)n1)[C@H]1CC[C@H](NC(=O)Cc2cccc(C#N)c2)CC1 ; CN(c1nccc(Nc2cc(C3CC3)[nH]n2)n1)[C@H]1CC[C@H](NC(=O)Cc2cccc(S(C)(=O)=O)c2)CC1]

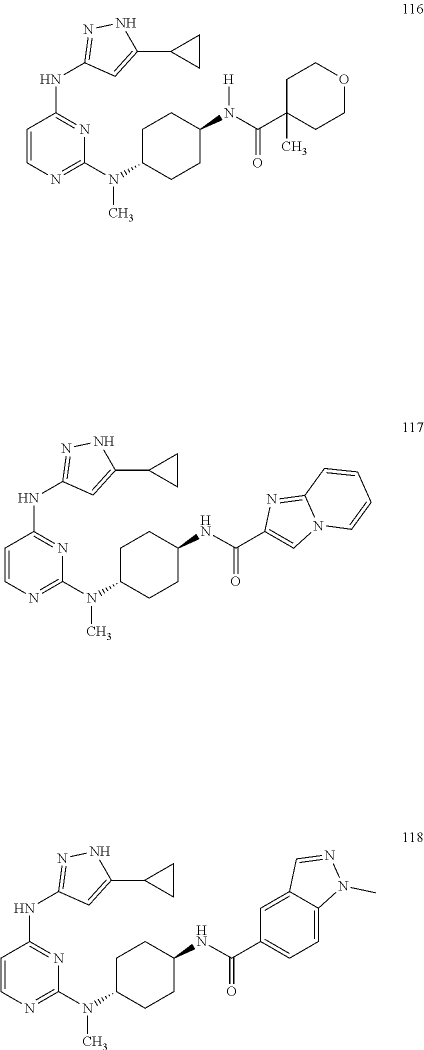 [SMILES: CN(c1nccc(Nc2cc(C3CC3)[nH]n2)n1)[C@H]1CC[C@H](NC(=O)C2(C)CCOCC2)CC1 ; CN(c1nccc(Nc2cc(C3CC3)[nH]n2)n1)[C@H]1CC[C@H](NC(=O)c2cn3ccccc3n2)CC1 ; Cn1ncc2cc(C(=O)N[C@H]3CC[C@H](N(C)c4nccc(Nc5cc(C6CC6)[nH]n5)n4)CC3)ccc21]

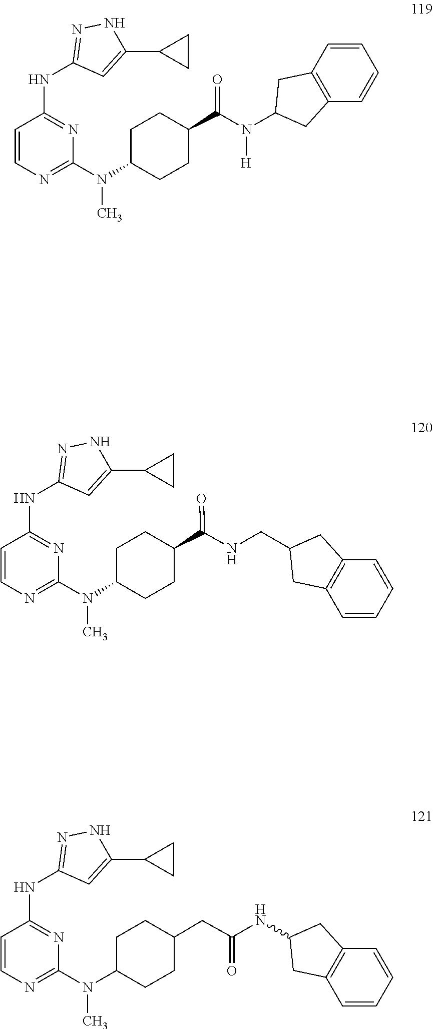 [SMILES: CN(c1nccc(Nc2cc(C3CC3)[nH]n2)n1)[C@H]1CC[C@H](C(=O)NC2Cc3ccccc3C2)CC1 ; CN(c1nccc(Nc2cc(C3CC3)[nH]n2)n1)[C@H]1CC[C@H](C(=O)NCC2Cc3ccccc3C2)CC1 ; CN(c1nccc(Nc2cc(C3CC3)[nH]n2)n1)C1CCC(CC(=O)NC2Cc3ccccc3C2)CC1]

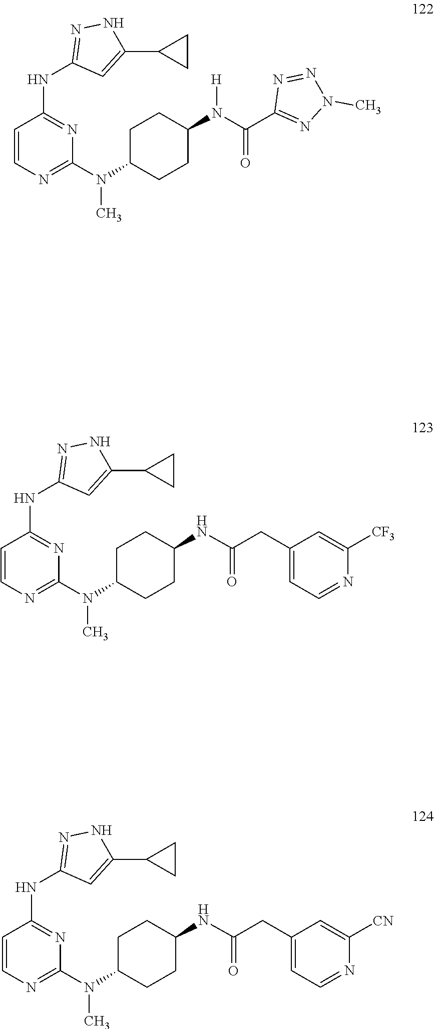 [SMILES: Cn1nnc(C(=O)N[C@H]2CC[C@H](N(C)c3nccc(Nc4cc(C5CC5)[nH]n4)n3)CC2)n1 ; CN(c1nccc(Nc2cc(C3CC3)[nH]n2)n1)[C@H]1CC[C@H](NC(=O)Cc2ccnc(C(F)(F)F)c2)CC1 ; CN(c1nccc(Nc2cc(C3CC3)[nH]n2)n1)[C@H]1CC[C@H](NC(=O)Cc2ccnc(C#N)c2)CC1]

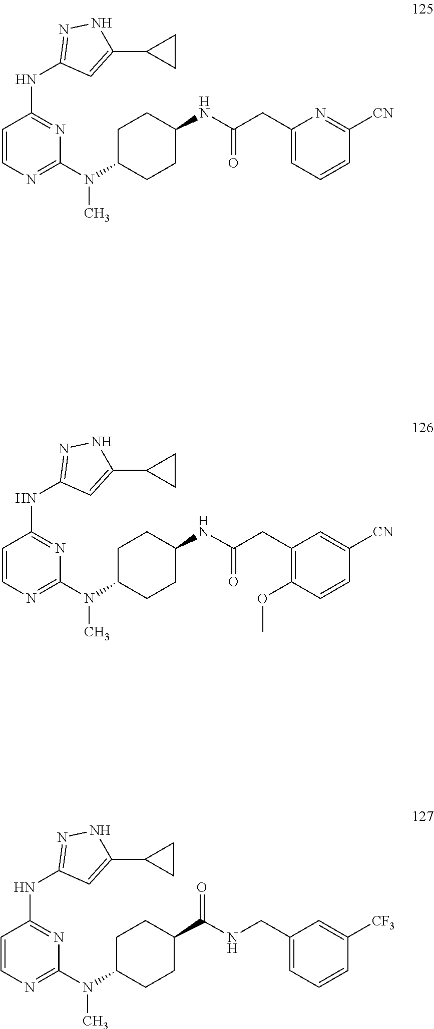 [SMILES: CN(c1nccc(Nc2cc(C3CC3)[nH]n2)n1)[C@H]1CC[C@H](NC(=O)Cc2cccc(C#N)n2)CC1 ; COc1ccc(C#N)cc1CC(=O)N[C@H]1CC[C@H](N(C)c2nccc(Nc3cc(C4CC4)[nH]n3)n2)CC1 ; CN(c1nccc(Nc2cc(C3CC3)[nH]n2)n1)[C@H]1CC[C@H](C(=O)NCc2cccc(C(F)(F)F)c2)CC1]

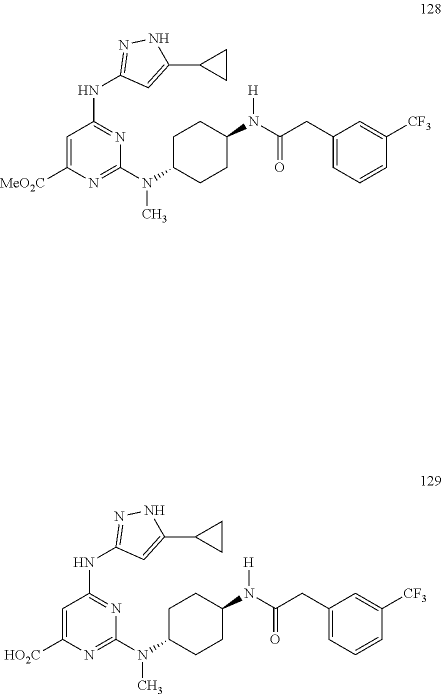 [SMILES: COC(=O)c1cc(Nc2cc(C3CC3)[nH]n2)nc(N(C)[C@H]2CC[C@H](NC(=O)Cc3cccc(C(F)(F)F)c3)CC2)n1 ; CN(c1nc(Nc2cc(C3CC3)[nH]n2)cc(C(=O)O)n1)[C@H]1CC[C@H](NC(=O)Cc2cccc(C(F)(F)F)c2)CC1]

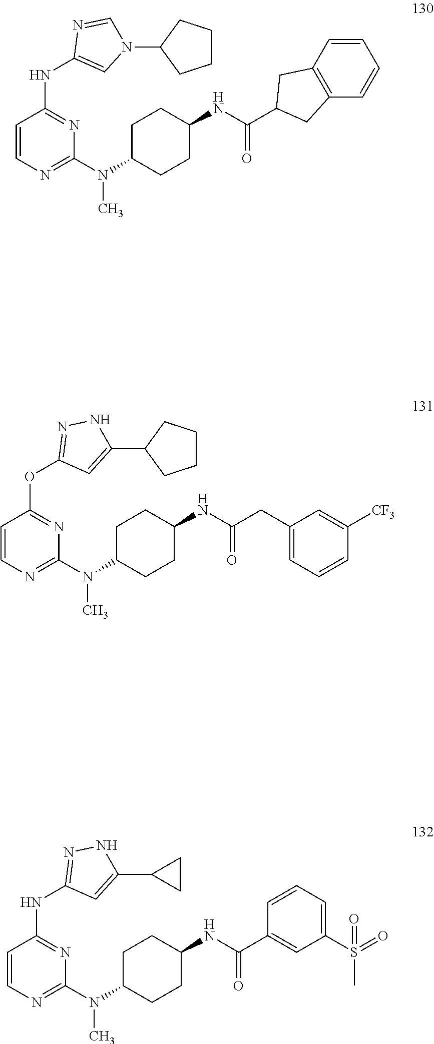

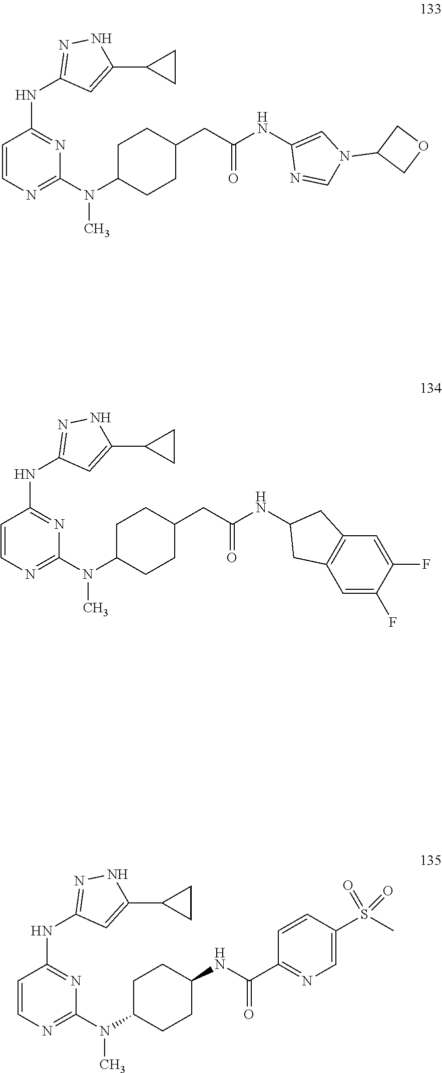 [SMILES: CN(c1nccc(Nc2cc(C3CC3)[nH]n2)n1)C1CCC(CC(=O)Nc2cn(C3COC3)cn2)CC1 ; CN(c1nccc(Nc2cc(C3CC3)[nH]n2)n1)C1CCC(CC(=O)NC2Cc3cc(F)c(F)cc3C2)CC1 ; CN(c1nccc(Nc2cc(C3CC3)[nH]n2)n1)[C@H]1CC[C@H](NC(=O)c2ccc(S(C)(=O)=O)cn2)CC1]

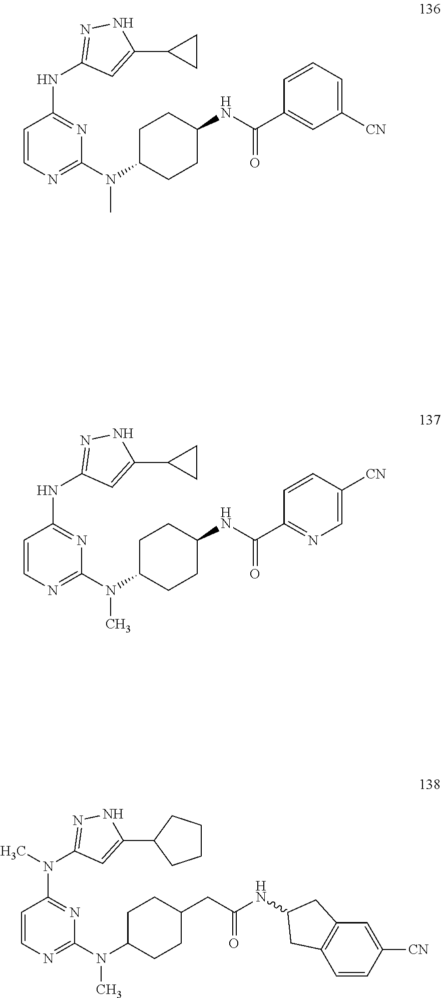 [SMILES: CN(c1nccc(Nc2cc(C3CC3)[nH]n2)n1)[C@H]1CC[C@H](NC(=O)c2cccc(C#N)c2)CC1 ; CN(c1nccc(Nc2cc(C3CC3)[nH]n2)n1)[C@H]1CC[C@H](NC(=O)c2ccc(C#N)cn2)CC1 ; CN(c1cc(C2CCCC2)[nH]n1)c1ccnc(N(C)C2CCC(CC(=O)NC3Cc4ccc(C#N)cc4C3)CC2)n1]

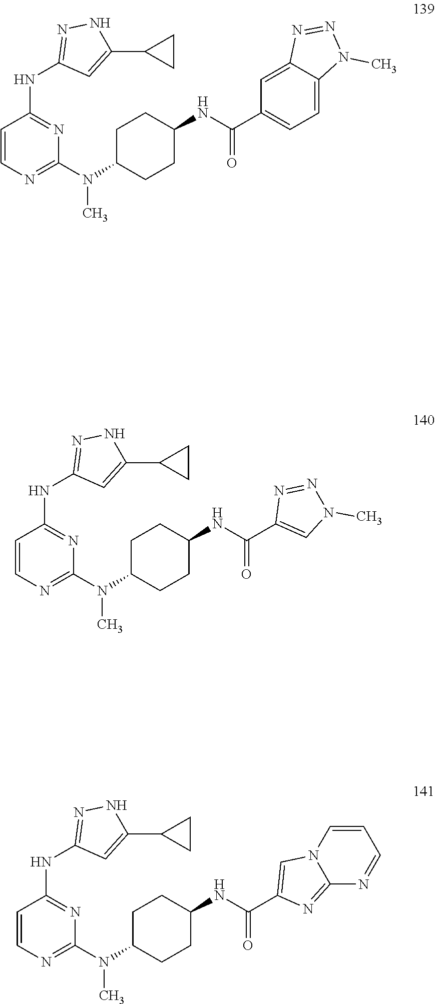 [SMILES: Cn1nnc2cc(C(=O)N[C@H]3CC[C@H](N(C)c4nccc(Nc5cc(C6CC6)[nH]n5)n4)CC3)ccc21 ; Cn1cc(C(=O)N[C@H]2CC[C@H](N(C)c3nccc(Nc4cc(C5CC5)[nH]n4)n3)CC2)nn1 ; CN(c1nccc(Nc2cc(C3CC3)[nH]n2)n1)[C@H]1CC[C@H](NC(=O)c2cn3cccnc3n2)CC1]

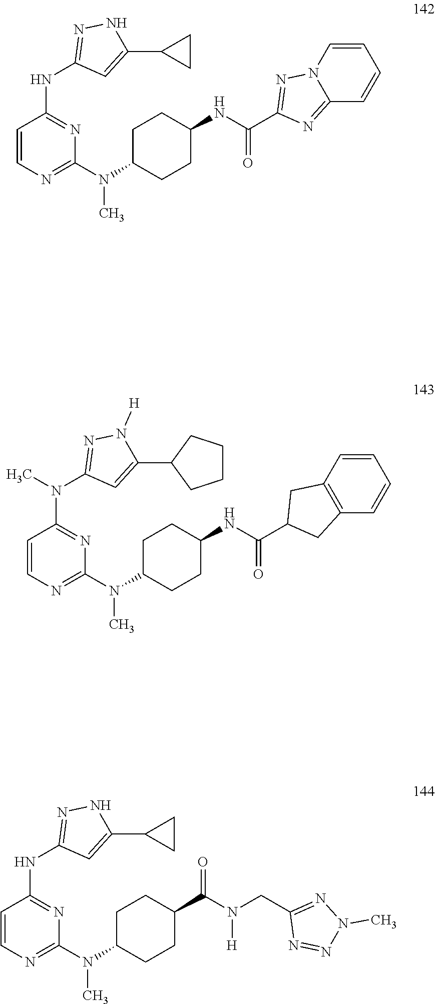

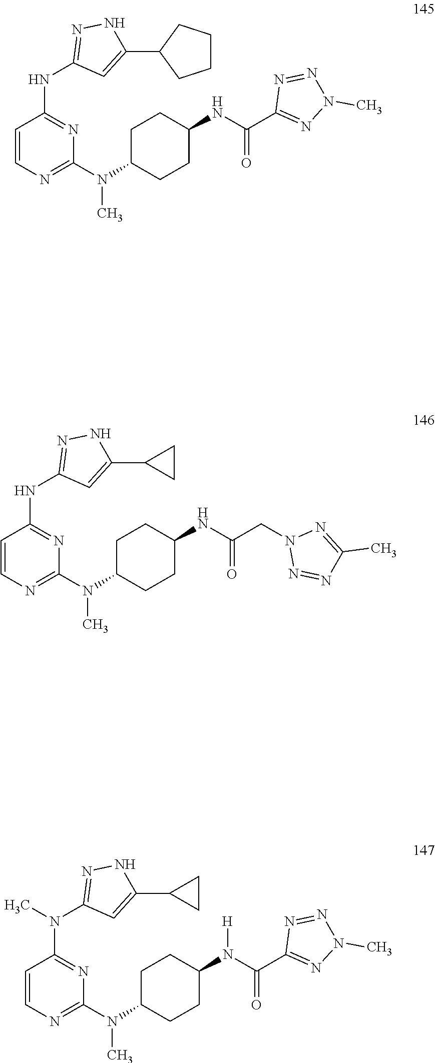 [SMILES: Cn1nnc(C(=O)N[C@H]2CC[C@H](N(C)c3nccc(Nc4cc(C5CCCC5)[nH]n4)n3)CC2)n1 ; Cc1nnn(CC(=O)N[C@H]2CC[C@H](N(C)c3nccc(Nc4cc(C5CC5)[nH]n4)n3)CC2)n1 ; CN(c1cc(C2CC2)[nH]n1)c1ccnc(N(C)[C@H]2CC[C@H](NC(=O)c3nnn(C)n3)CC2)n1]

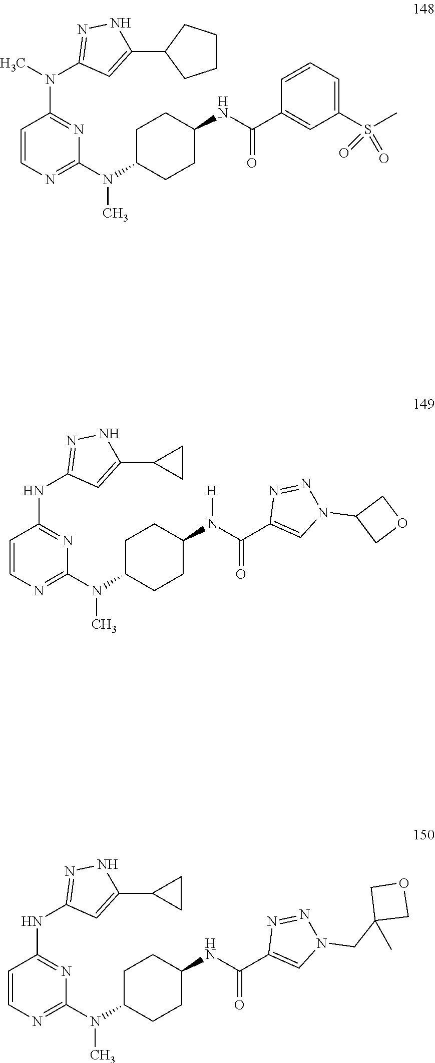 [SMILES: CN(c1cc(C2CCCC2)[nH]n1)c1ccnc(N(C)[C@H]2CC[C@H](NC(=O)c3cccc(S(C)(=O)=O)c3)CC2)n1 ; CN(c1nccc(Nc2cc(C3CC3)[nH]n2)n1)[C@H]1CC[C@H](NC(=O)c2cn(C3COC3)nn2)CC1 ; CN(c1nccc(Nc2cc(C3CC3)[nH]n2)n1)[C@H]1CC[C@H](NC(=O)c2cn(CC3(C)COC3)nn2)CC1]

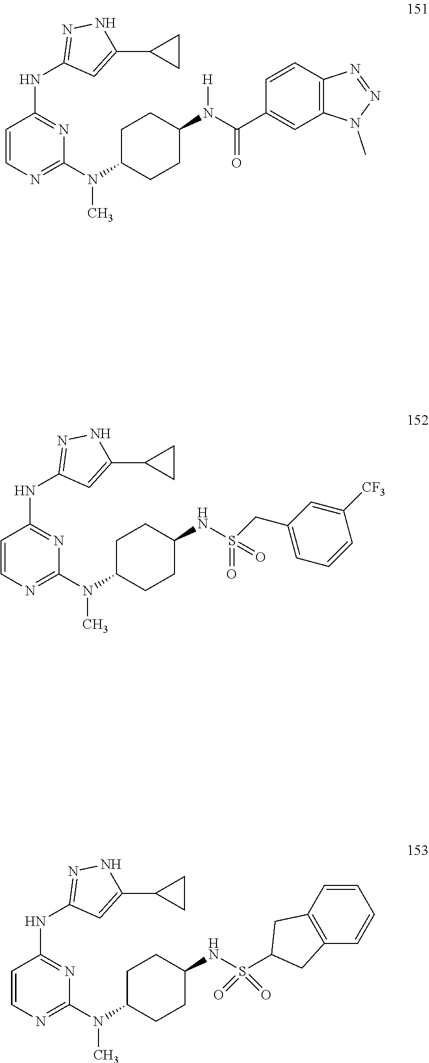 [SMILES: Cn1nnc2ccc(C(=O)N[C@H]3CC[C@H](N(C)c4nccc(Nc5cc(C6CC6)[nH]n5)n4)CC3)cc21 ; CN(c1nccc(Nc2cc(C3CC3)[nH]n2)n1)[C@H]1CC[C@H](NS(=O)(=O)Cc2cccc(C(F)(F)F)c2)CC1 ; CN(c1nccc(Nc2cc(C3CC3)[nH]n2)n1)[C@H]1CC[C@H](NS(=O)(=O)C2Cc3ccccc3C2)CC1]

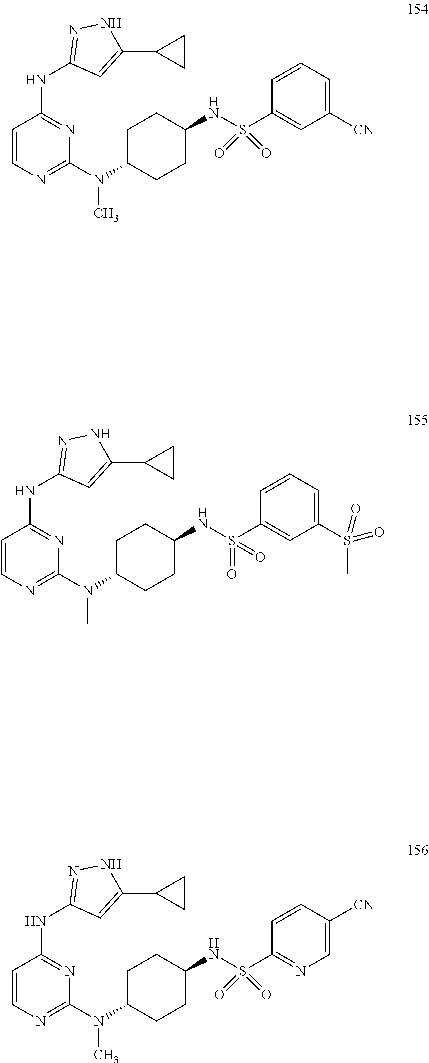 [SMILES: CN(c1nccc(Nc2cc(C3CC3)[nH]n2)n1)[C@H]1CC[C@H](NS(=O)(=O)c2cccc(C#N)c2)CC1 ; CN(c1nccc(Nc2cc(C3CC3)[nH]n2)n1)[C@H]1CC[C@H](NS(=O)(=O)c2cccc(S(C)(=O)=O)c2)CC1 ; CN(c1nccc(Nc2cc(C3CC3)[nH]n2)n1)[C@H]1CC[C@H](NS(=O)(=O)c2ccc(C#N)cn2)CC1]

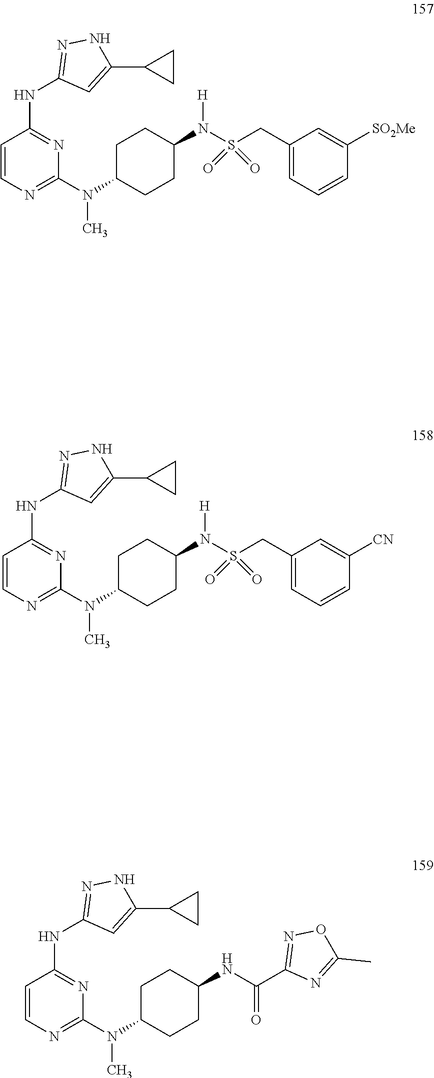 [SMILES: CN(c1nccc(Nc2cc(C3CC3)[nH]n2)n1)[C@H]1CC[C@H](NS(=O)(=O)Cc2cccc(S(C)(=O)=O)c2)CC1 ; CN(c1nccc(Nc2cc(C3CC3)[nH]n2)n1)[C@H]1CC[C@H](NS(=O)(=O)Cc2cccc(C#N)c2)CC1 ; Cc1nc(C(=O)N[C@H]2CC[C@H](N(C)c3nccc(Nc4cc(C5CC5)[nH]n4)n3)CC2)no1]

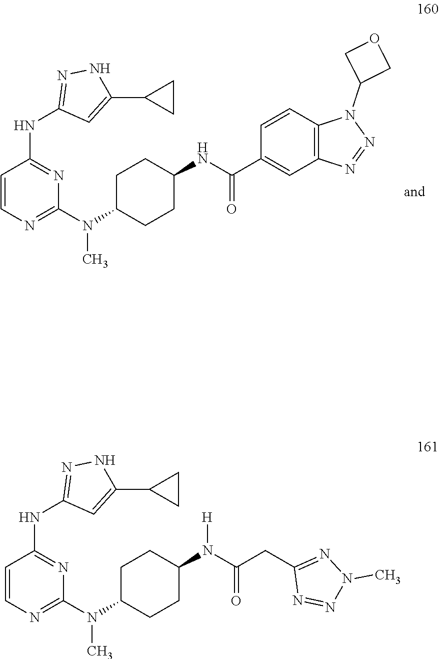 [SMILES: CN(c1nccc(Nc2cc(C3CC3)[nH]n2)n1)[C@H]1CC[C@H](NC(=O)c2ccc3c(c2)nnn3C2COC2)CC1 ; Cn1nnc(CC(=O)N[C@H]2CC[C@H](N(C)c3nccc(Nc4cc(C5CC5)[nH]n4)n3)CC2)n1]

and a pharmaceutically acceptable salt, stereoisomer, and/or N-oxide thereof.

Disclosed compounds described herein may be present in a salt form, and the salt form of the compound is a pharmaceutically acceptable salt, and/or compounds described herein may be present in a prodrug form. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)). Compounds described herein may be present in a solvate form.

In some embodiments, the compounds, or a prodrug form thereof, are provided in the form of pharmaceutically acceptable salts. Compounds containing an amine functional group or a nitrogen-containing heteroaryl group may be basic in nature and may react with any number of inorganic and organic acids to from the corresponding pharmaceutically acceptable salts. Inorganic acids commonly employed to form such salts include hydrochloric, hydrobromic, hydroiodic, sulfuric, and phosphoric acids, and related inorganic acids. Organic acids commonly employed to form such salts include para-toluenesulfonic, methanesulfonic, oxalic, para-bromophenylsulfonic, fumaric, maleic, carbonic, succinic, citric, benzoic and acetic acid, and related organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the related salts.

It is understood that all variations of salts, solvates, hydrates, prodrugs and stereoisomers are meant to be encompassed by the present disclosure.

### Pharmaceutical Compositions and Formulations

The compounds, prodrugs, and compositions described herein can be useful as pharmaceutical compositions for administration to a subject in need thereof.

Accordingly, pharmaceutical compositions are presented that can comprise at least a compound described herein, a pharmaceutically acceptable salt thereof, or a prodrug thereof, and at least one pharmaceutically acceptable carriers, diluent, stabilizers, excipients, dispersing agents, suspending agents, or thickening agents. For example, a disclosed pharmaceutical compositions may include one or more of the disclosed compounds, pharmaceutically acceptable salts, or prodrugs described herein. Contemplated compositions may include a compound, a pharmaceutically acceptable salt thereof, or a prodrug thereof in a therapeutically effective amount, for example, a disclosed pharmaceutical composition may be formulated for parenteral administration to a subject in need thereof, formulated for intravenous administration to a subject in need thereof, or formulated for subcutaneous administration to a subject in need thereof.

### Methods of Treatment

As described above, embodiments of the present disclosure include the use of compounds, prodrugs, and pharmaceutical compositions described herein to treat a Myc protein associated proliferative disease in a subject in need thereof. Such proliferative diseases include cancer, for example, a cancer selected from a group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

In some embodiments, a contemplated method of treating includes treating a cancer that is a Myc protein associated cancer, e.g., wherein the Myc protein is selected from the group consisting of a N-Myc protein, a c-MYc protein, a L-Myc protein, a human N-Myc protein, a human c-Myc protein, and a human L-Myc protein.

For example, provided herein is a method of treating a cancer selected from the group consisting of neuroblastoma, small cell lung carcinoma, breast cancer or a hematopoietic cancer.

In some embodiments, a disclosed method to treat cancer further comprises a second therapy, wherein the secondary therapy is an antineoplastic therapy, e.g., a contemplated method may further comprise administering an antineoplastic therapy such as one or more agents selected from a DNA topoisomerase I or II inhibitor, a DNA damaging agent, an immunotherapeutic agent (e.g., an antibody, cytokine, immune checkpoint inhibitor or cancer vaccine), an antimetabolite or a thymidylate synthase (TS) inhibitor, a microtubule targeted agent, ionizing radiation, an inhibitor of a mitosis regulator or a mitotic checkpoint regulator, an inhibitor of a DNA damage signal transducer, and an inhibitor of a DNA damage repair enzyme. For example, additional antineoplastic therapy may be selected from the group consisting of immunotherapy (e.g., immuno-oncologic therapy), radiation therapy, photodynamic therapy, gene-directed enzyme prodrug therapy (GDEPT), antibody-directed enzyme prodrug therapy (ADEPT), gene therapy, and controlled diets.

The present disclosure also contemplates the use of compounds, prodrugs, and pharmaceutical compositions described herein to modulate the amount and activity of a Myc protein (in vitro or in a patient), where the Myc protein may be for example a N-Myc protein, a c-MYc protein, a L-Myc protein, a human N-Myc protein, a human c-Myc protein, and/or a human L-Myc protein.

For example, the disclosure provides a method of modulating the amount (e.g., the concentration) and/or activity of a Myc protein such as (e.g. degrading a Myc protein, or modulating the rate of degradation of a Myc protein) that comprises contacting a Myc protein with an effective amount of a compound described herein, or a pharmaceutically acceptable salt, stereoisomer, and/or N-oxide thereof, including embodiments or from any examples, tables or figures.

Contemplated methods include methods of modulating the protein-protein interactions of the Myc family protein, or a method of decreasing the amount and decreasing the level of activity of a Myc protein.

A disclosed method of modulating the amount and activity of a Myc protein may include co-administering a compound described herein, or a pharmaceutically acceptable salt thereof, and a therapeutically effective amount of a second agent, e.g., therapeutic agent.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (i.e., amounts, temperatures, etc.), but some experimental error and deviation should, of course, be allowed for.

### General Experimental

Final compounds were confirmed by HPLC/MS analysis and determined to be ≥90% pure by weight. ¹H and ¹³C NMR spectra were recorded in CDCl₃ (residual internal standard CHCl₃=δ 7.26), DMSO-d₆ (residual internal standard CD₃SOCD₂H=δ 2.50), methanol-d₄ (residual internal standard CD₂HOD=δ 3.20), or acetone-d₆ (residual internal standard CD₃COCD₂H=δ 2.05). The chemical shifts (δ) reported are given in parts per million (ppm) and the coupling constants (J) are in Hertz (Hz). The spin multiplicities are reported as s=singlet, bs=broad singlet, bm=broad multiplet, d=doublet, t=triplet, q=quartet, p=pentuplet, dd=doublet of doublet, ddd=doublet of doublet of doublet, dt=doublet of triplet, td=triplet of doublet, tt=triplet of triplet, and m=multiplet.

HPLC-MS analysis was carried out with gradient elution. Medium pressure liquid chromatography (MPLC) was performed with silica gel columns in both the normal phase and reverse phase. It will be appreciated that compounds reported as a salt form (e.g., a TFA salt) may or may not have a 1:1 stoichiometry, and/or for example, reported potency concentrations or other assay results may be, e.g., slightly higher or lower.

### Example 1: Synthesis of Compounds 1 & 2

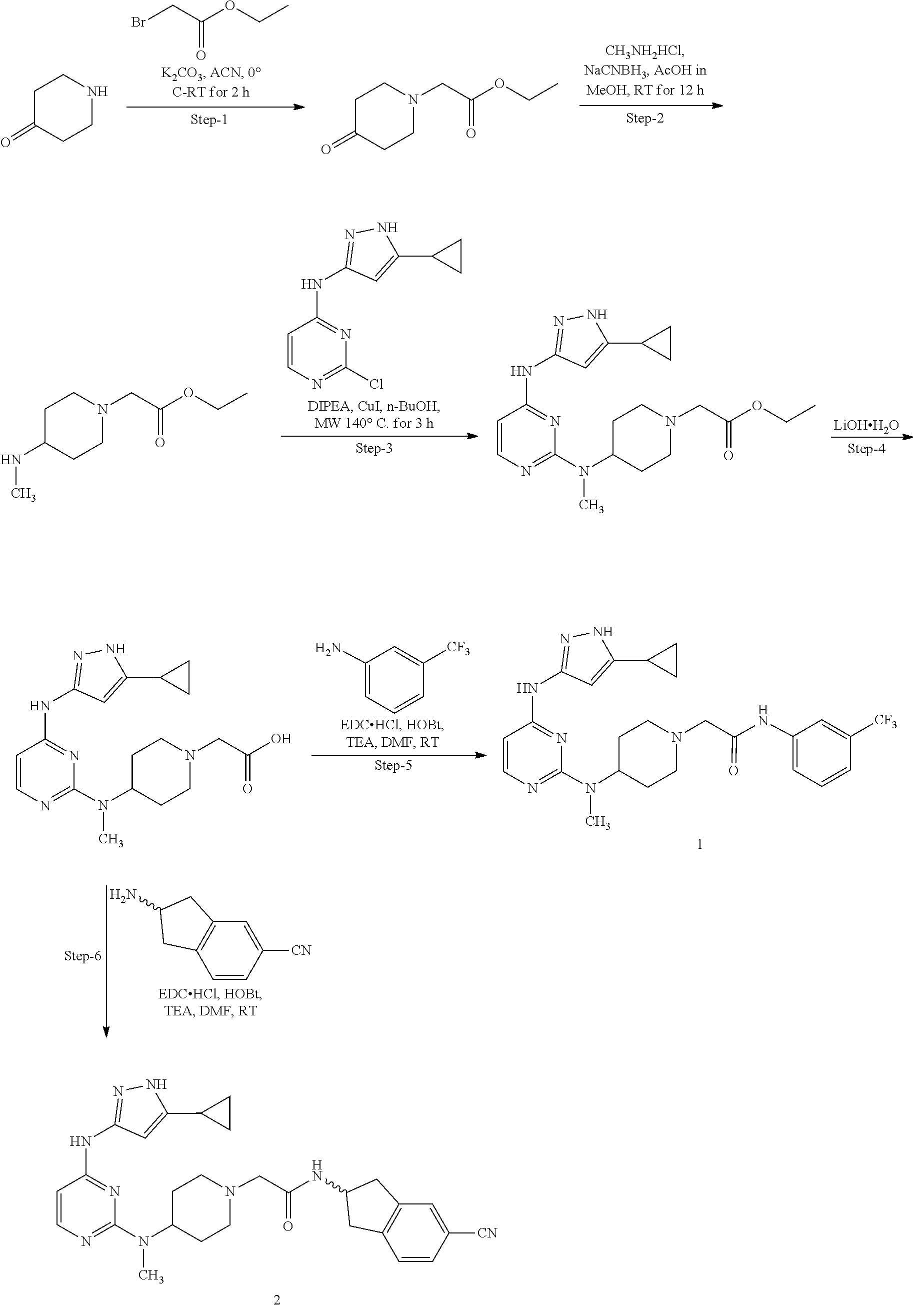

### Step-1: Synthesis of ethyl 2-(4-oxopiperidin-1-yl)acetate

To a solution of piperidin-4-one (5 g, 50.5 mmol) in dry DMF (50 mL) was added potassium carbonate (13.9 g, 101.01 mmol) and ethyl 2-bromoacetate (6.02 mL, 50.5 mmol). The reaction mixture was stirred at RT for 2 h. The progress of the reaction was monitored by TLC. The reaction mixture was diluted with water and extracted with ethyl acetate. The organic layer was dried over anhydrous sodium sulfate, filtered and concentrated under reduced pressure to get ethyl 2-(4-oxopiperidin-1-yl)acetate, which was taken to next step without further purification. LC purity: 90.71%; m/z: 186.2 [M+H]⁺ (Mol. formula C₉H₁₅NO₃ calcd. mol. wt. 185.02).

### Step-2: Synthesis of ethyl 2-(4-(methylamino)piperidin-1-yl)acetate

To a solution of ethyl 2-(4-oxopiperidin-1-yl)acetate (7.7 g, 41.62 mmol) in methanol (80 mL) was added methyl amine hydrochloride (3.62 g, 54.10 mmol) and 2 drops of acetic acid. The reaction mixture was stirred at RT for 2 h. Then sodium cyanoborohydride (5.16 g, 83.42 mmol) was added at 0° C. and the reaction mixture was stirred at RT for 12 h. The progress of the reaction was monitored by TLC. The reaction mixture was concentrated under reduced pressure to obtain the residue. The residue was dissolved in 30% methanol in dichloromethane and washed with a saturated solution of sodium bicarbonate. The organic layer was dried over anhydrous sodium sulfate, filtered and concentrated under reduced pressure to obtain ethyl 2-(4-(methylamino)piperidin-1-yl)acetate (5.2 g, 62.6%). LC purity: 97.97%; m/z: 201.2 [M+H]⁺ (Mol. formula C₁₀H₂₀N₂O₂ calcd. mol. wt. 200.98).

### Step-3: Synthesis of ethyl 2-(4-((4-((5-cyclopropyl-1H-pyrazol-3-yl)amino)pyrimidin-2-yl)(methyl)amino)piperidin-1-yl)acetate

To a solution of 2-chloro-N-(5-cyclopropyl-1H-pyrazol-3-yl)pyrimidin-4-amine (2 g, 8.51 mmol) in n-butanol (20 mL) in a 20 mL microwave vial was added ethyl 2-(4-(methylamino)piperidin-1-yl)acetate (3.4 g, 17.01 mmol), DIPEA (4.45 mL, 25.5 mmol) and copper iodide (200 mg). The reaction mixture was heated at 140° C. in a microwave reactor for 3 h. The progress of the reaction was monitored by TLC, After complete consumption of starting material, the reaction mixture was cooled to ambient temperature and concentrated to remove solvent. The crude was purified by Biotage Isolera using silica gel (230-400) with gradient elution of 0-5% methanol in dichloromethane to obtain ethyl 2-(4-((4-((5-cyclopropyl-1H-pyrazol-3-yl)amino)pyrimidin-2-yl)(methyl)amino)piperidin-1-yl)acetate (1 g, 30.3%). LC purity: 63.8%; m/z: 400.3 [M+H]⁺ (Mol. formula C₂₀H₂₉N₇O₂ calcd. mol. wt. 399.50).

### Step-4: Synthesis of 2-(4-((4-((5-cyclopropyl-1H-pyrazol-3-yl)amino)pyrimidin-2-yl)(methyl) amino)piperidin-1-yl) acetic acid

To a solution of ethyl 2-(4-((4-((5-cyclopropyl-1H-pyrazol-3-yl)amino)pyrimidin-2-yl)(methyl)amino)piperidin-1-yl)acetate (0.950 g, 2.3 mmol) in THF:MeOH:H₂O (1:1:1) (15 mL) was added lithium hydroxide monohydrate (0.199 g, 4.7 mmol). The reaction mixture was heated at 50° C. for 2 h. The progress of the reaction was monitored by TLC. After complete consumption of starting material, the reaction mixture was cooled and concentrated under reduced pressure to obtain the residue. The residue was purified by reverse phase preparative HPLC to get 2-(4-((4-((5-cyclopropyl-1H-pyrazol-3-yl)amino)pyrimidin-2-yl)(methyl)amino) piperidin-1-yl)acetic acid (430 mg, 51.6%). LC purity: 98.02%; m/z: 372 [M+H]⁺ (Mol. formula C₁₈H₂₅N₇O₂ calcd. mol. wt. 371.45).

### Step-5: Synthesis of 2-(4-((4-((5-cyclopropyl-1H-pyrazol-3-yl)amino)pyrimidin-2-yl)(methyl) amino)piperidin-1-yl)-N-(3-(trifluoromethyl)phenyl)acetamide

To a solution of 2-(4-((4-((5-cyclopropyl-1H-pyrazol-3-yl)amino)pyrimidin-2-yl)(methyl) amino)piperidin-1-yl)acetic acid (0.150 g, 0.4 mmol) in dry DMF (2 mL) was added triethylamine (0.2 mL, 2 mmol) followed by EDC.HCl (0.115 g, 0.6 mmol) and HOBt (0.027 g, 0.2 mmol). The reaction mixture was stirred at RT for 15 min. Then 3-(trifluoromethyl)aniline (0.052 g, 0.32 mmol) was added. The reaction mixture was stirred at room temperature for 16 h. The progress of the reaction was monitored by TLC. After complete consumption of starting material, the reaction mixture was diluted with water and extracted with DCM. The organic layer was dried over anhydrous sodium sulfate, filtered and concentrated under reduced pressure. The crude product was purified by reverse phase preparative HPLC to yield 2-(4-((4-((5-cyclopropyl-1H-pyrazol-3-yl)amino)pyrimidin-2-yl)(methyl)amino)piperidin-1-yl)-N-(3-(trifluoromethyl)phenyl) acetamide (30 mg, 20.78%) as the free base. LC purity: 99.28%; m/z: 515.2 [M+H]⁺ (Mol. Formula C₂₅H₂₉F₃N₈O calcd. mol. wt. 514.6). ¹H NMR (400 MHz, CD₃OD): δ 8.11 (s, 1H), 7.85 (t, J=7.6 Hz, 2H), 7.5 (t, J=8 Hz, 1H), 7.42 (d, J=8 Hz, 1H), 6.24-6.19 (m, 1H), 6.14 (s, 1H), 4.67-4.61 (m, 1H), 3.28 (s, 2H), 3.15-3.10 (m, 2H), 3.05 (s, 3H), 2.48-2.43 (m, 2H), 2.12-2.05 (m, 2H), 2.03-1.92 (m, 1H), 1.75-1.70 (m, 2H), 1.04-0.99 (m, 2H), 0.78-0.74 (m, 2H).

### Step-6: Synthesis of N-(5-cyano-2,3-dihydro-1H-inden-2-yl)-2-(4-((4-((5-cyclopropyl-1H-pyrazol-3-yl)amino)pyrimidin-2-yl)(methyl)amino)piperidin-1-yl)acetamide

To a solution of 2-(4-((4-((5-cyclopropyl-1H-pyrazol-3-yl)amino)pyrimidin-2-yl)(methyl)amino)piperidin-1-yl)acetic acid (0.150 g, 0.4 mmol) in dry DMF (5 mL) was added triethylamine (0.2 mL, 2 mmol) followed by EDC.HCl (0.115 g, 0.6 mmol) and HOBt (0.027 g, 0.026 mmol). The reaction mixture was stirred at room temperature for 15 min. Then 2-amino-2,3-dihydro-1H-indene-5-carbonitrile (0.062 g, 0.32 mmol) was added. The reaction mixture was stirred at room temperature for 16 h. The progress of the reaction was monitored by TLC. After complete consumption of the starting material, the reaction was diluted with water and extracted with DCM. The organic layer was dried over anhydrous sodium sulfate, filtered and concentrated under reduced pressure to obtain the crude product. The crude product was purified by reverse phase preparative HPLC to obtain N-(5-cyano-2,3-dihydro-1H-inden-2-yl)-2-(4-((4-((5-cyclopropyl-1H-pyrazol-3-yl)amino)pyrimidin-2-yl)(methyl)amino)piperidin-1-yl)acetamide (40 mg, 19.4%) as the free base. LC purity: 99.51%; m/z: 512.3 [M+H]⁺ (Mol. formula C₂₈H₃₃N₉O calcd. mol. wt. 511.63). ¹H NMR (400 MHz, CD₃OD): δ 7.85 (d, J=5.6 Hz, 1H), 7.62 (s, 1H), 7.56 (d, J=7.6 Hz, 1H), 7.44 (d, J=8 Hz, 1H), 6.23-6.15 (m, 2H), 4.75-4.68 (m, 1H), 4.61 (s, 1H), 3.40-3.32 (m, 2H), 3.07 (s, 3H), 3.05-2.97 (m, 6H), 2.36-2.30 (m, 2H), 1.94-1.87 (m, 3H), 1.67-1.62 (m, 2H), 1.04-0.99 (m, 2H), 0.77-0.74 (m, 2H).

### Example 2: Synthesis of Compound 3

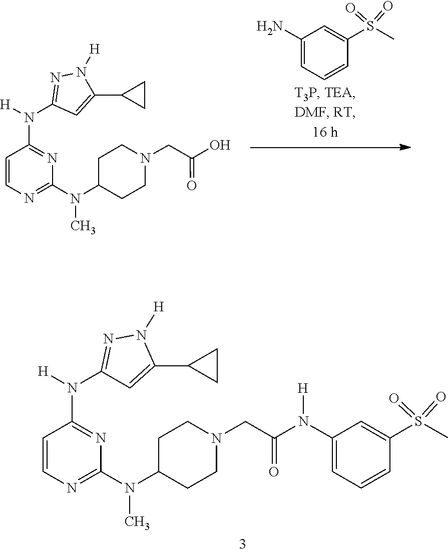 [SMILES: CN(c1nccc(Nc2cc(C3CC3)[nH]n2)n1)C1CCN(CC(=O)O)CC1 ; CS(=O)(=O)c1cccc(N)c1 ; CN(c1nccc(Nc2cc(C3CC3)[nH]n2)n1)C1CCN(CC(=O)Nc2cccc(S(C)(=O)=O)c2)CC1]

### Step-1: Synthesis of 2-(4-((4-((5-cyclopropyl-1H-pyrazol-3-yl)amino)pyrimidin-2-yl)(methyl)amino)piperidin-1-yl)-N-(3-(methylsulfonyl)phenyl)acetamide

To a solution of 2-(4-((4-((5-cyclopropyl-1H-pyrazol-3-yl)amino)pyrimidin-2-yl)(methyl)amino)piperidin-1-yl)acetic acid (0.250 g, 0.67 mmol) in dry DMF (5 mL) was added 3-(methylsulfonyl)aniline (0.115 g, 0.67 mmol), triethylamine (0.46 mL, 3.35 mmol) followed by T3P (50% solution in ethylacetate) (1.28 mL, 2.02 mmol). The reaction mixture was stirred at RT for 16 h. The progress of the reaction was monitored by UPLC. After complete consumption of the starting material, the reaction mixture was diluted water and extracted with 30% DCM in methanol. The organic layer was concentrated under reduced pressure to obtain the crude product. The crude product was purified by reverse phase preparative HPLC to yield 2-(4-((4-((5-cyclopropyl-1H-pyrazol-3-yl)amino)pyrimidin-2-yl)(methyl)amino)piperidin-1-yl)-N-(3-(methylsulfonyl)phenyl)acetamide (100 mg, 28.32%) as the free base. LC purity: 97.85%; m/z: 356.6 [M+H]⁺ (Mol. Formula C₂₅H₃₂N₈O₃S calcd. mol. wt. 524.64). ¹H NMR (400 MHz, CD₃OD): δ 8.38 (s, 1H), 7.86 (d, J=8.4 Hz, 2H), 7.75 (d, J=7.6 Hz, 1H), 7.65 (t, J=8 Hz, 1H), 6.58-6.32 (m, 2H), 4.32-4.26 (m, 2H), 3.96-3.89 (m, 2H), 3.39-3.32 (m, 2H), 3.14 (s, 6H), 2.36-2.23 (m, 2H), 2.14-2.07 (m, 2H), 2.05-1.95 (m, 1H), 1.73-1.64 (m, 1H), 1.08-1.03 (m, 2H), 0.80-0.76 (m, 2H).

### Example 3: Synthesis of Compound 4

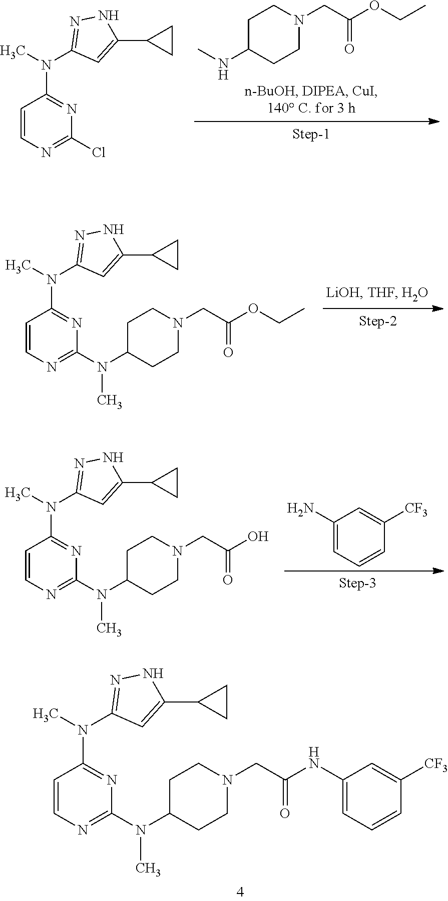

### Step-1: Synthesis of ethyl 2-(4-((4-((5-cyclopropyl-1H-pyrazol-3-yl)(methyl)amino) pyrimidin-2-yl)(methyl)amino)piperidin-1-yl)acetate

To a solution of 2-chloro-N-(5-cyclopropyl-1H-pyrazol-3-yl)-N-methylpyrimidin-4-amine (1 g, 4.0 mmol) in n-BuOH (5 mL) in a 20 mL microwave vial was added CuI (76 mg, 0.4 mmol) and DIPEA (2.15 mL, 1.2 mmol). The reaction mixture was stirred for 5 min and then ethyl 2-(4-(methylamino)piperidin-1-yl)acetate (1.2 g, 6.0 mmol) was added. The reaction mixture was stirred at 140° C. in a microwave reactor for 3 h. The progress of the reaction was monitored by TLC. After completion of the reaction, the reaction mixture was cooled to room temperature, diluted with water and extracted with dichloromethane. The combined organic layer was washed with water and brine, dried over anhydrous Na₂SO₄ and concentrated to remove solvent to provide crude compound. The crude was purified by biotage Isolera using 230-400 silica gel eluted with 0-10% ethyl acetate in pet ether to yield ethyl 2-(4-((4-((5-cyclopropyl-1H-pyrazol-3-yl)(methyl)amino)pyrimidin-2-yl)(methyl)amino)piperidin-1-yl)acetate (1.2 g, 75% yield). LC purity: 65.54%; m/z: 414.2 [M+H]⁺ (Mol. formula C₂₁H₃₁N₇O₂, calcd. mol. wt. 413.53).

### Step-2: Synthesis of 2-(4-((4-((5-cyclopropyl-1H-pyrazol-3-yl)(methyl)amino)pyrimidin-2-yl)(methyl)amino)piperidin-1-yl)acetic acid

To a stirred solution of ethyl 2-(4-((4-((5-cyclopropyl-1H-pyrazol-3-yl)(methyl)amino)pyrimidin-2-yl)(methyl)amino)piperidin-1-yl)acetate (1.2 g, 2.9 mmol) in THF:MeOH:H₂O (3 mL) was added LiOH·H₂O (610 mg, 14.5 mmol) and the reaction was heated at 60° C. for 3 h. The progress of the reaction was monitored by TLC. After completion of the reaction, the reaction mixture was cooled to room temperature and concentrated to obtain 2-(4-((4-((5-cyclopropyl-1H-pyrazol-3-yl)(methyl)amino)pyrimidin-2-1)(methyl)amino)piperidin-1-yl)acetic acid (1.2 g, crude). LC purity: 85.18%; m/z: 386.3 [M+H]⁺ (Mol. formula C₁₉H₂₇N₇O₂, calcd. mol. wt. 385.47).

### Step-3: Synthesis of 2-(4-((4-((5-cyclopropyl-1H-pyrazol-3-yl)(methyl)amino)pyrimidin-2-yl)(methyl)amino)piperidin-1-yl)-N-(3-(trifluoromethyl)phenyl)acetamide

To a solution of 2-(4-((4-((5-cyclopropyl-1H-pyrazol-3-yl)(methyl)amino)pyrimidin-2-yl)(methyl)amino)piperidin-1-yl)acetic acid (100 mg, 0.26 mmol) in dry DMF (3 mL) was added DIPEA (0.1 mL, 0.76 mmol) drop-wise at 0° C. followed by the addition of HATU (198 mg, 0.52 mmol). The reaction mixture was stirred at RT for 5 min and then 3-(trifluoromethyl)aniline (42 mg, 0.26 mmol) was added. The reaction mixture was warmed to room temperature and stirred for 16 h. The progress of the reaction was monitored by TLC. After completion of the reaction, the reaction mixture was diluted with dichloromethane washed with water and brine and dried over anhydrous Na₂SO₄ and concentrated to obtain the crude product. The crude product was purified by reverse phase preparative HPLC to yield 2-(4-((4-((5-cyclopropyl-1H-pyrazol-3-yl)(methyl)amino)pyrimidin-2-yl)(methyl)amino)piperidin-1-yl)-N-(3-(trifluoromethyl)phenyl)acetamide (10 mg, 7.3%) as the free base. LC purity: 97.16%; m/z: 529.3 [M+H]⁺ (Mol. formula C₂₆H₃₁F₃N₈O, calcd. mol. wt. 528.58). ¹H NMR (400 MHz, CD₃OD): δ 8.07 (s, 1H), 7.82-7.76 (m, 2H), 7.54 (t, J=8.0 Hz, 1H), 7.42 (d, J=7.6 Hz, 1H), 5.98 (d, J=6.0 Hz, 1H), 5.91 (s, 1H), 3.40 (s, 3H), 3.25 (s, 2H), 3.11-3.08 (m, 3H), 3.05 (s, 3H), 2.44-2.37 (m, 2H), 2.07-1.93 (m, 3H), 1.72-1.70 (m, 2H), 1.04-1.00 (m, 2H), 0.80-0.77 (m, 2H).

### Example 4: Synthesis of Compound 5

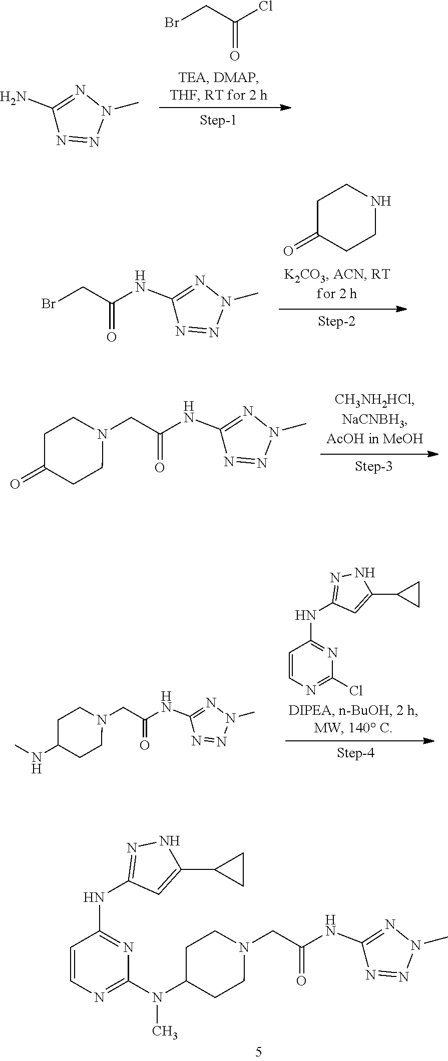

### Step-1: Synthesis of 2-bromo-N-(2-methyl-2H-tetrazol-5-yl)acetamide

To a cooled 0° C. solution of 2-methyl-2H-tetrazol-5-amine (0.600 g, 6.06 mmol) in dry THF (10 mL) was added DMAP (0.073 g, 0.606 mmol), triethylamine (1.26 mL, 9.09 mmol) followed by dropwise addition of 2-bromoacetyl chloride (0.58 mL, 6.06 mmol). The reaction mixture was stirred at RT for 2 h. The progress of the reaction was monitored by TLC. After completion of the reaction, the reaction mixture was diluted with dichloromethane and washed with water. The organic layer was dried over anhydrous sodium sulphate, filtered and concentrated under reduced pressure to get 2-bromo-N-(2-methyl-2H-tetrazol-5-yl)acetamide (700 mg, Crude). The crude as such was taken to next step without further purification. LC purity: 76.4%; m/z: 222.4 [M+H]⁺ (Mol. Formula C₄H₆BrN₅O calcd. mol. wt. 220.2).

### Step-2: Synthesis of N-(2-methyl-2H-tetrazol-5-yl)-2-(4-oxopiperidin-1-yl)acetamide

To a solution of 2-bromo-N-(2-methyl-2H-tetrazol-5-yl)acetamide (0.700 g, 3.18 mmol) in ACN (10 mL) was added potassium carbonate (1.09 g, 7.95 mmol) and piperidin-4-one (0.315 g, 3.18 mmol). The reaction mixture was stirred at RT for 2 h. The progress of the reaction was monitored by TLC. After completion of the reaction, the reaction mixture was diluted with dichlorometahne and washed with water. The organic layer was dried over anhydrous sodium sulphate, filtered and concentrated under reduced pressure to obtain N-(2-methyl-2H-tetrazol-5-yl)-2-(4-oxopiperidin-1-yl)acetamide (500 mg, Crude). The crude was taken to next step without any further purification. LC purity: 74.9%; m/z: 239.1 [M+H]⁺ (Mol. Formula C₉H₁₄N₆O₂ calcd. mol. wt. 238.25).

### Step-3: Synthesis of N-(2-methyl-2H-tetrazol-5-yl)-2-(4-(methylamino)piperidin-1-yl)acetamide

To a solution of N-(2-methyl-2H-tetrazol-5-yl)-2-(4-oxopiperidin-1-yl)acetamide (0.490 g, 2.05 mmol) in methanol (6 mL) was added methyl amine hydrochloride (0.181 g, 2.67 mmol) and (0.1 mL) of acetic acid. The reaction mixture was stirred at RT for 2 h. Then sodium cyanoborohydride (0.259 g, 4.1 mmol) was added at 0° C. and he reaction mixture was stirred at RT for 16 h. The progress of the reaction was monitored by TLC. After completion of the reaction, the reaction mixture was concentrated under reduced pressure to remove methanol. The crude was purified by reverse phase preparative HPLC to get N-(2-methyl-2H-tetrazol-5-yl)-2-(4-(methylamino)piperidin-1-yl)acetamide (130 mg, 24.16%). LC purity: 96.1%; m/z: 254.1 [M+H]⁺ (Mol. Formula C₁₀H₁₉N₇O calcd. mol. wt. 253.1).

### Step-4: Synthesis of 2-(4-((4-((5-cyclopropyl-1H-pyrazol-3-yl) amino) pyrimidin-2-yl)(methyl)amino)piperidin-1-yl)-N-(2-methyl-2H-tetrazol-5-yl) acetamide

To a solution of N-(2-methyl-2H-tetrazol-5-yl)-2-(4-(methylamino)piperidin-1-yl)acetamide (120 mg, 0.47 mmol) in n-Butanol (2 mL) in a 8 mL microwave vial was added 2-chloro-N-(5-cyclopropyl-1H-pyrazol-3-yl)pyrimidin-4-amine (33 mg, 0.14 mmol) and DIPEA (0.2 mL, 1.14 mmol). The reaction mixture was heated at 140° C. in a microwave reactor for 2 h. The reaction was monitored by LCMS (50% starting material remailing). The reaction mixture was concentrated to obtain the crude product which was purified by reverse phase preparative HPLC to obtain 2-(4-((4-((5-cyclopropyl-1H-pyrazol-3-yl) amino) pyrimidin-2-yl)(methyl)amino)piperidin-1-yl)-N-(2-methyl-2H-tetrazol-5-yl) acetamide (10 mg, 4.6%) as the free base. LC purity: 99.22%; m/z: 453.3 [M+H]⁺ (Mol. Formula C₂₀H₂₈N₁₂O calcd. mol. wt. 452.53). ¹H NMR (400 MHz, CD₃OD): δ 7.84 (d, J=6 Hz, 1H), 6.18 (d, J=5.6 Hz, 2H), 4.65-4.57 (m, 1H), 4.30 (s, 3H), 3.35-3.32 (m, 2H), 3.14-3.08 (m, 2H), 3.05 (s, 3H), 2.49-2.44 (m, 2H), 2.08-2.02 (m, 2H), 1.96-1.91 (m, 1H), 1.75-1.72 (m, 2H), 1.04-0.99 (m, 2H), 0.78-0.74 (m, 2H).

### Example 5: Synthesis of Compound 6

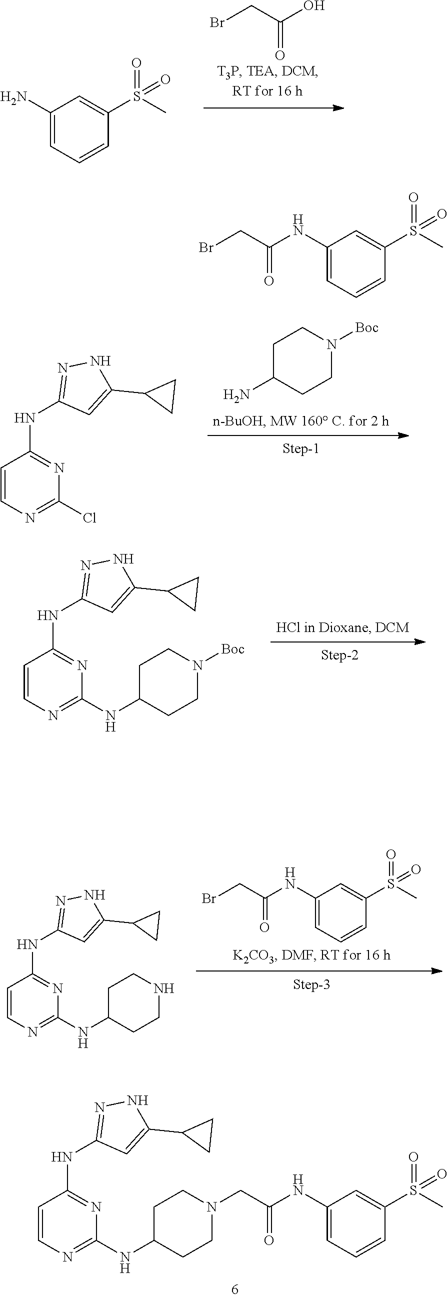

### Synthesis of 2-bromo-N-(3-(methylsulfonyl)phenyl)acetamide

To a solution of 2-bromoacetic acid (0.250 g, 1.81 mmol) in DCM (2 mL) was added 3-(methylsulfonyl)aniline (309 mg, 1.81 mmol), triethylamine (0.5 mL, 3.62 mmol) followed by T3P (50% solution in ethyl acetate) (1.7 mL, 2.71 mmol). The reaction was stirred at RT for 16 h. The progress of the reaction was monitored by UPLC, and after complete consumption of starting material, the reaction mixture was diluted water and extracted with DCM. The organic layer was washed with brine solution then dried over anhydrous Na₂SO₄ and concentrated to obtain the crude product. The crude product was purified by Biotage-Isolera using silica gel (230-400 mesh) with a gradient elution of 0-11% ethyl acetate in pet ether to yield 2-bromo-N-(3-(methylsulfonyl)phenyl)acetamide (220 mg, 41.66%). LC purity: 75.28%; m/z=292 [M−H]⁻ (Mol. formula C₉H₁₀BrNO₃S, calcd. mol. wt. 292.15).

### Step-1: Synthesis of tert-butyl 4-((4-((5-cyclopropyl-1H-pyrazol-3-yl)amino)pyrimidin-2-yl)amino)piperidine-1-carboxylate

To a solution of 2-chloro-N-(5-cyclopropyl-1H-pyrazol-3-yl)pyrimidin-4-amine (587 mg, 2.5 mmol) in n-butanol (10 mL) in a 20 mL microwave vial was added tert-butyl 4-aminopiperidine-1-carboxylate (1 g, 5 mmol). The reaction mixture was heated at 160° C. in a microwave reactor for 2 h. The progress of the reaction was monitored by TLC. After complete consumption of starting material, the reaction mixture was cooled to room temperature and concentrated to remove n-butanol. The residue obtained was triturated with dichloromethane, and pet ether to obtain tert-butyl 4-((4-((5-cyclopropyl-1H-pyrazol-3-yl)amino)pyrimidin-2-yl)amino)piperidine-1-carboxylate (400 mg, 20%), which was used for the next step without purification. LC purity: 65.74%; m/z: 300.3 [M-Boc]⁺ (Mol. formula C₂₀H₂₉N₇O₂, calcd. mol. wt. 399.50).

### Step-2: Synthesis of N₄-(5-cyclopropyl-1H-pyrazol-3-yl)-N2-(piperidin-4-yl)pyrimidine-2,4-diamine

A solution of tert-butyl 4-((4-((5-cyclopropyl-1H-pyrazol-3-yl)amino)pyrimidin-2-yl)amino)piperidine-1-carboxylate (100 mg, 0.25 mmol) in DCM (1 mL) was cooled to 0° C. and 4 M hydrochloric acid in 1,4 dioxane (1 mL) was added. The reaction mixture was allowed to stir at room temperature for 2 h. The reaction was monitored by TLC, and after consumption of starting material, the reaction mixture was concentrated to obtain N4-(5-cyclopropyl-1H-pyrazol-3-yl)-N2-(piperidin-4-yl)pyrimidine-2,4-diamine (100 mg, quantitative yield) as the HCl salt. LC purity: 88.25%; m/z: 300.2 [M+H]⁺ (Mol. formula C₁₅H₂₁N₇, calcd. mol. wt. 299.38)

### Step-3: Synthesis of 2-(4-((4-((5-cyclopropyl-1H-pyrazol-3-yl)amino)pyrimidin-2-yl)amino) piperidin-1-yl)-N-(3-(methylsulfonyl)phenyl)acetamide

To a stirred solution of N4-(5-cyclopropyl-1H-pyrazol-3-yl)-N₂-(piperidin-4-yl)pyrimidine-2,4-diamine (100 mg, 0.334 mmol) in dry DMF (2 mL) was added K₂CO₃ (92 mg, 0.668 mmol) and 2-bromo-N-(3-(methylsulfonyl)phenyl)acetamide (97 mg, 0.334 mmol). The reaction mixture was stirred at RT for 16 h. The progress of the reaction was monitored by TLC, and after complete consumption of starting material, the reaction mixture was diluted with water and the obtained solid was filtered. The solid was washed with pet ether and dried under vacuum to obtain the crude product. The crude product was purified by reverse phase prep HPLC to yield 2-(4-((4-((5-cyclopropyl-1H-pyrazol-3-yl)amino)pyrimidin-2-yl)amino)piperidin-1-yl)-N-(3-(methylsulfonyl)phenyl)acetamide (25 mg, 18%) as the free base. LC purity: 98.60%; m/z: 511.3 [M+H]⁺ (Mol. formula C₂₄H₃₀N₈O₃S, calcd. mol. wt. 510.62). ¹H NMR (400 MHz, CD₃OD): δ 8.34 (s, 1H), 7.93 (d, J=5.2 Hz, 1H), 7.91-7.80 (m, 1H), 7.72-7.69 (m, 1H), 7.63-7.59 (m, 1H), 6.20-6.10 (m, 2H), 3.82-3.78 (m, 1H), 3.27 (m, 2H), 3.14 (s, 3H), 3.03-2.97 (m, 2H), 2.48-2.43 (m, 2H), 2.09-2.05 (m, 2H), 1.94-1.91 (m, 1H), 1.90-1.69 (m, 2H), 0.99-0.83 (m, 2H), 0.75-0.67 (m, 2H).

### Example 6: Synthesis of Compound 7

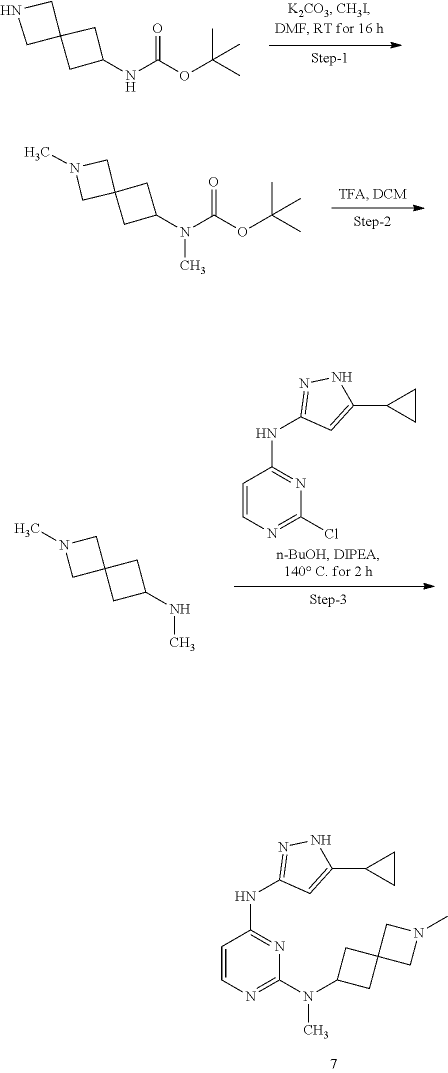

### Step-1: Synthesis of tert-butyl methyl(2-methyl-2-azaspiro[3.3]heptan-6-yl)carbamate

To a stirred solution of tert-butyl (2-azaspiro[3.3]heptan-6-yl)carbamate (0.300 g, 1.41 mmol) in dry DMF (6 mL) was added potassium carbonate (0.488 g, 3.5 mmol). The reaction mixture was stirred at RT for 10 min. Then methyl iodide (0.17 mL, 2.83 mmol) was added dropwise at 0° C. The reaction mixture was allowed to warmed to room temperature and stirred for 16 h. After completion of the reaction (monitored by TLC), the reaction mixture was diluted with water and extracted with ethyl acetate. The organic layer was dried over anhydrous sodium sulfate, filtered and concentrated under reduced pressure to obtain tert-butyl methyl(2-methyl-2-azaspiro[3.3]heptan-6-yl)carbamate (400 mg, quantitative yield). LC purity: 99.58%; m/z: 241.3 [M+H]⁺ (Mol. formula C₁₃H₂₄N₂O₂ calcd. mol. wt. 240.35).

### Step-2: Synthesis of N,2-dimethyl-2-azaspiro[3.3]heptan-6-amine

To a stirred solution of tert-butyl methyl(2-methyl-2-azaspiro[3.3]heptan-6-yl)carbamate (0.400 g, 1.66 mmol) in dry DCM (2 mL) cooled to 0° C. was added TFA (2 mL). The reaction was allowed to stir at room temperature for 1 h. The progress of the reaction was monitored by TLC, and after complete consumption of starting material, the resulting mixture was concentrated and triturated with pet ether and concentrated under high vacuum to yield N,2-dimethyl-2-azaspiro[3.3]heptan-6-amine as a TFA salt (300 mg, quantitative yield). LC purity: 97.9%; m/z: 141.3 [M+H]⁺ (Mol. formula C₈H₁₆N₂ calcd. mol. wt. 140.23).

### Step-3: Synthesis of N4-(5-cyclopropyl-1H-pyrazol-3-yl)-N2-methyl-N2-(2-methyl-2-azaspiro[3.3]heptan-6-yl)pyrimidine-2,4-diamine

To a solution of N,2-dimethyl-2-azaspiro[3.3]heptan-6-amine (0.300 g, 2.14 mmol) in n-Butanol (5 mL) in a 20 mL microwave vial was added 2-chloro-N-(5-cyclopropyl-1H-pyrazol-3-yl)pyrimidin-4-amine (0.251 g, 1.07 mmol) and DIPEA (1.12 mL, 6.42 mmol). The reaction mixture was heated at 140° C. in a microwave reactor for 2 h. The progress of the reaction was monitored by LCMS. After complete consumption of starting material, the reaction mixture was cooled to room temperature and concentrated to remove n-butanol. The crude thus obtained was purified by reverse phase preparative HPLC to obtain N4-(5-cyclopropyl-1H-pyrazol-3-yl)-N2-methyl-N2-(2-methyl-2-azaspiro[3.3]heptan-6-yl)pyrimidine-2,4-diamine (80 mg, 11.09%) as the free base. LC purity: 97.64%; m/z: 340.3 [M+H]⁺ (Mol. formula C₁₈H₂₅N₇ calcd. mol. wt. 339.45). ¹H NMR (400 MHz, CD₃OD): δ 7.85 (d, J=5.6 Hz, 1H), 6.16 (d, J=6 Hz, 1H), 6.06 (s, 1H), 4.78 (s, 1H), 3.50 (s, 2H), 2.70 (s, 2H), 2.30 (s, 6H), 1.98-1.91 (m, 3H), 1.59-1.57 (m, 2H), 0.98-0.94 (m, 2H), 0.79-0.74 (m, 2H).

### Example 7: Synthesis of Compound 8

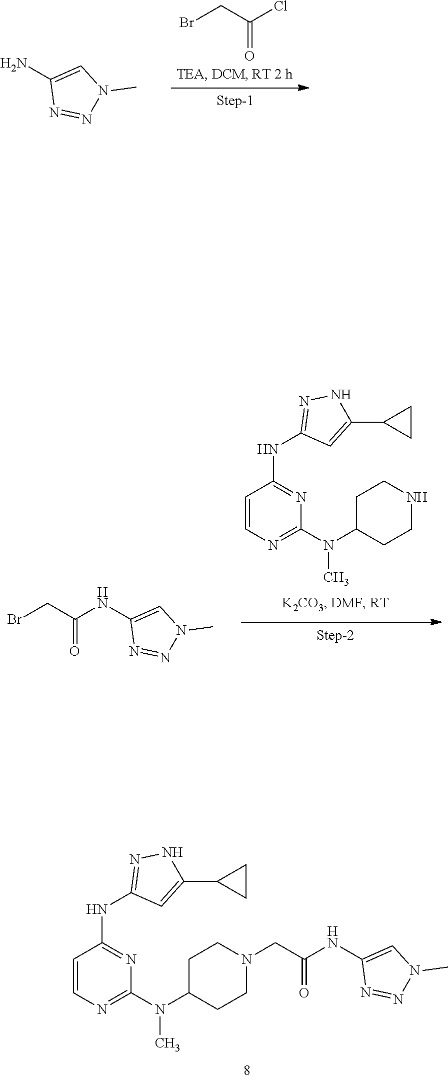

### Step-1: Synthesis of 2-bromo-N-(1-methyl-1H-1,2,3-triazol-4-yl)acetamide

To a cooled (0° C.) solution of 1-methyl-1H-1,2,3-triazol-4-amine (200 mg, 2.03 mmol) in dichloromethane (3 mL) was added trimethylamine (0.48 mL, 3.45 mmol) and 2-bromoacetyl chloride (0.19 mL, 2.23 mmol) dropwise. The reaction mixture was stirred at RT for 2 h. The progress of the reaction was monitored by TLC. After complete consumption of starting material, the resulting reaction mixture was quenched by addition of water and extracted with DCM. The organic layer was dried over anhydrous sodium sulfate, filtered, concentrated under reduced pressure to obtain 2-bromo-N-(1-methyl-1H-1,2,3-triazol-4-yl)acetamide (150 mg, 33%) which was used for the next step without purification. LC purity: 88.9%; m/z: 221.0 [M+2)+H]⁺ (Mol. formula C₅H₇BrN₄O, calcd. mol. wt. 219.04).

### Step-2: Synthesis of 2-(4-((4-((5-cyclopropyl-1H-pyrazol-3-yl)amino)pyrimidin-2-yl)(methyl)amino)piperidin-1-yl)-N-(1-methyl-1H-1,2,3-triazol-4-yl)acetamide

To a stirred solution of N4-(5-cyclopropyl-1H-pyrazol-3-yl)-N2-methyl-N2-(piperidin-4-yl) pyrimidine-2,4-diamine (200 mg, 0.638 mmol)) in dry DMF (4 mL) was added K₂CO₃ (176 mg, 1.276 mmol) and 2-bromo-N-(1-methyl-1H-1,2,3-triazol-4-yl)acetamide (140 mg, 0.638 mmol). The reaction mixture was stirred at RT for 16 h. The progress of the reaction was monitored by TLC, and after complete consumption of starting material, the reaction mixture was concentrated, diluted with water and extracted using 10% methanol in dichloromethane. The combined organic layers were washed with brine, dried over anhydrous Na₂SO₄ and concentrated. The crude compound was purified by reverse phase prep HPLC to yield 2-(4-((4-((5-cyclopropyl-1H-pyrazol-3-yl)amino)pyrimidin-2-yl)(methyl)amino)piperidin-1-yl)-N-(1-methyl-1H-1,2,3-triazol-4-yl)acetamide (23 mg, 8%) as the free base. LC purity: 99.5%; m/z: 452.1 [M+H]⁺ (Mol. formula C₂₁H₂₉N₁₁O, calcd. mol. wt. 451.54). ¹H NMR (400 MHz, CD₃OD): δ 8.15 (s, 1H), 7.86 (d, J=4.8 Hz, 1H), 6.25-6.14 (m, 2H), 4.66-4.58 (m, 1H), 4.11 (s, 3H), 3.29 (s, 2H), 3.11-3.09 (m, 2H), 3.05 (s, 3H), 2.47-2.42 (m, 2H), 2.07-2.03 (m, 2H), 1.94-1.91 (m, 1H), 1.74-1.71 (m, 2H), 1.02-0.98 (m, 2H), 0.79-0.74 (m, 2H).

### Example 8: Synthesis of Compound 9

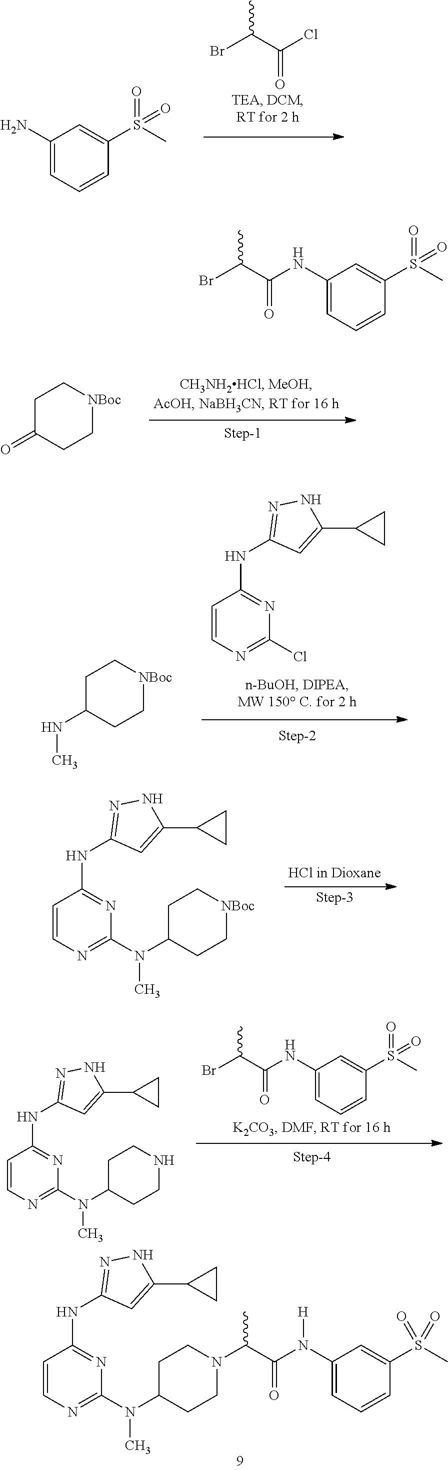

### Synthesis of 2-bromo-N-(3-(methylsulfonyl)phenyl)propenamide

To a cooled (0° C.) solution of 3-(methylsulfonyl) aniline (500 mg, 2.9 mmol) in DCM was added triethyl amine (0.6 mL, 4.35 mmol) and 2-bromopropanoyl chloride (540 mg, 3.1 mmol) dropwise. The reaction mixture was stirred at RT for 2 h. The progress of the reaction was monitored by LCMS, and after complete consumption of starting material, the reaction mixture was diluted with water and extracted with DCM. The organic layer was dried over sodium sulfate, filtered, concentrated under reduced pressure to obtain the residue. The residue was purified by Biotage Isolera using silica gel (230-400 mesh) column chromatography with gradient elution of 0-40% ethyl acetate in pet ether to obtain 2-bromo-N-(3-(methylsulfonyl)phenyl)propenamide (400 mg, 44%). LC purity: 65%; m/z: 308.0 [M+2H]⁺ (Mol. Formula C₁₀H₁₂BrNO₃S, calcd. Mol. Wt. 306.17).

### Step-1: Synthesis of tert-butyl 4-(methylamino)piperidine-1-carboxylate

To a stirred solution of tert-butyl 4-oxopiperidine-1-carboxylate (2 g, 10.0 mmol) in methanol (20 mL) was added catalytic acetic acid (0.2 mL) and methylamine hydrochloride (1 g, 15 mmol). The reaction was stirred at RT for 2 h. Then the reaction mixture was cooled to 0° C. and sodium cyanoborohydride (1.2 g, 20 mmol) was added portion wise. The reaction mixture was stirred at RT for 16 h. The completion of the starting material was monitored by LCMS. The reaction mixture was quenched by adding saturated sodium carbonate solution and extracted with DCM. The organic layer was dried over sodium sulfate, filtered and concentrated under reduced pressure to obtain tert-butyl 4-(methylamino)piperidine-1-carboxylate (2.1 g, 97%), which was used in the next step without purification. LC purity: 97.43%; m/z: 215.3 [M+H]⁺ (Mol. Formula C₁₁H₂₂N₂O₂, calcd. Mol. Wt. 214.31).

### Step-2: Synthesis of tert-butyl 4-((4-((5-cyclopropyl-1H-pyrazol-3-yl)amino)pyrimidin-2-yl)(methyl)amino)piperidine-1-carboxylate

To a mixture of 2-chloro-N-(5-cyclopropyl-1H-pyrazol-3-yl)pyrimidin-4-amine (1.08 g, 4.6 mmol) and tert-butyl 4-(methylamino)piperidine-1-carboxylate (2 g, 9.3 mmol) in n-Butanol (20 mL) in a 20 mL microwave vial was added DIPEA (2.3 mL, 13.94 mmol). The reaction mixture was heated at 150° C. in a microwave reactor for 2 h. The progress of the reaction was monitored by TLC. After complete consumption of starting material, the reaction mixture was cooled to room temperature and concentrated to remove n-butanol. The obtained residue was triturated with dichloromethane, and pet ether to obtain tert-butyl 4-((4-((5-cyclopropyl-1H-pyrazol-3-yl)amino)pyrimidin-2-yl)(methyl)amino)piperidine-1-carboxylate (700 mg, crude), which was used in the next step without purification. LC purity: 38%; m/z: 414.4 [M+H]⁺ (Mol. Formula C₂₁H₃₁N₇O₂, calcd. Mol. Wt. 413.53).

### Step-3: Synthesis of N4-(5-cyclopropyl-1H-pyrazol-3-yl)-N2-methyl-N2-(piperidin-4-yl)pyrimidine-2,4-diamine

To a cooled (0° C.) solution of tert-butyl 4-((4-((5-cyclopropyl-1H-pyrazol-3-yl)amino)pyrimidin-2-yl)(methyl)amino)piperidine-1-carboxylate (700 mg, 1.69 mmol) in DCM (10 mL) was added 4M Hydrochloric acid in 1,4 dioxane (8 mL). The reaction mixture was allowed to stir at room temperature for 2 h. The reaction was monitored by TLC, and after consumption of starting material, the reaction mixture was concentrated to obtain the residue. The residue was washed with DCM and concentrated to obtain N4-(5-cyclopropyl-1H-pyrazol-3-yl)-N2-methyl-N2-(piperidin-4-yl)pyrimidine-2,4-diamine (450 mg,) as the HCl salt. LC purity: 98.7%; m/z: 314.0[M+H]⁺ (Mol. Formula C₁₆H₂₃N₇, calcd. Mol. Wt. 313.41).

### Step-4: Synthesis of 2-(4-((4-((5-cyclopropyl-1H-pyrazol-3-yl)amino)pyrimidin-2-yl)(methyl)amino)piperidin-1-yl)-N-(3-(methylsulfonyl)phenyl)propenamide

To a stirred solution of N4-(5-cyclopropyl-TH-pyrazol-3-yl)-N2-methyl-N2-(piperidin-4-yl) pyrimidine-2,4-diamine (200 mg, 0.638 mmol) in dry DMF (4 mL) was added K₂CO₃ (176 mg, 1.276 mmol) and 2-bromo-N-(3-(methylsulfonyl)phenyl)propenamide (195 mg, 0.638 mmol). The reaction mixture was stirred at RT for 16 h. The progress of the reaction was monitored by TLC, and after complete consumption of starting material, the reaction mixture was concentrated, diluted with water and extracted using 10% methanol in dichloromethane. The combined organic layers were washed with brine, dried over anhydrous Na₂SO₄ and concentrated. The crude compound was purified by reverse phase preparative HPLC to yield 2-(4-((4-((5-cyclopropyl-1H-pyrazol-3-yl)amino)pyrimidin-2-yl)(methyl)amino)piperidin-1-yl)-N-(3-(methylsulfonyl)phenyl)propenamide (24 mg, 7%) as the free base. LC purity: 97.4%; m/z: 539.2 [M+H]⁺ (Mol. Formula C₂₆H₃₄N₈O₃S, calcd. Mol. Wt. 538.67). ¹H NMR (400 MHz, CD₃OD): δ 8.34 (s, 1H), 7.93-7.86 (m, 2H), 7.71-7.59 (m, 2H), 6.23-6.12 (m, 2H), 3.38-3.33 (m, 1H), 3.14 (s, 3H), 3.13-3.03 (m, 5H), 2.68-2.61 (m, 1H), 2.48-2.44 (m, 1H), 2.09-1.98 (m, 3H), 1.89-1.90 (m, 2H), 1.57-1.36 (m, 4H), 0.97-0.94 (m, 2H), 0.77-0.74 (m, 2H).

### Example 9: Synthesis of Compound 10

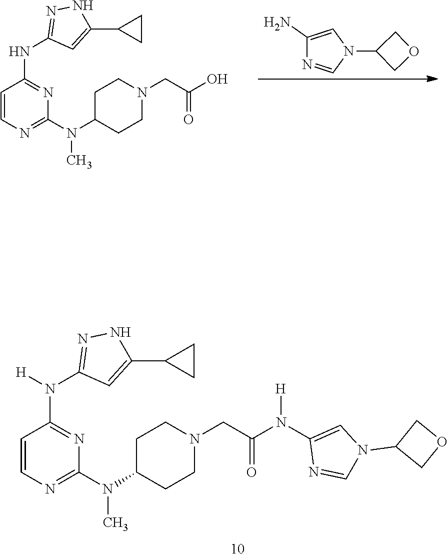

### Step-1: Synthesis of 2-(4-((4-((5-cyclopropyl-1H-pyrazol-3-yl)amino)pyrimidin-2-yl)(methyl)amino)piperidin-1-yl)-N-(1-(oxetan-3-yl)-1H-imidazol-4-yl)acetamide

To a solution of 2-(4-((4-((5-cyclopropyl-1H-pyrazol-3-yl)amino)pyrimidin-2-yl)(methyl)amino)piperidin-1-yl)acetic acid (70 mg, 0.485 mmol) in DMF (4 mL) was added TEA (0.2 mL, 1.45 mmol) followed by the addition of T₃P (1 mL, 1.45 mmol, 50% in EtOAc). The reaction mixture was stirred at RT for 5 min then 1-(oxetan-3-yl)-1H-imidazol-4-amine (180 mg, 0.485 mmol) was added. The reaction mixture was stirred at RT for 16 h. The progress of the reaction was monitored by LCMS. After completion of the reaction, the reaction mixture was diluted with dichloromethane and washed with water. The organic layer was dried over anhydrous Na₂SO₄ and concentrated to obtain the crude product. The crude product was purified by reverse phase preparative HPLC to yield 2-(4-((4-((5-cyclopropyl-1H-pyrazol-3-yl)amino)pyrimidin-2-yl)(methyl)amino)piperidin-1-yl)-N-(1-(oxetan-3-yl)-1H-imidazol-4-yl)acetamide (11 mg, 4.6%) as the free base. LC purity: 97.16%; m/z: 493.3 [M+H]⁺ (Mol. formula C₂₄H₃₂N₁₀O₂, calcd. mol. wt. 492.59). ¹H VTNMR (400 MHz, CD₃OD): δ 7.87 (d, J=5.6 Hz, 1H), 7.69-7.61 (m, 2H), 6.15 (d, J=6.0 Hz, 1H), 6.13 (s, 1H), 5.45-5.40 (m, 1H), 5.10 (t, J=7.2 Hz, 2H), 4.87 (t, J=6.4 Hz, 2H), 4.70-4.60 (m, 1H), 3.24 (s, 2H), 3.11-3.07 (m, 2H), 3.04 (s, 3H), 2.51-2.46 (m, 2H), 2.15-2.02 (m, 2H), 1.95-1.90 (m, 1H), 1.80-1.73 (m, 2H), 1.02-0.98 (m, 2H), 0.77-0.74 (m, 2H).

### Example 10: Synthesis of Compound 11

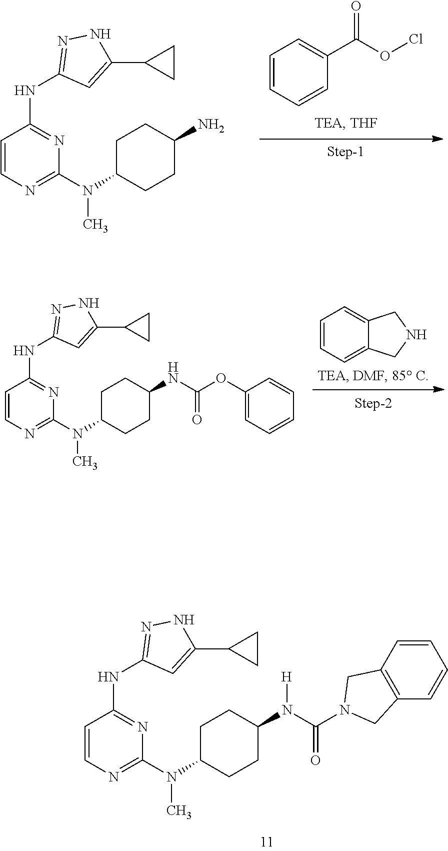 [SMILES: CN(c1nccc(Nc2cc(C3CC3)[nH]n2)n1)[C@H]1CC[C@H](N)CC1 ; O=C(OCl)c1ccccc1 ; CN(c1nccc(Nc2cc(C3CC3)[nH]n2)n1)[C@H]1CC[C@H](NC(=O)Oc2ccccc2)CC1 ; c1ccc2c(c1)CNC2 ; CN(c1nccc(Nc2cc(C3CC3)[nH]n2)n1)[C@H]1CC[C@H](NC(=O)N2Cc3ccccc3C2)CC1]

### Step-1: Synthesis of phenyl ((1R,4R)-4-((4-((5-cyclopropyl-1H-pyrazol-3-yl)amino) pyrimidin-2-yl)(methyl)amino)cyclohexyl)carbamate

To a stirred solution of N2-((1R,4R)-4-aminocyclohexyl)-N4-(5-cyclopropyl-1H-pyrazol-3-yl)-N2-methylpyrimidine-2,4-diamine (335 mg, 1.024 mmol) in dry THF (30 mL) was added triethylamine (1.29 mL, 9.220 mmol). The reaction mixture was stirred at 0° C. for 1 h and then benzoic hypochlorous anhydride (0.133 mL, 1.024 mmol) in THF (5 mL) was added. The reaction mixture was stirred at 0° C. for 0.5 h. After completion of the reaction (monitored by TLC), the reaction mixture was diluted with water and extracted with ethyl acetate. The resulting organic layer was washed with brine solution then dried over anhydrous Na₂SO₄ and concentrated under reduced pressure to yield the crude product. The obtained crude product was purified by biotage isolera using 230-400 silica mesh eluted with 0-100% pet ether in ethyl acetate to yield phenyl ((1R,4R)-4-((4-((5-cyclopropyl-1H-pyrazol-3-yl)amino)pyrimidin-2-yl)(methyl)amino)cyclohexyl)carbamate (100 mg, 22.22%). LC purity: 75.13%; m/z: 448.3 [M+H]⁺ (Mol. formula C₂₄H₂₉N₇O2, calcd. mol. wt. 447.5).

### Step-2: Synthesis of N-((1R,4R)-4-((4-((5-cyclopropyl-1H-pyrazol-3-yl)amino)pyrimidin-2-yl)(methyl)amino)cyclohexyl)isoindoline-2-carboxamide

To a stirred solution of phenyl ((1R,4R)-4-((4-((5-cyclopropyl-1H-pyrazol-3-yl)amino)pyrimidin-2-yl)(methyl)amino)cyclohexyl)carbamate (90 mg, 0.201 mmol) in dry DMF (1 mL) was added triethylamine (0.084 mL, 0.604 mmol). The reaction mixture was stirred at 65° C. to room temperature for 1 h. The reaction was cooled to room temperature and isoindoline (23.95 mg, 0.201 mmol) was added. The reaction mixture was heated to 85° C. for 16 h. After complete conversion of the starting material (monitored by UPLC), the reaction mixture was diluted with water and extracted with dichloromethane. The resulting organic layer was washed with brine solution then dried over anhydrous Na₂SO₄ and concentrated under reduced pressure to obtain the crude product. The crude product was purified by reverse phase preparative HPLC (0.1% TFA in water/acetonitrile) to yield N-((1R,4R)-4-((4-((5-cyclopropyl-1H-pyrazol-3-yl)amino) pyrimidin-2-yl)(methyl)amino)cyclohexyl)isoindoline-2-carboxamide (15 mg, 15.78%). LC purity: 97.60%; m/z: 473.2 [M+H]⁺ (Mol. formula C₂₆H₃₂N₈O, calcd. mol. wt. 472.6). ¹H NMR (400 MHz, MeOD): δ 7.73 (d, J=6.8 Hz, 1H), 7.35-7.29 (m, 4H), 6.38-6.36 (m, 2H), 4.69 (s, 4H), 4.66-4.55 (m, 1H), 3.72-3.64 (m, 1H), 3.10 (s, 3H), 2.19-2.17 (m, 2H), 2.05-2.00 (m, 1H), 1.88-1.86 (m, 4H), 1.61-1.51 (m, 2H), 1.07-1.05 (m, 2H), 0.79-0.73 (m, 2H).

### Example 11: Synthesis of Compound 12

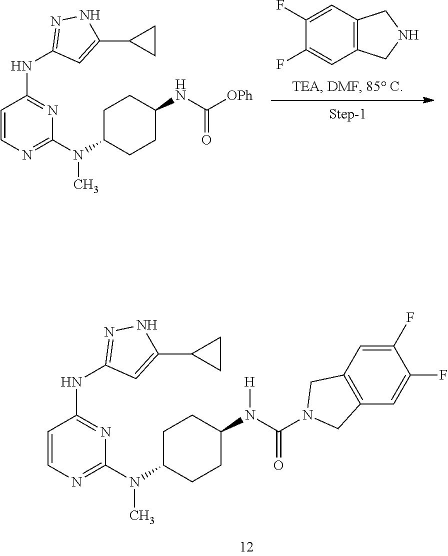 [SMILES: CN(c1nccc(Nc2cc(C3CC3)[nH]n2)n1)[C@H]1CC[C@H](NC(=O)Oc2ccccc2)CC1 ; Fc1cc2c(cc1F)CNC2 ; CN(c1nccc(Nc2cc(C3CC3)[nH]n2)n1)[C@H]1CC[C@H](NC(=O)N2Cc3cc(F)c(F)cc3C2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-5,6-difluoroisoindoline-2-carboxamide

To a stirred solution of phenyl ((1R,4R)-4-((4-((5-cyclopropyl-1H-pyrazol-3-yl)amino)pyrimidin-2-yl)(methyl)amino)cyclohexyl)carbamate (80 mg, 0.178 mmol) in dry DMF (2 mL) was added triethylamine (0.075 mL, 0.536 mmol). The reaction mixture was stirred at 65° C. for 1 h. The reaction was cooled to room temperature and 5,6-difluoroisoindoline (34.18 mg, 0.223 mmol) was added. The reaction mixture was heated to 85° C. for 16 h. After complete conversion of the starting material (monitored by UPLC), the reaction mixture was diluted with water and extracted with dichloromethane. The organic layer was washed with brine solution then dried over anhydrous Na₂SO₄ and concentrated under reduced pressure to obtain the crude product. The crude product was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-5,6-difluoroisoindoline-2-carboxamide (20 mg, 22.22%) as the free base. LC purity: 97.45%; m/z: 509.2 [M+H]⁺ (Mol. formula C₂₆H₃₀F₂N₈O, calcd. mol. wt. 508.58). ¹H NMR (400 MHz, DMSO-d₆): δ 11.92 (s, 1H), 9.31 (s, 1H), 7.85 (d, J=5.6 Hz, 1H), 7.46-7.41 (m, 2H), 6.18-6.04 (m, 3H), 4.58 (s, 4H), 4.57-4.51 (m, 1H), 3.53-3.46 (m, 1H), 2.96 (s, 3H), 1.95-1.85 (m, 3H), 1.70-1.62 (m, 4H), 1.47-1.37 (m, 2H), 0.93-0.90 (m, 2H), 0.71-0.67 (m, 2H).

### Example 12: Synthesis of Compound 13

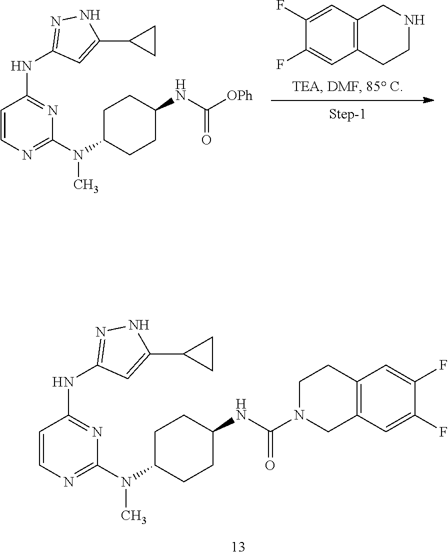 [SMILES: CN(c1nccc(Nc2cc(C3CC3)[nH]n2)n1)[C@H]1CC[C@H](NC(=O)Oc2ccccc2)CC1 ; Fc1cc2c(cc1F)CNCC2 ; CN(c1nccc(Nc2cc(C3CC3)[nH]n2)n1)[C@H]1CC[C@H](NC(=O)N2CCc3cc(F)c(F)cc3C2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-6,7-difluoro-3,4-dihydroisoquinoline-2(1H)-carboxamide

To a stirred solution of phenyl ((1R,4R)-4-((4-((5-cyclopropyl-1H-pyrazol-3-yl)amino)pyrimidin-2-yl)(methyl)amino)cyclohexyl)carbamate (80 mg, 0.178 mmol) in dry DMF (2 mL) was added triethylamine (0.075 mL, 0.536 mmol). The reaction mixture was stirred at 65° C. for 1 h. The reaction was cooled to room temperature and added 6,7-difluoro-1,2,3,4-tetrahydroisoquinoline (30.24 mg, 0.178 mmol). The reaction mixture was heated to 85° C. for 16 h. After complete conversion of the starting material (monitored by UPLC), the reaction mixture was diluted with water and extracted with dichloromethane. The organic layer was washed with brine solution then dried over anhydrous Na₂SO₄ and concentrated under reduced pressure to obtain the crude product. The crude product was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-6,7-difluoro-3,4-dihydroisoquinoline-2(1H)-carboxamide (25 mg, 26.88%) as the free base. LC purity: 99.20%; m/z: 522.9 [M+H]⁺ (Mol. formula C₂₇H₃₂F₂N₈O, calcd. mol. wt. 522.60). ¹H NMR (400 MHz, MeOD): δ 7.86 (d, J=5.6 Hz, 1H), 7.12-7.06 (m, 2H), 6.20-6.16 (m, 2H), 4.63 (s, 2H), 3.66-3.63 (m, 3H), 3.01 (s, 3H), 2.85-2.82 (m, 2H), 2.09-2.06 (m, 2H), 1.95-1.91 (m, 1H), 1.79-1.75 (m, 4H), 1.54-1.50 (m, 3H), 1.01-0.96 (m, 2H), 0.77-0.74 (m, 2H).

### Example 13: Synthesis of Compound 14

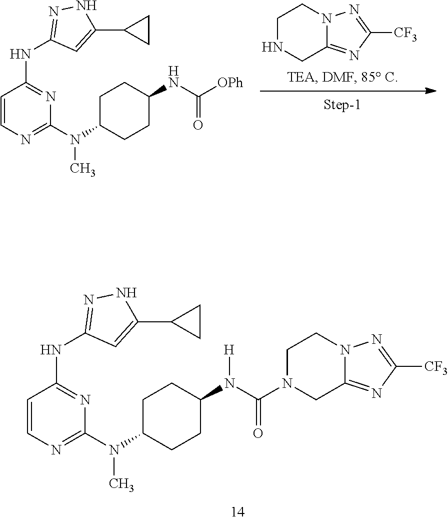 [SMILES: CN(c1nccc(Nc2cc(C3CC3)[nH]n2)n1)[C@H]1CC[C@H](NC(=O)Oc2ccccc2)CC1 ; FC(F)(F)c1nc2n(n1)CCNC2 ; CN(c1nccc(Nc2cc(C3CC3)[nH]n2)n1)[C@H]1CC[C@H](NC(=O)N2CCn3nc(C(F)(F)F)nc3C2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-2-(trifluoromethyl)-5,6-dihydro-[1,2,4]triazolo[1,5-a]pyrazine-7(8H)-carboxamide

To a stirred solution of phenyl ((1R,4R)-4-((4-((5-cyclopropyl-1H-pyrazol-3-yl)amino)pyrimidin-2-yl)(methyl)amino)cyclohexyl)carbamate (130 mg, 0.290 mmol) in dry DMF (2 mL) was added triethylamine (0.12 mL, 0.872 mmol). The reaction mixture was stirred at 65° C. for 1 h. The reaction was cooled to room temperature and 2-(trifluoromethyl)-5,6-dihydro-8H-7l2-[1,2,4]triazolo[5,1-c]pyrazine (55.5 mg, 0.290 mmol) was added. The reaction mixture was heated to 85° C. for 16 h. After complete conversion of the starting material (monitored by UPLC), the reaction mixture was diluted with water and extracted with dichloromethane. The organic layer was washed with brine solution then dried over anhydrous Na₂SO₄ and concentrated under reduced pressure to obtain crude compound. The crude compound was purified by reverse phase preparative HPLC (0.1% TFA in water/acetonitrile) to yield N-((1R,4R)-4-((4-((5-cyclopropyl-1H-pyrazol-3-yl)amino) pyrimidin-2-yl)(methyl)amino)cyclohexyl)-2-(trifluoromethyl)-5,6-dihydro-[1,2,4]triazolo[1,5-a]pyrazine-7(8H)-carboxamide (40 mg, 25.31%). LC purity: 99.89%; m/z: 546.2 [M+H]⁺ (Mol. formula C₂₄H₃₀F₃N₁₁O, calcd. mol. wt. 545.58). ¹H NMR (400 MHz, CD₃OD): δ 7.72 (d, J=7.6 Hz, 1H), 6.50-6.29 (m, 2H), 4.79 (s, 2H), 4.57-4.51 (m, 1H), 4.30 (t, J=5.2 Hz, 2H), 4.01 (t, J=5.6 Hz, 2H), 3.75-3.62 (m, 1H), 3.09 (s, 3H), 2.16-2.13 (m, 2H), 1.98-1.95 (m, 1H), 1.88-1.82 (m, 4H), 1.53-1.49 (m, 2H), 1.05-1.00 (m, 2H), 0.78-0.74 (m, 2H).

### Example 14: Synthesis of Compounds 15 F1 & 15 F2

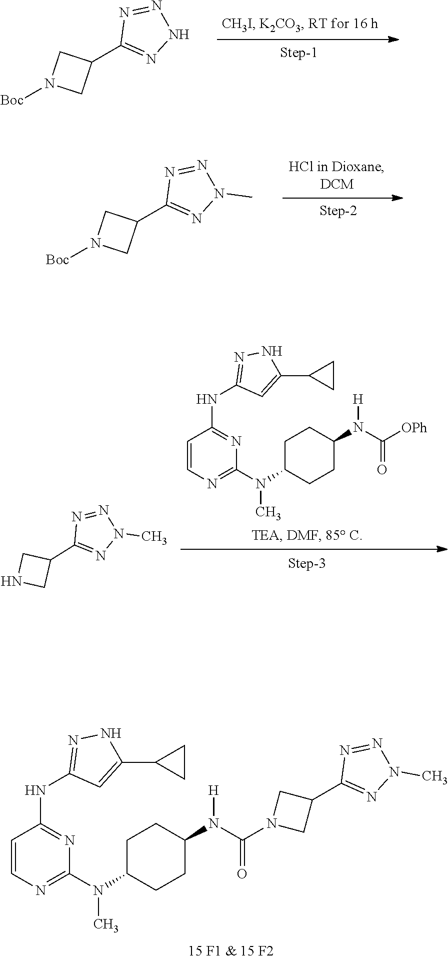

### Step-1: Synthesis of tert-butyl 3-(2-methyl-2H-tetrazol-5-yl)azetidine-1-carboxylate

To a stirred solution of tert-butyl 3-(2H-tetrazol-5-yl)azetidine-1-carboxylate (200 mg, 0.888 mmol) in acetonitrile (3 mL) was added potassium carbonate (122.6 mg, 0.888 mmol) followed by the addition of methyl iodide dropwise at 0° C. (0.05 mL, 0.924 mmol). The reaction mixture was stirred at room temperature for 16 h. After completion of the reaction (monitored by LCMS), the reaction mixture was filtered off through a bed of celite. The organic layer was concentrated under reduced pressure to yield tert-butyl 3-(2-methyl-2H-tetrazol-5-yl)azetidine-1-carboxylate (200 mg, 94.33%). LC purity: 52.47%, 30.69%; m/z: 240.2 [M+H]⁺ (Mol. formula C₁₀H₁₇N₅O₂ calcd. mol. wt. 239.28).

### Step-2: Synthesis of 5-(azetidin-3-yl)-2-methyl-2H-tetrazole

To a stirred solution of tert-butyl 3-(2-methyl-2H-tetrazol-5-yl)azetidine-1-carboxylate (200 mg, 0.836 mmol) in dichloromethane (3 mL) was added 4 M HCl in dioxane (2 mL). Then reaction mixture was allowed to stir at room temperature for 1 h. After completion of the reaction (monitored by TLC), the mixture was concentrated under reduced pressure to obtain 5-(azetidin-3-yl)-2-methyl-2H-tetrazole (150 mg, quantitative yield). LC purity: 39.6 & 59%; m/z: 140.2 [M+H]⁺ (Mol. formula C₅H₉N₅ calcd. mol. wt. 139.16).

### Step-3: Synthesis of N-((1R,4R)-4-((4-((5-cyclopropyl-1H-pyrazol-3-yl)amino)pyrimidin-2-yl)(methyl)amino)cyclohexyl)-3-(2-methyl-2H-tetrazol-5-yl)azetidine-1-carboxamide

To a stirred solution of phenyl ((1R,4R)-4-((4-((5-cyclopropyl-1H-pyrazol-3-yl)amino)pyrimidin-2-yl)(methyl)amino)cyclohexyl)carbamate (200 mg, 0.447 mmol) in dry DMF (3 mL) was added triethylamine (0.18 mL, 1.342 mmol). The reaction mixture was stirred at 65° C. for 1 h. The reaction was cooled to room temperature and 5-(azetidin-3-yl)-2-methyl-2H-tetrazole (62.1 mg, 0.447 mmol) was added. The reaction mixture was heated to 85° C. for 16 h. After completion of the of the reaction (monitored by UPLC), the reaction mixture was diluted with water and extracted with dichloromethane. The organic layer was dried over anhydrous Na₂SO₄ and concentrated under reduced pressure to obtain the crude product. The crude product was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-3-(2-methyl-2H-tetrazol-5-yl)azetidine-1-carboxamide (15 F1, 12 mg) and (15 F2, 14 mg) as the free base.

Compound 15 F1 Data: LC purity: 99.46%; m/z: 493.3 [M+H]⁺ (Mol. formula C₂₃H₃₂N₁₂O, calcd. mol. wt. 492.59). ¹H VTNMR (400 MHz, CD₃OD): δ 7.88 (d, J=5.8 Hz, 1H), 6.13-6.09 (m, 2H), 4.44-4.24 (m, 3H), 4.22-4.02 (m, 3H), 3.56 (s, 3H), 3.66-3.55 (m, 1H), 3.01 (s, 3H), 2.07-2.04 (m, 2H), 1.93-1.90 (m, 1H), 1.89-1.73 (m, 4H), 1.51-1.47 (m, 2H), 0.99-0.95 (m, 2H), 0.75-0.72 (m, 2H).

Compound 15 F2 Data: LC purity: 99.31%; m/z: 493.3 [M+H]⁺ (Mol. formula C₂₃H₃₂N₁₂O, calcd. mol. wt. 492.59). ¹H VTNMR (400 MHz, CD₃OD): δ 7.88 (d, J=6 Hz, 1H), 6.11 (d, J=6 Hz, 1H), 6.04 (s, 1H), 4.40-4.35 (m, 5H), 4.19-4.12 (m, 3H), 3.66-3.56 (m, 1H), 3.0 (s, 3H), 2.92-2.87 (m, 1H), 2.07-2.04 (m, 2H), 1.92-1.89 (m, 1H), 1.78-1.72 (m, 4H), 1.50-1.46 (m, 2H), 0.98-0.95 (m, 2H), 0.75-0.71 (m, 2H).

### Example 15: Synthesis of Compound 16

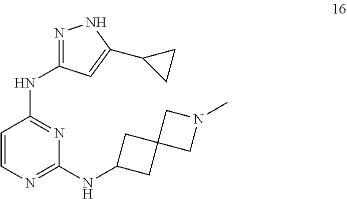 [SMILES: CN1CC2(CC(Nc3nccc(Nc4cc(C5CC5)[nH]n4)n3)C2)C1]

### N4-(5-Cyclopropyl-1H-pyrazol-3-yl)-N2-(2-methyl-2-azaspiro[3.3]heptan-6-yl)pyrimidine-2,4-diamine

A mixture of 2-chloro-N-(5-cyclopropyl-1H-pyrazol-3-yl)pyrimidin-4-amine (100 mg, 0.42 mmol, 1.0 eq), 2-methyl-2-azaspiro[3.3]heptan-6-amine) (107 mg, 0.85 mmol, 2.0 eq) and ethylbis(propan-2-yl)amine (0.22 mL, 1.27 mmol, 3.0 eq) and ethanol (6.0 mL) was heated in a microwave reactor at 120° C. for 12 h. The volatiles were removed under reduced pressure to afford a foam, which was purified by reverse phase HPLC under basic conditions to afford the desired compound as a solid (14 mg, 0.04 mmol, 10%). UPLC-MS (Basic Method, 4 min): rt 1.15 min, m/z=326.2 [M+H]+. 1H-NMR (400 MHz, DMSO) δ 11.89 (s, 1H), 9.31 (s, 1H), 7.83 (d, J=5.7 Hz, 1H), 6.19 (d, J=41.0 Hz, 2H), 4.68 (d, J=1.8 Hz, 1H), 2.75 (s, 2H), 2.32 (s, 3H), 1.86 (tt, J=8.7, 4.9 Hz, 1H), 1.79 (d, J=4.2 Hz, 2H), 1.37 (dd, J=4.3, 1.8 Hz, 2H), 0.90 (dt, J=8.7, 3.2 Hz, 2H), 0.69-0.63 (m, 2H).

### Example 16: Synthesis of Compounds 17 and 18

N4-(5-Cyclopentyl-1H-pyrazol-3-yl)-N2-methyl-N2-(piperidin-4-yl)pyrimidine-2,4-diamine and N4-(5-Cyclopentyl-1H-pyrazol-3-yl)-N2-(1-cyclopropylpiperidin-4-yl)-N2-methylpyrimidine-2,4-diamine

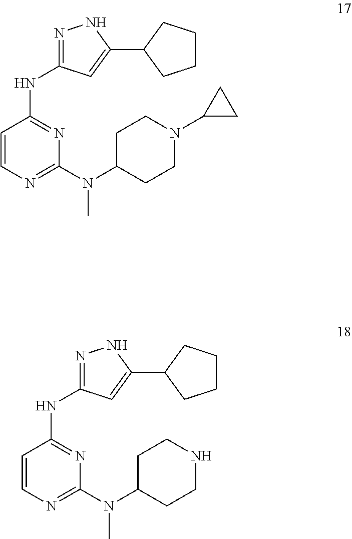 [SMILES: CN(c1nccc(Nc2cc(C3CCCC3)[nH]n2)n1)C1CCN(C2CC2)CC1 ; CN(c1nccc(Nc2cc(C3CCCC3)[nH]n2)n1)C1CCNCC1]

A mixture of 2-chloro-N-(5-cyclopentyl-1H-pyrazol-3-yl)pyrimidin-4-amine (100 mg, 0.38 mmol, 1.0 eq), 1-cyclopropyl-N-methylpiperidin-4-amine (117 mg, 0.76 mmol, 2.0 eq) and N,N-diisopropylethylamine (198 μL, 1.14 mmol, 3.0 eq) in ethanol (3 mL) was heated in a microwave reactor at 120° C. power=50 for 15 h. The solvent was then removed under reduced pressure and the crude residue was purified by reverse phase prep-HPLC to afford two products as solids: 17 (43.0 mg, 0.113 mmol, 29.7%) UPLC-MS (Basic Method, 4 min): rt 1.76 min, m/z=382.5 [M+H]⁺. ¹H NMR (400 MHz, DMSO) δ 11.92 (s, 1H), 9.43 (br, 1H), 7.83 (d, J=5.6 Hz, 1H), 6.39 (br, 1H), 6.10 (br, 1H), 4.65-4.54 (m, 1H), 3.05 (s, 3H), 2.90 (s, 3H), 2.31-2.21 (m, 2H), 2.02 (s, 2H), 1.76-1.49 (m, 11H), 0.42 (dt, J=6.1, 3.0 Hz, 2H), 0.30 (p, J=4.0 Hz, 2H). 18 (21 mg, 0.062 mmol, 16%) UPLC-MS (Basic Method, 4 min): rt 1.31 min, m/z=342.1 [M+H]⁺. ¹H NMR (400 MHz, DMSO) δ 11.92 (s, 1H), 9.35 (s, 1H), 7.85 (d, J=5.8 Hz, 1H), 6.22 (s, 2H), 4.44 (d, J=13.1 Hz, 2H), 3.08-2.87 (m, 4H), 2.28 (s, 3H), 1.99 (s, 2H), 1.80 (d, J=12.3 Hz, 2H), 1.74-1.50 (m, 6H), 1.12 (q, J=13.1, 11.1 Hz, 2H).

### Example 17: Synthesis of Compound 19

### N4-(5-Cyclopentyl-1H-pyrazol-3-yl)-N2,N4-dimethyl-N2-(2-methyl-2-azaspiro[3.3]heptan-6-yl)pyrimidine-2,4-diamine

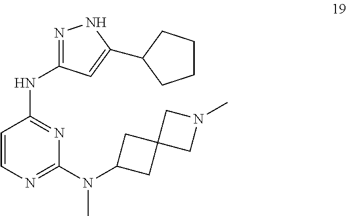 [SMILES: CN1CC2(CC(N(C)c3nccc(Nc4cc(C5CCCC5)[nH]n4)n3)C2)C1]

Paraformaldehyde (20 mg, 0.42 mmol, 1.5 eq) was added to a solution of N₂-{2-azaspiro[3.3]heptan-6-yl}-N4-(5-cyclopentyl-1H-pyrazol-3-yl)-N2-methylpyrimidine-2,4-diamine) (100 mg, 0.28 mmol, 1.0 eq) in MeOH (6 mL). The resulting mixture was stirred at 50° C. for 18 h, before being treated with an additional charge of NaBH₃CN (53 mg, 0.85 mmol, 3.0 eq). The resulting mixture was stirred at room temperature for 2 h. The solvent was removed under reduced pressure and the crude residue was purified by reverse phase HPLC (acid conditions). The resulting material was loaded onto an SCX column, washed with methanol, then eluted using ammonia in methanol. The solvent was removed under reduced pressure to afford the desired compound as a solid (45 mg, 0.12 mmol, 42%). UPLC-MS (Basic Method, 4 min) rt 1.55 min, m/z=382.5=[M+H]⁺. ¹H NMR (400 MHz, DMSO) δ 11.51 (s, 1H), 7.17 (d, J=7.5 Hz, 1H), 6.11 (s, 1H), 5.76 (s, 1H), 3.91 (d, J=10.4 Hz, 3H), 3.17 (s, 3H), 2.90 (s, 1H), 2.32 (s, 2H), 2.22-2.14 (m, 2H), 2.10 (s, 6H), 1.92 (d, J=11.3 Hz, 4H), 1.69-1.54 (m, 6H).

### Example 18: General procedure for the N-alkylation of N2-{2-Azaspiro[3.3]heptan-6-yl}-N4-(5-cyclopropyl-1H-pyrazol-3-yl)-N2-methylpyrimidine-2,4-diamine; bis(trifluoroacetic acid)

To a solution of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) (150 mg, 0.27 mmol, 1.0 eq) in THF (6.0 mL) was added potassium carbonate (149 mg, 1.08 mmol, 4.0 eq), and the resulting mixture was stirred at room temperature for 1 h. The mixture was treated with a solution of alkyl/benzyl halide (1.0 eq) in THF (2 mL), before being heated at 70° C. for 20 h. The reaction mixture was diluted with water (20 mL), basified with 1 M NaOH (10 mL) and extracted with EtOAc (3×20 mL). The combined organic extracts were dried over Na₂SO₄, filtered and concentrated under vacuum to afford the crude product, which was purified by automated flash column chromatography over silica gel (4 g Tellos cartridge) eluting with a solvent gradient of MeOH in DCM to afford the desired product.

### Example 19: Synthesis of Compound 20

### N4-(5-Cyclopropyl-1H-pyrazol-3-yl)-N2-methyl-N2-{2-[(oxan-4-yl)methyl]-2-azaspiro[3.3]heptan-6-yl}pyrimidine-2,4-diamine)

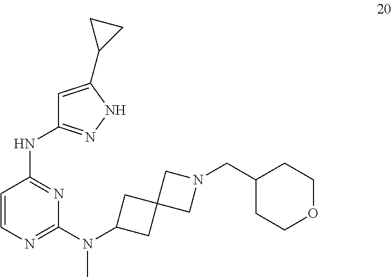 [SMILES: CN(c1nccc(Nc2cc(C3CC3)[nH]n2)n1)C1CC2(C1)CN(CC1CCOCC1)C2]

Compound 20 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) to afford the desired product as a solid (41 mg, 0.2 mmol, 36%). UPLC-MS (Basic Method, 4 min): rt 1.47 min, m/z 424.4 [M+H]⁺. ¹H NMR (400 MHz, DMSO-d6) δ ppm: 11.94 (s, 1H), 9.33 (s, 1H), 7.83 (d, J=5.7 Hz, 1H), 6.18 (s, 2H), 5.04 (p, J=8.9 Hz, 1H), 3.80 (dd, J=11.1, 2.8 Hz, 1H), 3.27-3.19 (m, 4H), 3.06 (s, 2H), 2.96 (s, 3H), 2.25 (s, 2H), 2.23 (s, 2H), 2.21 (d, J=6.7 Hz, 2H), 1.86 (tt, J=8.5, 4.9 Hz, 1H), 1.54 (d, J=12.5 Hz, 2H), 1.50-1.40 (m, 1H), 1.10 (qd, J=11.6, 4.1 Hz, 2H), 0.95-0.88 (m, 2H), 0.69-0.63 (m, 2H).

### Example 20: Synthesis of Compound 21

### N4-(5-Cyclopropyl-1H-pyrazol-3-yl)-N2-(2-(cyclopropylmethyl)-2-azaspiro[3.3]heptan-6-yl)-N2-methylpyrimidine-2,4-diamine

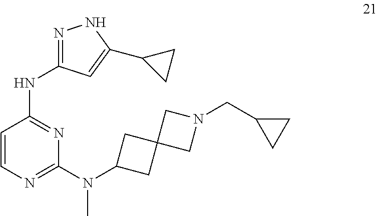 [SMILES: CN(c1nccc(Nc2cc(C3CC3)[nH]n2)n1)C1CC2(C1)CN(CC1CC1)C2]

Compound 21 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using (bromomethyl)cyclopropane, to afford the desired product as a solid (44 mg, 0.12 mmol, 43%). UPLC-MS (Basic Method, 4 min): rt 1.50 min, m/z=380.3 [M+H]⁺. ¹H-NMR (400 MHz, DMSO) δ 11.94 (s, 1H), 9.34 (s, 1H), 7.84 (d, J=5.7 Hz, 1H), 6.17 (s, 2H), 5.05 (p, J=8.8 Hz, 1H), 3.22 (s, 2H), 3.07 (s, 2H), 2.96 (s, 3H), 2.24 (d, J=8.8 Hz, 4H), 2.18 (d, J=6.6 Hz, 2H), 1.90-1.80 (m, 1H), 0.96-0.88 (m, 2H), 0.73-0.63 (m, 3H), 0.39-0.31 (m, 2H), 0.06 (d, J=4.1 Hz, 2H).

### Example 21: Synthesis of Compound 22

### N4-(5-Cyclopropyl-1H-pyrazol-3-yl)-N2-(2-(2-(2-methoxyethoxy)ethyl)-2-azaspiro[3.3]heptan-6-yl)-N2-methylpyrimidine-2,4-diamine

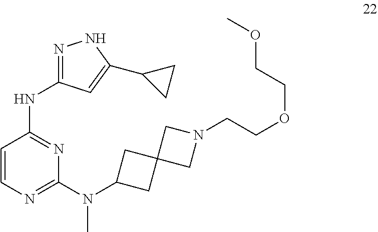 [SMILES: COCCOCCN1CC2(CC(N(C)c3nccc(Nc4cc(C5CC5)[nH]n4)n3)C2)C1]

Compound 22 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-bromo-2-(2-methoxyethoxy)ethane, to afford the desired product as a solid (22 mg, 0.05 mmol, 19%). UPLC-MS (Basic Method, 4 min): rt 1.39 min, m/z=428.3 [M+H]⁺. ¹H-NMR (400 MHz, MeOD) δ 7.84 (d, J=5.9 Hz, 1H), 6.14 (s, 2H), 5.05-4.91 (m, 1H), 3.59-3.46 (m, 8H), 3.36 (s, 3H), 3.03 (s, 3H), 2.67 (t, J=5.6 Hz, 2H), 2.46-2.29 (m, 4H), 1.90 (td, J=8.4, 4.4 Hz, 1H), 0.98 (d, J=7.7 Hz, 2H), 0.76-0.68 (m, 2H).

### Example 22: Synthesis of Compound 23

### 2-(6-((4-((5-Cyclopropyl-1H-pyrazol-3-yl)amino)pyrimidin-2-yl)(methyl)amino)-2-azaspiro[3.3]heptan-2-yl)-N-(3-(methylsulfonyl)phenyl)acetamide

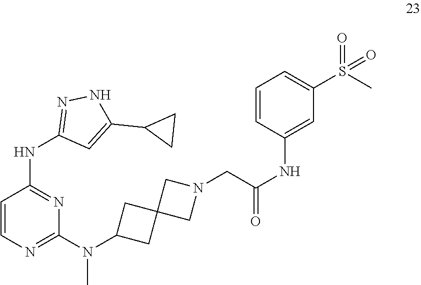 [SMILES: CN(c1nccc(Nc2cc(C3CC3)[nH]n2)n1)C1CC2(C1)CN(CC(=O)Nc1cccc(S(C)(=O)=O)c1)C2]

Compound 23 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 2-chloro-N-(3-(methylsulfonyl)phenyl)acetamide, to afford the desired product as a solid (35 mg, 0.07 mmol, 52%). UPLC-MS (Basic Method, 4 min): rt 1.46 min, m/z=537.2 [M+H]⁺. ¹H NMR (400 MHz, DMSO) δ 11.92 (s, 1H), 10.08 (s, 1H), 9.36 (s, 1H), 8.30-8.28 (m, 1H), 7.92 (dt, J=6.8, 2.2 Hz, 1H), 7.84 (d, J=5.7 Hz, 1H), 7.63-7.55 (m, 2H), 6.19 (s, 2H), 5.07 (p, J=8.9 Hz, 1H), 3.44 (s, 2H), 3.30 (s, 2H), 3.24 (s, 2H), 3.19 (s, 3H), 2.97 (s, 3H), 2.31 (d, J=8.9 Hz, 4H), 1.83 (td, J=8.6, 4.3 Hz, 1H), 0.89-0.82 (m, 2H), 0.72-0.57 (m, 2H).

### Example 23: Synthesis of Compound 24

### 2-(6-((4-((5-Cyclopropyl-1H-pyrazol-3-yl)amino)pyrimidin-2-yl)(methyl)amino)-2-azaspiro[3.3]heptan-2-yl)-N-(4-(methylsulfonyl)phenyl)acetamide

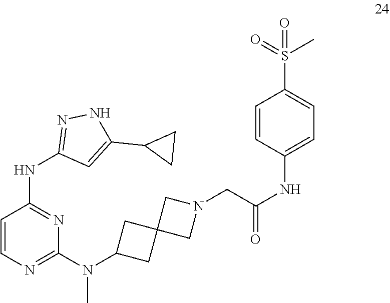 [SMILES: CN(c1nccc(Nc2cc(C3CC3)[nH]n2)n1)C1CC2(C1)CN(CC(=O)Nc1ccc(S(C)(=O)=O)cc1)C2]

Compound 24 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 2-chloro-N-(4-(methylsulfonyl)phenyl)acetamide, to afford the desired product as a solid (46 mg, 0.09 mmol, 48%). UPLC-MS (Basic Method, 2 min): rt 1.45 min, 537.2 [M+H]⁺. ¹H NMR (400 MHz, DMSO) δ 11.95 (s, 1H), 10.17 (s, 1H), 9.40 (s, 1H), 7.93-7.79 (m, 5H), 6.20 (s, 2H), 5.08 (p, J=9.0 Hz, 1H), 3.44 (s, 2H), 3.30 (s, 2H), 3.26 (s, 2H), 3.17 (s, 3H), 2.98 (s, 3H), 2.31 (d, J=8.8 Hz, 4H), 1.83 (td, J=8.5, 4.3 Hz, 1H), 0.86 (d, J=7.9 Hz, 2H), 0.68-0.60 (m, 2H).

### Example 24: Synthesis of Compound 25

### N4-(5-Cyclopropyl-1H-pyrazol-3-yl)-N2-(2-(2-fluorobenzyl)-2-azaspiro[3.3]heptan-6-yl)-N2-methylpyrimidine-2,4-diamine

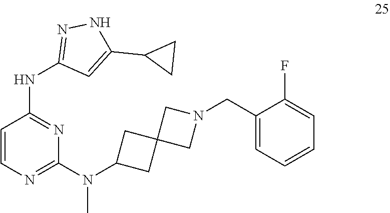 [SMILES: CN(c1nccc(Nc2cc(C3CC3)[nH]n2)n1)C1CC2(C1)CN(Cc1ccccc1F)C2]

Compound 25 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-(bromomethyl)-2-fluorobenzene, to afford the desired product as a solid (13 mg, 0.03 mmol, 17%). UPLC-MS (Basic Method, 4 min): rt 1.80 min, m/z: 434.3 [M+H]⁺. ¹H NMR (400 MHz, DMSO-d₆) δ 11.92 (s, 1H), 9.36 (s, 1H), 7.83 (d, J=5.7 Hz, 1H), 7.41-7.32 (m, 1H), 7.32-7.25 (m, 1H), 7.19-7.09 (m, 2H), 6.17 (s, 2H), 3.28 (s, 2H), 3.15 (s, 2H), 2.96 (s. 3H). 2.27 (s, 3H), 2.25 (s, 2H), 1.79 (tt, J=8.6, 5.0 Hz, 1H), 0.88-0.79 (m, 1H), 0.66-0.58 (m, 2H).

### Example 25: Synthesis of Compound 26

### N4-(5-Cyclopropyl-1H-pyrazol-3-yl)-N2-(2-(3,5-difluorobenzyl)-2-azaspiro[3.3]heptan-6-yl)-N2-methylpyrimidine-2,4-diamine

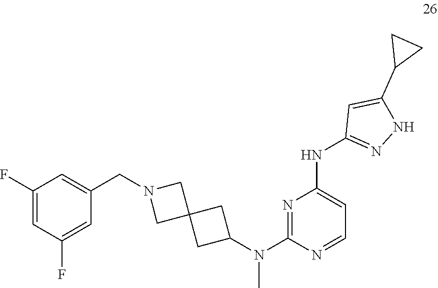 [SMILES: CN(c1nccc(Nc2cc(C3CC3)[nH]n2)n1)C1CC2(C1)CN(Cc1cc(F)cc(F)c1)C2]

Compound 26 was prepared according to the general procedure for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-(bromomethyl)-3,5-difluorobenzene, to afford the desired product as a solid (6 mg, 0.01 mmol, 7.5%). UPLC-MS (Basic Method, 4 min): rt 1.87 min, m/z: 452.3 [M+H]⁺. ¹H NMR (400 MHz, DMSO-d₆) δ 11.90 (s, 1H), 9.34 (s, 1H), 7.83 (d, J=5.6 Hz, 1H), 7.07 (tt, J=9.4, 2.4 Hz, 1H), 6.98 (d, J=6.5 Hz, 2H), 6.16 (s, 2H), 5.05 (p, J=8.8 Hz, 1H), 3.55 (s, 2H), 3.28 (s, 2H), 3.15 (s, 2H), 2.96 (s, 3H), 2.29 (s, 2H), 2.26 (s, 2H), 1.80 (tt, J=8.6, 4.9 Hz, 1H), 0.87-0.80 (m, 2H), 0.62 (dt, J=6.6, 3.2 Hz, 2H).

### Example 26: Synthesis of Compound 27

### N4-(5-Cyclopropyl-1H-pyrazol-3-yl)-N2-methyl-N2-(2-(3-(trifluoromethoxy)benzyl)-2-azaspiro[3.3]heptan-6-yl)pyrimidine-2,4-diamine

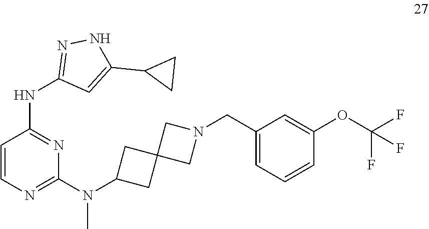 [SMILES: CN(c1nccc(Nc2cc(C3CC3)[nH]n2)n1)C1CC2(C1)CN(Cc1cccc(OC(F)(F)F)c1)C2]

Compound 27 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-(bromomethyl)-3-(trifluoromethoxy)benzene, to afford the desired product as a solid (32 mg, 0.06 mmol, 35%). UPLC-MS (Basic Method, 4 min): rt 2.03 min, m/z: 500.3 [M+H]⁺. ¹H NMR (400 MHz, DMSO-d₆) δ 11.91 (s, 1H), 9.37 (s, 1H), 7.83 (d, J=5.7 Hz, 1H), 7.47-7.40 (m, 1H), 7.30 (d, J=7.7 Hz, 1H), 7.24-7.20 (m, 2H), 6.16 (s, 1H), 5.05 (p, J=8.7 Hz, 1H), 3.58 (s, 2H), 3.27 (s, 2H), 3.14 (s, 2H), 2.96 (s, 3H), 2.28 (s, 2H), 2.26 (s, 2H), 1.78 (tt, J=8.7, 5.1 Hz, 1H), 0.85-0.76 (m, 2H), 0.65-0.58 (m, 2H).

### Example 27: Synthesis of Compound 28

### N4-(5-Cyclopropyl-1H-pyrazol-3-yl)-N2-methyl-N2-(2-(3-(methylsulfonyl)benzyl)-2-azaspiro[3.3]heptan-6-yl)pyrimidine-2,4-diamine

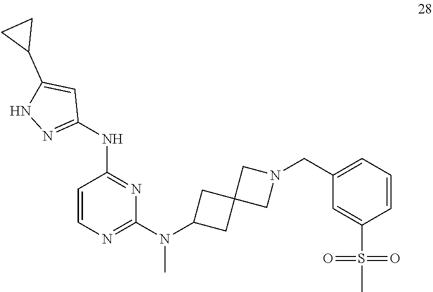 [SMILES: CN(c1nccc(Nc2cc(C3CC3)[nH]n2)n1)C1CC2(C1)CN(Cc1cccc(S(C)(=O)=O)c1)C2]

Compound 28 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-(bromomethyl)-3-(methylsulfonyl)benzene, to afford the desired product as a solid (15 mg, 0.03 mmol, 26%). UPLC-MS (Basic Method, 4 min): rt 1.52 min, m/z 494.2 [M+H]⁺. ¹H-NMR (400 MHz, DMSO) δ 11.90 (s, 1H), 9.33 (s, 1H), 7.86-7.77 (m, 3H), 7.64-7.56 (m, 2H), 6.17 (s, 2H), 5.06 (p, J=8.8 Hz, 1H), 3.65 (s, 2H), 3.29 (s, 2H), 3.20 (s, 3H), 3.16 (s, 2H), 2.96 (s, 3H), 2.28 (d, J=8.7 Hz, 4H), 1.78 (td, J=8.5, 4.3 Hz, 1H), 0.87-0.78 (m, 2H), 0.66-0.56 (m, 2H).

### Example 28: Synthesis of Compound 29

### N2-(2-(3-(Benzo[d]oxazol-2-yl)propyl)-2-azaspiro[3.3]heptan-6-yl)-N4-(5-cyclopropyl-1H-pyrazol-3-yl)-N2-methylpyrimidine-2,4-diamine

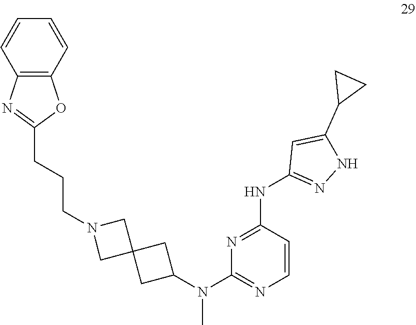 [SMILES: CN(c1nccc(Nc2cc(C3CC3)[nH]n2)n1)C1CC2(C1)CN(CCCc1nc3ccccc3o1)C2]

Compound 29 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 2-(3-chloropropyl)benzo[d]oxazole, to afford the desired product as a solid (4 g, 0.01 mmol, 3%). UPLC-MS (Basic Method, 4 min): rt 1.74 min, m/z 485.3 [M+H]⁺. ¹H-NMR (400 MHz, DMSO) δ 11.93 (s, 1H), 9.33 (s, 1H), 7.83 (d, J=5.7 Hz, 1H), 7.66 (tt, J=7.6, 2.6 Hz, 2H), 7.37-7.29 (m, 2H), 6.18 (s, 2H), 5.03 (p, J=8.7 Hz, 1H), 3.20 (s, 2H), 3.06 (s, 2H), 2.95 (s, 3H), 2.92 (d, J=7.3 Hz, 2H), 2.43 (t, J=6.8 Hz, 2H), 2.26-2.16 (m, 4H), 1.86 (td, J=8.5, 4.3 Hz, 1H), 1.78 (p, J=7.2 Hz, 2H), 1.24 (s, 1H), 0.91 (d, J=8.0 Hz, 2H), 0.69-0.61 (m, 2H).

### Example 29: Synthesis of Compound 30

### 2-(6-((4-((5-Cyclopropyl-1H-pyrazol-3-yl)amino)pyrimidin-2-yl)(methyl)amino)-2-azaspiro[3.3]heptan-2-yl)-1-(4-methylpiperazin-1-yl)ethan-1-one

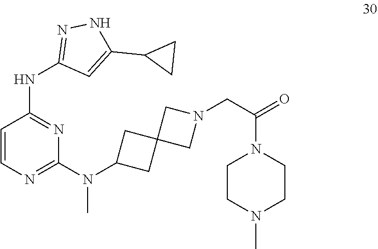 [SMILES: CN1CCN(C(=O)CN2CC3(CC(N(C)c4nccc(Nc5cc(C6CC6)[nH]n5)n4)C3)C2)CC1]

Compound 30 was prepared according to the general procedure according to Example 18 for the N-alkylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-(4-methylpiperazin-1-yl)propan-1-one hydrochloride, to afford the desired product as a solid (2.5 mg, 0.01 mmol, 4%). UPLC-MS: (Basic Method, 4 min) rt=1.26 min, m/z 466.4 [M+H]⁺. ¹H NMR (400 MHz, DMSO) δ 11.92 (s, 1H), 9.32 (s, 1H), 7.83 (d, J=5.7 Hz, 1H), 6.18 (s, 2H), 5.04 (p, J=8.8 Hz, 1H), 3.39 (s, 4H), 3.30 (s, 2H), 3.21 (s, 2H), 3.15 (s, 2H), 2.96 (s, 3H), 2.30-2.18 (m, 8H), 2.16 (s, 3H), 1.85 (td, J=8.6, 4.4 Hz, 1H), 0.92 (d, J=8.3 Hz, 2H), 0.65 (dt, J=6.7, 3.3 Hz, 2H).

### Example 30: General procedure for the N-acylation of N2-{2-Azaspiro[3.3]heptan-6-yl}-N4-(5-cyclopropyl-1H-pyrazol-3-yl)-N2-methylpyrimidine-2,4-diamine; bis(trifluoroacetic acid) from carboxylic acids

To a stirred solution of N2-{2-azaspiro[3.3]heptan-6-yl}-N4-(5-cyclopropyl-1H-pyrazol-3-yl)-N2-methylpyrimidine-2,4-diamine) (150 mg, 0.46 mmol, 1.0 eq) in N,N-dimethylformamide (0.8 mL) was added N,N-diisopropylethylamine followed by 1-methyl-1H-1,2,3-triazole-4-carboxylic acid (59 mg, 0.46 mmol, 1.0 eq) and hexafluoro-λ⁵-phosphanuide 1-[bis(dimethylamino)methylidene]-1H-1λ⁵-[1,2,3]triazolo[4,5-b]pyridin-3-ium-1-ylium-3-olate (210 mg, 0.55 mmol, 1.2 eq). The reaction mixture was left to stir overnight and was then purified by reverse phase prep HPLC to afford the desired product.

### Example 31: General procedure for the N-acylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) from acid chlorides

To a solution of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) (100 mg, 0.18 mmol, 1.0 eq) in THF (5.3 mL) was added potassium carbonate (100 mg, 0.72 mmol, 4.0 eq) and the resulting mixture was stirred at room temperature for 1 h. The mixture was then cooled to 0° C. and treated with solution of acid chloride (1.0 eq) in THF (1 mL). The ice bath was then removed and stirring was continued until the reaction was complete, as determined by LCMS analysis (1-18 h). The reaction mixture was partitioned between water (25 mL) and ethyl acetate (3×25 mL) and the combined organics were dried over Na₂SO₄, filtered, and concentrated to dryness. The crude residue was purified by reverse phase HPLC chromatography with a basic modifier, to afford the desired compound.

### Example 32. Synthesis of Compound 31

### (6-((4-((5-Cyclopropyl-1H-pyrazol-3-yl)amino)pyrimidin-2-yl)(methyl)amino)-2-azaspiro[3.3]heptan-2-yl)(1-methyl-1H-1,2,3-triazol-4-yl)methanone

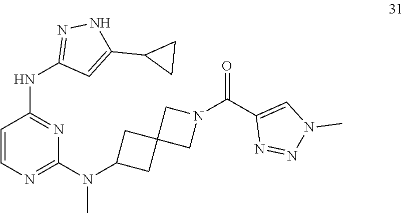 [SMILES: CN(c1nccc(Nc2cc(C3CC3)[nH]n2)n1)C1CC2(C1)CN(C(=O)c1cn(C)nn1)C2]

Compound 31 was prepared according to the general procedure according to Example 30 for the N-acylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-methyl-1H-1,2,3-triazole-4-carboxylic acid to afford the desired product as a solid (57 mg, 0.13 mmol, 29%). UPLC-MS (Basic Method, 4 min): rt 1.28 min, m/z 435.3 [M+H]⁺. ¹H-NMR (400 MHz, DMSO) δ 11.90 (s, 1H), 9.37 (s, 1H), 8.50 (d, J=2.6 Hz, 1H), 7.86 (dd, J=5.7, 1.6 Hz, 1H), 6.20 (s, 2H), 5.10 (p, J=8.9 Hz, 1H), 4.68 (s, 1H), 4.51 (s, 1H), 4.17 (s, 1H), 4.07 (s, 3H), 4.02 (s, 1H), 3.00 (s, 3H), 2.42 (dd, J=8.9, 3.3 Hz, 4H), 1.89 (tt, J=8.6, 4.2 Hz, 1H), 0.92 (ddt, J=10.6, 6.3, 3.1 Hz, 2H), 0.71-0.63 (m, 2H).

### Example 33. Synthesis of Compound 32

### (6-((4-((5-Cyclopropyl-1H-pyrazol-3-yl)amino)pyrimidin-2-yl)(methyl)amino)-2 azaspiro[3.3]heptan-2-yl)(tetrahydro-2H-pyran-4-yl)methanone

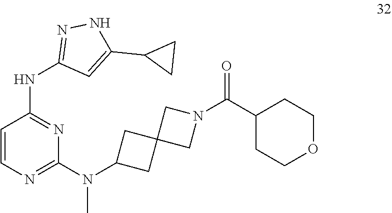 [SMILES: CN(c1nccc(Nc2cc(C3CC3)[nH]n2)n1)C1CC2(C1)CN(C(=O)C1CCOCC1)C2]

Compound 32 was prepared according to the general procedure according to Example 31 for the N-acylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using tetrahydro-2H-pyran-4-carbonyl chloride to afford the desired product as a solid (40 mg, 0.09 mmol, 51%). UPLC-MS (Basic Method, 4 min) rt 1.32 min, m/z 438.3 [M+H]⁺. ¹H NMR (400 MHz, DMSO) δ 11.96 (d, J=8.7 Hz, 1H), 9.38 (s, 1H), 7.87-7.80 (m, 1H), 6.19 (s, 2H), 5.07 (p, J=8.8 Hz, 1H), 4.30 (s, 1H), 4.14 (s, 1H), 3.96 (s, 1H), 3.85 (ddd, J=10.8, 6.8, 3.3 Hz, 2H), 3.80 (s, 1H), 3.31 (dt, J=6.4, 3.5 Hz, 2H), 2.98 (s, 3H), 2.43 (dd, J=8.8, 6.4 Hz, 1H), 2.37 (d, J=8.8 Hz, 4H), 1.87 (dd, J=8.9, 4.6 Hz, 1H), 1.51 (qd, J=8.6, 8.2, 4.0 Hz, 4H), 0.93 (d, J=8.1 Hz, 2H), 0.67 (tt, J=6.5, 3.1 Hz, 2H).

### Example 34. Synthesis of Compound 33

### (6-((4-((5-Cyclopropyl-1H-pyrazol-3-yl)amino)pyrimidin-2-yl)(methyl)amino)-2-azaspiro[3.3]heptan-2-yl)(4-methyl-1,2,3-thiadiazol-5-yl)methanone

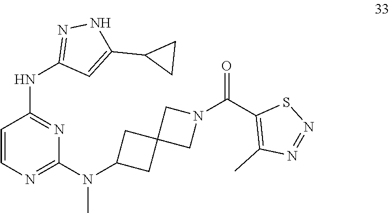 [SMILES: Cc1nnsc1C(=O)N1CC2(CC(N(C)c3nccc(Nc4cc(C5CC5)[nH]n4)n3)C2)C1]

Compound 33 was prepared according to the general procedure according to Example 31 for the N-acylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 4-methyl-1,2,3-thiadiazole-5-carbonyl chloride to afford the desired product as a solid (14 mg, 0.03 mmol, 17%). UPLC-MS (Basic Method, 4 min): rt 1.46 min, m/z 452.3 [M+H]⁺. ¹H NMR (400 MHz, DMSO) δ 11.93 (s, 1H), 9.35 (s, 1H), 7.85 (s, 1H), 6.18 (s, 2H), 5.14-5.01 (m, 1H), 4.34 (s, 1H), 4.20 (d, J=4.9 Hz, 2H), 4.05 (s, 1H), 3.01-2.96 (m, 3H), 2.78 (s, 3H), 2.42 (d, J=8.6 Hz, 4H), 1.88 (s, 1H), 0.93 (s, 1H), 0.84 (d, J=6.9 Hz, 1H), 0.66 (d, J=11.9 Hz, 2H).

### Example 35. Synthesis of Compound 34

### (6-((4-((5-Cyclopropyl-1H-pyrazol-3-yl)amino)pyrimidin-2-yl)(methyl)amino)-2 azaspiro[3.3]heptan-2-yl)(3-(trifluoromethyl)phenyl)methanone

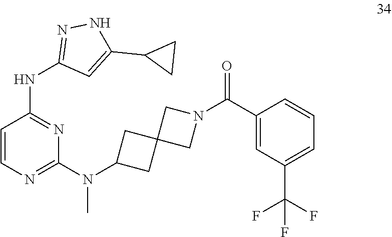 [SMILES: CN(c1nccc(Nc2cc(C3CC3)[nH]n2)n1)C1CC2(C1)CN(C(=O)c1cccc(C(F)(F)F)c1)C2]

Compound 34 was prepared according to the general procedure according to Example 31 for the N-acylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 3-(trifluoromethyl)benzoyl chloride to afford the desired product as a solid (39 mg, 0.08 mmol, 44%). UPLC-MS (Basic Method, 4 min): rt 1.76 min, m/z 498.2 [M+H]⁺. ¹H NMR (400 MHz, DMSO) δ 11.95 (s, 1H), 9.39 (s, 1H), 7.98-7.79 (m, 4H), 7.70 (t, J=7.7 Hz, 1H), 6.17 (s, 2H), 5.08 (dt, J=23.5, 8.7 Hz, 1H), 4.47 (s, 1H), 4.32 (s, 1H), 4.22 (s, 1H), 4.06 (s, 1H), 2.98 (d, J=8.2 Hz, 3H), 2.41 (d, J=8.6 Hz, 4H), 1.91-1.70 (m, 1H), 0.94 (d, J=8.2 Hz, 1H), 0.74 (s, 1H), 0.71-0.65 (m, 1H), 0.62-0.56 (m, 1H).

### Example 36. General procedure for the N-sulfonylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid)

Potassium carbonate (100 mg, 0.72 mmol, 4.0 eq) was added to a solution of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) (100 mg, 0.18 mmol, 1.0 eq) in THF (5 mL) at ambient temperature, and the resulting mixture was stirred for 1 h. The sulphonyl chloride (1.0 eq) was then added in a single portion at 0° C., and the resulting mixture was stirred at ambient temperature for 30 min. The reaction mixture was diluted with water (20 mL), basified with 1 M NaOH (10 mL) and extracted with EtOAc (3×20 mL). The combined organic extracts were dried over Na₂SO₄, filtered and concentrated under vacuum to afford the crude product, which was purified by automated flash column chromatography over silica gel (4 g Tellos cartridge) eluting with a solvent gradient of MeOH in DCM to afford the desired product.

### Example 37. Synthesis of Compound 35

### N4-(5-Cyclopropyl-1H-pyrazol-3-yl)-N2-methyl-N2-{2-[(1-methyl-1H-pyrazol-4-yl)sulfonyl]-2-azaspiro[3.3]heptan-6-yl}pyrimidine-2,4-diamine)

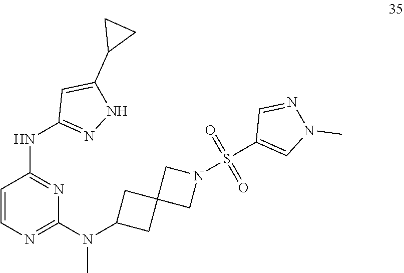 [SMILES: CN(c1nccc(Nc2cc(C3CC3)[nH]n2)n1)C1CC2(C1)CN(S(=O)(=O)c1cnn(C)c1)C2]

Compound 35 was prepared according to the general procedure according to Example 36 for the N-sulfonylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 1-methyl-1H-pyrazole-4-sulfonyl chloride) to afford the desired product as a solid (53 mg, 0.11 mmol, 63%). UPLC-MS (Basic Method, 4 min): rt 1.39 min, m/z 470.3 [M+H]⁺. ¹H NMR (400 MHz, DMSO) δ 11.95 (s, 1H), 9.35 (s, 1H), 8.44 (s, 1H), 7.89 (d, J=0.7 Hz, 1H), 7.83 (d, J=5.7 Hz, 1H), 6.16 (s, 2H), 4.95 (p, J=8.8 Hz, 1H), 3.93 (s, 3H), 3.79 (s, 2H), 3.64 (s, 2H), 2.92 (s, 3H), 2.28-2.18 (m, 2H), 2.11 (td, J=8.6, 2.8 Hz, 2H), 1.86 (td, J=8.5, 4.4 Hz, 1H), 0.94 (dd, J=8.4, 2.3 Hz, 2H), 0.73-0.61 (m, 2H).

### Example 38. Synthesis of Compound 36

### N4-(5-Cyclopropyl-1H-pyrazol-3-yl)-N2-methyl-N2-(2-((5,6,7,8-tetrahydroimidazo[1,2-a]pyridin-2-yl)sulfonyl)-2-azaspiro[3.3]heptan-6-yl)pyrimidine-2,4-diamine

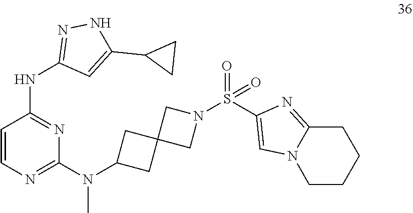 [SMILES: CN(c1nccc(Nc2cc(C3CC3)[nH]n2)n1)C1CC2(C1)CN(S(=O)(=O)c1cn3c(n1)CCCC3)C2]

Compound 36 was prepared according to the general procedure according to Example 36 for the N-sulfonylation of N2-{2-azaspiro[3.3]heptan-6-yl}-N4-(5-cyclopropyl-1H-pyrazol-3-yl)-N2-methylpyrimidine-2,4-diamine; bis(trifluoroacetic acid) using 5,6,7,8-tetrahydroimidazo[1,2-a]pyridine-2-sulfonyl chloride to afford the desired product as a solid (54 mg, 0.11 mmol, 59%). UPLC-MS (Basic Method, 4 min): rt 1.40 min, m/z 510.2 [M+H]⁺. ¹H NMR (400 MHz, DMSO) δ 11.95 (s, 1H), 9.36 (s, 1H), 7.82 (d, J=5.7 Hz, 1H), 7.75 (s, 1H), 6.17 (s, 2H), 4.93 (t, J=8.6 Hz, 1H), 4.02 (t, J=5.8 Hz, 2H), 3.95 (s, 2H), 3.81 (s, 2H), 2.92 (s, 3H), 2.76 (t, J=6.2 Hz, 2H), 2.27-2.17 (m, 2H), 2.11 (t, J=9.9 Hz, 2H), 1.92-1.81 (m, 5H), 0.96-0.89 (m, 2H), 0.68-0.60 (m, 2H).

### Example 39. Synthesis of Compound 37

### N4-(5-Cyclopropyl-1H-pyrazol-3-yl)-N2-(1-methylpiperidin-4-yl)pyrimidine-2,4-diamine

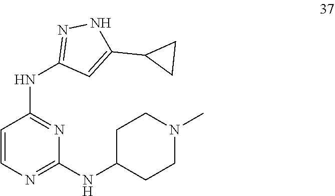 [SMILES: CN1CCC(Nc2nccc(Nc3cc(C4CC4)[nH]n3)n2)CC1]

A mixture of 2-chloro-N-(5-cyclopropyl-1H-pyrazol-3-yl)pyrimidin-4-amine (200 mg, 0.849 mmol, 1.0 eq), 1-methylpiperidin-4-amine (3.2 ml, 26 mmol, 30 eq) was heated in a microwave reactor at 130° C. for 30 min. The reaction mixture was purified directly by reverse phase HPLC chromatography under basic conditions to afford the desired compound as a solid (43 mg, 0.14 mmol, 16%). UPLC-MS (Basic Method, 4 min): rt 1.18 min, m/z 314.0 [M+H]⁺. ¹H-NMR (400 MHz, DMSO) δ 11.94 (s, 1H), 9.40 (s, 1H), 7.76 (s, 1H), 6.68-5.75 (m, 3H), 3.62 (s, 1H), 2.75 (s, 2H), 2.16 (s, 3H), 1.88 (d, J=41.2 Hz, 5H), 1.47 (d, J=12.5 Hz, 2H), 0.90 (s, 2H), 0.67 (s, 2H).

### Example 40. Synthesis of Compound 38

### N4-(5-Cyclopropyl-1H-pyrazol-3-yl)-N2-(1-cyclopropylpiperidin-4-yl)-N2-methylpyrimidine-2,4 diamine

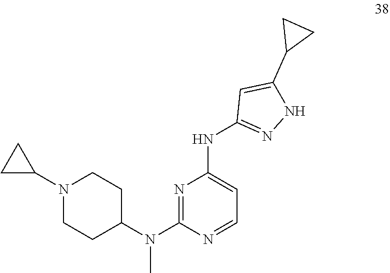 [SMILES: CN(c1nccc(Nc2cc(C3CC3)[nH]n2)n1)C1CCN(C2CC2)CC1]

A mixture of 2-chloro-N-(5-cyclopropyl-1H-pyrazol-3-yl)pyrimidin-4-amine (250 mg, 1.06 mmol, 1.0 eq), 1-cyclopropyl-N-methylpiperidin-4-amine (245 mg, 1.6 mmol, 1.5 eq), n-butanol (2.7 mL) and N,N-diisopropylethylamine (0.37 mL, 2.12 mmol, 2.0 eq) was heated at 125° C. for 4 h. The reaction mixture was diluted with water (20 mL) and extracted with EtOAc (3×20 mL). The combined organic extracts were dried over Na₂SO₄ and concentrated under reduced pressure to afford a black oil which was purified by reverse phase HPLC with a basic modifier to afford the desired compound as a solid (41 mg, 0.12 mmol, 11%). UPLC-MS (Basic Method, 4 min): rt 1.54 min, m/z 354.3 [M+H]⁺. ¹H NMR (400 MHz, DMSO-d₆) δ 11.95 (s, 1H), 9.35 (s, 1H), 7.83 (d, J=5.7 Hz, 1H), 6.33-5.97 (m, 2H), 4.63-4.50 (m, 1H), 3.04 (d, J=11.1 Hz, 2H), 2.90 (s, 3H), 2.26 (t, J=11.3 Hz, 2H), 1.87 (tt, J=8.7, 5.0 Hz, 1H), 1.73-1.49 (m, 5H), 0.98-0.89 (m, 2H), 0.72-0.66 (m, 2H), 0.43 (dt, J=6.1, 3.0 Hz, 2H), 0.32-0.27 (m, 2H).

### Example 41. Synthesis of Compound 39

### N4-(5-Cyclopropyl-1H-pyrazol-3-yl)-N2-methyl-N2-(1-(3-(methylsulfonyl)benzyl)piperidin-4-yl)pyrimidine-2,4-diamine

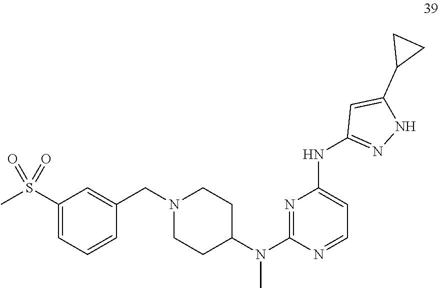 [SMILES: CN(c1nccc(Nc2cc(C3CC3)[nH]n2)n1)C1CCN(Cc2cccc(S(C)(=O)=O)c2)CC1]

Potassium carbonate (153 mg, 1.11 mmol, 4.0 eq) was added to a solution of N4-(5-cyclopropyl-1H-pyrazol-3-yl)-N2-methyl-N2-(piperidin-4-yl)pyrimidine-2,4-diamine; bis(trifluoroacetic acid) (150.0 mg, 0.277 mmol, 1.0 eq) in anhydrous DMF (3.0 mL) and the reaction mixture was stirred at room temperature for 10 min. 1-(Bromomethyl)-3-methanesulfonylbenzene (76 mg, 0.31 mmol, 1.1 eq) was added to the reaction at 0° C., and the resulting mixture was stirred at 0° C. for 1 h. The reaction mixture was diluted with water (20 mL), basified with a saturated aqueous NaHCO₃ solution (10 mL) and extracted with EtOAc (3×20 mL). The combined organic extracts were dried over Na₂SO₄ and concentrated under reduced pressure to give the crude product as a residue. This material was dissolved in MeOH (5 mL) and loaded on to an SCX cartridge, which was washed with MeOH (20 mL). The SCX cartridge was then flushed with 2 M NH₃ in MeOH (20 mL). The eluent was concentrated to afford the compound as an oil which was purified by reverse phase HPLC chromatography with a basic modifier to afford the desired compound (42.5 mg, 0.09 mmol, 32%). UPLC-MS (Basic Method, 4 min): rt 1.49 min, m/z 482.2 [M+H]⁺. ¹H NMR (400 MHz, DMSO-d₆) δ 11.92 (s, 1H), 9.36 (s, 1H), 7.87-7.80 (m, 3H), 7.71-7.66 (m, 1H), 7.62 (t, J=7.6 Hz, 1H), 6.29-6.00 (m, 2H), 4.62-4.50 (m, 1H), 3.62 (s, 2H), 3.22 (s, 3H), 2.98-2.90 (m, 5H), 2.11 (t, J=11.5 Hz, 2H), 1.90-1.71 (m, 3H), 1.57 (d, J=11.8 Hz, 2H), 0.98-0.91 (m, 2H), 0.71-0.65 (m, 2H).

### Example 42. Synthesis of Compound 40

### N4-(5-Cyclopropyl-1H-pyrazol-3-yl)-N2-(1-(3,5-difluorobenzyl)piperidin-4-yl)-N2-methylpyrimidine-2,4-diamine

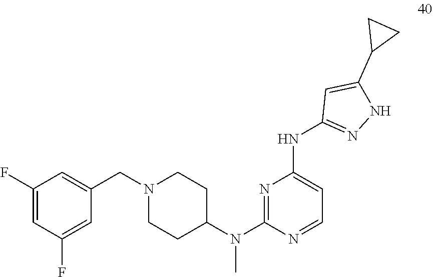 [SMILES: CN(c1nccc(Nc2cc(C3CC3)[nH]n2)n1)C1CCN(Cc2cc(F)cc(F)c2)CC1]

Potassium carbonate (102 mg, 0.74 mmol, 4.0 eq) was added to a solution of N4-(5-cyclopropyl-1H-pyrazol-3-yl)-N2-methyl-N2-(piperidin-4-yl)pyrimidine-2,4-diamine; bis(trifluoroacetic acid) (100 mg, 0.19 mmol, 1.0 eq) in anhydrous DMF (2.0 mL) and the reaction mixture was stirred at room temperature for 10 min. 1-(Bromomethyl)-3,5-difluorobenzene (26 μL, 0.20 mmol, 1.1 eq) was added to the reaction at 0° C., and the resulting mixture was stirred at 0° C. for 1 h. The reaction mixture was diluted with water (20 mL), basified with a saturated NaHCO₃ solution (10 mL) then extracted with EtOAc (3×20 mL). The combined organic extracts were dried over Na₂SO₄ and concentrated under reduced pressure to afford the crude product as a residue. This material was dissolved in methanol and loaded onto an SCX cartridge, which was washed with methanol (20 ml) and then eluted with 2M NH₃ in MeOH (20 mL). The eluent was concentrated to dryness under reduced pressure and the residue was purified by reverse phase HPLC purification with a basic modifier to afford the desired compound as a solid (34 mg, 0.08 mmol, 42%). UPLC-MS (Basic Method, 4 min): rt 1.95 min, m/z 440.3 [M+H]⁺. ¹H NMR (400 MHz, DMSO-d₆) δ 11.93 (s, 1H), 9.34 (s, 1H), 7.83 (d, J=5.7 Hz, 1H), 7.17-6.99 (m, 3H), 6.28-6.03 (m, 2H), 4.62-4.48 (m, 1H), 3.54 (s, 2H), 2.97-2.87 (m, 5H), 2.10 (t, J=11.4 Hz, 2H), 1.90-1.72 (m, 3H), 1.56 (d, J=11.7 Hz, 2H), 0.94 (d, J=8.2 Hz, 2H), 0.71-0.63 (m, 2H).

### Example 43: General Exemplary Schemes for the Preparation of Compounds of Formula I

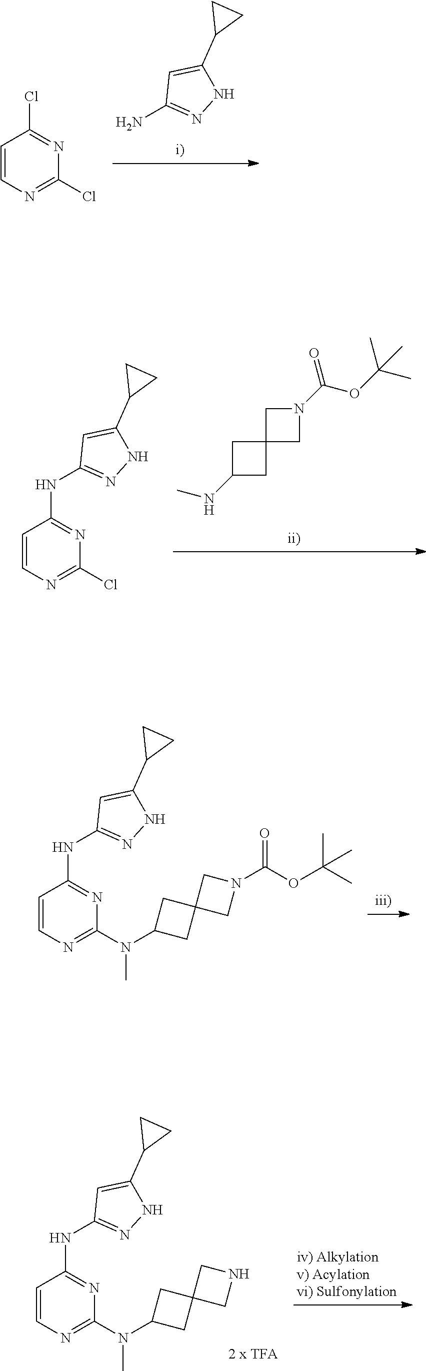 [SMILES: Clc1ccnc(Cl)n1 ; Nc1cc(C2CC2)[nH]n1 ; Clc1nccc(Nc2cc(C3CC3)[nH]n2)n1 ; CNC1CC2(C1)CN(C(=O)OC(C)(C)C)C2 ; CN(c1nccc(Nc2cc(C3CC3)[nH]n2)n1)C1CC2(C1)CN(C(=O)OC(C)(C)C)C2 ; CN(c1nccc(Nc2cc(C3CC3)[nH]n2)n1)C1CC2(CNC2)C1]

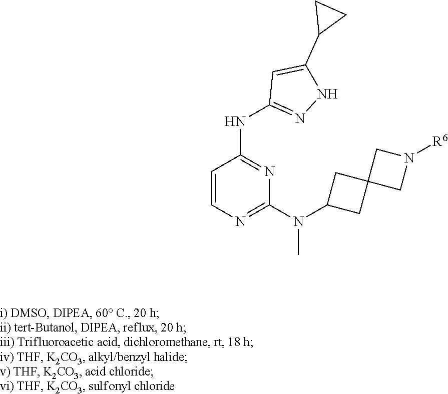

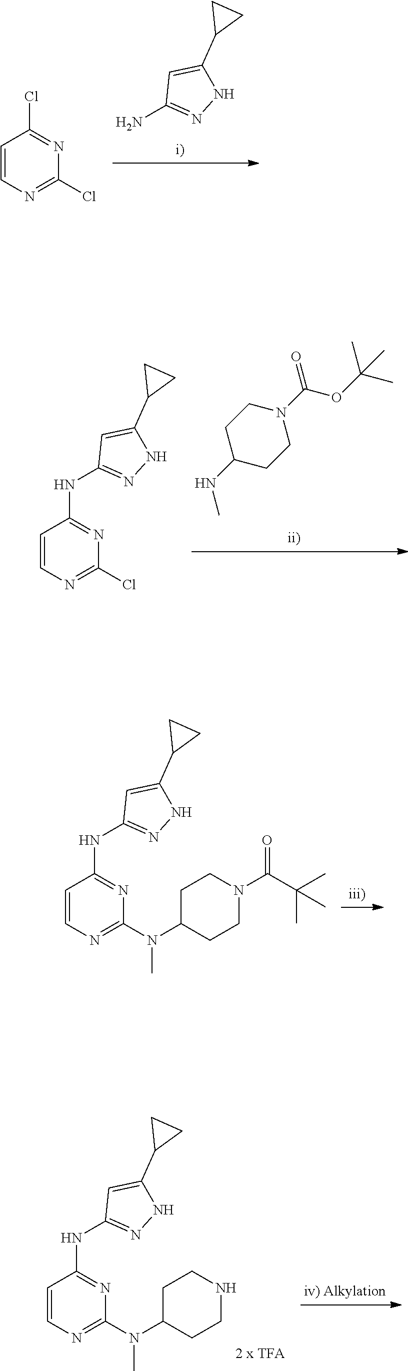 [SMILES: Clc1ccnc(Cl)n1 ; Nc1cc(C2CC2)[nH]n1 ; Clc1nccc(Nc2cc(C3CC3)[nH]n2)n1 ; CNC1CCN(C(=O)OC(C)(C)C)CC1 ; CN(c1nccc(Nc2cc(C3CC3)[nH]n2)n1)C1CCN(C(=O)C(C)(C)C)CC1 ; CN(c1nccc(Nc2cc(C3CC3)[nH]n2)n1)C1CCNCC1]

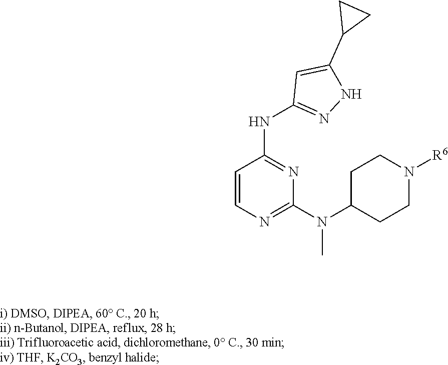

### Example 44: Synthesis of Compound 41

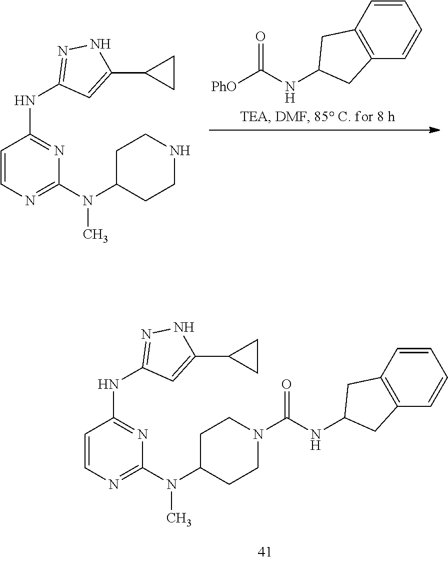 [SMILES: CN(c1nccc(Nc2cc(C3CC3)[nH]n2)n1)C1CCNCC1 ; O=C(NC1Cc2ccccc2C1)Oc1ccccc1 ; CN(c1nccc(Nc2cc(C3CC3)[nH]n2)n1)C1CCN(C(=O)NC2Cc3ccccc3C2)CC1]

Compound 41 was prepared from N4-(5-Cyclopropyl-1H-pyrazol-3-yl)-N2-methyl-N2-(piperidin-4-yl)pyrimidine-2,4-diamine according to general scheme shown in Example 43.

### Example 45: Synthesis of Compound 42

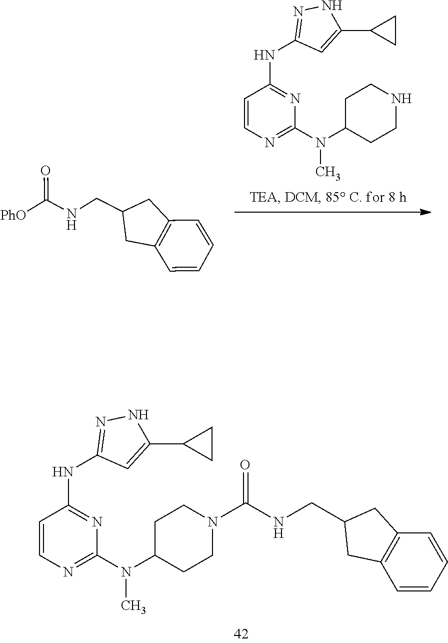 [SMILES: O=C(NCC1Cc2ccccc2C1)Oc1ccccc1 ; CN(c1nccc(Nc2cc(C3CC3)[nH]n2)n1)C1CCNCC1 ; CN(c1nccc(Nc2cc(C3CC3)[nH]n2)n1)C1CCN(C(=O)NCC2Cc3ccccc3C2)CC1]

Compound 42 was prepared from N4-(5-Cyclopropyl-1H-pyrazol-3-yl)-N2-methyl-N2-(piperidin-4-yl)pyrimidine-2,4-diamine according to general scheme shown in Example 43.

### Example 46: Synthesis of Compound 43

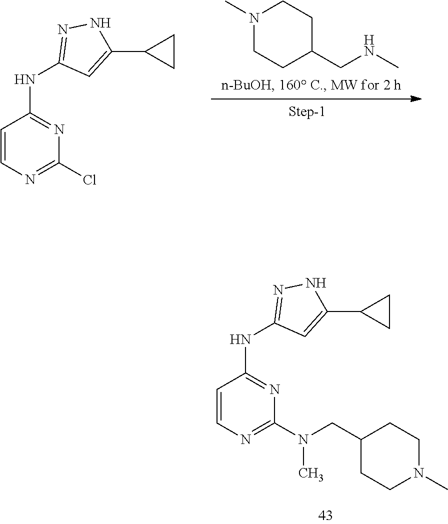

Compound 43 was prepared from 2-chloro-N-(5-cyclopropyl-1H-pyrazol-3-yl)pyrimidin-4-amine and N-methyl-1-(1-methylpiperidin-4-yl)methanamine. Yield: 80 mg. Purity (HPLC) 98.4%, MS (m/e 342).

### Example 47: Synthesis of Compound 44

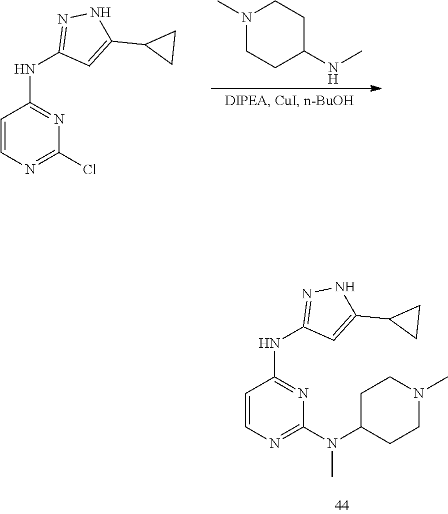

Compound 44 was prepared from 2-chloro-N-(5-cyclopropyl-1H-pyrazol-3-yl)pyrimidin-4-amine and N,1-dimethylpiperidin-4-amine. Yield: 25 mg. Purity (LCMS) 96.5%, MS (m/e 328).

### Example 48: Synthesis of Compound 45

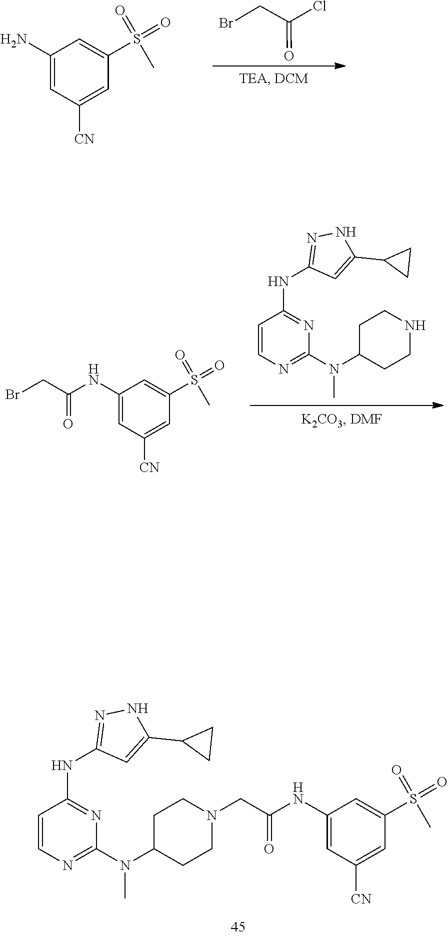

Compound 45 was prepared from N4-(5-Cyclopropyl-1H-pyrazol-3-yl)-N2-methyl-N2-(piperidin-4-yl)pyrimidine-2,4-diamine according to general scheme shown in Example 43.

### Synthesis of Intermediates

### Synthesis of 2-Chloro-N-(5-cyclopropyl-1H-pyrazol-3-yl)pyrimidin-4-amine

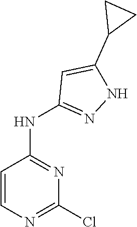 [SMILES: Clc1nccc(Nc2cc(C3CC3)[nH]n2)n1]

A solution of 2,4-dichloropyrimidine (1.0 g, 6.7 mmol, 1.0 eq) in anhydrous DMSO (10.0 mL) was treated sequentially with 5-cyclopropyl-1H-pyrazol-3-amine (0.79 mL, 7.38 mmol, 1.10 eq) and N,N-diisopropylethylarine (1.8 mL, 10.07 mmol, 1.50 eq), and the resulting solution was stirred at 60° C. for 20 h. The reaction mixture was cooled to ambient temperature and poured into ice water, affording a suspension, which was stirred for 5 min. The mixture was filtered to afford a solid, which was washed with water (50 mL) before being dried under reduced pressure to constant weight to give the desired compound as a solid (1.29 g, 5.47 mmol, 81%). UPLC-MS (Basic Method, 2 min): rt 0.87, m/z 236.3 [M+H]⁺. ¹H NMR (400 MHz, DMSO) δ 12.19 (s, 1H), 10.29 (s, 1H), 8.15 (s, 1H), 1.89 (tt, J=8.5, 5.1 Hz, 1H), 0.97-0.84 (m, 2H), 0.72-0.64 (m, 2H).

### Synthesis of 2-Chloro-N-(5-cyclopentyl-1H-pyrazol-3-yl)pyrimidin-4-amine

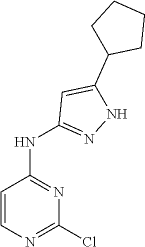 [SMILES: Clc1nccc(Nc2cc(C3CCCC3)[nH]n2)n1]

A solution of 2,4-dichloropyrimidine (450 mg, 3.0 mmol, 1.0 eq) in anhydrous DMSO (10.0 mL) was treated sequentially with 5-cyclopentyl-1H-pyrazol-3-amine (502 mg, 3.2 mmol, 1.10 eq) and N,N-diisopropylethylamine (0.79 mL, 4.5 mmol, 1.50 eq), and the resulting solution was stirred at 60° C. for 20 h. The reaction mixture was cooled to ambient temperature and poured into ice water, affording a suspension, which was stirred for 5 min. The mixture was filtered to afford a solid, which was washed with water (50 mL) before being dried under vacuum to constant weight to give the desired compound as a solid (675 mg, 2.56 mmol, 85%). UPLC-MS (Basic Method, 2 min): rt 0.98 min, m/z=263.3 [M+H]⁺. ¹H NMR (400 MHz, DMSO) δ 12.19 (s, 1H), 10.31 (s, 1H), 8.16 (s, 1H), 3.02 (q, J=8.3 Hz, 1H), 2.05-1.94 (m, 2H), 1.74-1.66 (m, 2H), 1.58 (ddt, J=20.3, 15.7, 7.0 Hz, 4H).

### Synthesis of tert-Butyl 6-((4-((5-cyclopentyl-1H-pyrazol-3-yl)amino)pyrimidin-2-yl)(methyl)amino)-2-azaspiro[3.3]heptane-2-carboxylate

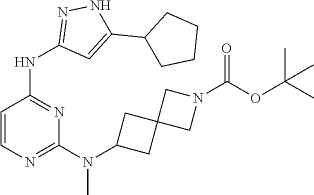 [SMILES: CN(c1nccc(Nc2cc(C3CCCC3)[nH]n2)n1)C1CC2(C1)CN(C(=O)OC(C)(C)C)C2]

A mixture of 2-chloro-N-(5-cyclopropyl-1H-pyrazol-3-yl)pyrimidin-4-amine (1.5 g, 6.37 mmol, 1.0 eq) tert-butyl 6-(methylamino)-2-azaspiro[3.3]heptane-2-carboxylate (2.9 g, 12.73 mmol, 2.0 eq) and N,N-diisopropylethylamine (3.3 mL, 19.09 mmol, 3.0 eq) in ethanol (15 mL) was heated in a microwave reactor at 120° C. power=50 for 15 h (The reaction was performed in 3 batches of 0.5 g each). The solvent was removed under reduced pressure and the crude residue was purified by Teledyne (silica) DCM:MeOH 0 to 15% over 15 CV to afford the desired product as a solid (2.5 g, 5.88 mmol, 92%). UPLC-MS (Basic Method, 2 min): rt 1.11 min, 426.4 [M+H]⁺. ¹H NMR (400 MHz, DMSO) δ 12.00 (s, 1H), 9.51 (s, 1H), 7.84 (d, J=5.8 Hz, 1H), 6.20 (s, 1H), 5.02 (t, J=8.9 Hz, 1H), 3.97 (s, 2H), 3.80 (s, 2H), 2.97 (s, 3H), 2.34 (d, J=8.8 Hz, 4H), 1.86 (dq, J=8.8, 5.2, 4.5 Hz, 1H), 1.37 (s, 9H), 0.97-0.89 (m, 2H), 0.72-0.63 (m, 2H).

### Synthesis of N4-(5-Cyclopentyl-1H-pyrazol-3-yl)-N2-methyl-N2-(2-azaspiro[3.3]heptan-6-yl)pyrimidine-2,4-diamine

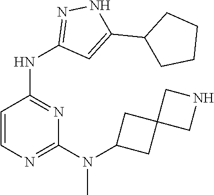 [SMILES: CN(c1nccc(Nc2cc(C3CCCC3)[nH]n2)n1)C1CC2(CNC2)C1]

To a solution of tert-butyl 6-({4-[(5-cyclopentyl-1H-pyrazol-3-yl)amino]pyrimidin-2-yl}(methyl)amino)-2-azaspiro[3.3]heptane-2-carboxylate (385 mg, 0.85 mmol, 1.0 eq) in dichloromethane (5 mL) was added trifluoroacetic acid (0.5 mL, 6.53 mmol, 7.69 eq). The resulting solution was stirred at room temperature for 18 h. The reaction mixture was then concentrated to dryness under reduced pressure and the residue was loaded onto an SCX column, washed with methanol and then eluted with ammonia in methanol. The solvent was removed under reduced pressure to afford the desired compound as a semi-solid (300 mg, 0.85 mmol, 100%). UPLC-MS (Basic Method, 2 min): rt 0.88 min, m/z 354.3 [M+H]⁺. ¹H NMR (400 MHz, DMSO) δ 11.93 (s, 1H), 9.40 (s, 1H), 7.84 (d, J=5.7 Hz, 1H), 6.24 (d, J=53.2 Hz, 2H), 5.12-4.99 (m, 1H), 3.54 (s, 2H), 3.42 (s, 2H), 3.00 (d, J=8.0 Hz, 1H), 2.96 (s, 3H), 2.34-2.19 (m, 4H), 2.06-1.94 (m, 2H), 1.75-1.67 (m, 2H), 1.66-1.51 (m, 4H).

### Synthesis of tert-Butyl 6-({4-[(5-cyclopropyl-1H-pyrazol-3-yl)amino]pyrimidin-2-yl}(methyl)amino)-2-azaspiro[3.3]heptane-2-carboxylate

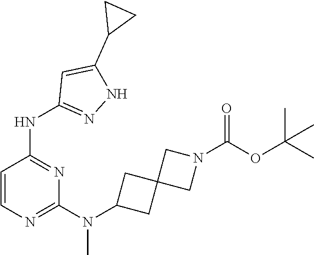 [SMILES: CN(c1nccc(Nc2cc(C3CC3)[nH]n2)n1)C1CC2(C1)CN(C(=O)OC(C)(C)C)C2]

A mixture of 2-chloro-N-(5-cyclopropyl-1H-pyrazol-3-yl)pyrimidin-4-amine (400 mg, 1.7 mmol, 1.0 eq), tert-butyl 6-(methylamino)-2-azaspiro[3.3]heptane-2-carboxylate (768 mg, 3.4 mmol, 2.0 eq) and N,N-diisopropylethylanine (0.89 mL, 5.1 mmol, 3.0 eq) in tert-butanol (15 mL) was heated at reflux for 20 h. The reaction was cooled to ambient temperature and evaporated to dryness under vacuum to give the crude product. Purification by automated column chromatography over silica, eluting with a gradient of MeOH in DCM (0 to 15%) afforded the desired product as a semi-solid (530 mg, 1.17 mmol, 73%). UPLC-MS (Basic Method, 2 min): rt 1.11 min, m/z 426.4 [M+H]⁺. ¹H NMR (400 MHz, DMSO) δ 12.00 (s, 1H), 9.51 (s, 1H), 7.84 (d, J=5.8 Hz, 1H), 6.20 (s, 1H), 5.02 (t, J=8.9 Hz, 1H), 3.97 (s, 2H), 3.80 (s, 2H), 2.97 (s, 3H), 2.34 (d, J=8.8 Hz, 4H), 1.86 (dq, J=8.8, 5.2, 4.5 Hz, 1H), 1.37 (s, 9H), 0.97-0.89 (m, 2H), 0.72-0.63 (m, 2H).

### Synthesis of N2-{2-Azaspiro[3.3]heptan-6-yl}-N4-(5-cyclopropyl-1H-pyrazol-3-yl)-N2-methylpyrimidine-2,4-diamine; bis(trifluoroacetic acid)

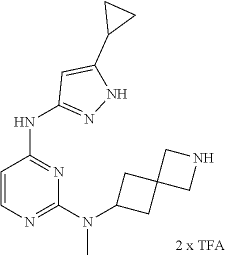 [SMILES: CN(c1nccc(Nc2cc(C3CC3)[nH]n2)n1)C1CC2(CNC2)C1]

Trifluoroacetic acid (1.7 mL, 22 mmol, 7.7 eq) was added to a solution of tert-butyl 6-({4-[(5-cyclopropyl-1H-pyrazol-3-yl)amino]pyrimidin-2-yl}(methyl)amino)-2-azaspiro[3.3]heptane-2-carboxylate (1.07 g, 2.5 mmol, 1.0 eq) in DCM (15 mL) at ambient temperature, and the resulting solution was stirred for 18 h. The solvent was removed under vacuum to afford the crude product as a solid, which was triturated with diethyl ether (20 mL) to afford the desired product as a solid (1.4 g, 2.5 mmol, 90%). UPLC-MS (Basic Method, 2 min): rt 0.74 min, 326.4 [M+H]⁺. ¹H NMR (400 MHz, DMSO) δ12.45 (s, 1H) 11.05 (s, 1H), 8.68 (s, 2H), 7.85 (s, 1H), 6.34 (s, 2H), 4.75 (s, 1H), 4.07 (t, J=6.2 Hz, 2H), 3.95 (t, J=6.1 Hz, 2H), 3.06 (s, 3H), 2.55-2.45 (m, 4H), 1.93 (tt, J=8.7, 4.8 Hz, 1H), 1.02-0.92 (m, 2H), 0.75-0.67 (m, 2H).

### Synthesis of 1-(4-((4-((5-Cyclopropyl-1H-pyrazol-3-yl)amino)pyrimidin-2-yl)(methyl)amino)piperidin-1-yl)-2,2-dimethylpropan-1-one

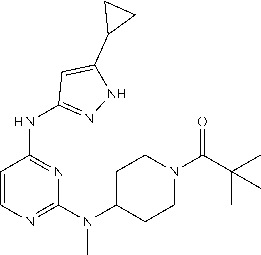 [SMILES: CN(c1nccc(Nc2cc(C3CC3)[nH]n2)n1)C1CCN(C(=O)C(C)(C)C)CC1]

To a solution of 2-chloro-N-(5-cyclopropyl-1H-pyrazol-3-yl)pyrimidin-4-amine (2.16 g, 9.19 mmol, 1 eq) and tert-butyl 4-(methylamino)piperidine-1-carboxylate (2.95 g, 13.8 mmol, 1.5 eq) in n-Butanol (25 mL) was added N,N-diisopropylethylamine (3.2 mL, 18.4 mmol, 2.0 eq). The reaction mixture was heated to reflux and stirred for 28 h. The reaction mixture was diluted with H₂O (100 mL) and extracted with EtOAc (3×100 mL). The combined organic extracts were dried over Na₂SO₄, then concentrated in vacuo to afford an amorphous solid which was purified by reverse phase flash column chromatography (C18 400 g cartridge), using a gradient of 5:95 MeCN:H₂O to 95:5 MeCN:H₂O, over 15 column volumes, to afford the desired compound as a solid (770 mg, 1.94 mmol, 20%). UPLC-MS (Basic Method, 4 min): rt 1.80 min, m/z 414.3 [M+H]⁺. ¹H NMR (400 MHz, DMSO-d₆) δ 11.93 (s, 1H), 9.34 (s, 1H), 7.85 (d, J=5.7 Hz, 1H), 6.18 (s, 2H), 4.72 (p, J=7.4 Hz, 1H), 4.09 (d, J=12.8 Hz, 2H), 2.91 (s, 3H), 2.78 (s, 3H), 1.86 (tt, J=8.4, 5.0 Hz, 1H), 1.62-1.54 (m, 4H), 1.41 (s, 9H), 0.95-0.87 (m, 2H), 0.67-0.62 (m, 2H).

### Synthesis of N4-(5-Cyclopropyl-1H-pyrazol-3-yl)-N2-methyl-N2-(piperidin-4-yl)pyrimidine-2,4-diamine; bis(trifluoroacetic acid)

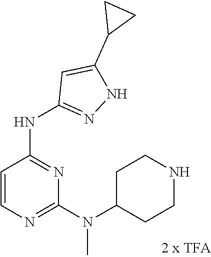 [SMILES: CN(c1nccc(Nc2cc(C3CC3)[nH]n2)n1)C1CCNCC1]

To a solution of tert-butyl 1-(4-((4-((5-cyclopropyl-1H-pyrazol-3-yl)amino)pyrimidin-2-yl)(methyl)amino)piperidin-1-yl)-2,2-dimethylpropan-1-one (755 mg, 1.83 mmol, 1.0 eq) in dichloromethane (10 mL) was added trifluoroacetic acid (10 mL) over 2 min at 0° C. The reaction was kept at 0° C. and stirred for 30 min. The reaction mixture was concentrated under reduced pressure and the resulting gum was suspended in diethyl ether Et₂O (15 mL) and sonicated until it became a free-flowing precipitate which was then isolated by filtration to afford the desired compound as a solid (960 mg, 1.77 mmol, 97%). UPLC-MS (Basic Method, 4 min): rt: 1.10 min, m/z 314.2 [M+H]⁺. ¹H NMR (400 MHz, DMSO-d₆, 90° C.) δ 10.26 (s, 1H), 7.87 (d, J=6.8 Hz, 1H), 6.50 (d, J=6.7 Hz, 1H), 6.14 (s, 1H), 4.64 (tt, J=11.8, 4.1 Hz, 1H), 3.54-3.45 (m, 2H), 3.03 (s, 3H), 2.97 (td, J=12.8, 3.1 Hz, 2H), 2.04 (qd, J=13.1, 4.3 Hz, 2H), 1.97-1.88 (m, 3H), 0.99-0.92 (m, 2H), 0.74-0.69 (m, 2H).

### Example 101: Synthesis of Compound 101

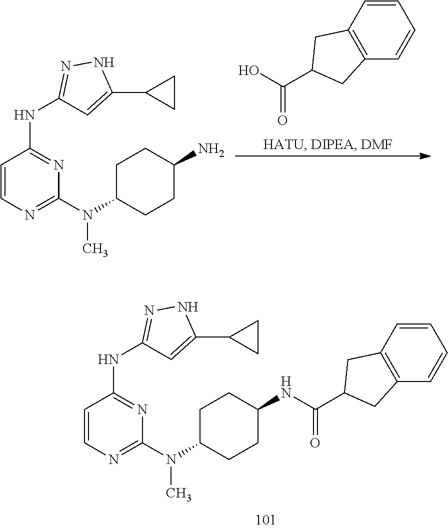 [SMILES: CN(c1nccc(Nc2cc(C3CC3)[nH]n2)n1)[C@H]1CC[C@H](N)CC1 ; O=C(O)C1Cc2ccccc2C1 ; CN(c1nccc(Nc2cc(C3CC3)[nH]n2)n1)[C@H]1CC[C@H](NC(=O)C2Cc3ccccc3C2)CC1]

### Synthesis of N-((1R,4R)-4-((4-((5-cyclopropyl-1H-pyrazol-3-yl)amino)pyrimidin-2-yl)(methyl)amino)cyclohexyl)-2,3-dihydro-1H-indene-2-carboxamide

To a stirred solution of N2-((1R,4R)-4-aminocyclohexyl)-N4-(5-cyclopropyl-1H-pyrazol-3-yl)-N2-methylpyrimidine-2,4-diamine (150 mg, 0.458 mmol), 2,3-dihydro-1H-indene-2-carboxylic acid (74.3 mg, 0.458 mmol) in dry DMF (2 mL) was added DIPEA (0.239 mL, 1.376 mmol) drop-wise at 0° C. followed by HATU (348.6 mg, 0.917 mmol). The reaction was stirred at room temperature for 16 h. After completion of the reaction (monitored by LCMS), the reaction mixture was diluted with dichloromethane, washed with water and brine, dried over anhydrous Na₂SO₄ and concentrated to yield the residue. The residue was purified by reverse phase preparative HPLC using Sunfire C18 column with mobile phase 0.1% TFA in water/acetonitrile to yield N-((1R,4R)-4-((4-((5-cyclopropyl-1H-pyrazol-3-yl) amino)pyrimidin-2-yl)(methyl)amino)cyclohexyl)-2,3-dihydro-1H-indene-2-carboxamide (50 mg, 23.14%). LC purity: 98.87%; m/z: 472.2 [M+H]⁺ (Mol. formula C₂₇H₃₃N₇O, calcd. mol. wt. 471.61). ¹H NMR (400 MHz, CD₃OD): δ 7.73 (d, J=6.7 Hz, 1H), 7.20-7.17 (m, 2H), 7.15-7.12 (m, 2H), 6.38-6.36 (m, 2H), 3.75-3.69 (m, 1H), 3.27-3.12 (m, 5H), 3.09 (s, 3H), 2.17-2.14 (m, 2H), 1.96-1.87 (m, 5H), 1.53-1.47 (m, 3H), 1.07-1.05 (m, 2H), 0.93-0.87 (m, 2H).

### Example 102: Synthesis of Compound 102

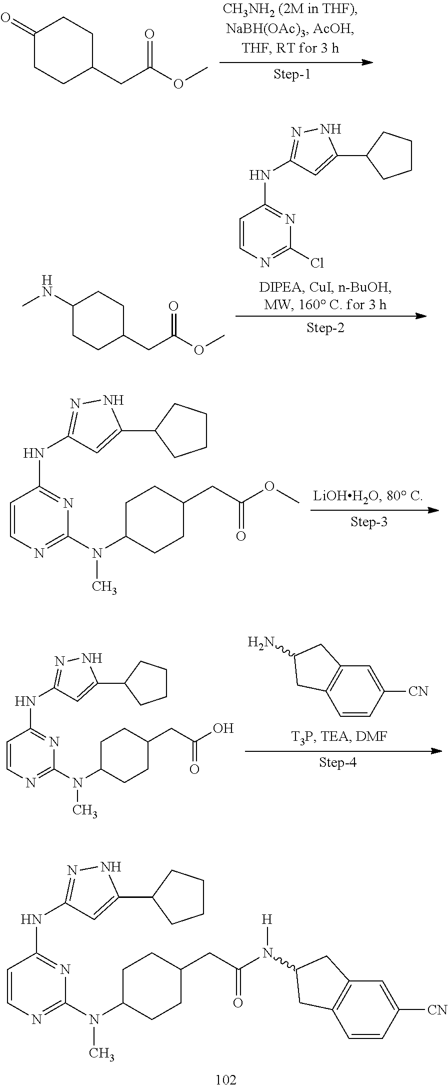

### Step-1: Synthesis of methyl 2-(4-(methylamino)cyclohexyl)acetate

Methyl 2-(4-oxocyclohexyl)acetate (1 g, 5.882 mmol) in THF (20 mL) was added methyl amine (5.8 mL, 11.764 mmol, 2M solution in THF), acetic acid (0.35 mL, 5.882 mmol) and NaBH(OAc)₃ (1.24 g, 5.882 mmol) at room temperature and the reaction mixture was stirred for 3 h at room temperature. The progress of the reaction was monitored by LCMS. After completion of the starting material, the reaction was quenched with saturated sodium bicarbonate and extracted with DCM, washed with brine, dried over anhydrous Na₂SO₄ and concentrated to obtain methyl 2-(4-(methylamino)cyclohexyl)acetate) (700 mg, crude). LC purity: 99%; m/z: 186.14 [M+H]⁺ (Mol. formula C₁₀H₁₉NO₂, calcd. mol. wt. 185.27).

### Step-2: Synthesis of methyl 2-(4-((4-((5-cyclopentyl-1H-pyrazol-3-yl)amino)pyrimidin-2-yl)(methyl)amino)cyclohexyl)acetate

To a stirred solution of methyl 2-(4-(methylamino)cyclohexyl)acetate (500 mg, 2.702 mmol) in n-BuOH (5 mL) in a 20 mL microwave vial was added DIPEA (0.68 mL, 4.053 mmol), 2-chloro-N-(5-cyclopentyl-1H-pyrazol-3-yl)pyrimidin-4-amine (355.4 mg, 1.351 mmol) and copper iodide (50 mg). The reaction mixture was heated in a microwave reactor at 160° C. for 3 h. After completion of the reaction, (monitored by LCMS), the reaction mixture was concentrated to obtain crude compound. The crude product was purified by Biotage Isolera using silica gel (230-400 mesh) with gradient elution of 0-100% ethyl acetate in pet ether to afford methyl 2-(4-((4-((5-cyclopentyl-1H-pyrazol-3-yl)amino)pyrimidin-2-yl)(methyl)amino)cyclohexyl)acetate (250 mg, 22%). LC purity: 93.1%; m/z: 413.26 [M+H]⁺ (Mol. formula C₂₂H₃₂N₆O₂, calcd. mol. wt. 412.54).

### Step-3: Synthesis of methyl 2-(4-((4-((5-cyclopentyl-1H-pyrazol-3-yl)amino)pyrimidin-2-yl)(methyl)amino)cyclohexyl)acetic acid

To a stirred solution of methyl 2-(4-((4-((5-cyclopentyl-1H-pyrazol-3-yl)amino)pyrimidin-2-yl)(methyl)amino)cyclohexyl)acetate (250 mg, 0.606 mmol) in water, THF and methanol (1:1:1, 3 mL) was added lithium hydroxide monohydrate (127.42 mg, 3.033 mmol). The reaction was heated to 80° C. for 16 h. After completion of the reaction (monitored by LCMS), the reaction mixture was concentrated to obtain 2-(4-((4-((5-cyclopentyl-1H-pyrazol-3-yl)amino)pyrimidin-2-yl)(methyl)amino)cyclohexyl)acetic acid (200 mg, crude). LC purity: 90.0%; m/z: 399.25 [M+H]⁺ (Mol. formula C₂₁H₃₀N₆O₂, calcd. mol. wt. 398.51).

### Step-4: Synthesis of N-(5-cyano-2,3-dihydro-1H-inden-2-yl)-2-(4-((4-((5-cyclopentyl-1H-pyrazol-3-yl)amino)pyrimidin-2-yl)(methyl)amino)cyclohexyl)acetamide

To a stirred solution of methyl 2-(4-((4-((5-cyclopentyl-1H-pyrazol-3-yl)amino)pyrimidin-2-yl)(methyl)amino)cyclohexyl)acetic acid (200 mg, 0.52 mmol) in dry DMF (2 mL) was added triethylamine (0.21 mL, 1.56 mmol) and 2-amino-2,3-dihydro-1H-indene-5-carbonitrile (97.48 mg, 0.52 mmol), T3P (0.23 mL, 0.78 mmol, 50% solution in EtOAc). The reaction mixture was stirred at room temperature for 16 h. The progress of the reaction was monitored by TLC, and after complete consumption of starting material, the reaction mixture was diluted with water and extracted with dichloromethane. The resulting organic layer was washed with brine then dried over anhydrous Na₂SO₄ and concentrated to obtain crude compound. The crude compound was purified by reverse phase preparative HPLC using Sunfire C18 column with mobile phase 0.1% TFA in water/acetonitrile to yield N-(5-cyano-2,3-dihydro-1H-inden-2-yl)-2-(4-((4-((5-cyclopentyl-1H-pyrazol-3-yl)amino)pyrimidin-2-yl)(methyl)amino)cyclohexyl)acetamide (50 mg, 18%). LC purity: 99.2%; m/z: 539.32 [M+H]⁺ (Mol. formula C₃₁H₃₈N₈O, calcd. mol. wt. 538.70). ¹H NMR (400 MHz, CD₃OD): δ 7.72 (d, J=7.2 Hz, 1H), 7.61-7.54 (m, 2H), 7.0-6.49 (m, 1H), 6.51-5.34 (m, 2H), 4.68-4.64 (m, 1H), 3.39-3.32 (m, 1H), 3.15-3.08 (m, 4H), 2.97-2.90 (m, 2H), 2.36 (d, J=7.2 Hz, 1H), 2.32-2.11 (m, 4H), 1.84-1.59 (m, 15H), 1.42-1.30 (m, 1H).

### Example 103: Synthesis of Compound 103

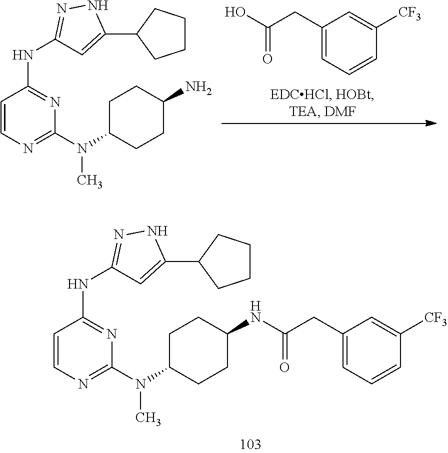 [SMILES: CN(c1nccc(Nc2cc(C3CCCC3)[nH]n2)n1)[C@H]1CC[C@H](N)CC1 ; O=C(O)Cc1cccc(C(F)(F)F)c1 ; CN(c1nccc(Nc2cc(C3CCCC3)[nH]n2)n1)[C@H]1CC[C@H](NC(=O)Cc2cccc(C(F)(F)F)c2)CC1]

### Synthesis of N-((1R,4R)-4-((4-((5-cyclopentyl-1H-pyrazol-3-yl)amino)pyrimidin-2-yl)(methyl)amino)cyclohexyl)-2-(3-(trifluoromethyl)phenyl)acetamide

To a solution of 2-(3-(trifluoromethyl)phenyl)acetic acid (172 mg, 0.845 mmol) in dry DMF (6 mL) was added triethylamine (0.23 mL, 1.69 mmol) drop-wise followed by the addition of EDC.HCl (214 mg, 1.12 mmol) and HOBt (114 mg, 0.845 mmol). The reaction mixture was stirred for 15 min and then N2-((1R,4R)-4-aminocyclohexyl)-N4-(5-cyclopentyl-1H-pyrazol-3-yl)-N2-methylpyrimidine-2,4-diamine (200 mg, 0.563 mmol) was added. The reaction mixture was stirred at room temperature for 16 h. The progress of the reaction was monitored by TLC. After completion of the reaction, the reaction mixture was diluted with dichloromethane washed with water and brine and dried over anhydrous Na₂SO₄ and concentrated to obtain the crude compound. The crude product was purified by reverse phase preparative HPLC using Sunfire C18 column with mobile phase 0.1% TFA in water/acetonitrile to yield N-((1R,4R)-4-((4-((5-cyclopentyl-1H-pyrazol-3-yl)amino)pyrimidin-2-yl)(methyl)amino)cyclohexyl)-2-(3-(trifluoromethyl)phenyl)acetamide (25 mg, 8.2% yield) as a solid. LC purity: 99.75%; m/z: 542.1 [M+H]⁺ (Mol. formula C₂₈H₃₄F₃N₇O, calcd. mol. wt. 541.62). ¹H NMR (400 MHz, CD₃OD): δ 7.85 (d, J=6.0 Hz, 1H), 7.64 (s, 1H), 7.59-7.50 (m, 3H), 6.28 (s, 1H), 6.18 (d, J=5.2 Hz, 1H), 4.62-4.57 (m, 1H), 3.69-3.63 (m, 1H), 3.59 (s, 2H), 3.15-3.06 (m, 1H), 3.01 (s, 3H), 2.11-2.04 (m, 4H), 1.80-1.65 (m, 10H), 1.52-1.42 (m, 2H).

### Example 104: Synthesis of Compound 104

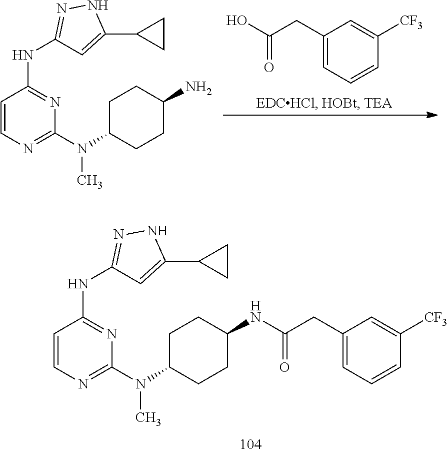 [SMILES: CN(c1nccc(Nc2cc(C3CC3)[nH]n2)n1)[C@H]1CC[C@H](N)CC1 ; O=C(O)Cc1cccc(C(F)(F)F)c1 ; CN(c1nccc(Nc2cc(C3CC3)[nH]n2)n1)[C@H]1CC[C@H](NC(=O)Cc2cccc(C(F)(F)F)c2)CC1]

### Synthesis of N-((1R,4R)-4-((4-((5-cyclopropyl-1H-pyrazol-3-yl)amino)pyrimidin-2-yl)(methyl)amino)cyclohexyl)-2-(3-(trifluoromethyl)phenyl)acetamide

To a solution of N2-((1R,4R)-4-aminocyclohexyl)-N4-(5-cyclopropyl-1H-pyrazol-3-yl)-N2-methylpyrimidine-2,4-diamine (150 mg, 0.458 mmol) and 2-(3-(trifluoromethyl)phenyl)acetic acid (140 mg, 0.687 mmol) in dry DMF (6 mL) was added triethylamine (0.19 mL, 1.374 mmol) drop-wise followed by the addition of EDC.HCl (176 mg, 0.916 mmol) and HOBt (62 mg, 0.458 mmol). The reaction mixture was stirred for 16 h. After the completion of reaction, the reaction mixture was diluted with dichloromethane washed with water and brine and dried over anhydrous Na₂SO₄ and concentrated to yield the crude product. The crude compound was purified by reverse phase preparative HPLC using X-SELECT C18 column with mobile phase 0.10% TFA in water/acetonitrile to obtain N-((1R,4R)-4-((4-((5-cyclopropyl-1H-pyrazol-3-yl)amino)pyrimidin-2-yl)(methyl)amino)cyclohexyl)-2-(3-(trifluoromethyl)phenyl)acetamide (40 mg, 17%). LC purity: 99.78%; m/z: 514.2 [M+H]⁺ (Mol. formula C₂₆H₃₀F₃N₇O, calcd. mol. wt. 513.57). ¹H NMR (400 MHz, CD₃OD): δ 7.72 (d, J=6.52 Hz, 1H), 7.64 (s, 1H), 7.59-7.51 (m, 3H), 6.38-6.36 (m, 2H), 4.71-4.66 (m, 1H), 3.68-3.65 (m, 1H), 3.60 (s, 2H), 3.07 (s, 3H), 2.11-2.05 (m, 2H), 1.97-1.85 (m, 5H), 1.48-1.46 (m, 2H), 1.01-0.97 (m, 2H), 0.77-0.75 (m, 2H).

### Example 105: Synthesis of Compound 105

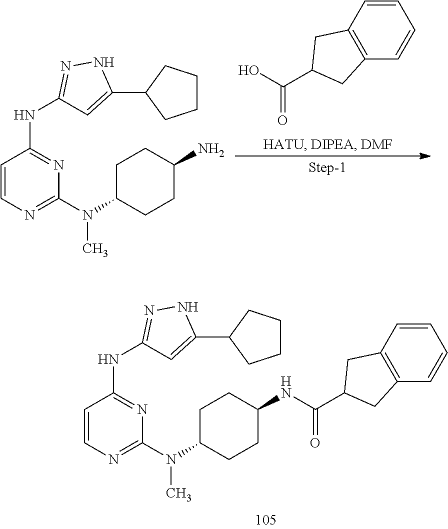 [SMILES: CN(c1nccc(Nc2cc(C3CCCC3)[nH]n2)n1)[C@H]1CC[C@H](N)CC1 ; O=C(O)C1Cc2ccccc2C1 ; CN(c1nccc(Nc2cc(C3CCCC3)[nH]n2)n1)[C@H]1CC[C@H](NC(=O)C2Cc3ccccc3C2)CC1]

### Step-1: Synthesis of N-((1R,4R)-4-((4-((5-cyclopentyl-1H-pyrazol-3-yl)amino)pyrimidin-2-yl)(methyl)amino)cyclohexyl)-2,3-dihydro-1H-indene-2-carboxamide

To a solution of 2,3-dihydro-1H-indene-2-carboxylic acid (48 mg, 0.295 mmol) in DMF (2 mL) was added DIPEA (0.14 mL, 0.845 mmol) and HATU (240 mg, 0.633 mmol). The reaction was stirred at ambient temperature for 30 min. Then N2-((1R,4R)-4-aminocyclohexyl)-N4-(5-cyclopentyl-1H-pyrazol-3-yl)-N2-methylpyrimidine-2,4-diamine (150 mg, 0.422 mmol) was added and the reaction mixture was stirred for 16 h at room temperature. The progress of the reaction was monitored by TLC, and after complete consumption of the starting material, the reaction mixture was diluted with water and extracted with DCM. The organic layer separated was dried over anhydrous sodium sulphate and concentrated to obtain crude. The crude compound was purified by reverse phase preparative HPLC using Sunfire C18 column with mobile phase 0.1% TFA in water/acetonitrile to yield N-((1R,4R)-4-((4-((5-cyclopentyl-1H-pyrazol-3-yl)amino)pyrimidin-2-yl)(methyl)amino)cyclohexyl)-2,3-dihydro-1H-indene-2-carboxamide (68 mg, 32%). LC purity: 99.87%; m/z: 500.2 [M+H]⁺ (Mol. formula C₂₉H₃₇N₇O, calcd. mol. wt. 499.66). ¹H NMR (400 MHz, CD₃OD): δ 7.74 (s, 1H), 7.19-7.17 (m, 2H), 7.13-7.11 (m, 2H), 6.50 (s, 1H), 6.36 (d, J=6.8 Hz, 1H), 3.70-3.65 (m, 1H), 3.25-3.10 (m, 10H), 2.16 (s, 4H), 1.89-1.83 (m, 6H), 1.73-1.68 (m, 4H), 1.49-1.45 (m, 2H).

### Example 106: Synthesis of Compound 106

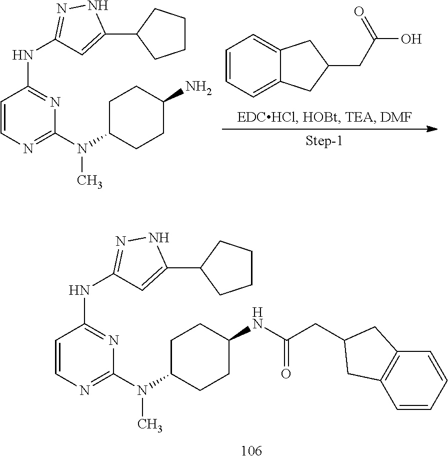

### Step-1: Synthesis of N-((1R,4R)-4-((4-((5-cyclopentyl-1H-pyrazol-3-yl)amino)pyrimidin-2-yl)(methyl)amino)cyclohexyl)-2-(2,3-dihydro-1H-inden-2-yl)acetamide

To a stirred solution of 2-(2,3-dihydro-1H-inden-2-yl)acetic acid (39.66 mg, 0.225 mmol) in dry DMF (2 mL) was added triethylamine (0.11 mL, 0.845 mmol) followed by the addition of the EDC.HCl (80.7 mg, 0.422 mmol) and HOBt (19 mg, 0.140 mmol). The reaction mixture was stirred at room temperature for 10 min, then N2-((1R,4R)-4-aminocyclohexyl)-N4-(5-cyclopentyl-1H-pyrazol-3-yl)-N2-methylpyrimidine-2,4-diamine (100 mg, 0.281 mmol) was added. The reaction mixture was stirred at room temperature for 4 h. After completion of the reaction (monitored by UPLC), the reaction mixture was diluted with dichloromethane washed with water and brine, dried over anhydrous Na₂SO₄ and concentrated under reduced pressure to yield the crude compound. The crude was purified by reverse phase preparative HPLC using X BRIDGE C18 column with mobile phase 0.10% TFA in water/acetonitrile to yield N-((1R,4R)-4-((4-((5-cyclopentyl-1H-pyrazol-3-yl)amino)pyrimidin-2-yl)(methyl)amino)cyclohexyl)-2-(2,3-dihydro-1H-inden-2-yl)acetamide (60 mg, 42.85%). LC purity: 99.64%; m/z: 514.3 [M+H]⁺ (Mol. formula C₃₀H₃₉N₇O, calcd. mol. wt. 513.69). ¹H NMR (400 MHz, CD₃OD): δ 7.73 (d, J=6.8 Hz, 1H), 7.19-7.16 (m, 2H), 7.13-7.01 (m, 2H), 6.49 (s, 1H), 5.96 (s, 1H), 3.69 (m, 1H), 3.13-3.06 (m, 3H), 3.05 (s, 3H), 2.89-2.82 (m, 1H), 2.68-2.63 (m, 2H), 2.36 (d, J=7.6 Hz, 2H), 2.15 (m, 4H), 1.86-1.82 (m, 6H), 1.77-1.70 (m, 5H), 1.44-1.30 (m, 2H).

### Example 107: Synthesis of Compound 107

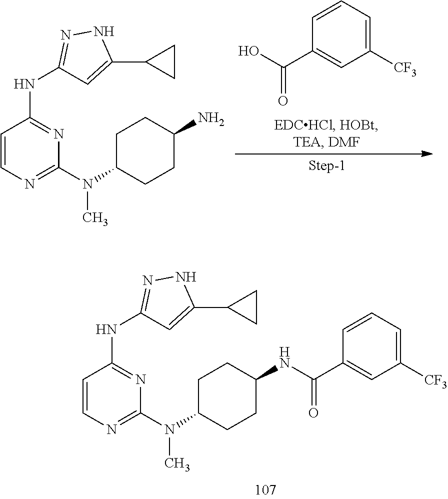 [SMILES: CN(c1nccc(Nc2cc(C3CC3)[nH]n2)n1)[C@H]1CC[C@H](N)CC1 ; O=C(O)c1cccc(C(F)(F)F)c1 ; CN(c1nccc(Nc2cc(C3CC3)[nH]n2)n1)[C@H]1CC[C@H](NC(=O)c2cccc(C(F)(F)F)c2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-3-(trifluoromethyl)benzamide)

To a solution of N²-((1R,4R)-4-aminocyclohexyl)-N⁴-(5-cyclopropyl-1H-pyrazol-3-yl)-N²-methylpyrimidine-2,4-diamine (200 mg, 0.611 mmol) in dry DMF (2 mL) was added TEA (0.2 mL, 1.833 mmol), EDC.HCl (175 mg, 0.916 mmol), HOBt (41.2 mg, 0.305 mmol) and 3-(trifluoromethyl)benzoic acid (92.9 mg, 0.488 mmol). The reaction mixture was stirred for 5 h at room temperature. The progress of the reaction was monitored by TLC. After complete consumption of starting material, water was added and the mixture was extracted with DCM. The organic layer was separated dried over anhydrous sodium sulfate and concentrated to obtain the crude compound. The crude product was purified by reverse phase preparative HPLC using X-bridge C8 column with mobile phase 0.1% Ammonia in water/acetonitrile to yield N-((1R,4R)-4-((4-((5-cyclopropyl-1H-pyrazol-3-yl)amino)pyrimidin-2-yl)(methyl)amino)cyclohexyl)-3-(trifluoromethyl)benzamide (60 mg, 19% yield) as the free base. LC purity: 99.83%; m/z: 500.23 [M+H]⁺ (Mol. formula C₂₇H₃₄N₈O, calcd. mol. wt. 499.54).

### Example 108: Synthesis of Compound 108

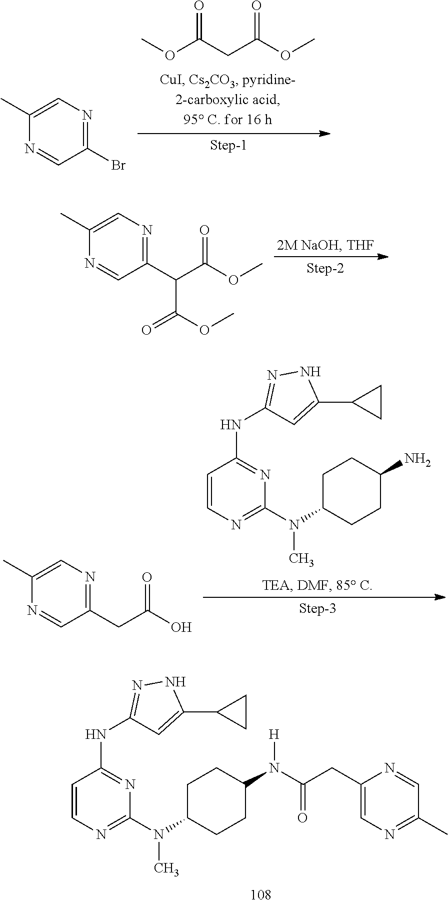

### Step-1: Synthesis of dimethyl 2-(5-methylpyrazin-2-yl)malonate

To a solution of 2-bromo-5-methylpyrazine (1.1 g, 6.358 mmol) in 1,4-dioxane (35 mL) were added dimethyl propanedioate (2.18 mL, 19.07 mmol), pyridine-2-carboxylic acid (156 mg, 1.271 mmol), copper(I) iodide (483 mg, 2.543 mmol), and cesium carbonate (6.19 g, 19.075 mmol). The reaction mixture was stirred at 95° C. for 16 h. The progress of the reaction was monitored by TLC. After completion of the reaction, the reaction mixture was cooled to RT diluted with ethyl acetate, washed with saturated aqueous sodium chloride solution, dried over anhydrous Na₂SO₄, filtered and concentrated under reduced pressure to get the crude product. The obtained crude product was purified by biotage Isolera using 230-400 silica mesh eluted with 0-80% ethyl acetate in pet ether as a eluent to yield dimethyl 2-(5-methylpyrazin-2-yl)malonate as a solid (1.1 g, 77.46%). LC purity: 93.28%; m/z: 225.1 [M+H]⁺ (Mol. formula C₁₀H₁₂N₂O₄, calcd. mol. wt. 224.2).

### Step-2: Synthesis of 2-(5-methylpyrazin-2-yl) acetic acid

To a stirred solution of dimethyl 2-(5-methylpyrazin-2-yl) malonate (1.1 g, 4.910 mmol) in THF (35 mL) was added 2M aqueous sodium hydroxide solution (9.82 mL, 19.642 mmol). The reaction mixture was stirred at room temperature for 16 h. After completion of the reaction (monitored by UPLC), reaction mixture was washed with diethyl ether. The aqueous layer was then adjusted to pH 3 via addition of 6 M aqueous hydrochloric acid, while the temperature of the reaction mixture was maintained 25° C. Then the reaction mixture was extracted with dichloromethane. The organic layer was washed with brine then dried over anhydrous Na₂SO₄ and concentrated under reduced pressure to yield 2-(5-methylpyrazin-2-yl)acetic acid as a solid (290 mg, 39.18%). LC purity: 96.6%; m/z: 153.1 [M+H]⁺ (Mol. formula C₇H₈N₂O₂, calcd. mol. wt. 152.15).

### Step-3: Synthesis of N-((1R,4R)-4-((4-((5-cyclopropyl-1H-pyrazol-3-yl)amino)pyrimidin-2-yl)(methyl)amino)cyclohexyl)-2-(5-methylpyrazin-2-yl)acetamide

To a stirred solution of 2-(5-methylpyrazin-2-yl)acetic acid (260 mg, 1.712 mmol) in dry DMF (5 mL) was added triethylamine (0.9 mL, 6.422 mmol) followed by the addition of the EDC.HCl (613 mg, 3.211 mmol) and HOBt (144 mg 1.070 mmol). The reaction mixture was stirred at room temperature for 10 min and then N2-((1R,4R)-4-aminocyclohexyl)-N4-(5-cyclopropyl-1H-pyrazol-3-yl)-N2-methylpyrimidine-2,4-diamine (700 mg, 2.140 mmol) was added. Then the reaction mixture was stirred at room temperature for 4 h. After completion of the reaction (monitored by UPLC), the reaction mixture was diluted with dichloromethane washed with water and brine, dried over anhydrous Na₂SO₄ and concentrated under reduced pressure to yield the crude compound. The crude compound was purified by reverse phase preparative HPLC using X-bridge C18 column with mobile phase 0.1% ammonia in water/acetonitrile to yield N-((1R,4R)-4-((4-((5-cyclopropyl-1H-pyrazol-3-yl)amino)pyrimidin-2-yl)(methyl)amino)cyclohexyl)-2-(5-methylpyrazin-2-yl)acetamide (90 mg, 10.88%) as the free base. LC purity: 98.75%; m/z: 462.1 [M+H]⁺ (Mol. formula C₂₄H₃₁N₉O, calcd. mol. wt. 461.57). ¹H NMR (400 MHz, CD₃OD): δ 8.48 (s, 2H), 7.87 (d, J=5.6 Hz, 1H), 6.23-6.16 (m, 2H), 4.61-4.57 (m, 1H), 3.74 (s, 2H), 3.73-3.67 (m, 1H), 3.0 (s, 3H), 2.55 (s, 3H), 2.08-2.06 (m, 2H), 1.95-1.90 (m, 1H), 1.79-1.73 (m, 4H), 1.51-1.47 (m, 2H), 1.0-0.97 (m, 2H), 0.75-0.73 (m, 2H).

### Example 109: Synthesis of Compounds 109 & 110

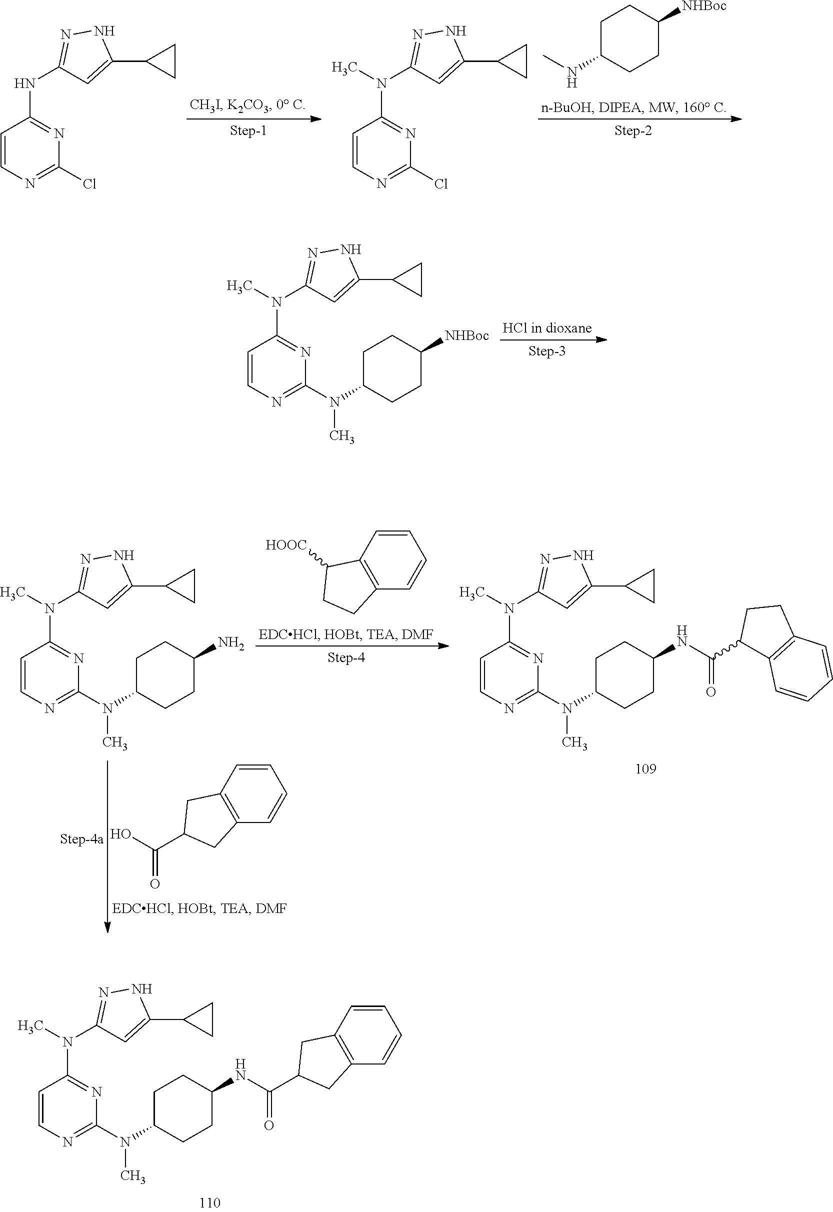

### Step-1: Synthesis of 2-chloro-N-(5-cyclopropyl-1H-pyrazol-3-yl)-N-methylpyrimidin-4-amine

To a cooled (0° C.) solution of 2-chloro-N-(5-cyclopropyl-1H-pyrazol-3-yl)pyrimidin-4-amine (2.0 g, 8.44 mmol) in dry DMF (20 mL) was added potassium carbonate (4.65 g, 33.76 mmol). The reaction mixture was stirred for 10 min at RT. The reaction mixture was cooled to 0° C. followed by addition of methyl iodide (0.63 mL, 10.13 mmol) dropwise. The reaction mixture was then stirred at RT for 2 h. The progress of the reaction was monitored by TLC, after consumption of the starting material, the reaction mixture was diluted with water and the compound was extracted with ethyl acetate. The organic layer was dried over anhydrous sodium sulfate, filtered and concentrated under reduced pressure. The crude was purified by Biotage Isolera using silica gel (230-400 mesh) with gradient elution of 0-5% MeOH in DCM to obtain 2-chloro-N-(5-cyclopropyl-1H-pyrazol-3-yl)-N-methylpyrimidin-4-amine (850 mg, 40%). LC purity: 98%; m/z: 250.1 [M+H]⁺ (Mol. formula C₁₁H₁₂ClN₅, calcd. mol. wt. 249.70).

### Step-2: Synthesis of tert-butyl ((1R,4R)-4-((4-((5-cyclopropyl-1H-pyrazol-3-yl)(methyl)amino)pyrimidin-2-yl)(methyl)amino)cyclohexyl)carbamate

To a solution of 2-chloro-N-(5-cyclopropyl-1H-pyrazol-3-yl)-N-methylpyrimidin-4-amine (500 mg, 2.002 mmol) in n-BuOH (5.0 mL) in a 20 mL microwave vial was added DIPEA (0.7 mL, 4.004 mmol) and tert-butyl ((1R,4R)-4-(methylamino)cyclohexyl)carbamate (690 mg, 3.003 mmol). The reaction mixture was heated in microwave at 160° C. for 2 h. The progress of the reaction was monitored by TLC analysis. After completion of the reaction, the reaction mixture was concentrated and purified by Biotage Isolera using silica gel (230-400 mesh) with gradient elution of 0-100% ethyl acetate in pet ether to obtain tert-butyl ((1R,4R)-4-((4-((5-cyclopropyl-1H-pyrazol-3-yl)(methyl)amino)pyrimidin-2-yl)(methyl)amino)cyclohexyl)carbamate (410 mg, 46%). LC purity: 97%; m/z: 442.3 [M+H]⁺ (Mol. formula C₂₃H₃₅N₇O₂, calcd. mol. wt. 441.58).

### Step-3: Synthesis of N2-((1R,4R)-4-aminocyclohexyl)-N4-(5-cyclopropyl-1H-pyrazol-3-yl)-N2,N4-dimethylpyrimidine-2,4-diamine

A well-stirred solution of tert-butyl ((1R,4R)-4-((4-((5-cyclopropyl-1H-pyrazol-3-yl)(methyl)amino)pyrimidin-2-yl)(methyl)amino)cyclohexyl)carbamate (0.4 g) in DCM (4.0 mL) was cooled to 0° C. and 4M hydrochloric acid in 1,4 dioxane (2.0 mL) was added dropwise. After complete addition, the reaction mixture was stirred at ambient temperature for 3 h. The progress of the reaction was monitored by TLC. After completion of the reaction, the reaction mixture was concentrated under reduced pressure and the residue thus obtained was washed with petroleum ether then dried well to afford N2-((1R,4R)-4-aminocyclohexyl)-N4-(5-cyclopropyl-1H-pyrazol-3-yl)-N2,N4-dimethylpyrimidine-2,4-diamine (0.4 g, quantitative) as the HCl salt. LC purity: 94%; m/z: 342.0 [M+H]⁺ (Mol. formula C₁₈H₂₇N₇, calcd. mol. wt. 341.46).

### Step-4: Synthesis of N-((1R,4R)-4-((4-((5-cyclopropyl-1H-pyrazol-3-yl)(methyl)amino)pyrimidin-2-yl)(methyl)amino)cyclohexyl)-2,3-dihydro-1H-indene-1-carboxamide

To a solution of 2,3-dihydro-1H-indene-1-carboxylic acid (95 mg, 0.587 mmol) in dry DMF (4 mL) was added triethylamine triethylamine (0.4 mL, 2.93 mmol) drop-wise followed by the addition of EDC·HCl (168 mg, 0.88 mmol) and HOBt (63 mg, 0.469 mmol). The reaction mixture was stirred for 15 min and then N2-((1R,4R)-4-aminocyclohexyl)-N4-(5-cyclopropyl-1H-pyrazol-3-yl)-N2,N4-dimethylpyrimidine-2,4-diamine (200 mg, 0.587 mmol) was added. The reaction mixture was stirred at room temperature for 16 h. The progress of the reaction was monitored by TLC. After completion of the reaction, the reaction mixture was diluted with dichloromethane washed with ice-cold water and brine, dried over anhydrous Na₂SO₄ and concentrated to get the crude compound. The crude product was purified by Biotage Isolera using silica gel (230-400 mesh) with gradient elution of 0-100% ethyl acetate in pet ether to obtain N-((1R,4R)-4-((4-((5-cyclopropyl-TH-pyrazol-3-yl)(methyl)amino)pyrimidin-2-yl)(methyl)amino)cyclohexyl)-2,3-dihydro-1H-indene-1-carboxamide (55 mg, 20%) as a solid. LC purity: 98.5%; m/z: 486.2 [M+H]⁺ (Mol. formula C₂₈H₃₅F₃N₇O, calcd. mol. wt. 485.64). ¹H VTNMR (400 MHz, DMSO-d₆): δ 12.04 (s, 1H), 8.07 (d, J=8 Hz, 1H), 7.84 (d, J=6 Hz, 1H), 7.23-7.13 (m, 4H), 6.08 (s, 1H), 5.98 (s, 1H), 4.56-4.52 (m, 1H), 3.86-3.83 (m, 1H), 3.36-3.34 (m, 1H), 3.32 (s, 3H), 3.07-3.02 (m, 1H), 3.01 (s, 3H), 2.96-2.94 (m, 1H), 2.25-2.15 (m, 2H), 1.93-1.89 (m, 3H), 1.68-1.62 (m, 4H), 1.41-1.37 (m, 2H), 0.95-0.91 (m, 2H), 0.73-0.70 (m, 2H).

### Step-4a: Synthesis of N-((1R,4R)-4-((4-((5-cyclopropyl-1H-pyrazol-3-yl)(methyl)amino)pyrimidin-2-yl)(methyl)amino)cyclohexyl)-2,3-dihydro-1H-indene-2-carboxamide

To a solution of 2,3-dihydro-1H-indene-2-carboxylic acid (95 mg, 0.587 mmol) in dry DMF (4 mL) was added triethylamine triethylamine (0.4 mL, 2.93 mmol) drop-wise followed by the addition of EDC·HCl (a (168 mg, 0.88 mmol) and HOBt (63 mg, 0.469 mmol). The reaction mixture was stirred for 15 min and then N2-((1R,4R)-4-aminocyclohexyl)-N4-(5-cyclopropyl-1H-pyrazol-3-yl)-N2,N4-dimethylpyrimidine-2,4-diamine (200 mg, 0.587 mmol) was added. The reaction mixture was stirred at room temperature for 16 h. The progress of the reaction was monitored by TLC. After completion of the reaction, the reaction mixture was diluted with dichloromethane, washed with ice-cold water and brine and dried over anhydrous Na₂SO₄ and concentrated to obtain the crude compound. The crude product was purified by Biotage Isolera using silica gel (230-400 mesh) with gradient elution of 0-100% ethyl acetate in pet ether to obtain N-((1R,4R)-4-((4-((5-cyclopropyl-1H-pyrazol-3-yl)(methyl)amino)pyrimidin-2-yl)(methyl)amino)cyclohexyl)-2,3-dihydro-1H-indene-2-carboxamide (25 mg, 9%) as a solid. LC purity: 98.2%; m/z: 486.0 [M+H]⁺ (Mol. formula C₂₈H₃₅F₃N₇O, calcd. mol. wt. 485.64). ¹H VTNMR (400 MHz, DMSO-d₆): δ 12.07 (s, 1H), 7.84 (d, J=6 Hz, 1H), 7.52 (d, J=7.6 Hz, 1H), 7.19-7.10 (m, 4H), 6.08 (d, J=5.6 Hz, 1H), 5.92 (s, 1H), 4.54-4.50 (m, 1H), 3.59-3.54 (m, 1H), 3.35 (s, 3H), 3.16-3.10 (m, 2H), 3.05-3.03 (m, 3H), 2.94 (s, 3H), 1.93-1.87 (m, 3H), 1.64-1.63 (m, 4H), 1.36-1.32 (m, 2H), 0.95-0.91 (m, 2H), 0.73-0.69 (m, 2H).

### Example 110: Synthesis of Compound 111

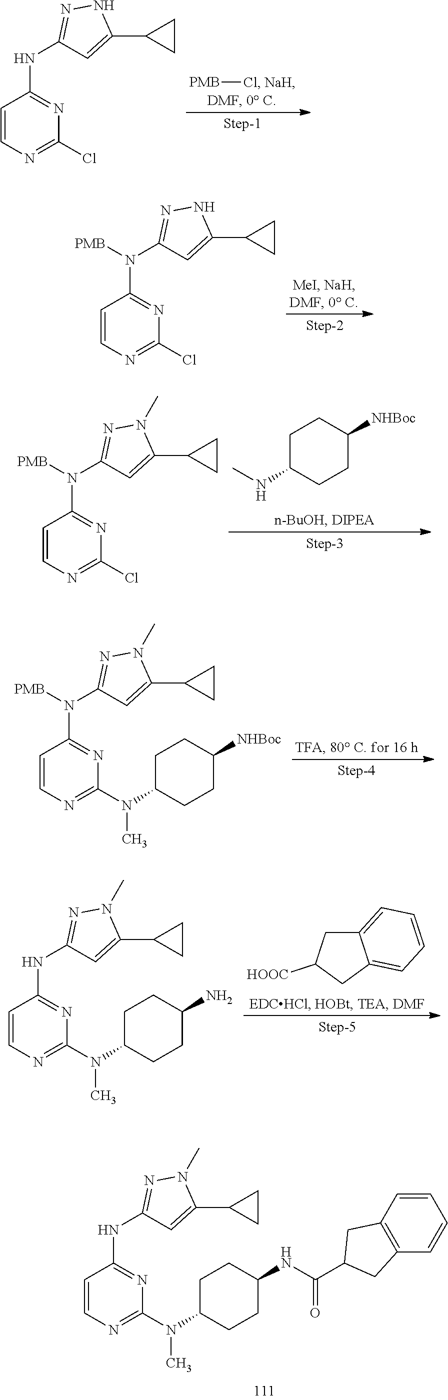

### Step-1: Synthesis of 2-chloro-N-(5-cyclopropyl-1H-pyrazol-3-yl)-N-(4-methoxybenzyl) pyrimidin-4-amine

To a solution of 2-chloro-N-(5-cyclopropyl-1H-pyrazol-3-yl)pyrimidin-4-amine (2.0 g, 8.511 mmol) in dry DMF (20.0 mL) was cooled to 0° C. and added NaH (60% pure) (0.2 g, 8.511 mmol) portion wise. The reaction mixture was stirred for 15 min and then 1-(chloromethyl)-4-methoxybenzene (1.16 mL, 8.511 mmol) was added. The ice bath was removed, and the reaction mixture allowed to stir at room temperature for 1 h. The reaction mixture was quenched by addition of cold water and extracted with ethyl acetate. The combined organic layer was washed with brine solution, dried over anhydrous sodium sulphate, filtered off and concentrated under vacuo to get the residue. The crude material was purified by Biotage Isolera using silica gel (230-400 mesh) with gradient elution of 20-30% ethyl acetate in pet ether to afford 2-chloro-N-(5-cyclopropyl-1H-pyrazol-3-yl)-N-(4-methoxybenzyl)pyrimidin-4-amine (0.45 g, 15%). LC purity: 74%; m/z: 356.1 [M+H]⁺ (Mol. formula C₁₉H₁₈ClN₅O, calcd. mol. wt. 355.83).

### Step-2: Synthesis of 2-chloro-N-(5-cyclopropyl-1-methyl-1H-pyrazol-3-yl)-N-(4-methoxybenzyl)pyrimidin-4-amine

To a solution of 2-chloro-N-(5-cyclopropyl-1H-pyrazol-3-yl)-N-(4-methoxybenzyl)pyrimidin-4-amine (450 mg, 1.26 mmol) in dry DMF (9.0 mL) was cooled to 0° C. and added NaH (60% pure) (46 mg, 1.89 mmol). The reaction mixture stirred for 15 minutes and then methyl iodide (0.08 mL, 1.26 mmol) was added. The ice bath was removed, and reaction mixture allowed to stir at room temperature for 1 h. The reaction mixture was quenched by addition of cold water and extracted with ethyl acetate. The combined organic layer was washed with brine solution, dried over anhydrous sodium sulphate, filtered off and concentrated under vacuo to get the residue. The crude product was purified by Biotage Isolera using silica gel (230-400 mesh) with gradient elution of 20-30% ethyl acetate in pet ether to afford 2-chloro-N-(5-cyclopropyl-1-methyl-1H-pyrazol-3-yl)-N-(4-methoxybenzyl)pyrimidin-4-amine (270 mg, 58%). LC purity: 95%; m/z: 370.3 [M+H]⁺ (Mol. formula C₁₉H₂₀ClN₅O, calcd. mol. wt. 369.85).

### Step-3: Synthesis of tert-butyl ((1R,4R)-4-((4-((5-cyclopropyl-1-methyl-1H-pyrazol-3-yl)(4-methoxybenzyl)amino)pyrimidin-2-yl)(methyl)amino)cyclohexyl)carbamate

To a solution of 2-chloro-N-(5-cyclopropyl-1-methyl-1H-pyrazol-3-yl)-N-(4-methoxybenzyl)pyrimidin-4-amine (250 mg, 0.68 mmol) in n-BuOH (5.0 mL) In a 20 mL microwave vial was added DIPEA (0.24 mL, 1.36 mmol) and tert-butyl ((1R,4R)-4-(methylamino)cyclohexyl)carbamate (310 mg, 1.36 mmol). The reaction mixture was heated in microwave at 160° C. for 2 h. The progress of the reaction was monitored by TLC analysis. After completion of the reaction, the reaction mixture was concentrated to get the residue. The residue was purified by Biotage Isolera using silica gel (230-400 mesh) with gradient elution of 20-30% ethyl acetate in pet ether to afford tert-butyl ((1R,4R)-4-((4-((5-cyclopropyl-1-methyl-1H-pyrazol-3-yl)(4-methoxybenzyl)amino)pyrimidin-2-yl)(methyl)amino)cyclohexyl)carbamate (250 mg, 66%) as a gummy solid. LC purity: 87%; m/z: 562.3 [M+H]⁺ (Mol. formula C₃₁H₄₃N₇O₃, calcd. mol. wt. 561.73).

### Step-4: Synthesis of N2-((1R,4R)-4-aminocyclohexyl)-N4-(5-cyclopropyl-1-methyl-1H-pyrazol-3-yl)-N2-methylpyrimidine-2,4-diamine

To a solution of tert-butyl ((1R,4R)-4-((4-((5-cyclopropyl-1-methyl-1H-pyrazol-3-yl)(4-methoxybenzyl)amino)pyrimidin-2-yl)(methyl)amino)cyclohexyl)carbamate (250 mg, 0.445 mmol) in trifluoro acetic acid (5.0 mL) was heated at 80° C. and stirred for 6 h. The progress of the reaction was monitored by TLC analysis. After completion of the reaction, the reaction mixture was concentrated under reduced pressure to get the residue. The residue thus obtained was washed with petroleum ether and dried well to afford N2-((1R,4R)-4-aminocyclohexyl)-N4-(5-cyclopropyl-1-methyl-1H-pyrazol-3-yl)-N2-methylpyrimidine-2,4-diamine (300 mg, quantitative yield) as a TFA salt. LC purity: 71%; m/z: 342.2 [M+1]⁺ (Mol. formula C₁₈H₂₇N₇, calcd. mol. wt. 341.46).

### Step-5: Synthesis of N-((1R,4R)-4-((4-((5-cyclopropyl-1-methyl-1H-pyrazol-3-yl)amino)pyrimidin-2-yl)(methyl)amino)cyclohexyl)-2,3-dihydro-1H-indene-2-carboxamide

To a solution of 2,3-dihydro-1H-indene-2-carboxylic acid (71 mg, 0.440 mmol) in dry DMF (5 mL) was added triethylamine (0.51 mL, 3.666 mmol) drop-wise followed by the addition of EDC·HCl (210 mg, 1.10 mmol) and HOBt (59 mg, 0.440 mmol). The reaction mixture was stirred for 15 min and then N2-((1R,4R)-4-aminocyclohexyl)-N4-(5-cyclopropyl-1-methyl-1H-pyrazol-3-yl)-N2-methylpyrimidine-2,4-diamine (250 mg, 0.733 mmol) was added. The reaction mixture was stirred at room temperature for 16 h. The progress of the reaction was monitored by TLC. After completion of the reaction, the reaction mixture was diluted with dichloromethane washed with ice cold water, brine and dried over anhydrous Na₂SO₄ and concentrated to get the crude compound. The crude compound was purified by reverse phase preparative HPLC using X-SELECT C18 column with mobile phase 0.1% TFA in water/acetonitrile to yield N-((1R,4R)-4-((4-((5-cyclopropyl-1-methyl-1H-pyrazol-3-yl)amino)pyrimidin-2-yl)(methyl)amino)cyclohexyl)-2,3-dihydro-TH-indene-2-carboxamide (45 mg, 12%). LC purity: 97%; m/z: 486.0 [M+H]⁺ (Mol. formula C₂₈H₃₉N₇O, calcd. mol. wt. 485.64).

### Example 111: Synthesis of Compound 112

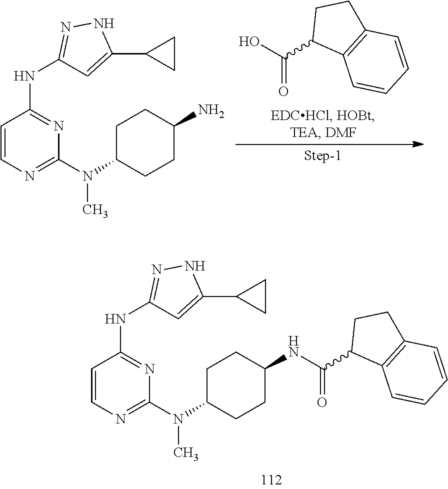 [SMILES: CN(c1nccc(Nc2cc(C3CC3)[nH]n2)n1)[C@H]1CC[C@H](N)CC1 ; O=C(O)C1CCc2ccccc21 ; CN(c1nccc(Nc2cc(C3CC3)[nH]n2)n1)[C@H]1CC[C@H](NC(=O)C2CCc3ccccc32)CC1]

### Step-1: N-((1R,4R)-4-((4-((5-cyclopropyl-1H-pyrazol-3-yl)amino)pyrimidin-2-yl)(methyl) amino)cyclohexyl)-2,3-dihydro-1H-indene-1-carboxamide

A solution of N²-((1R,4R)-4-aminocyclohexyl)-N⁴-(5-cyclopropyl-1H-pyrazol-3-yl)-N²-methylpyrimidine-2,4-diamine (200 mg, 0.611 mmol) in dry DMF (2 mL) was added TEA (0.2 mL, 1.833 mmol), EDC·HCl (175 mg, 0.916 mmol), HOBt (41.2 mg, 0.305 mmol) and 2,3-dihydro-TH-indene-1-carboxylic acid (79.18 mg, 0.488 mmol). The reaction mixture was stirred for 5 h at room temperature. The progress of the reaction was monitored by TLC. After complete consumption of starting material, water was added and extracted with DCM. The organic layer separated was dried over anhydrous sodium sulphate and concentrated. The crude compound was purified by reverse phase preparative HPLC (with mobile phase 0.1% TFA in water/acetonitrile) to get N-((1R,4R)-4-((4-((5-cyclopropyl-1H-pyrazol-3-yl)amino) pyrimidin-2-yl)(methyl)amino)cyclohexyl)-2,3-dihydro-1H-indene-2-carboxamide (60 mg, 20.3%). LC purity: 99.60%; m/z: 472.28 [M+H]⁺ (Mol. formula C₂₇H₃₃N₇O, calcd. mol. wt. 471.67). ¹H NMR (400 MHz, CD₃OD): δ 7.72 (d, J=7.2 Hz, 1H), 7.25-7.14 (m, 4H), 6.38-6.23 (m, 2H), 4.67-4.58 (m, 1H), 3.97-3.93 (m, 1H), 3.89-3.73 (m, 1H), 3.14-3.11 (m, 1H), 3.08 (s, 3H), 2.95-2.91 (m, 1H), 2.37-2.30 (m, 2H), 2.17-2.09 (m, 2H), 1.88-1.83 (m, 5H), 1.55-1.50 (m, 2H), 1.06-1.00 (m, 2H), 0.79-0.76 (m, 2H).

### Example 112: Synthesis of Compounds 113 F1 & 113 F2

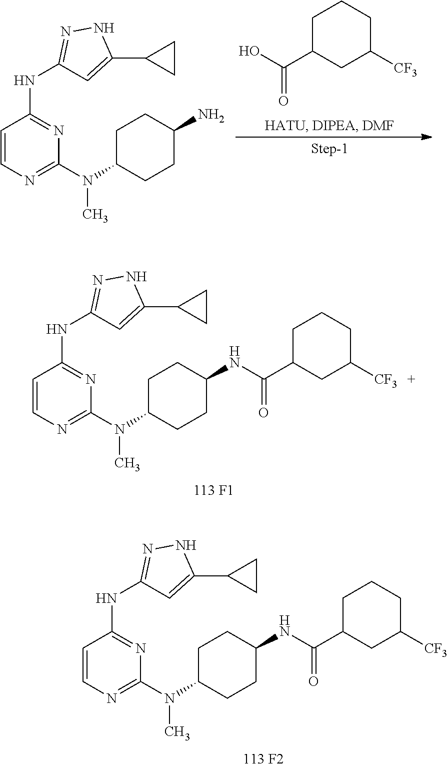 [SMILES: CN(c1nccc(Nc2cc(C3CC3)[nH]n2)n1)[C@H]1CC[C@H](N)CC1 ; O=C(O)C1CCCC(C(F)(F)F)C1 ; CN(c1nccc(Nc2cc(C3CC3)[nH]n2)n1)[C@H]1CC[C@H](NC(=O)C2CCCC(C(F)(F)F)C2)CC1 ; CN(c1nccc(Nc2cc(C3CC3)[nH]n2)n1)[C@H]1CC[C@H](NC(=O)C2CCCC(C(F)(F)F)C2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-3-(trifluoromethyl)cyclohexane-1-carboxamide

To a solution of (1R,3R)-3-(trifluoromethyl)cyclohexane-1-carboxylic acid (119 mg, 0.611 mmol) in DMF (6 mL) was added DIPEA (0.65 mL, 3.66 mmol) drop-wise at 0° C. followed by HATU (697 mg, 1.83 mmol). The reaction mixture was stirred for 5 min and added N2-((1R,4R)-4-aminocyclohexyl)-N4-(5-cyclopropyl-1H-pyrazol-3-yl)-N2-methylpyrimidine-2,4-diamine (400 mg, 1.22 mmol). The reaction mixture was warmed to room temperature and stirred for 1 h. The reaction was monitored by TLC. After completion of the reaction, the reaction mixture was diluted with dichloromethane, washed with water and brine and dried over anhydrous Na₂SO₄ and concentrated to yield crude compound which was then purified by reverse phase preparative HPLC to obtain two diastereomers of N-((1R,4R)-4-((4-((5-cyclopropyl-1H-pyrazol-3-yl)amino)pyrimidin-2-yl)(methyl)amino)cyclohexyl)-3-(trifluoromethyl)cyclohexane-1-carboxamide (113 F1=30 mg, 113 F2=30 mg, 10%) as the free bases. 113 F1: LC purity: 94.05%; m/z: 505.9 [M+H]⁺ (Mol. formula C₂₅H₃₄F₃N₇O, calcd. mol. wt. 505.59). ¹H NMR (400 MHz, CD₃OD): δ 7.87 (s, 1H), 6.32-6.15 (m, 2H), 4.66-4.57 (m, 1H), 3.62-3.56 (m, 1H), 3.00 (s, 3H), 2.26-2.23 (m, 2H), 2.05-1.96 (m, 6H), 1.94-1.81 (m, 5H), 1.65-1.57 (m, 5H), 1.38-1.35 (m, 1H), 1.00-0.98 (m, 2H), 0.75-0.72 (m, 2H). 113 F2: LC purity: 95.75%; m/z: 505.9 [M+H]⁺ (Mol. formula C₂₅H₃₄F₃N₇O, calcd. mol. wt. 505.59).

### Example 113: Synthesis of Compound 114

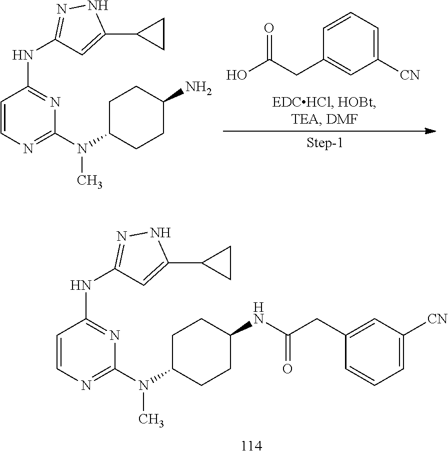 [SMILES: CN(c1nccc(Nc2cc(C3CC3)[nH]n2)n1)[C@H]1CC[C@H](N)CC1 ; N#Cc1cccc(CC(=O)O)c1 ; CN(c1nccc(Nc2cc(C3CC3)[nH]n2)n1)[C@H]1CC[C@H](NC(=O)Cc2cccc(C#N)c2)CC1]

### Step-1: Synthesis of 2-(3-cyanophenyl)-N-((1R,4R)-4-((4-((5-cyclopropyl-1H-pyrazol-3-yl)amino)pyrimidin-2-yl)(methyl)amino)cyclohexyl)acetamide

A solution of N²-((1R,4R)-4-aminocyclohexyl)-N⁴-(5-cyclopropyl-1H-pyrazol-3-yl)-N²-methylpyrimidine-2,4-diamine (200 mg, 0.611 mmol) in dry DMF (2 mL) was added TEA (0.02 mL, 0.183 mmol), EDC·HCl (175 mg, 0.916 mmol), HOBt (41.2 mg, 0.305 mmol) and 2-(3-cyanophenyl)acetic acid (78.7 mg, 0.488 mmol). The reaction mixture was stirred for 5 h at room temperature. The progress of the reaction was monitored by TLC. After complete consumption of starting material, water was added and extracted with DCM. The organic layer separated was dried over anhydrous sodium sulphate and concentrated to obtain the crude product. The crude product was purified by reverse phase preparative HPLC to yield 2-(3-cyanophenyl)-N-((1R,4R)-4-((4-((5-cyclopropyl-1H-pyrazol-3-yl)amino)pyrimidin-2-yl)(methyl)amino)cyclohexyl)acetamide (90 mg, 32%) as the free base. LC purity: 99.76%; m/z: 471.26 [M+H]⁺ (Mol. formula C₂₆H₃₀N₈O, calcd. mol. wt. 470.58). ¹H NMR (400 MHz, CD₃OD): δ 7.86 (s, 1H), 7.68-7.62 (m, 3H), 7.52 (t, J=7.6 Hz, 1H), 6.23-6.14 (m, 2H), 4.56-4.50 (m, 1H), 3.66-3.61 (m, 1H), 3.57 (s, 2H), 2.99 (s, 3H), 2.06-2.03 (m, 2H), 1.94-1.89 (m, 1H), 1.78-1.72 (m, 4H), 1.49-1.43 (m, 2H), 1.03-0.98 (m, 2H), 0.75-0.73 (m, 2H).

### Example 114: Synthesis of Compound 115

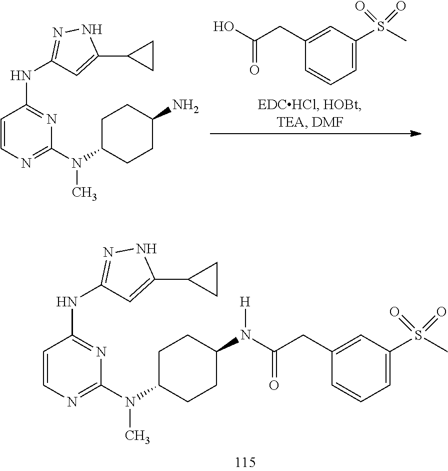 [SMILES: CN(c1nccc(Nc2cc(C3CC3)[nH]n2)n1)[C@H]1CC[C@H](N)CC1 ; CS(=O)(=O)c1cccc(CC(=O)O)c1 ; CN(c1nccc(Nc2cc(C3CC3)[nH]n2)n1)[C@H]1CC[C@H](NC(=O)Cc2cccc(S(C)(=O)=O)c2)CC1]

### Synthesis of N-((1R,4R)-4-((4-((5-cyclopropyl-1H-pyrazol-3-yl)amino)pyrimidin-2-yl)(methyl)amino)cyclohexyl)-2-(3-(methylsulfonyl)phenyl)acetamide

To a solution of N2-((1R,4R)-4-aminocyclohexyl)-N4-(5-cyclopropyl-1H-pyrazol-3-yl)-N2-methylpyrimidine-2,4-diamine (200 mg, 0.610 mmol) and 2-(3-(methylsulfonyl)phenyl)acetic acid (131 mg, 0.610 mmol) in dry DMF (3 mL) was added triethylamine (0.17 mL, 1.22 mmol) drop-wise followed by the addition of EDC·HCl (176 mg, 0.916 mmol) and HOBt (41 mg, 0.305 mmol). The reaction mixture was stirred for 16 h. After the completion of reaction, the reaction mixture was diluted with dichloromethane, washed with water and brine and dried over anhydrous Na₂SO₄ and concentrated to yield the crude product. The crude compound was purified by reverse phase preparative HPLC to obtain N-((1R,4R)-4-((4-((5-cyclopropyl-1H-pyrazol-3-yl)amino)pyrimidin-2-yl)(methyl)amino)cyclohexyl)-2-(3-(methylsulfonyl)phenyl) acetamide (60 mg, 18.8%) as the free base. LC purity: 99.65%; m/z: 524.3 [M+H]⁺ (Mol. formula C₂₆H₃₃N₇O₃S, calcd. mol. wt. 523.66). ¹H NMR (400 MHz, DMSO-d₆): δ 11.92 (s, 1H), 9.32 (s, 1H), 8.10 (d, J=7.6 Hz, 1H), 7.85-7.78 (m, 3H), 7.59 (t, J=7.6 Hz, 2H), 6.32-6.12 (m, 2H), 4.52-4.42 (m, 1H), 3.54 (s, 3H), 3.20 (s, 3H), 2.91 (s, 3H), 1.92-1.84 (m, 3H), 1.65-1.61 (m, 4H), 1.34-1.23 (m, 2H), 0.92-0.90 (m, 2H), 0.67-0.63 (m, 2H).

### Example 115: Synthesis of Compound 116

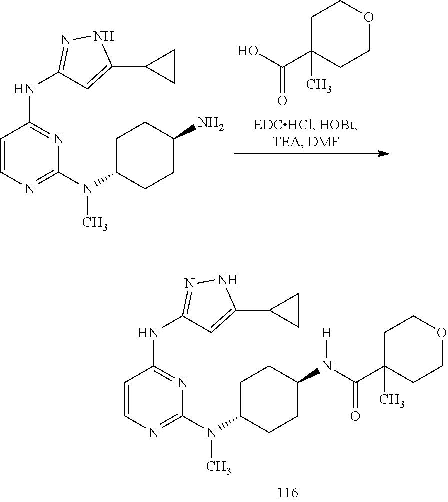

### Synthesis of N-((1R,4R)-4-((4-((5-cyclopropyl-1H-pyrazol-3-yl)amino)pyrimidin-2-yl)(methyl)amino)cyclohexyl)-4-methyltetrahydro-2H-pyran-4-carboxamide

To a solution of 4-methyltetrahydro-2H-pyran-4-carboxylic acid (106 mg, 0.733 mmol) in DMF (2 mL) was added TEA (0.41 mL, 3.0543 mmol), HOBt (82 mg, 0.6108 mmol) and EDC·HCl (210 mg, 1.099 mmol) the reaction was stirred for 30 min at room temperature. Then N2-((1R,4R)-4-aminocyclohexyl)-N4-(5-cyclopropyl-1H-pyrazol-3-yl)-N2-methylpyrimidine-2,4-diamine (200 mg, 0.6108 mmol) was added. The reaction was stirred at room temperature for 8 h. The progress of the reaction was monitored by TLC, and after complete consumption of the starting material, water was added and the mixture was extracted with DCM. The organic layer was separated and dried over anhydrous sodium sulphate and concentrated to obtain crude, the crude compound was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-4-methyltetrahydro-2H-pyran-4-carboxamide (40 mg, 14.8%) as the free base. LC purity: 97.52%; m/z: 454.2 [M+H]⁺ (Mol. formula C₂₄H₃₅N₇O₂, calcd. mol. wt. 453.6).

### Example 116: Synthesis of Compound 117

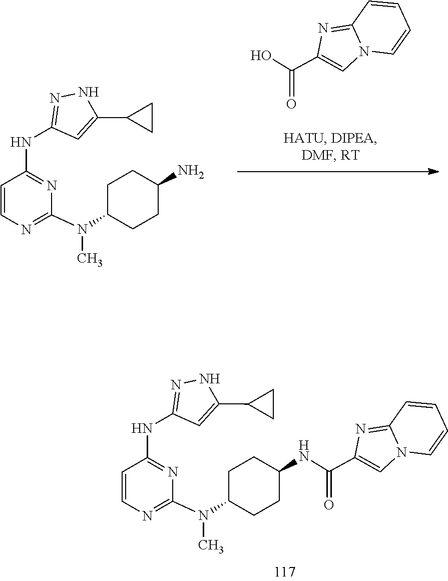

### Synthesis of N-((1R,4R)-4-((4-((5-cyclopropyl-1H-pyrazol-3-yl)amino)pyrimidin-2-yl)(methyl)amino)cyclohexyl)imidazo[1,2-a]pyridine-2-carboxamide

To a solution of imidazo[1,2-a]pyridine-2-carboxylic acid (0.099 g, 0.61 mmol) in dry DMF (6 mL) was added DIPEA (0.31 mL, 1.83 mmol) followed by HATU (0.464 g, 1.22 mmol). The reaction mixture was stirred for 15 min and then N2-((1R,4R)-4-aminocyclohexyl)-N4-(5-cyclopropyl-1H-pyrazol-3-yl)-N2-methylpyrimidine-2,4-diamine (0.200 g, 0.61 mmol) was added. The reaction mixture was stirred at room temperature for 12 h. After completion of the reaction (monitored by UPLC), the reaction mixture was diluted with dichloromethane washed with water, brine, dried over anhydrous Na₂SO₄ and concentrated to yield the residue. The residue was purified by reverse phase preparative HPLC to yield N-((1R,4R)-4-((4-((5-cyclopropyl-1H-pyrazol-3-yl)amino)pyrimidin-2-yl)(methyl)amino)cyclohexyl) imidazo[1,2-a]pyridine-2-carboxamide (30 mg, 10.4%) as the free base. LC purity: 99.26%; m/z: 472.9 [M+H]⁺ (Mol. formula C₂₅H₂₉N₉O calcd. mol. wt. 471.57). ¹H NMR (400 MHz, CD₃OD): δ 8.50-8.48 (m, 1H), 8.31 (s, 1H), 8.37 (d, J=6 Hz, 1H), 7.61-7.58 (m, 1H), 7.42-7.38 (m, 1H), 7.01-6.97 (m, 1H), 6.29-6.17 (m, 2H), 4.66-4.62 (m, 1H), 3.98-3.92 (m, 1H), 3.04 (s, 3H), 2.24-2.12 (m, 2H), 1.97-1.91 (m, 1H), 1.87-1.85 (m, 4H), 1.78-1.69 (m, 2H), 1.03-0.99 (m, 2H), 0.77-0.75 (m, 2H).

### Example 117: Synthesis of Compound 118

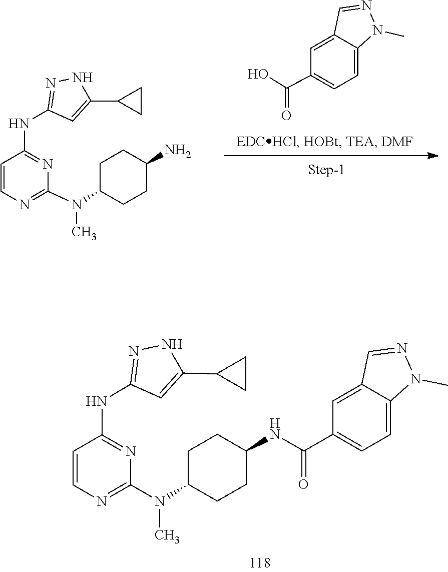

### Step-1: Synthesis of N-((1R,4R)-4-((4-((5-cyclopropyl-1H-pyrazol-3-yl)amino)pyrimidin-2-yl)(methyl)amino)cyclohexyl)-1-methyl-1H-indazole-5-carboxamide

To a stirred solution of 1-methyl-1H-indazole-5-carboxylic acid (77.5 mg, 0.440 mmol) in dry DMF (2 mL) was added triethylamine (0.23 mL, 1.651 mmol) followed by the addition of the EDC·HCl (157 mg, 0.825 mmol) and HOBt (37.15 mg, 0.275 mmol). The reaction mixture was stirred at room temperature for 10 min and then N2-((1R,4R)-4-aminocyclohexyl)-N4-(5-cyclopropyl-1H-pyrazol-3-yl)-N2-methylpyrimidine-2,4-diamine (180 mg, 0.550 mmol) was added. The reaction mixture was stirred at room temperature for 4 h. After completion of the reaction (monitored by UPLC), the reaction mixture was diluted with dichloromethane washed with water and brine, dried over anhydrous Na₂SO₄ and concentrated under reduced pressure to get the crude product. The crude product obtained was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-1-methyl-1H-indazole-5-carboxamide (40 mg, 15.38%) as the free base. LC purity: 99.75%; m/z: 486.2 [M+H]⁺ (Mol. formula C₂₆H₃₁N₉O, calcd. mol. wt. 485.60). ¹H NMR (400 MHz, CD₃OD): δ 8.30 (s, 1H), 8.11 (s, 1H), 7.93-7.89 (m, 2H), 7.60 (d, J=8.8 Hz, 1H), 6.18-6.12 (m, 2H), 4.57-4.50 (m, 1H), 4.10 (s, 3H), 3.99-3.92 (m, 1H), 3.04 (s, 3H), 2.20-2.17 (m, 2H), 2.03-1.99 (m, 1H), 1.96-1.78 (m, 4H), 1.70-1.63 (m, 2H), 1.00-0.95 (m, 2H), 0.77-0.73 (m, 2H).

### Example 118: Synthesis of Compound 119

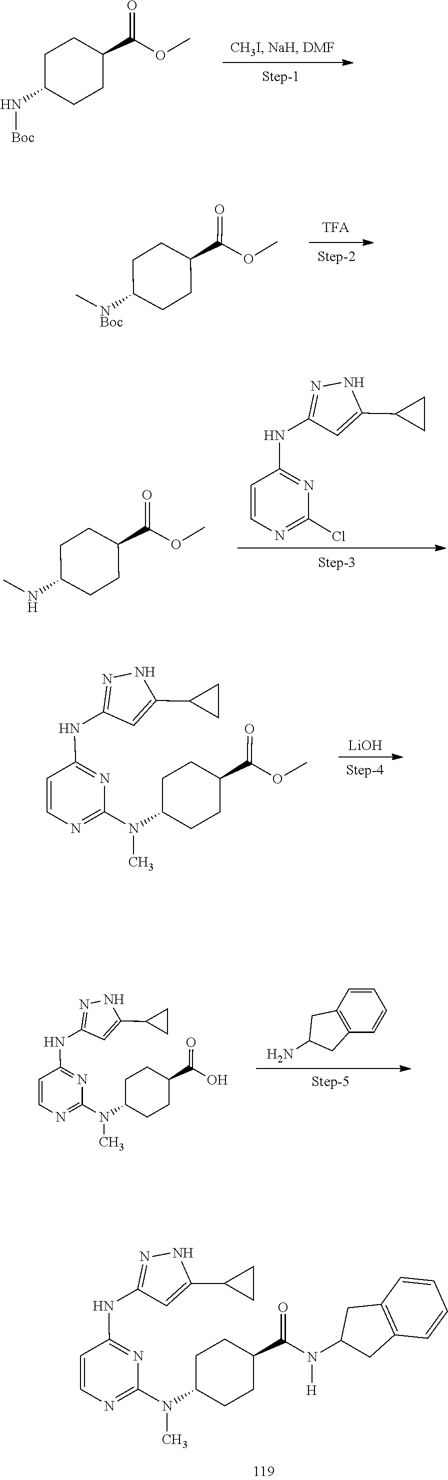

### Step-1: Synthesis of methyl (1R,4R)-4-((tert-butoxycarbonyl)(methyl)amino)cyclohexane-1-carboxylate

To a stirred solution of methyl (1R,4R)-4-((tert-butoxycarbonyl)amino)cyclohexane-1-carboxylate (2 g, 7.7 mmol) in DMF (20 mL) was added sodium hydride (0.778 g, 15.5 mmol, 60% in mineral oil) portion wise at 0° C. The reaction mixture was stirred at 0° C. for 30 min. Then methyl iodide (1.2 mL, 15.5 mmol) was added dropwise at 0° C. The reaction mixture was allowed to warm to room temperature and stirred for 16 h. After completion of the reaction (monitored by TLC), the reaction mixture was quenched with ice and extracted with ethyl acetate. The organic layer was dried over anhydrous sodium sulphate, filtered and concentrated under reduced pressure to obtain methyl (1R,4R)-4-((tert-butoxycarbonyl)(methyl)amino)cyclohexane-1-carboxylate (2.1 g, quantitative yield). LC purity: 99.67%; m/z: 272 [M+H]⁺ (Mol. formula C₁₄H₂₅NO₄ calcd. mol. wt. 271.36).

### Step-2: Synthesis of methyl (1R,4R)-4-(methylamino)cyclohexane-1-carboxylate

To a solution of methyl (1R,4R)-4-((tert-butoxycarbonyl)(methyl)amino)cyclohexane-1-carboxylate (2.1 g, 7.74 mmol) in dichloromethane (20 mL) was added 20 mL of trifluoro acetic acid at 0° C. The reaction mixture was stirred at room temperature for 1 h. After complete consumption of the starting material (monitored by TLC), the reaction mixture was concentrated under reduced pressure to get methyl (1R,4R)-4-(methylamino)cyclohexane-1-carboxylate (3.58 g, quantitative yield) as the TFA salt. This was taken to the next step without further purification. LC purity: 89.21%; m/z: 172.2 [M+H]⁺ (Mol. formula C₉H₁₇NO₂ calcd. mol. wt. 171.24).

### Step-3: Synthesis of methyl (1R,4R)-4-((4-((5-cyclopropyl-1H-pyrazol-3-yl)amino)pyrimidin-2-yl)(methyl)amino)cyclohexane-1-carboxylate

To a solution of 2-chloro-N-(5-cyclopropyl-1H-pyrazol-3-yl)pyrimidin-4-amine (2 g, 8.5 mmol) in n-Butanol (20 mL) in a 100 mL sealed tube was added methyl (1R,4R)-4-(methylamino)cyclohexane-1-carboxylate (3.63 g, 21.27 mmol) and DIPEA (4.45 mL, 25.5 mmol). The reaction mixture was heated at 110° C. for 16 h. The progress of the reaction was monitored by TLC. After completion of the reaction, the reaction mixture was cooled to ambient temperature and concentrated to remove solvent. The crude product thus obtained was purified by Biotage Isolera using neutral alumina with gradient elution of 0-10% methanol in DCM to obtain methyl (1R,4R)-4-((4-((5-cyclopropyl-1H-pyrazol-3-yl)amino)pyrimidin-2-yl)(methyl)amino)cyclohexane-1-carboxylate (1 g, 32.25%). LC purity: 83.27%; m/z: 371 [M+H]⁺ (Mol. formula C₁₉H₂₆N₆O₂ calcd. mol. wt. 370.46).

### Step-4: Synthesis of (1R,4R)-4-((4-((5-cyclopropyl-1H-pyrazol-3-yl)amino)pyrimidin-2-yl)(methyl)amino)cyclohexane-1-carboxylic acid

To a stirred solution of methyl (1R,4R)-4-((4-((5-cyclopropyl-1H-pyrazol-3-yl)amino)pyrimidin-2-yl)(methyl)amino)cyclohexane-1-carboxylate (1 g, 2.699 mmol) in a mixture of solvents methanol: THF: water (8:8:8 mL) was added LiOH·H₂O (453 mg, 10.797 mmol). The reaction mixture was heated to 80° C. for 16 h. The completion of the reaction was monitored by TLC. The reaction mixture was concentrated to get the crude compound. The crude compound was neutralized with 1N HCl solution and then extracted with 10% solution of methanol in dichloromethane to get (1R,4R)-4-((4-((5-cyclopropyl-1H-pyrazol-3-yl)amino)pyrimidin-2-yl)(methyl)amino)cyclohexane-1-carboxylic acid (800 mg, 83%). LC purity: 79.85%; m/z: 357.0 [M+H]⁺ (Mol. formula C₁₈H₂₄N₆O₂, calcd. mol. wt. 356.43).

### Step-5: Synthesis of (1R,4R)-4-((4-((5-cyclopropyl-1H-pyrazol-3-yl)amino)pyrimidin-2-yl)(methyl)amino)-N-(2,3-dihydro-1H-inden-2-yl)cyclohexane-1-carboxamide

To a solution of (1R,4R)-4-((4-((5-cyclopropyl-1H-pyrazol-3-yl)amino)pyrimidin-2-yl)(methyl)amino)cyclohexane-1-carboxylic acid (200 mg, 0.561 mmol) in dry DCM (3 mL) was added triethylamine (0.23 mL, 1.683 mmol) drop-wise at 0° C. followed by T3P (0.45 mL, 1.402 mmol, 50% solution in ethyl acetate) and 2,3-dihydro-1H-inden-2-amine (75 mg, 0.561 mmol). The reaction was stirred at room temperature for 16 h. After completion of the reaction, the reaction mixture was diluted with dichloromethane washed with water and brine, and dried over anhydrous Na₂SO₄ and concentrated to yield the crude product. The crude compound was purified by reverse phase preparative HPLC to provide (1R,4R)-4-((4-((5-cyclopropyl-1H-pyrazol-3-yl)amino)pyrimidin-2-yl)(methyl)amino)-N-(2,3-dihydro-1H-inden-2-yl)cyclohexane-1-carboxamide (30 mg, 11.3%) as a TFA salt. LC purity: 95.67%; m/z: 472.2 [M+H]⁺ (Mol. formula C₂₇H₃₃N₇O, calcd. mol. wt. 471.61). ¹H NMR (400 MHz, CD₃OD): δ 7.72 (d, J=6.8 Hz, 1H), 7.24-7.20 (m, 2H), 7.17-7.14 (m, 2H), 6.37-6.33 (m, 2H), 4.61-4.57 (m, 1H), 3.32-3.24 (m, 2H), 3.06 (s, 3H), 2.84 (d, J=16, 15.6 Hz, 2H), 2.23-2.13 (m, 2H), 1.98-1.87 (m, 5H), 1.75-1.66 (m, 4H), 1.07-0.92 (m, 2H), 0.78-0.62 (m, 2H).

### Example 119: Synthesis of Compound 120

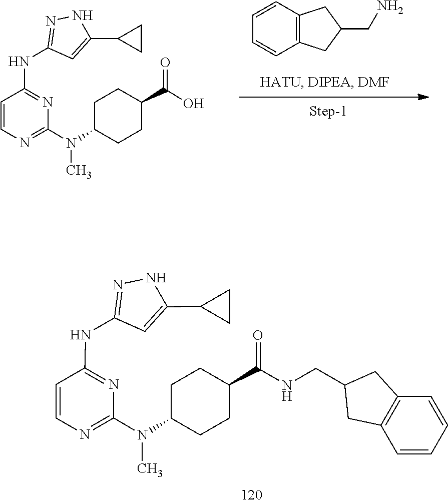 [SMILES: CN(c1nccc(Nc2cc(C3CC3)[nH]n2)n1)[C@H]1CC[C@H](C(=O)O)CC1 ; NCC1Cc2ccccc2C1 ; CN(c1nccc(Nc2cc(C3CC3)[nH]n2)n1)[C@H]1CC[C@H](C(=O)NCC2Cc3ccccc3C2)CC1]

### Step-1: Synthesis of (1R,4R)-4-((4-((5-cyclopropyl-1H-pyrazol-3-yl)amino)pyrimidin-2-yl)(methyl)amino)-N-((2,3-dihydro-1H-inden-2-yl)methyl)cyclohexane-1-carboxamide

To a stirred solution of (1R,4R)-4-((4-((5-cyclopropyl-1H-pyrazol-3-yl)amino)pyrimidin-2-yl)(methyl)amino)cyclohexane-1-carboxylic acid (200 mg, 0.561 mmol) in dry DMF (3 mL) was added DIPEA (0.24 mL, 1.404 mmol) followed by HATU (320 mg, 0.842 mmol). The reaction mixture was stirred at RT for 15 min, and then (2,3-dihydro-1H-inden-2-yl)methanamine (0.08 mL, 0.561 mmol) was added. The reaction mixture was stirred at room temperature for 16 h. After completion of the reaction (monitored by UPLC), the reaction mixture was diluted with dichloromethane washed with water, brine, dried over anhydrous Na₂SO₄ and concentrated to get the residue. The residue was purified by reverse phase preparative HPLC to yield (1R,4R)-4-((4-((5-cyclopropyl-1H-pyrazol-3-yl)amino)pyrimidin-2-yl)(methyl)amino)-N-((2,3-dihydro-1H-inden-2-yl)methyl)cyclohexane-1-carboxamide (50 mg, 18.38%) as the TFA salt. LC purity: 99.70%; m/z: 486.3 [M+H]⁺ (Mol. formula C₂₈H₃₅N₇O, calcd. mol. wt. 485.64). ¹H NMR (400 MHz, CD₃OD): δ 7.73 (d, J=7.2 Hz, 1H), 7.19-7.16 (m, 2H), 7.12-7.09 (m, 2H), 6.37 (s, 1H), 6.34 (d, J=7.2 Hz, 1H), 3.27 (d, J=6.4 Hz, 2H), 3.07 (s, 3H), 3.06-3.02 (m, 2H), 2.72-2.67 (m, 3H), 2.30-2.26 (m, 1H), 1.33-1.30 (m, 1H), 1.99-1.88 (m, 5H), 1.86-1.73 (m, 4H), 1.06-1.01 (m, 2H), 0.78-0.76 (m, 2H).

### Example 120: Synthesis of Compound 121

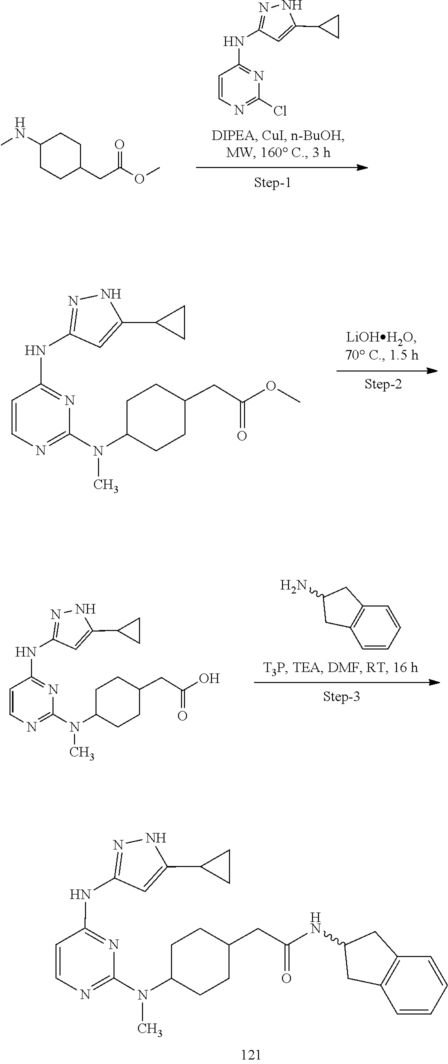

### Step-1: Synthesis of methyl 2-(4-((4-((5-cyclopropyl-1H-pyrazol-3-yl)amino)pyrimidin-2-yl)(methyl)amino)cyclohexyl)acetate

To a solution of methyl 2-(4-(methylamino)cyclohexyl)acetate (530 mg, 2.864 mmol) in n-BuOH (6 mL) In a 20 mL microwave vial was added DIPEA (0.74 mL, 4.296 mmol), 2-chloro-N-(5-cyclopropyl-1H-pyrazol-3-yl)pyrimidin-4-amine (336.6 mg, 1.432 mmol) and copper iodide (53 mg). The reaction was heated in a MW reactor at 160° C. for 2 h. After completion of the reaction (monitored by TLC), the reaction mixture was concentrated to obtain the crude compound. The crude compound was purified by Biotage Isolera using silica gel (230-400 mesh) with gradient elution of 0-100% ethyl acetate in pet ether to afford methyl 2-(4-((4-((5-cyclopropyl-1H-pyrazol-3-yl)amino)pyrimidin-2-yl)(methyl)amino)cyclohexyl)acetate (240 mg, 21%). LC purity: 95.5%; m/z: 385.23 [M+H]⁺ (Mol. formula C₂₀H₂₈N₆O₂, calcd. mol. Wt 384.28).

### Step-2: Synthesis of 2-(4-((4-((5-cyclopropyl-1H-pyrazol-3-yl)amino)pyrimidin-2-yl)(methyl)amino)cyclohexyl)acetic acid

To a stirred solution of methyl 2-(4-((4-((5-cyclopropyl-1H-pyrazol-3-yl)amino)pyrimidin-2-yl)(methyl)amino)cyclohexyl)acetate (240 mg, 0.625 mmol) in water, THF, and methanol (1:1:1, 3 mL) was added lithium hydroxide monohydrate (131.2 mg, 3.125 mmol). The reaction was heated at 70° C. for 1.5 h. After completion of the reaction (monitored by TLC), the reaction mixture was concentrated to obtain 2-(4-((4-((5-cyclopropyl-1H-pyrazol-3-yl)amino)pyrimidin-2-yl)(methyl)amino)cyclohexyl)acetic acid (200 mg, crude). LC purity: 89.4%; m/z: 371.22 [M+H]⁺ (Mol. formula C₁₉H₂₆N₆O₂, calcd. mol. wt. 370.46).

### Step-3: Synthesis of 2-(4-((4-((5-cyclopropyl-1H-pyrazol-3-yl)amino)pyrimidin-2-yl)(methyl)amino)cyclohexyl)-N-(2,3-dihydro-1H-inden-2-yl)acetamide

To a stirred solution of 2-(4-((4-((5-cyclopropyl-1H-pyrazol-3-yl)amino)pyrimidin-2-yl)(methyl)amino)cyclohexyl)acetic acid (200 mg, 0.54 mmol) in dry DMF (2 mL) was added TEA (0.22 mL, 1.621 mmol), T3P (0.24 ml, 0.81 mmol, 50% solution in ethyl acetate) and stirred the reaction for 15 minutes at room temperature. Then 2,3-dihydro-1H-inden-2-amine (71.82 mg, 0.54 mmol) was added. The reaction mixture was stirred at room temperature for 16 h. The progress of the reaction was monitored by TLC. After complete consumption of starting material, the reaction mixture was diluted with water and extracted with dichloromethane. The resulting organic layer was washed with brine solution then dried over anhydrous Na₂SO₄ and concentrated to obtain crude. The crude compound was purified by reverse phase preparative HPLC to give 2-(4-((4-((5-cyclopropyl-1H-pyrazol-3-yl)amino)pyrimidin-2-yl)(methyl) amino)cyclohexyl)-N-(2,3-dihydro-1H-inden-2-yl)acetamide (50 mg, 19%) as a free base. LC purity: 98.78%; m/z: 486.29 [M+H]⁺ (Mol. formula C₂₈H₃₅N₇O, calcd. mol. wt. 485.64). ¹H VTNMR (400 MHz, CD₃OD): δ 7.85 (s, 1H), 7.22-7.13 (m, 4H), 6.27-6.13 (s, 2H), 3.33-3.24 (m, 3H), 3.00 (s, 3H), 2.98-2.83 (m, 2H), 2.37-2.12 (m, 3H), 1.91-1.76 (m, 8H), 1.52-1.47 (m, 1H), 1.39-1.33 (m, 1H), 0.99-0.96 (m, 2H), 0.75-0.71 (m, 2H).

### Example 121: Synthesis of Compound 122

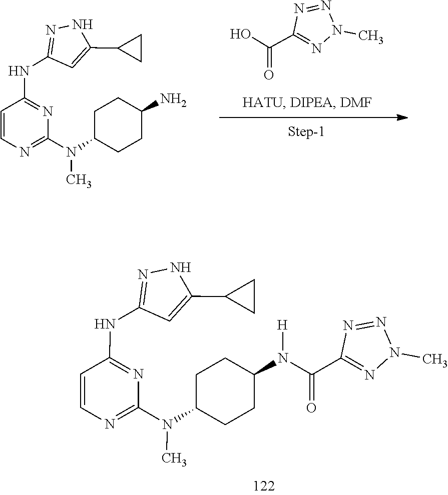

### Step-1: Synthesis of N-((1R,4R)-4-((4-((5-cyclopropyl-1H-pyrazol-3-yl)amino)pyrimidin-2-yl)(methyl)amino)cyclohexyl)-2-methyl-2H-tetrazole-5-carboxamide

To a stirred solution of 2-methyl-2H-tetrazole-5-carboxylic acid (78.2 mg, 0.611 mmol) in dry DMF (2 mL) was added DIPEA (0.31 mL, 1.834 mmol) followed by HATU (464 mg, 1.223 mmol) and stirred the reaction mixture for 15 min then added N2-((1R,4R)-4-aminocyclohexyl)-N4-(5-cyclopropyl-1H-pyrazol-3-yl)-N2-methylpyrimidine-2,4-diamine (200 mg, 0.611 mmol). The reaction was stirred at room temperature for 16 h. After completion of the reaction (monitored by UPLC), the reaction mixture was diluted with dichloromethane, washed with water and brine, dried over anhydrous Na₂SO₄ and concentrated to yield the residue. The residue was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-2-methyl-2H-tetrazole-5-carboxamide (70 mg, 26.92%) as the free base. LC purity: 99.92%; m/z: 438.3 [M+H]⁺ (Mol. formula C₂₀H₂₇N₁₁O, calcd. mol. wt. 437.51). ¹H NMR (400 MHz, CD₃OD): δ 7.87 (s, 1H), 6.33-6.16 (m, 2H), 4.64-4.60 (m, 1H), 4.45 (s, 3H), 4.01-3.95 (m, 1H), 3.11 (s, 3H), 2.23-2.00 (m, 2H), 1.99-1.89 (m, 5H), 1.72-1.64 (m, 2H), 1.02-0.99 (m, 2H), 0.76-0.72 (m, 2H).

### Example 122: Synthesis of Compound 123

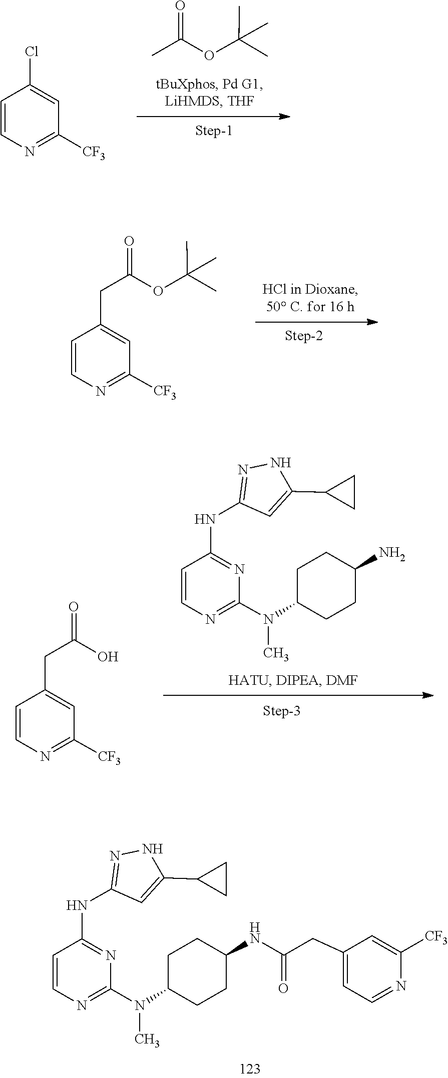

### Step-1: Synthesis of tert-butyl 2-(2-(trifluoromethyl)pyridin-4-yl)acetate

To a stirred solution of 4-chloro-2-(trifluoromethyl)pyridine (1 g, 5.524 mmol), tert-butyl acetate (1.5 mL, 11.049 mmol) and ^{t}BuXPhos Pd G1 (75.8 mg, 0.110 mmol) in dry THF (10 mL) was added LiHMDS (16.5 mL, 16.574 mmol, 1M in THF) slowly dropwise at 0° C. The reaction mixture was slowly allowed to room temperature and the reaction was stirred for 16 h. The progress of the reaction was monitored by TLC. After completion of the reaction, the reaction mixture was quenched with saturated NH₄Cl solution, extracted with ethyl acetate and the organic layer was washed with water. The organic layer was dried over anhydrous sodium sulphate, filtered and concentrated under reduced pressure to yield tert-butyl 2-(2-(trifluoromethyl)pyridin-4-yl)acetate (1.2 g, 83.33%). LC purity: 52%; m/z: 262.1 [M+H]⁺ (Mol. formula C₁₂H₁₄NO₂F₃ calcd. mol. wt. 261.14).

### Step-2: Synthesis of 2-(2-(trifluoromethyl)pyridin-4-yl)acetic acid

To a stirred solution of tert-butyl 2-(2-(trifluoromethyl)pyridin-4-yl)acetate (160 mg, 0.613 mmol) was added HCl in dioxane (2 mL, 4M solution). The reaction mixture was stirred at 50° C. for 16 h. After completion of the reaction (monitored by UPLC), the reaction mixture was concentrated under reduced pressure to get 2-(2-(trifluoromethyl)pyridin-4-yl)acetic acid (80 mg, 64.0%) which was directly used for the next step without further purification. (Mol. formula C₈H₆NO₂F₃ calcd. mol. wt. 205.14).

### Step-3: Synthesis of N-((1R,4R)-4-((4-((5-cyclopropyl-1H-pyrazol-3-yl)amino)pyrimidin-2-yl)(methyl)amino)cyclohexyl)-2-(2-(trifluoromethyl)pyridin-4-yl)acetamide

To a stirred solution of 2-(2-(trifluoromethyl)pyridin-4-yl)acetic acid (80.2 mg, 0.391 mmol) in dry DMF (3 mL) was added triethylamine (0.2 mL, 1.467 mmol) followed by the addition of the EDC·HCl (140.18 mg, 0.733 mmol) and HOBt (33.02 mg, 0.244 mmol). The reaction mixture was stirred at room temperature for 15 minutes, and then N2-((1R,4R)-4-aminocyclohexyl)-N4-(5-cyclopropyl-1H-pyrazol-3-yl)-N2-methylpyrimidine-2,4-diamine (160 mg, 0.489 mmol) was added. The reaction mixture was stirred at room temperature for 16 h. After completion of the reaction (monitored by UPLC), the reaction mixture was diluted with dichloromethane washed with water and brine, dried over anhydrous Na₂SO₄ and concentrated under reduced pressure to yield the crude compound. The crude compound was purified by reverse phase preparative HPLC (0.1% TFA in water/acetonitrile) to yield N-((1R,4R)-4-((4-((5-cyclopropyl-1H-pyrazol-3-yl)amino)pyrimidin-2-yl)(methyl)amino)cyclohexyl)-2-(2-(trifluoromethyl)pyridin-4-yl)acetamide (20 mg, 7.96%). LC purity: 94.6%; m/z: 515.2 [M+H]⁺ (Mol. formula C₂₅H₂₉F₃N₈O, calcd. mol. wt. 514.56). ¹H NMR (400 MHz, CD₃OD): δ 8.65 (d, J=5.2 Hz, 1H), 7.79-7.74 (m, 2H), 7.60 (d, J=4.8 Hz, 1H), 6.40-6.36 (m, 2H), 4.66-4.50 (m, 1H), 3.72-3.69 (m, 1H), 3.66 (s, 2H), 3.08 (s, 3H), 2.17-2.12 (m, 2H), 1.98-1.94 (m, 1H), 1.90-1.85 (m, 4H), 1.49-1.41 (m, 2H), 1.03-0.92 (m, 2H), 0.77-0.73 (m, 2H).

### Example 123: Synthesis of Compound 124

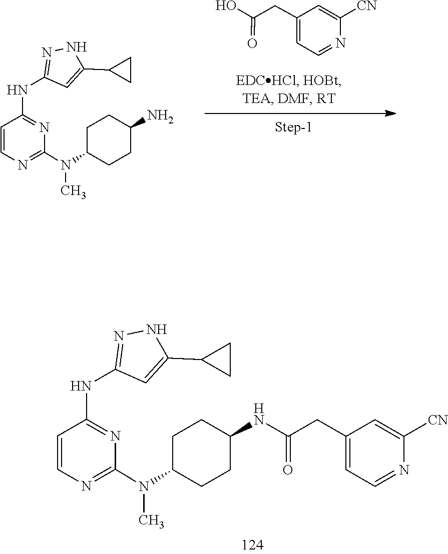 [SMILES: CN(c1nccc(Nc2cc(C3CC3)[nH]n2)n1)[C@H]1CC[C@H](N)CC1 ; N#Cc1cc(CC(=O)O)ccn1 ; CN(c1nccc(Nc2cc(C3CC3)[nH]n2)n1)[C@H]1CC[C@H](NC(=O)Cc2ccnc(C#N)c2)CC1]

### Step-1: Synthesis of 2-(2-cyanopyridin-4-yl)-N-((1R,4R)-4-((4-((5-cyclopropyl-1H-pyrazol-3-yl)amino)pyrimidin-2-yl)(methyl)amino)cyclohexyl)acetamide

To a stirred solution of 2-(2-cyanopyridin-4-yl)acetic acid (79.2 mg, 0.489 mmol) in dry DMF (3 mL) was added triethylamine (0.25 mL, 1.834 mmol) followed by the addition of EDC·HCl (175 mg, 0.917 mmol) and HOBt (41.2 mg, 0.305 mmol). The reaction mixture was stirred at room temperature for 15 minutes and then N2-((1R,4R)-4-aminocyclohexyl)-N4-(5-cyclopropyl-1H-pyrazol-3-yl)-N2-methylpyrimidine-2,4-diamine (200 mg, 0.611 mmol) was added. The reaction mixture was stirred at room temperature for 16 h. After completion of the reaction (monitored by UPLC), the mixture was diluted with dichloromethane washed with water, brine, dried over anhydrous Na₂SO₄ and concentrated under reduced pressure to get the crude compound. The crude was purified by reverse phase preparative HPLC with mobile phase 0.1% TFA in water/acetonitrile to yield 2-(2-cyanopyridin-4-yl)-N-((1R,4R)-4-((4-((5-cyclopropyl-1H-pyrazol-3-yl)amino)pyrimidin-2-yl)(methyl)amino)cyclohexyl)acetamide (100 mg, 35.71%). LC purity: 99.74%; m/z: 472.2 [M+H]⁺ (Mol. formula C₂₅H₂₉N₉O, calcd. mol. wt. 471.57). ¹H VTNMR (400 MHz, CD₃OD): δ 8.62 (d, J=5.2 Hz, 1H), 7.81 (s, 1H), 7.72 (d, J=7.2 Hz, 1H), 7.61-7.59 (m, 1H), 6.39-6.26 (m, 2H), 4.55-3.95 (m, 1H), 3.70-3.68 (m, 1H), 3.65 (s, 2H), 3.08 (s, 3H), 2.16-2.11 (m, 2H), 1.98-1.93 (m, 1H), 1.88-1.82 (m, 4H), 1.49-1.45 (m, 2H), 1.04-0.99 (m, 2H), 0.77-0.73 (m, 2H).

### Example 124: Synthesis of Compound 125

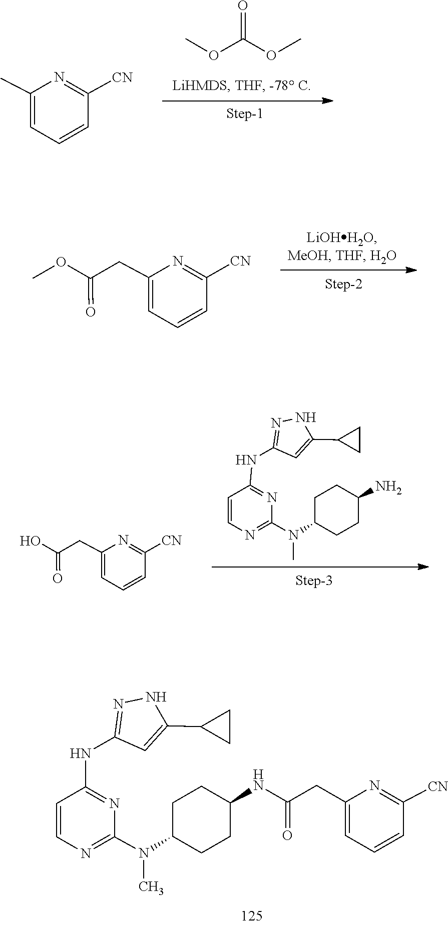

### Step-1: Synthesis of methyl 2-(6-cyanopyridin-2-yl)acetate

To a stirred solution of 6-methylpicolinonitrile (500 mg, 4.237 mmol) in dry THF (5 mL) was added LiHMDS (6.35 mL, 6.355 mmol, 1M in THF) dropwise at −78° C. and the reaction mixture was stirred for 1 h. Then dimethyl carbonate (0.42 mL, 5.084 mmol) was added to the reaction mixture at −78° C. and the reaction mixture was allowed to stir t at ambient temperature for 2.5 h. The progress of the reaction was monitored by TLC, then the reaction mixture was quenched with saturated NH₄CI solution and extracted with ethyl acetate. The organic layer was dried over anhydrous sodium sulfate, filtered and concentrated under reduced pressure to yield methyl 2-(6-cyanopyridin-2-yl)acetate (450 mg, 60.40%). LC purity: 77.05%; m/z: 177.1 [M+H]⁺ (Mol. formula C₉H₈N₂O₂ calcd. mol. wt. 176.18).

### Step-2: Synthesis of 2-(6-cyanopyridin-2-yl)acetic acid

To a stirred solution of methyl 2-(6-cyanopyridin-2-yl) acetate (400 mg, 2.272 mmol) in THF, methano and water (1:1:1, 5 mL) was added lithium hydroxide monohydrate (47.68 mg, 1.136 mmol). The reaction mixture was allowed to stir at room temperature for 1 h. The progress of the reaction was monitored by UPLC. After completion of the reaction, the reaction mixture was concentrated under reduced pressure to obtain 2-(6-cyanopyridin-2-yl)acetic acid (300 mg, 81.52%). LC purity: 59.44%; m/z: 163.0 [M+H]⁺ (Mol. formula C₈H₆N₂O₂ calcd. mol. wt. 162.15)

### Step-3: Synthesis of 2-(6-cyanopyridin-2-yl)-N-((1R,4R)-4-((4-((5-cyclopropyl-1H-pyrazol-3-yl)amino)pyrimidin-2-yl)(methyl)amino)cyclohexyl)acetamide

To a stirred solution of 2-(6-cyanopyridin-2-yl)acetic acid (99.08 mg, 0.611 mmol) in dry DMF (3 mL) was added triethylamine (0.32 mL, 2.293 mmol) followed by the addition of the EDC·HCl (219.03 mg, 1.146 mmol) and HOBt (51.60 mg, 0.382 mmol). The reaction mixture was stirred at room temperature for 15 minutes, and then N2-((1R,4R)-4-aminocyclohexyl)-N4-(5-cyclopropyl-1H-pyrazol-3-yl)-N2-methylpyrimidine-2,4-diamine (250 mg, 0.764 mmol) was added. The reaction mixture was stirred at room temperature for 16 h. After completion of the reaction (monitored by UPLC), the reaction mixture was diluted with dichloromethane washed with water, and brine, dried over anhydrous Na₂SO₄ and concentrated under reduced pressure to yield the crude compound. The crude product was purified by reverse phase preparative HPLC to yield 2-(6-cyanopyridin-2-yl)-N-((1R,4R)-4-((4-((5-cyclopropyl-1H-pyrazol-3-yl)amino) pyrimidin-2-yl)(methyl)amino)cyclohexyl)acetamide (40 mg, 11.11%). LC purity: 98.62%; m/z: 472.3 [M+H]⁺ (Mol. formula C₂₅H₂₉N₉O calcd. mol. wt. 471.57). ¹H NMR (400 MHz, CD₃OD): δ 8.31 (d, J=7.2 Hz, 1H), 7.99-7.95 (m, 1H), 7.83-7.67 (m, 2H), 6.40-6.37 (m, 2H), 4.83-4.81 (m, 1H), 3.79 (s, 2H), 3.75-3.67 (m, 1H), 3.08 (s, 3H), 2.20-2.13 (m, 2H), 1.98-1.95 (m, 1H), 1.85-1.80 (m, 4H), 1.54-1.44 (m, 2H), 1.06-1.05 (m, 2H), 0.79-0.75 (m, 2H).

### Example 125: Synthesis of Compound 126

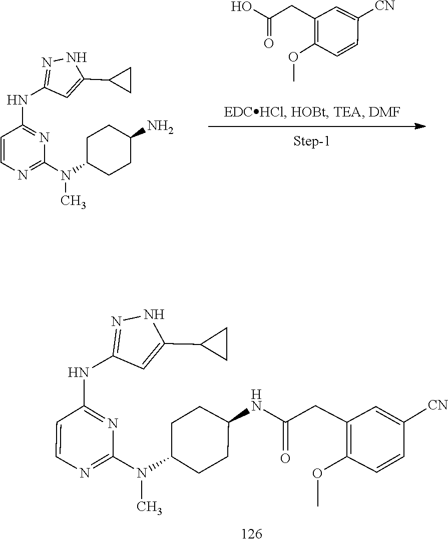 [SMILES: CN(c1nccc(Nc2cc(C3CC3)[nH]n2)n1)[C@H]1CC[C@H](N)CC1 ; COc1ccc(C#N)cc1CC(=O)O ; COc1ccc(C#N)cc1CC(=O)N[C@H]1CC[C@H](N(C)c2nccc(Nc3cc(C4CC4)[nH]n3)n2)CC1]

### Step-1: Synthesis of 2-(5-cyano-2-methoxyphenyl)-N-((1R,4R)-4-((4-((5-cyclopropyl-1H-pyrazol-3-yl)amino)pyrimidin-2-yl)(methyl)amino)cyclohexyl)acetamide

To a stirred solution of 2-(5-cyano-2-methoxyphenyl)acetic acid (70 mg, 0.366 mmol) in dry DMF (2 mL) was added triethylamine (0.19 mL, 1.376 mmol) followed by the addition of the EDC·HCl (131 mg, 0.688 mmol) and HOBt (30.9 mg, 0.229 mmol). The reaction mixture was stirred at room temperature for 15 min then N2-((1R,4R)-4-aminocyclohexyl)-N4-(5-cyclopropyl-1H-pyrazol-3-yl)-N2-methylpyrimidine-2,4-diamine (150 mg, 0.458 mmol) was added. Then reaction mixture was stirred at room temperature for 7 h. After completion of the reaction (monitored by UPLC), the reaction mixture was diluted with dichloromethane washed with water and brine, dried over anhydrous Na₂SO₄ and concentrated under reduced pressure to yield the crude compound. The crude product was purified by reverse phase preparative HPLC with mobile phase 0.1% TFA in water/acetonitrile to yield 2-(5-cyano-2-methoxyphenyl)-N-((1R,4R)-4-((4-((5-cyclopropyl-1H-pyrazol-3-yl)amino)pyrimidin-2-yl)(methyl)amino)cyclohexyl)acetamide (40 mg, 17.46%). LC purity: 99.53%; m/z: 501.9 [M+H]⁺ (Mol. formula C₂₇H₃₂N₈O₂, calcd. mol. wt. 500.61). ¹H NMR (400 MHz, CD₃OD): δ 7.73-7.67 (m, 2H), 7.57-7.54 (m, 1H), 7.13 (d, J=8.4 Hz, 1H), 6.37-6.34 (m, 2H), 4.64-4.60 (m, 1H), 3.92 (s, 3H), 3.70-3.68 (m, 1H), 3.55 (s, 2H), 3.08 (s, 3H), 2.16-2.10 (m, 2H), 1.96-1.91 (m, 1H), 1.86-1.82 (m, 4H), 1.49-1.31 (m, 2H), 1.04-0.99 (m, 2H), 0.78-0.74 (m, 2H).

### Example 126: Synthesis of Compound 127

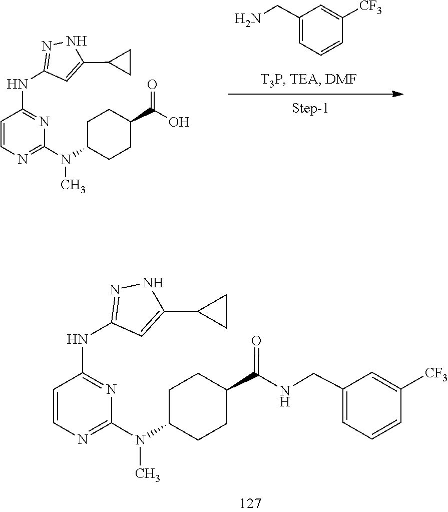 [SMILES: CN(c1nccc(Nc2cc(C3CC3)[nH]n2)n1)[C@H]1CC[C@H](C(=O)O)CC1 ; NCc1cccc(C(F)(F)F)c1 ; CN(c1nccc(Nc2cc(C3CC3)[nH]n2)n1)[C@H]1CC[C@H](C(=O)NCc2cccc(C(F)(F)F)c2)CC1]

### Step-1: Synthesis of (1R,4R)-4-((4-((5-cyclopropyl-1H-pyrazol-3-yl)amino)pyrimidin-2-yl)(methyl)amino)-N-(3-(trifluoromethyl)benzyl)cyclohexane-1-carboxamide

To a stirred solution of (1R,4R)-4-((4-((5-cyclopropyl-1H-pyrazol-3-yl)amino)pyrimidin-2-yl)(methyl)amino)cyclohexane-1-carboxylic acid (200 mg, 0.561 mmol) in dry DMF (3 mL) was added triethylamine (0.23 mL, 1.685 mmol) followed by the addition of the T3P (0.7 mL, 1.123 mmol, 50% solution in EtOAc). The reaction mixture was stirred at room temperature for 10 min then (3-(trifluoromethyl)phenyl)methanamine (0.8 mL, 0.561 mmol) was added. The reaction mixture was stirred at room temperature for 12 h. After completion of the reaction (monitored by UPLC), the reaction mixture was diluted with dichloromethane, washed with water and brine, dried over anhydrous Na₂SO₄ and concentrated under reduced pressure to yield the crude compound. The crude was purified by reverse phase preparative HPLC to yield (1R,4R)-4-((4-((5-cyclopropyl-1H-pyrazol-3-yl)amino)pyrimidin-2-yl)(methyl)amino)-N-(3-(trifluoromethyl)benzyl)cyclohexane-1-carboxamide (30 mg, 10.71%). LC purity: 99.57%; m/z: 514.2 [M+H]⁺ (Mol. formula C₂₆H₃₀N₇OF₃, calcd. mol. wt. 513.57).

### Example 127: Synthesis of Compounds 128 & 129

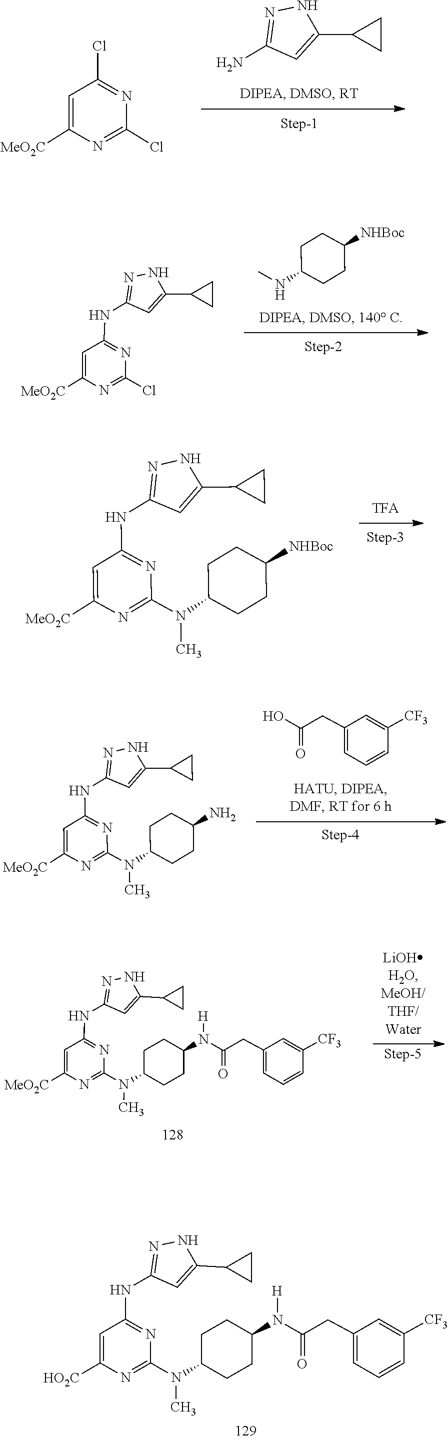

### Step-1: Synthesis of methyl 2-chloro-6-((5-cyclopropyl-1H-pyrazol-3-yl)amino)pyrimidine-4-carboxylate

To a stirred solution of methyl 2,6-dichloropyrimidine-4-carboxylate (2 g, 9.661 mmol) in DMSO (10 mL) was added DIPEA (3.36 mL, 19.322 mmol) and 5-cyclopropyl-1H-pyrazol-3-amine (1.3 g, 10.627 mmol). The reaction mixture was stirred at room temperature for 16 h. The progress of the reaction was monitored by UPLC, and after complete consumption of the starting material, the reaction mixture was quenched with water and the solid was filtered, washed with pet ether and dried under vacuum to yield methyl 2-chloro-6-((5-cyclopropyl-1H-pyrazol-3-yl)amino)pyrimidine-4-carboxylate (1.5 g, 53%). LC purity: 94.84%; m/z: 294.0 [M+H]⁺ (Mol. formula C₁₂H₁₂ClN₅O₂, calcd. mol. wt. 293.71).

### Step-2: Synthesis of methyl 2-(((1R,4R)-4-((tert-butoxycarbonyl)amino)cyclohexyl) (methyl)amino)-6-((5-cyclopropyl-1H-pyrazol-3-yl)amino)pyrimidine-4-carboxylate

To a mixture of methyl 2-chloro-6-((5-cyclopropyl-1H-pyrazol-3-yl)amino)pyrimidine-4-carboxylate (1.5 g, 5.107 mmol) and tert-butyl ((1R,4R)-4-(methylamino)cyclohexyl)carbamate (2.9 g, 12.76 mmol) in DMSO (15 mL) in a 20 mL microwave vial was added DIPEA (1.78 mL, 10.214 mmol). The reaction mixture was heated at 140° C. in a microwave for 4 h. The reaction mixture was quenched with ice-cold water and extracted with dichloromethane. The organic layer was dried over anhydrous sodium sulphate and evaporated to get crude. The obtained crude was purified by Biotage Isolera using 230-400 silica mesh eluted with 0-60% ethyl acetate in pet ether as a eluent to yield methyl 2-(((1R,4R)-4-((tert-butoxycarbonyl)amino)cyclohexyl)(methyl)amino)-6-((5-cyclopropyl-1H-pyrazol-3-yl)amino)pyrimidine-4-carboxylate (1.2 g, 48.5%). LC purity: 54.66% m/z: 486.2 [M+H]⁺ (Mol. formula C₂₄H₃₅N₇O₄, calcd. mol. wt. 485.59).

### Step-3: Synthesis of methyl 2-(((1R,4R)-4-aminocyclohexyl)(methyl)amino)-6-((5-cyclopropyl-1H-pyrazol-3-yl)amino)pyrimidine-4-carboxylate

To a cooled 0° C. solution of methyl 2-(((1R,4R)-4-((tert-butoxycarbonyl)amino) cyclohexyl) (methyl)amino)-6-((5-cyclopropyl-1H-pyrazol-3-yl)amino)pyrimidine-4-carboxylate (500 mg, 1.029 mmol) in dry DCM (10 mL) was added TFA (5 mL). The reaction was allowed to stir at room temperature for 3 h. The progress of the reaction was monitored by TLC, and after complete consumption of starting material, the resulting mixture was concentrated, triturated with pet ether, and concentrated again under high vacuume to yield methyl 2-(((1R,4R)-4-aminocyclohexyl)(methyl)amino)-6-((5-cyclopropyl-1H-pyrazol-3-yl)amino)pyrimidine-4-carboxylate as a TFA salt (500 mg, quantitative yield). LC purity: 66.94%; m/z: 386.2 [M+H]⁺ (Mol. formula C₁₉H₂₇N₇O₂, calcd. mol. wt. 385.47).

### Step-4: Synthesis of methyl 6-((5-cyclopropyl-1H-pyrazol-3-yl)amino)-2-(methyl((1R,4R)-4-(2-(3-(trifluoromethyl)phenyl)acetamido)cyclohexyl)amino)pyrimidine-4-carboxylate

To a solution of methyl 2-(((1R,4R)-4-aminocyclohexyl)(methyl)amino)-6-((5-cyclopropyl-1H-pyrazol-3-yl)amino)pyrimidine-4-carboxylate (700 mg, 1.816 mmol) in dry DMF (7 mL) was added DIPEA (0.79 mL, 4.54 mmol) drop-wise at 0° C. followed by HATU (1.03 g, 2.724 mmol) and 2-(3-(trifluoromethyl)phenyl)acetic acid (185 mg, 0.908 mmol). The reaction mixture was stirred at room temperature for 6 h. After completion of the reaction, the reaction mixture was diluted with dichloromethane washed with water and brine, dried over anhydrous Na₂SO₄ and concentrated to yield the crude product. The crude compound was purified by reverse phase prep HPLC to provide methyl 6-((5-cyclopropyl-1H-pyrazol-3-yl)amino)-2-(methyl((1R,4R)-4-(2-(3-(trifluoromethyl)phenyl)acetamido)cyclohexyl)amino)pyrimidine-4-carboxylate (400 mg, 38.8%) as a solid. LC purity: 95.95%; m/z: 572.2 [M+H]⁺ (Mol. formula C₂₈H₃₂F₃N₇O₃, calcd. mol. wt. 571.61).

### Step-5: Synthesis of 6-((5-cyclopropyl-1H-pyrazol-3-yl)amino)-2-(methyl((1R,4R)-4-(2-(3-(trifluoromethyl)phenyl)acetamido)cyclohexyl)amino)pyrimidine-4-carboxylic acid

To a stirred solution of methyl 6-((5-cyclopropyl-1H-pyrazol-3-yl)amino)-2-(methyl((1R,4R)-4-(2-(3-(trifluoromethyl)phenyl)acetamido)cyclohexyl)amino)pyrimidine-4-carboxylate (150 mg, 0.262 mmol) in a mixture of solvents methanol: THF: water (2:2:2 mL) was added LiOH·H₂O (22 mg, 0.524 mmol). The reaction mixture was heated to 80° C. for 6 h. The completion of the reaction was monitored by UPLC. The reaction mixture was concentrated to obtain the crude product. The crude product was purified by reverse phase prep HPLC with a mobile phase of 0.1% TFA in water/acetonitrile to yield 6-((5-cyclopropyl-1H-pyrazol-3-yl)amino)-2-(methyl((1R,4R)-4-(2-(3-(trifluoromethyl)phenyl)acetamido) cyclohexyl)amino)pyrimidine-4-carboxylic acid (30 mg, 20.5%). LC purity: 98.64%; m/z: 558.3 [M+H]⁺ (Mol. formula C₂₇H₃₀F₃N₇O₃, calcd. mol. wt. 557.58).

### Example 128: Synthesis of Compound 130

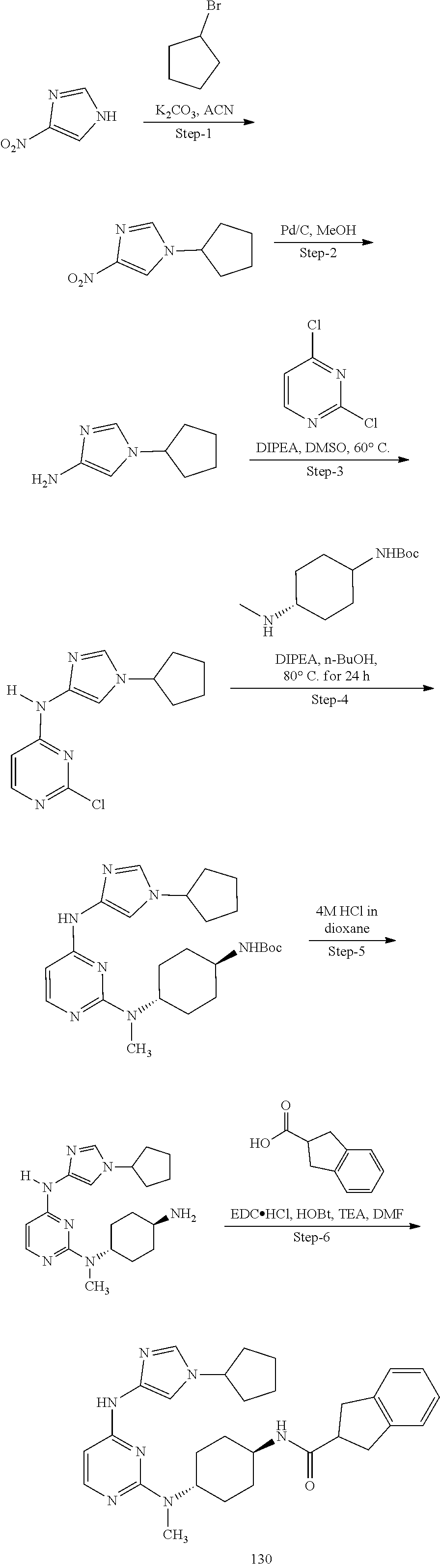

### Step-1: Synthesis of 1-cyclopentyl-4-nitro-1H-imidazole

To a solution of 4-nitro-1H-imidazole (5 g, 44.27 mmol) in ACN (200 mL) was added potassium carbonate (12.2 g, 88.54 mmol) and bromocyclopentane (5.6 mL, 53.097 mmol). The reaction mixture was stirred at room temperature for 16 h. The progress of the reaction was monitored by TLC. After completion of the reaction, the reaction mixture was filtered over bed of celite and the filtrate was concentrated to get the residue. The residue was purified by Biotage Isolera using silica gel (230-400 mesh) with gradient elution of 0-100% ethyl acetate in pet ether to afford 1-cyclopentyl-4-nitro-1H-imidazole (5 g, 62% yield). LC purity: 97.1%; m/z: 182.09 [M+H]⁺ (Mol. formula C₈H₁₁N₃O₂, calcd. mol. wt. 181.20).

### Step-2: Synthesis of 1-cyclopentyl-1H-imidazol-4-amine

To a solution of 1-cyclopentyl-4-nitro-1H-imidazole (5 g, 27.624 mmol) in methanol (50 mL) was added Pd/C (1.0 g) under N₂ atmosphere. The reaction mixture was stirred at room temperature for 16 h under hydrogen atmosphere. The progress of the reaction was monitored by TLC. After completion of the reaction, the reaction mixture filtered over a bed of celite and the filtrate was concentrated to obtain 1-cyclopentyl-1H-imidazol-4-amine. (4.5 g, crude). LC purity: 44.6%; m/z: 152.11 [M+H]⁺ (Mol. formula C₈H₁₃N₃, calcd. mol. wt. 151.21).

### Step-3: Synthesis of 2-chloro-N-(1-cyclopentyl-1H-imidazol-4-yl)pyrimidin-4-amine

To a solution of 1-cyclopentyl-1H-imidazol-4-amine (4.5 g, 29.801 mmol) in DMSO (45 mL) was added 2,4-dichloropyrimidine (5.2 g, 35.76 mmol) and DIPEA (10.3 mL, 72.846 mmol). The reaction mixture was stirred at 60° C. for 16 h. The progress of the reaction was monitored by TLC. After completion of the reaction, the reaction mixture was diluted with water, extracted with dichloromethane, washed with brine, dried over anhydrous Na₂SO₄ and concentrated to obtain the crude product. The crude product was purified by Biotage Isolera using silica gel (230-400 mesh) with gradient elution of 0-100% ethyl acetate in pet ether to afford 2-chloro-N-(1-cyclopentyl-1H-imidazol-4-yl)pyrimidin-4-amine (5.0 g, 64%). LC purity: 73.4%; m/z: 265.01 [M+H]⁺ (Mol. formula C₁₂H₁₄ClN₅, calcd. mol. wt. 263.73).

### Step-4: Synthesis of tert-butyl ((1R,4R)-4-((4-((1-cyclopentyl-1H-imidazol-4-yl)amino)pyrimidin-2-yl)(methyl)amino)cyclohexyl)carbamate

A mixture of 2-chloro-N-(1-cyclopentyl-1H-imidazol-4-yl)pyrimidin-4-amine (500 mg, 1.901 mmol), tert-butyl ((1R,4R)-4-(methylamino)cyclohexyl)carbamate (650 mg, 2.851 mmol) and DIPEA (0.99 mL, 5.703 mmol) in n-BuOH (5 mL) was stirred at 110° C. for 16 h. The progress of the reaction was monitored by TLC. After complete consumption of starting material, the reaction mixture was cooled to room temperature, diluted with water and extracted with DCM. The organic layer separated was dried over anhydrous sodium sulphate and concentrated. The obtained crude product was purified by Biotage Isolera using by using silica gel (230-400 mesh) with gradient elution of 0-100% ethyl acetate in pet ether to afford tert-butyl ((1R,4R)-4-((4-((1-cyclopentyl-1H-imidazol-4-yl)amino)pyrimidin-2-yl)(methyl)amino)cyclohexyl)carbamate (300 mg, 34% yield). LC purity: 79.1%; m/z: 456.30 [M+H]⁺ (Mol. formula C₂₄H₃₇N₇O₂, calcd. mol. Wt. 455.61).

### Step-5: Synthesis of N²-((1R,4R)-4-aminocyclohexyl)-N4-(1-cyclopentyl-1H-imidazol-4-yl)-N²-methylpyrimidine-2,4-diamine

A solution of tert-butyl ((1R,4R)-4-((4-((1-cyclopentyl-1H-imidazol-4-yl)amino)pyrimidin-2-yl)(methyl)amino)cyclohexyl)carbamate (300 mg, 0.659 mmol) in DCM (3 mL) was cooled to 0° C. and 4M HCl in 1,4 dioxane (3 mL) was added. The resulting mixture was stirred at room temperature for 1 h. The progress of the reaction was monitored by TLC, and after complete consumption of starting material, the reaction mixture was concentrated under vacuum to yield N²-((1R,4R)-4-aminocyclohexyl)-N4-(1-cyclopentyl-1H-imidazol-4-yl)-N²-methylpyrimidine-2,4-diamine (300 mg, quantitative yield). LC purity: 78.4%; m/z: 356.25 [M+H]⁺ (Mol. formula C₁₉H₂₉N₇, calcd. mol. Wt. 355.49).

### Step-6: Synthesis of N-((1R,4R)-4-((4-((1-cyclopentyl-1H-imidazol-4-yl)amino)pyrimidin-2-yl)(methyl)amino)cyclohexyl)-2,3-dihydro-1H-indene-2-carboxamide

To a mixture of N²-((1R,4R)-4-aminocyclohexyl)-N⁴-(1-cyclopentyl-1H-imidazol-4-yl)-N²-methylpyrimidine-2,4-diamine (300 mg, 0.845 mmol) in dry DMF (1 mL) was added TEA (0.35 mL, 2.535 mmol), EDC·HCl (242.09 mg, 1.267 mmol), HOBt (57.037 mg, 0.422 mmol) and 2,3-dihydro-1H-indene-2-carboxylic acid (136.90 mg, 0.845 mmol). The reaction mixture was stirred for 5 h at room temperature. The progress of the reaction was monitored by TLC, and after complete consumption of starting material, the reaction mixture was diluted with water and extracted with DCM. The organic layer was separated was dried over anhydrous sodium sulfate and concentrated to obtain the crude product. The crude product was purified by reverse phase preparative HPLC to N-((1R,4R)-4-((4-((1-cyclopentyl-1H-imidazol-4-yl)amino)pyrimidin-2-yl)(methyl)amino) cyclohexyl)-2,3-dihydro-1H-indene-2-carboxamide (50 mg, 11%) as the free base. LC purity: 99.16%; m/z: 500.31 [M+H]⁺ (Mol. formula C₂₉H₃₇N₇O, calcd. mol. wt. 499.66). ¹H NMR (400 MHz, CD₃OD): δ 7.82 (d, J=6.0 Hz, 1H), 7.51 (s, 1H), 7.37 (s, 1H), 7.20-7.12 (m, 4H), 6.04 (d, J=6.0 Hz, 1H), 4.60 (t, J=6.8 Hz, 2H), 3.70-3.62 (m, 1H), 3.25-3.14 (m, 6H), 3.04 (s, 1H), 2.26-2.20 (m, 2H), 2.10-2.07 (m, 2H), 1.93-1.90 (m, 5H), 1.81-1.75 (m, 6H), 1.50-1.47 (m, 2H).

### Example 129: Synthesis of Compound 131

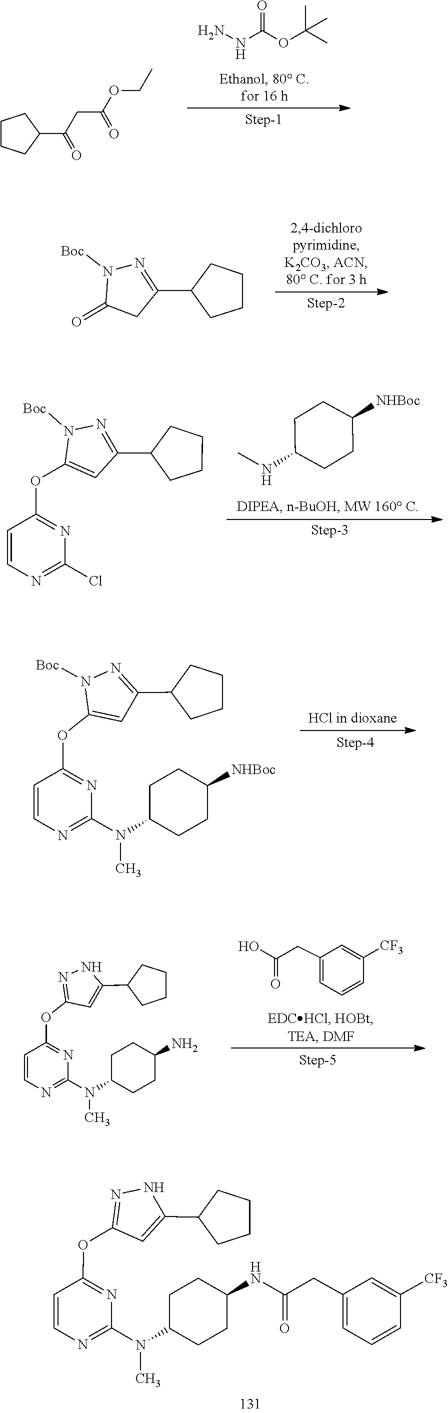

### Step-1: Synthesis of tert-butyl 3-cyclopentyl-5-oxo-4,5-dihydro-1H-pyrazole-1-carboxylate

To a stirred solution of ethyl 3-cyclopentyl-3-oxopropanoate (2 g, 10.86 mmol) in ethanol (20 mL) was added tert-butyl hydrazinecarboxylate (2.86 g, 21.73 mmol). The reaction mixture was heated to 80° C. for 16 h. The progress of the reaction was monitored by TLC, and after complete consumption of starting material, the reaction mixture was concentrated under reduced pressure to obtain the residue. The residue thus obtained was triturated with pet ether to yield tert-butyl 3-cyclopentyl-5-oxo-4,5-dihydro-1H-pyrazole-1-carboxylate (1.8 g, 65.9%). LC purity: 91.68%; m/z: 153.2 [M-Boc]⁺ (Mol. formula C₁₃H₂₀N₂O₃ calcd. mol. wt. 252.31).

### Step-2: Synthesis of tert-butyl 5-((2-chloropyrimidin-4-yl)oxy)-3-cyclopentyl-1H-pyrazole-1-carboxylate

To a solution of tert-butyl 3-cyclopentyl-5-oxo-4,5-dihydro-1H-pyrazole-1-carboxylate (1.8 g, 7.14 mmol) in ACN (25 mL) was added 2,4-dichloropyrimidine (1.26 g, 8.57 mmol) and potassium carbonate (2.95 g, 21.42 mmol). The reaction was heated at 80° C. for 3 h. The completion of the reaction was monitored by TLC. The reaction mixture was concentrated to remove acetonitrile, diluted with water and extracted with ethyl acetate. The organic layer was dried over anhydrous Na₂SO₄, filtered and concentrated under reduced pressure. The crude was purified by Biotage-Isolera using silica gel (230-400 mesh) with a gradient elution in 0-30% ethyl acetate in pet ether to obtain tert-butyl 5-((2-chloropyrimidin-4-yl)oxy)-3-cyclopentyl-1H-pyrazole-1-carboxylate (1.2 g, 46.15%). LC purity: 96.02%; m/z: 265 [M-Boc]⁺ (Mol. formula C₁₇H₂₁C₁N₄O₃ calcd. mol. wt. 364.83).

### Step-3: Synthesis of tert-butyl 5-((2-(((1R,4R)-4-((tert-butoxycarbonyl)amino) cyclohexyl) (methyl)amino)pyrimidin-4-yl)oxy)-3-cyclopentyl-1H-pyrazole-1-carboxylate

To a solution of tert-butyl 5-((2-chloropyrimidin-4-yl)oxy)-3-cyclopentyl-1H-pyrazole-1-carboxylate (1 g, 2.74 mmol) in n-Butanol (10 mL) in a 20 mL microwave vial was added tert-butyl ((1R,4R)-4-(methylamino)cyclohexyl)carbamate (1.25 g, 5.49 mmol) and DIPEA (1.43 mL, 8.22 mmol). The reaction mixture was subjected to microwave heating at 160° C. for 2 h. The progress of the reaction was monitored by TLC, and after complete consumption of the starting material, the reaction was cooled to room temperature and concentrated to remove n-butanol. The residue obtained was purified by Biotage-Isolera using silica gel (230-400 mesh) with a gradient elution in 0-30% pet ether in ethyl acetate to obtain tert-butyl 5-((2-(((1R,4R)-4-((tert-butoxycarbonyl)amino)cyclohexyl)(methyl)amino)pyrimidin-4-yl)oxy)-3-cyclopentyl-TH-pyrazole-1-carboxylate (1 g, 66.6%). LC purity: 93.57%; m/z: 457 [M-Boc]⁺ (Mol. formula C₂₉H₄₄N₆O₅ calcd. mol. wt. 556.71).

### Step-4: Synthesis of (1R,4R)—N1-(4-((5-cyclopentyl-1H-pyrazol-3-yl)oxy)pyrimidin-2-yl)-N1-methylcyclohexane-1,4-diamine

To a stirred and cooled (0° C.) solution of tert-butyl 5-((2-(((1R,4R)-4-((tert-butoxycarbonyl)amino) cyclohexyl)(methyl)amino)pyrimidin-4-yl)oxy)-3-cyclopentyl-TH-pyrazole-1-carboxylate (1 g, 1.79 mmol) in dry DCM (10 mL) was added HCl in dioxane (10 mL, 4M solution). The reaction mixture was allowed to stir at room temperature for 1 h. The progress of the reaction was monitored by TLC, and after complete consumption of starting material, the resulting mixture was concentrated, triturated with pet ether and concentrated under high vacuum to yield (1R,4R)—N1-(4-((5-cyclopentyl-1H-pyrazol-3-yl)oxy)pyrimidin-2-yl)-N1-methylcyclohexane-1,4-diamine as the HCl salt (1 g, quantitative yield). LC purity: 95.73%; m/z: 357 [M+H]⁺ (Mol. formula C₁₉H₂₈N₆O calcd. mol. wt. 356.47).

### Step-5: Synthesis of N-((1R,4R)-4-((4-((5-cyclopentyl-1H-pyrazol-3-yl)oxy)pyrimidin-2-yl)(methyl)amino)cyclohexyl)-2-(3-(trifluoromethyl)phenyl)acetamide

To a solution of 2-(3-(trifluoromethyl)phenyl)acetic acid (0.114 g, 0.56 mmol) in dry DMF (5 mL) was added triethylamine (0.39 mL, 2.8 mmol) followed by EDC·HCL (0.160 g, 0.84 mmol) and HOBt (0.037 g, 0.28 mmol). The reaction was stirred for 15 min. Then (1R,4R)—N1-(4-((5-cyclopentyl-1H-pyrazol-3-yl)oxy)pyrimidin-2-yl)-N1-methylcyclohexane-1,4-diamine (0.200 g, 0.56 mmol) was added. The reaction was stirred at room temperature for 5 h. The completion of the reaction was monitored by TLC, and after the starting material was consumed, the reaction mixture was diluted with water and extracted with dichloromethane. The resulting organic layer was washed with brine, dried over anhydrous Na₂SO₄ and concentrated to obtain crude compound. The crude product was purified by reverse phase preparative HPLC with mobile phase 0.1% TFA in water/acetonitrile to yield N-((1R,4R)-4-((4-((5-cyclopentyl-1H-pyrazol-3-yl)oxy)pyrimidin-2-yl)(methyl)amino)cyclohexyl)-2-(3-(trifluoromethyl)phenyl) acetamide (40 mg, 13.2%). LC purity: 99.14%; m/z: 543.3 [M+H]⁺ (Mol. formula C₂₈H₃₃F₃N₆O₂ calcd. mol. wt. 542.61). ¹H NMR (400 MHz, CD₃OD): δ 8.20 (d, J=6.8 Hz, 1H), 7.63 (s, 1H), 7.58-7.51 (m, 3H), 6.6 (d, J=5.6 Hz, 1H), 5.99 (s, 1H), 4.19 (s, 1H), 3.58 (s, 3H), 3.17-3.08 (m, 4H), 2.13-2.08 (m, 4H), 1.84-1.66 (m, 10H), 1.30 (s, 2H).

### Example 130: Synthesis of Compound 132

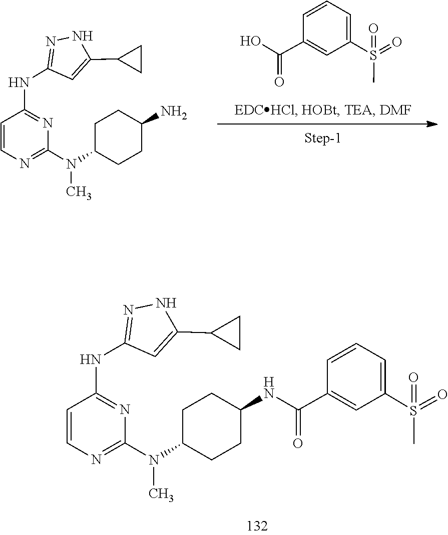 [SMILES: CN(c1nccc(Nc2cc(C3CC3)[nH]n2)n1)[C@H]1CC[C@H](N)CC1 ; CS(=O)(=O)c1cccc(C(=O)O)c1 ; CN(c1nccc(Nc2cc(C3CC3)[nH]n2)n1)[C@H]1CC[C@H](NC(=O)c2cccc(S(C)(=O)=O)c2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-3-(methylsulfonyl)benzamide

To a stirred solution of 3-(methylsulfonyl)benzoic acid (97.8 mg, 0.489 mmol) in dry DMF (3 mL) was added triethylamine (0.25 mL 1.834 mmol) followed by the addition of the EDC·HCl (175 mg, 0.917 mmol) and HOBt (41.2 mg 0.305 mmol). The reaction mixture was stirred at room temperature for 15 min. Then N2-((1R,4R)-4-aminocyclohexyl)-N4-(5-cyclopropyl-1H-pyrazol-3-yl)-N2-methylpyrimidine-2,4-diamine (200 mg, 0.611 mmol) was added. The reaction mixture was stirred at room temperature for 5 h, and after completion of the reaction (monitored by UPLC), the reaction mixture was diluted with dichloromethane washed with water and brine, and dried over anhydrous Na₂SO₄ and concentrated under reduced pressure to get the residue. The residue was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-3-(methylsulfonyl)benzamide (120 mg, 38.71%) as the free base. LC purity: 99.63%; m/z: 510.3 [M+H]⁺ (Mol. formula C₂₅H₃₁N₇O₃S, calcd. mol. wt. 509.63). ¹H NMR (400 MHz, CD₃OD): δ 8.39 (s, 1H), 8.17-8.13 (m, 2H), 7.79-7.74 (m, 2H), 6.38-6.37 (m, 2H), 4.59-4.67 (m, 1H), 3.95-3.92 (m, 1H), 3.18 (s, 3H), 3.09 (s, 3H), 2.19-2.16 (m, 2H), 1.98-1.93 (m, 1H), 1.86-1.81 (m, 4H), 1.67-1.65 (m, 2H), 1.01-0.99 (m, 2H), 0.76-0.74 (m, 2H).

### Example 131: Synthesis of Compound 133

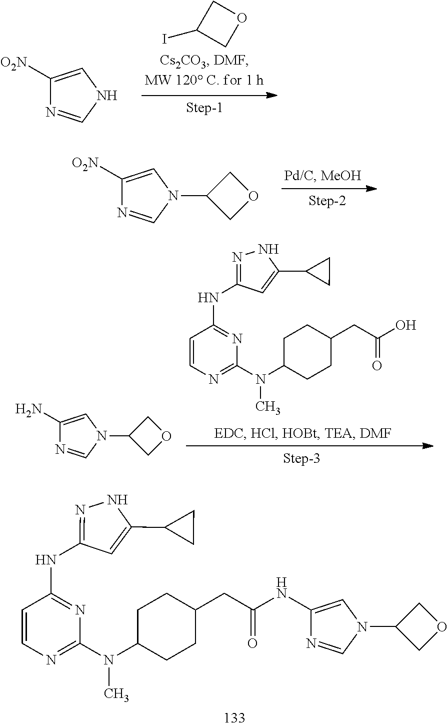

### Step-1: Synthesis of 4-nitro-1-(oxetan-3-yl)-1H-imidazole

To a solution of 4-nitro-1H-imidazole (1.0 g, 8.849 mmol) in dry DMF (10 mL) in a 20 mL microwave vial was added Cs₂CO₃ (5.7 g, 17.698 mmol) and 3-iodooxetane (4.8 g, 26.548 mmol). The reaction mixture was heated in a microwave reactor to 120° C. for 1 h. The progress of the reaction was monitored by LCMS. After complete consumption of starting material, the reaction mixture was diluted with water and extracted with DCM. The organic layer separated was dried over anhydrous sodium sulphate and concentrated to give 4-nitro-1-(oxetan-3-yl)-1H-imidazole. (600 mg, crude). LC purity: 93.5%; m/z: 170.1 [M+H]⁺ (Mol. formula C₆H₇N₃O₃, calcd. mol. wt. 169.14).

### Step-2: Synthesis of 1-(oxetan-3-yl)-1H-imidazol-4-amine

To a stirred solution of 4-nitro-1-(oxetan-3-yl)-1H-imidazole (200 mg, 1.183 mmol) in MeOH (3 mL) was added 10% Pd/C (100 mg). The reaction was stirred at room temperature under hydrogen balloon pressure for 4 h. The reaction mixture was monitored by TLC, and after complete consumption of the starting material, the reaction mixture was filtered over a bed of celite and washed with methanol. The resulting filtrate was concentrated to obtain 1-(oxetan-3-yl)-1H-imidazol-4-amine (120 mg, crude). LC purity: 83.1%; m/z: 140.1 [M+H]⁺ (Mol. formula C₆H₉N₃O, calcd. mol. wt. 139.16).

### Step-3: Synthesis of 2-((1R,4R)-4-((4-((5-cyclopropyl-1H-pyrazol-3-yl)amino)pyrimidin-2-yl)(methyl)amino)cyclohexyl)-N-(1-(oxetan-3-yl)-1H-imidazol-4-yl)acetamide

To a stirred solution of 2-((1R,4R)-4-((4-((5-cyclopropyl-1H-pyrazol-3-yl)amino)pyrimidin-2-yl)(methyl)amino)cyclohexyl)acetic acid (200 mg, 0.540 mmol) in pyridine (1 mL) was added EDC·HCl (515.7 mg, 2.7 mmol) and the reaction mixture was stirred for 15 minutes at room temperature. Then 1-(oxetan-3-yl)-1H-imidazol-4-amine (150.2 mg, 1.081 mmol) was added. The reaction mixture was stirred at 80° C. for 16 h. The progress of the reaction was monitored by UPLC. After completion of the reaction, the reaction mixture was concentrated, diluted with water and extracted with DCM. The resulting organic layer was washed with brine then dried over anhydrous Na₂SO₄ and concentrated to obtain the crude product. The crude product was purified by reverse phase preparative HPLC to obtain 2-((1R,4R)-4-((4-((5-cyclopropyl-1H-pyrazol-3-yl)amino)pyrimidin-2-yl)(methyl)amino)cyclohexyl)-N-(1-(oxetan-3-yl)-1H-imidazol-4-yl)acetamide (12 mg, 4.5%) as the free base. LC purity: 97.07%; m/z: 492.28 [M+H]⁺ (Mol. formula C₂₅H₃₃N₉O₂, calcd. mol. wt. 491.60). ¹H NMR (400 MHz, CD₃OD): δ 7.87-7.85 (m, 1H), 7.65-7.60 (m, 2H), 6.10 (d, J=6.0 Hz, 2H), 5.45-5.39 (m, 1H), 5.09 (t, J=7.2 Hz, 2H), 4.87 (t, J=6.4 Hz, 2H), 3.06 (s, 3H), 2.58 (d, J=7.6 Hz, 1H), 2.41-2.33 (m, 3H), 1.93-1.80 (m, 8H), 1.30-1.25 (m, 1H), 1.00-0.97 (m, 2H), 0.75-0.73 (m, 2H).

### Example 132: Synthesis of Compound 134

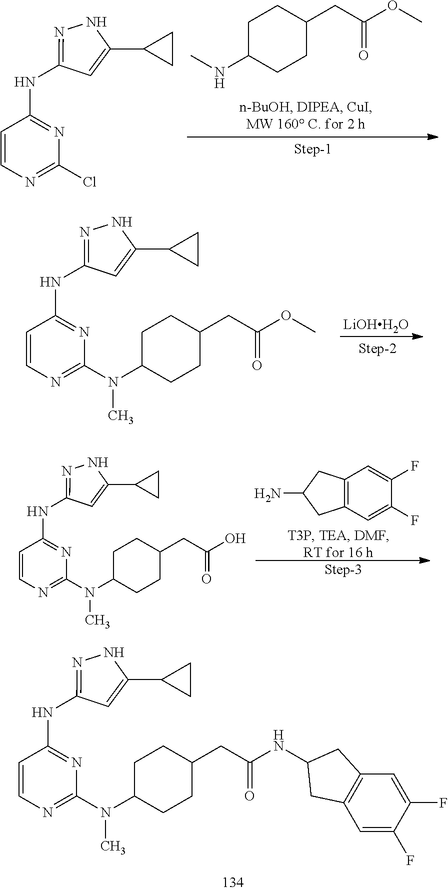

### Step-1: Synthesis of methyl 2-(4-((4-((5-cyclopropyl-1H-pyrazol-3-yl)amino)pyrimidin-2-yl)(methyl)amino)cyclohexyl)acetate

To a solution of 2-chloro-N-(5-cyclopropyl-1H-pyrazol-3-yl)pyrimidin-4-amine (317 mg, 1.35 mmol) in n-BuOH (5 mL) in a 20 mL MW vial was added CuI (51 mg, 0.27 mmol) and DIPEA (0.72 mL, 4.05 mmol). The reaction mixture was stirred for 5 min and then methyl 2-(4-(methylamino)cyclohexyl)acetate (500 mg, 2.7 mmol) was added. The reaction mixture was stirred in microwave at 160° C. for 2 h. The progress of the reaction was monitored by TLC. After completion of the reaction, the reaction mixture was cooled to room temperature diluted with water and extracted with dichloromethane. The combined organic layer was washed with water and brine, dried over anhydrous Na₂SO₄ and concentrated to obtain the crude product. The crude product was purified by Biotage Isolera using 230-400 silica gel eluted with 0-10% ethyl acetate in pet ether to obtain methyl 2-(4-((4-((5-cyclopropyl-1H-pyrazol-3-yl)amino)pyrimidin-2-yl)(methyl)amino)cyclohexyl)acetate (230 mg, 44.4%). LC purity: 75.85%; m/z: 385.2 [M+H]⁺ (Mol. formula C₂₀H₂₈N₆O₂, calcd. mol. wt. 384.48).

### Step-2: Synthesis of 2-(4-((4-((5-cyclopropyl-1H-pyrazol-3-yl)amino)pyrimidin-2-yl)(methyl)amino)cyclohexyl)acetic acid

To a stirred solution of methyl 2-(4-((4-((5-cyclopropyl-1H-pyrazol-3-yl)amino)pyrimidin-2-yl)(methyl)amino)cyclohexyl)acetate (230 mg, 0.598 mmol) in THF:MeOH:H₂O (3 mL) was added LiOH·H₂O (50 mg, 1.19 mmol) and the reaction mixture was heated to 60° C. for 16 h. The reaction was monitored by TLC. After completion of the reaction, the reaction mixture was cooled to room temperature and concentrated to obtain 2-(4-((4-((5-cyclopropyl-1H-pyrazol-3-yl)amino)pyrimidin-2-yl)(methyl)amino)cyclohexyl)acetic acid (200 mg, crude). LC purity: 76.26%; m/z: 371.3 [M+H]⁺ (Mol. formula C₁₉H₂₆N₆O₂, calcd. mol. wt. 370.46).

### Step-3: Synthesis of 2-(4-((4-((5-cyclopropyl-1H-pyrazol-3-yl)amino)pyrimidin-2-yl)(methyl)amino)cyclohexyl)-N-(5,6-difluoro-2,3-dihydro-1H-inden-2-yl)acetamide

To a solution of 2-(4-((4-((5-cyclopropyl-1H-pyrazol-3-yl)amino)pyrimidin-2-yl)(methyl)amino)cyclohexyl)acetic acid (200 mg, 0.540 mmol) in dry DMF (5 mL) was added triethylamine (0.23 mL, 1.62 mmol) drop-wise followed by the addition of T3P (0.515 mL, 1.62 mmol, 50% solution in ethyl acetate). The reaction mixture was stirred for 5 min and then 5,6-difluoro-2,3-dihydro-1H-inden-2-amine (91 mg, 0.540 mmol) was added. The reaction mixture was stirred at room temperature for 16 h. The progress of the reaction was monitored by TLC. After completion of the reaction, the reaction mixture was diluted with dichloromethane, washed with water and brine and dried over anhydrous Na₂SO₄ and concentrated to obtain the residue. The residue was purified by reverse phase preparative HPLC to yield 2-(4-((4-((5-cyclopropyl-1H-pyrazol-3-yl)amino)pyrimidin-2-yl)(methyl)amino)cyclohexyl)-N-(5,6-difluoro-2,3-dihydro-1H-inden-2-yl)acetamide (13 mg, 5%) as the free base. LC purity: 95.37%; m/z: 521.9 [M+H]⁺ (Mol. formula C₂₈H₃₃F₂N₇O, calcd. mol. wt. 521.62). ¹H NMR (400 MHz, CD₃OD): δ 7.84 (s, 1H), 7.15-7.10 (m, 2H), 6.20-6.09 (m, 2H), 4.67-4.64 (m, 2H), 3.33-3.32 (m, 2H), 3.00 (s, 3H), 2.86-2.79 (m, 2H), 2.38-2.35 (m, 2H), 2.14-2.12 (m, 1H), 1.93-1.88 (m, 2H), 1.80-1.77 (m, 5H), 1.52-1.47 (m, 2H), 1.01-0.97 (m, 2H), 0.75-0.72 (m, 2H).

### Example 133: Synthesis of Compound 135

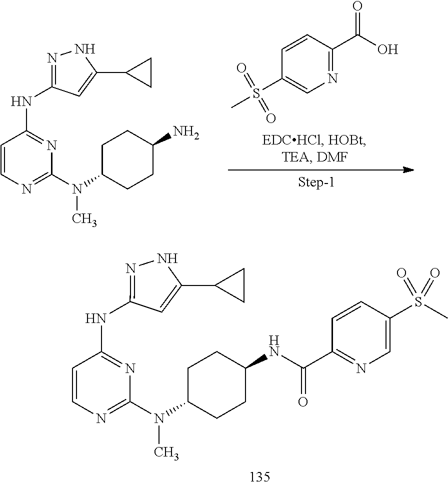 [SMILES: CN(c1nccc(Nc2cc(C3CC3)[nH]n2)n1)[C@H]1CC[C@H](N)CC1 ; CS(=O)(=O)c1ccc(C(=O)O)nc1 ; CN(c1nccc(Nc2cc(C3CC3)[nH]n2)n1)[C@H]1CC[C@H](NC(=O)c2ccc(S(C)(=O)=O)cn2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-5-(methylsulfonyl)picolinamide

To a stirred solution of 5-(methylsulfonyl)picolinic acid (98.3 mg, 0.489 mmol) in dry DMF (3 mL) was added triethylamine (0.25 mL, 1.834 mmol) followed by the addition of the EDC·HCl (175.2 mg, 0.917 mmol) and HOBt (41.2 mg, 0.530 mmol). The reaction mixture was stirred at room temperature for 15 min and then N₂-((1R,4R)-4-aminocyclohexyl)-N₄-(5-cyclopropyl-1H-pyrazol-3-yl)-N₂-methylpyrimidine-2,4-diamine (200 mg, 0.611 mmol) was added. The reaction mixture was stirred at room temperature for 6 h. After completion of the reaction (monitored by UPLC), the reaction mixture was diluted with dichloromethane washed with water and brine, dried over anhydrous Na₂SO₄ and concentrated under reduced pressure to obtain the crude compound. The crude product was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-5-(methylsulfonyl)picolinamide (30 mg, 9.64%) as the free base. LC purity: 96.47%; m/z: 511.2 [M+H]⁺ (Mol. formula C₂₄H₃₀N₈O₃S calcd. mol. wt. 510.62). ¹H NMR (400 MHz, CD₃OD): δ 9.15-9.14 (m, 1H), 8.53-8.50 (m, 1H), 8.34-8.32 (m, 1H), 7.89 (d, J=6 Hz, 1H), 6.13-6.12 (m, 2H), 4.62-4.10 (m, 1H), 3.99-3.98 (m, 1H), 3.26 (s, 3H), 3.04 (s, 3H), 2.20-2.17 (m, 2H), 2.03-1.93 (m, 1H), 1.87-1.81 (m, 4H), 1.70-1.68 (m, 2H), 1.00-0.98 (m, 2H), 0.77-0.73 (m, 2H).

### Example 134: Synthesis of Compound 136

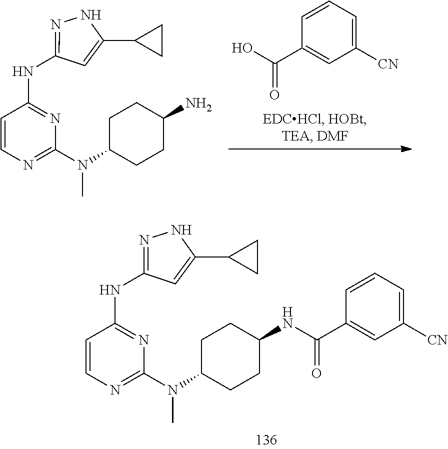 [SMILES: CN(c1nccc(Nc2cc(C3CC3)[nH]n2)n1)[C@H]1CC[C@H](N)CC1 ; N#Cc1cccc(C(=O)O)c1 ; CN(c1nccc(Nc2cc(C3CC3)[nH]n2)n1)[C@H]1CC[C@H](NC(=O)c2cccc(C#N)c2)CC1]

### Step-1: Synthesis of 3-cyano-N-((1R,4R)-4-((4-((5-cyclopropyl-1H-pyrazol-3-yl)amino)pyrimidin-2-yl)(methyl)amino)cyclohexyl)benzamide

To a solution of 3-cyanobenzoic acid (90 mg, 0.611 mmol) in dry DMF (3 mL) was added TEA (0.25 mL, 1.833 mmol) followed by EDC·HCl (175 mg, 0.916 mmol) and HOBt (82.4 mg, 0.611 mmol). The reaction mixture was stirred at room temperature for 10 min. Then N2-((1R,4R)-4-aminocyclohexyl)-N4-(5-cyclopropyl-1H-pyrazol-3-yl)-N2-methylpyrimidine-2,4-diamine (200 mg, 0.611 mmol) was added. The reaction mixture was stirred at room temperature for 16 h. After completion of the reaction, the reaction mixture was diluted with dichloromethane washed with water and brine, dried over anhydrous Na₂SO₄ and concentrated to yield the crude product. The crude product was purified by reverse phase prep HPLC to provide 3-cyano-N-((1R,4R)-4-((4-((5-cyclopropyl-1H-pyrazol-3-yl)amino)pyrimidin-2-yl)(methyl)amino)cyclohexyl)benzamide (30 mg, 10.7%) as the free base. LC purity: 94.77%; m/z: 457.2 [M+H]⁺ (Mol. formula C₂₅H₂₈N₈O, calcd. mol. wt. 456.55). ¹H VTNMR (400 MHz, CD₃OD): δ 8.18 (s, 1H), 8.13-8.11 (m, 1H), 7.90-7.86 (m, 2H), 7.66 (t, J=8 Hz, 1H), 6.19 (d, J=6 Hz, 1H), 6.09 (s, 1H), 3.94-3.82 (m, 1H), 3.05 (s, 3H), 2.18-2.15 (m, 2H), 1.93-1.81 (m, 6H), 1.65-1.53 (m, 2H), 1.00-0.99 (m, 2H), 0.77-0.74 (m, 2H).

### Example 135: Synthesis of Compound 137

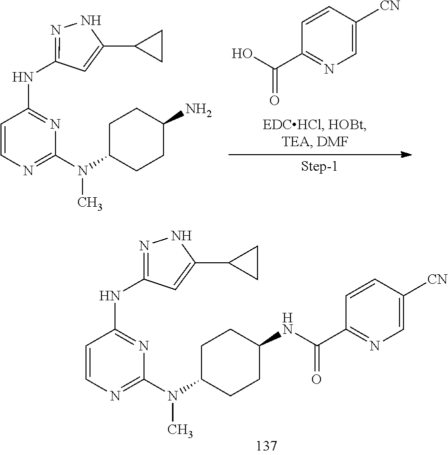 [SMILES: CN(c1nccc(Nc2cc(C3CC3)[nH]n2)n1)[C@H]1CC[C@H](N)CC1 ; N#Cc1ccc(C(=O)O)nc1 ; CN(c1nccc(Nc2cc(C3CC3)[nH]n2)n1)[C@H]1CC[C@H](NC(=O)c2ccc(C#N)cn2)CC1]

### Step-1: Synthesis of 5-cyano-N-((1R,4R)-4-((4-((5-cyclopropyl-1H-pyrazol-3-yl)amino) pyrimidin-2-yl)(methyl)amino)cyclohexyl)picolinamide

To a stirred solution of 5-cyanopicolinic acid (72.4 mg, 0.489 mmol) in dry DMF (3 mL) was added triethylamine (0.25 mL, 1.834 mmol) followed by the addition of the EDC·HCl (175.2 mg, 0.917 mmol) and HOBt (41.2 mg, 0.305 mmol). The reaction mixture was stirred at room temperature for 15 min, and added N2-((1R,4R)-4-aminocyclohexyl)-N4-(5-cyclopropyl-1H-pyrazol-3-yl)-N2-methylpyrimidine-2,4-diamine (200 mg, 0.611 mmol) was added. The reaction mixture was stirred at room temperature for 16 h. After completion of the reaction (monitored by UPLC), the reaction mixture was diluted with dichloromethane, washed with water and brine, dried over anhydrous Na₂SO₄ and concentrated under reduced pressure to yield the crude compound. The crude compound was purified by reverse phase preparative HPLC to yield 5-cyano-N-((1R,4R)-4-((4-((5-cyclopropyl-1H-pyrazol-3-yl)amino)pyrimidin-2-yl)(methyl)amino) cyclohexyl)picolinamide (45 mg, 16.12%) as the TFA salt. LC purity: 99.67%; m/z: 472.2 [M+H]⁺ (Mol. formula C₂₄H₂₇N₉O, calcd. mol. wt. 471.61). ¹H NMR (400 MHz, CD₃OD): δ 8.99 (s, 1H), 8.40-8.38 (m, 1H), 8.28-8.26 (m, 1H), 7.74 (d, J=6 Hz, 1H), 6.38-6.37 (m, 2H), 3.99-3.94 (m, 1H), 3.11 (s, 3H), 2.21-2.17 (m, 2H), 2.00-1.92 (m, 5H), 1.72-1.65 (m, 3H), 1.07-1.05 (m, 2H), 0.78-0.66 (m, 2H).

### Example 136: Synthesis of Compound 138

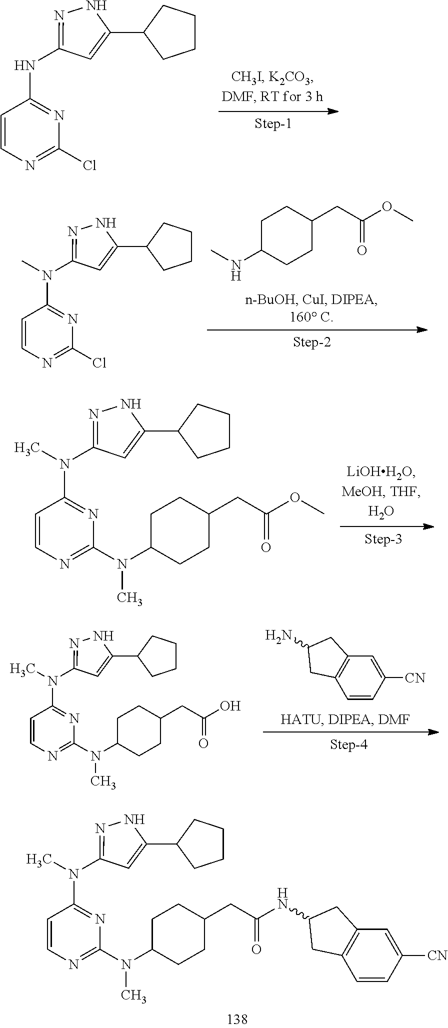

### Step-1: Synthesis of 2-chloro-N-(5-cyclopentyl-1H-pyrazol-3-yl)-N-methylpyrimidin-4-amine

To a stirred solution of 2-chloro-N-(5-cyclopentyl-1H-pyrazol-3-yl)pyrimidin-4-amine (1.5 g, 5.703 mmol) in dry DMF (6 mL) cooled at 0° C. was added potassium carbonate (1.57 g, 11.40 mmol). The reaction was stirred at 0° C. for 10 min. Then iodomethane (0.8 g, 5.703 mmol) was added dropwise and the reaction mixture was stirred at 0° C. for 1 h. The reaction was monitored by UPLC and showed 50% conversion. The reaction mixture was diluted with ethyl acetate and washed with water and brine solution. The organic layer was dried over anhydrous Na₂SO₄ then concentrated. The residue was purified by using Biotage Isolera (230-400 silica gel) with gradient elution of 20% ethyl acetate in pet ether to get 2-chloro-N-(5-cyclopentyl-1H-pyrazol-3-yl)-N-methylpyrimidin-4-amine (320 mg, 20.25%). LC purity: 91.85%; m/z: 278.1 [M+H]⁺ (Mol. formula C₁₃H₁₆ClN₅, calcd. mol. wt. 277.76).

### Step-2: Synthesis of methyl 2-(4-((4-((5-cyclopentyl-1H-pyrazol-3-yl)(methyl)amino) pyrimidin-2-yl)(methyl)amino)cyclohexyl)acetate

To a solution of methyl 2-(4-(methylamino)cyclohexyl)acetate (400 mg, 2.162 mmol) in n-butanol (5 mL) In a 20 mL microwave vial was added CuI (123 mg, 0.648 mmol) and 2-chloro-N-(5-cyclopentyl-1H-pyrazol-3-yl)-N-methylpyrimidin-4-amine (300 mg, 1.081 mmol). The reaction mixture was heated in a microwave reactor at 160° C. for 3 h. The progress of the reaction was monitored by TLC, and after complete consumption of starting material, the reaction mixture was cooled to room temperature and concentrated to obtain the crude product. The crude was purified by using Biotage Isolera (230-400 silica gel) with gradient elution of 0-50% ethyl acetate in Pet ether to obtain methyl 2-(4-((4-((5-cyclopentyl-1H-pyrazol-3-yl)(methyl)amino)pyrimidin-2yl)(methyl)amino)cyclohexyl)acetate (200 mg, 43.4% yield). LC purity: 82.9%; m/z: 427.3 [M+H]⁺ (Mol. formula C₂₃H₃₄N₆O₂, calcd. mol. wt. 426.57).

### Step-3: Synthesis of 2-(4-((4-((5-cyclopentyl-1H-pyrazol-3-yl)(methyl)amino)pyrimidin-2-yl)(methyl)amino)cyclohexyl)acetic acid

To a stirred solution of methyl 2-(4-((4-((5-cyclopentyl-1H-pyrazol-3-yl)(methyl)amino)pyrimidin-2-yl)(methyl)amino)cyclohexyl)acetate (180 mg, 0.422 mmol) in mixture of Water, THF, Methanol (1:1:1, 3 mL) was added lithium hydroxide monohydrate (18 mg, 0.422 mmol) and the reaction mixture was heated to 80° C. for 16 h. The completion of the reaction was monitored by TLC. The reaction mixture was concentrated to obtain 2-(4-((4-((5-cyclopentyl-1H-pyrazol-3-yl)(methyl)amino)pyrimidin-2-yl)(methyl)amino)cyclohexyl)acetic acid (150 mg, 86%). LC purity: 91.2%; m/z: 413.2 [M+H]⁺ (Mol. formula C₂₂H₃₂N₆O₂, calcd. mol. wt. 412.54).

### Step-4: Synthesis of N-(5-cyano-2,3-dihydro-1H-inden-2-yl)-2-(4-((4-((5-cyclopentyl-1H-pyrazol-3-yl)(methyl)amino)pyrimidin-2-yl)(methyl)amino)cyclohexyl)acetamide

To a solution of 2-(4-((4-((5-cyclopentyl-1H-pyrazol-3-yl)(methyl)amino)pyrimidin-2-yl)(methyl)amino)cyclohexyl)acetic acid (180 mg, 0.436 mmol) in dry DMF (2 mL) was added DIPEA (0.22 mL, 1.308 mmol) and HATU (328 mg, 0.654 mmol). The reaction was stirred for 30 min at ambient temperature. Then 2-amino-2,3-dihydro-1H-indene-5-carbonitrile (69 mg, 0.436 mmol) was added and the reaction mixture was stirred for 2 h at ambient temperature. The progress of the reaction was monitored by TLC. After complete consumption of starting material, water was added and the mixture was extracted with dichloromethane. The organic layer was separated and dried over anhydrous sodium sulphate and concentrated to obtain the crude product. The crude product was purified by reverse phase preparative HPLC (0.1% TFA in water/acetonitrile) to yield N-(5-cyano-2,3-dihydro-1H-inden-2-yl)-2-(4-((4-((5-cyclopentyl-1H-pyrazol-3-yl)(methyl)amino)pyrimidin-2-yl)(methyl)amino) cyclohexyl)acetamide (70 mg, 29%). LC purity: 99.6%; m/z: 553.3 [M+H]⁺ (Mol. formula C₃₂H₄₀N₈O, calcd. mol. wt. 552.73). ¹H NMR (400 MHz, CD₃OD): δ 7.63-7.60 (m, 2H), 7.56-7.54 (m, 1H), 7.44-7.41 (m, 1H), 6.21 (d, J=6.4 Hz, 1H), 6.15 (s, 1H), 4.67-4.65 (m, 1H), 3.53 (s, 3H), 3.37-3.34 (m, 2H), 3.17-3.09 (m, 4H), 2.96-2.90 (m, 2H), 2.37-2.35 (m, 2H), 2.16-2.11 (m, 3H), 1.90-1.64 (m, 14H), 1.30-1.15 (m, 1H).

### Example 137: Synthesis of Compound 139

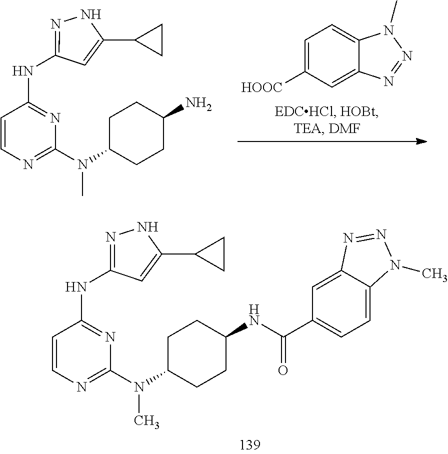 [SMILES: CN(c1nccc(Nc2cc(C3CC3)[nH]n2)n1)[C@H]1CC[C@H](N)CC1 ; Cn1nnc2cc(C(=O)O)ccc21 ; Cn1nnc2cc(C(=O)N[C@H]3CC[C@H](N(C)c4nccc(Nc5cc(C6CC6)[nH]n5)n4)CC3)ccc21]

### Synthesis of N-((1R,4R)-4-((4-((5-cyclopropyl-1H-pyrazol-3-yl)amino)pyrimidin-2-yl)(methyl)amino)cyclohexyl)-1-methyl-1H-benzo[d][1,2,3]triazole-5-carboxamide

To a solution of 1-methyl-1H-benzo[d][1,2,3]triazole-5-carboxylic acid (162.2 mg, 0.916 mmol) in dry DMF (6 mL) was added TEA (0.38 mL, 2.752 mmol) followed by EDC·HCl (262 mg, 1.376 mmol) and HOBt (124 mg, 0.916 mmol). The reaction mixture was stirred at room temperature for 10 min, and then N2-((1R,4R)-4-aminocyclohexyl)-N4-(5-cyclopropyl-1H-pyrazol-3-yl)-N2-methylpyrimidine-2,4-diamine (300 mg, 0.916 mmol) was added. The reaction mixture was stirred at room temperature for 16 h. After completion of the reaction, reaction mixture was diluted with dichloromethane washed with water and brine, dried over anhydrous Na₂SO₄ and concentrated to yield the crude product. The crude product was purified by reverse phase prep HPLC to provide N-((1R,4R)-4-((4-((5-cyclopropyl-1H-pyrazol-3-yl)amino)pyrimidin-2-yl)(methyl)amino)cyclohexyl)-1-methyl-1H-benzo[d][1,2,3]triazole-5-carboxamide (45 mg, 10%) as the free base. LC purity: 99.34%; m/z: 487.3 [M+H]⁺ (Mol. formula C₂₅H₃₀N₁₀O, calcd. mol. wt. 486.58). ¹H VTNMR (400 MHz, CD₃OD): δ 8.50 (s, 1H), 8.06 (dd, J=8.8, 8.4 Hz, 1H), 7.89 (d, J=5.6 Hz, 1H), 7.82 (d, J=8.8 Hz, 1H), 6.15-6.13 (m, 2H), 4.57-4.62 (m, 1H), 4.37 (s, 3H), 3.97-3.89 (m, 1H), 3.05 (s, 3H), 2.21-2.18 (m, 2H), 1.95-1.82 (m, 5H), 1.72-1.65 (m, 2H), 1.00-0.96 (m, 2H), 0.77-0.73 (m, 2H).

### Example 138: Synthesis of Compound 140

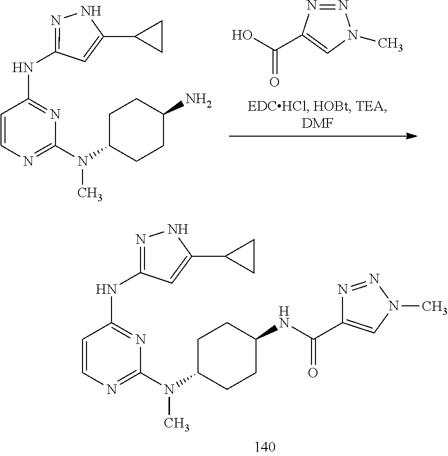

### Synthesis of N-((1R,4R)-4-((4-((5-cyclopropyl-1H-pyrazol-3-yl)amino)pyrimidin-2-yl)(methyl)amino)cyclohexyl)-1-methyl-1H-1,2,3-triazole-4-carboxamide

To a solution of 1-methyl-1H-1,2,3-triazole-4-carboxylic acid (58 mg, 0.458 mmol) in dry DMF (3 mL) was added TEA (0.2 mL, 1.374 mmol) followed by EDC·HCl (134 mg, 0.688 mmol) and HOBt (61.9 mg, 0.458 mmol). The reaction mixture was stirred at room temperature for 10 minutes and then N2-((1R,4R)-4-aminocyclohexyl)-N4-(5-cyclopropyl-1H-pyrazol-3-yl)-N2-methylpyrimidine-2,4-diamine (150 mg, 0.458 mmol) was added. The reaction mixture was stirred at room temperature for 16 h. After completion of the reaction, the reaction mixture was diluted with dichloromethane washed with water and brine, dried over anhydrous Na₂SO₄ and concentrated to yield the crude product. The crude product was purified by reverse phase prep HPLC to provide N-((1R,4R)-4-((4-((5-cyclopropyl-1H-pyrazol-3-yl)amino)pyrimidin-2-yl)(methyl)amino)cyclohexyl)-1-methyl-1H-1,2,3-triazole-4-carboxamide (50 mg, 25%) as the free base. LC purity: 99.56%; m/z: 437.4 [M+H]⁺ (Mol. formula C₂₁H₂₈N₁₀O, calcd. mol. wt. 436.52). ¹H VTNMR (400 MHz, CD₃OD): δ 8.30 (s, 1H), 7.73 (d, J=7.2 Hz, 1H), 6.55-6.29 (m, 2H), 4.16 (s, 3H), 3.97-3.91 (m, 1H), 3.11 (s, 3H), 2.24-2.20 (m, 2H), 2.00-1.98 (m, 1H), 1.97-1.87 (m, 5H), 1.67-1.57 (m, 2H), 1.07-1.02 (m, 2H), 0.79-0.75 (m, 2H).

### Example 139: Synthesis of Compound 141

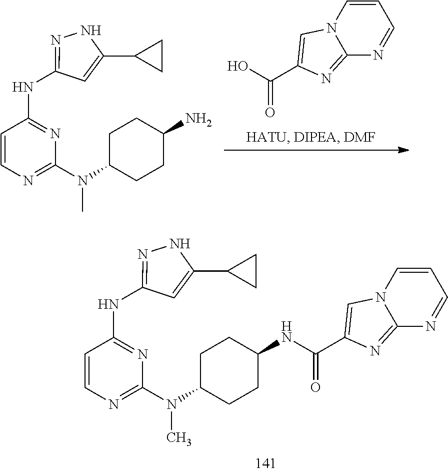 [SMILES: CN(c1nccc(Nc2cc(C3CC3)[nH]n2)n1)[C@H]1CC[C@H](N)CC1 ; O=C(O)c1cn2cccnc2n1 ; CN(c1nccc(Nc2cc(C3CC3)[nH]n2)n1)[C@H]1CC[C@H](NC(=O)c2cn3cccnc3n2)CC1]

### Synthesis of N-((1R,4R)-4-((4-((5-cyclopropyl-1H-pyrazol-3-yl)amino)pyrimidin-2-yl) (methyl)amino)cyclohexyl)imidazo[1,2-a]pyrimidine-2-carboxamide

To a solution of imidazo[1,2-a]pyrimidine-2-carboxylic acid (74.6 mg, 0.458 mmol) in dry DMF (3 mL) was added DIPEA (0.23 mL, 1.376 mmol) followed by HATU (261 mg, 0.687 mmol). The reaction was stirred at room temperature for 10 min. Then N2-((1R,4R)-4-aminocyclohexyl)-N4-(5-cyclopropyl-1H-pyrazol-3-yl)-N2-methylpyrimidine-2,4-diamine (150 mg, 0.458 mmol) was added. The reaction mixture was stirred at room temperature for 16 h. After completion of the reaction, the reaction mixture was diluted with dichloromethane washed with water and brine, dried over anhydrous Na₂SO₄ and concentrated to yield the crude product. The crude product was purified by reverse phase prep HPLC to provide N-((1R,4R)-4-((4-((5-cyclopropyl-1H-pyrazol-3-yl)amino)pyrimidin-2-yl)(methyl)amino)cyclohexyl)imidazo[1,2-a]pyrimidine-2-carboxamide (20 mg, 9.2%) as the free base. LC purity: 98.83%; m/z: 473.1 [M+H]⁺ (Mol. formula C₂₄H₂₈N₁₀O, calcd. mol. wt. 472.56). ¹H NMR (400 MHz, CD₃OD): δ 9.80 (dd, J=7.2, 7.2 Hz, 1H), 8.74-8.73 (m, 1H), 8.41 (s, 1H), 7.88 (d, J=6 Hz, 1H), 7.25-7.23 (m, 1H), 6.18 (d, J=5.6 Hz, 1H), 6.07 (s, 1H), 4.69-4.61 (m, 1H), 3.98-3.94 (m, 1H), 3.05 (s, 3H), 2.20-2.17 (m, 2H), 1.96-1.84 (m, 5H), 1.71-1.63 (m, 2H), 1.00-0.96 (m, 2H), 0.77-0.73 (m, 2H).

### Example 140: Synthesis of Compound 142

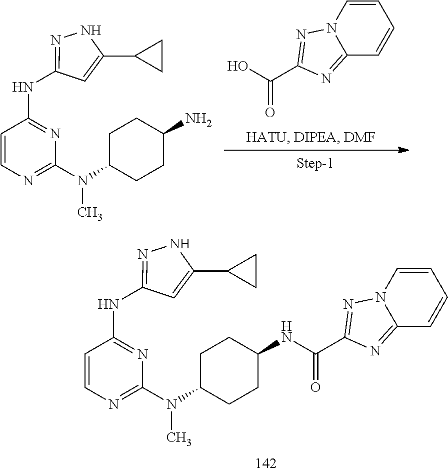 [SMILES: CN(c1nccc(Nc2cc(C3CC3)[nH]n2)n1)[C@H]1CC[C@H](N)CC1 ; O=C(O)c1nc2ccccn2n1 ; CN(c1nccc(Nc2cc(C3CC3)[nH]n2)n1)[C@H]1CC[C@H](NC(=O)c2nc3ccccn3n2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-[1,2,4]triazolo[1,5-a]pyridine-2-carboxamide

To a stirred solution of [1,2,4]triazolo[1,5-a]pyridine-2-carboxylic acid (200 mg, 0.550 mmol) in dry DMF (5 mL) was added DIPEA (0.28 mL, 1.650 mmol) followed by HATU (418 mg, 1.10 mmol). The reaction mixture was stirred at room temperature for 10 min and then N2-((1R,4R)-4-aminocyclohexyl)-N4-(5-cyclopropyl-1H-pyrazol-3-yl)-N2-methylpyrimidine-2,4-diamine (717 mg, 0.440 mmol) was added. The reaction mixture was stirred at room temperature for 16 h. The progress of the reaction was monitored by UPLC. After completion of the reaction, the reaction mixture was diluted with water and extracted with 10% methanol in dichloromethane. The combined organic layers were washed with brine, dried over anhydrous Na₂SO₄ and concentrated under reduced pressure to obtain the residue. The residue was purified by reverse phase preparative HPLC to afford N-((1R,4R)-4-((4-((5-cyclopropyl-1H-pyrazol-3-yl)amino)pyrimidin-2-yl)(methyl)amino)cyclohexyl)-[1,2,4]triazolo[1,5-a]pyridine-2-carboxamide (50 mg, 19.3%) as the free base. LC purity: 99.43%; m/z: 473.3 [M+H]⁺ (Mol. formula C₂₄H₂₈N₁₀O calcd. mol. wt. 472.56). ¹H NMR (400 MHz, MeOD): δ 8.89 (d, J=6.4 Hz, 1H), 7.89-7.74 (m, 3H), 7.32 (t, J=6.8 Hz, 1H), 6.41-6.35 (m, 2H), 4.65-4.58 (m, 1H), 4.04-3.98 (m, 1H), 3.12 (s, 3H), 2.25-2.18 (m, 2H), 2.05-1.94 (m, 5H), 1.74-1.64 (m, 2H), 1.06-0.97 (m, 2H), 0.79-0.67 (m, 2H).

### Example 141: Synthesis of Compound 143

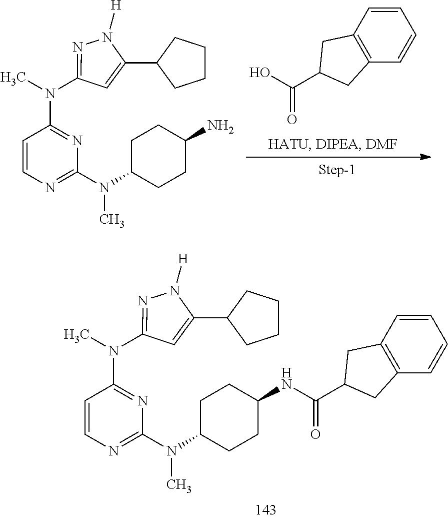 [SMILES: CN(c1cc(C2CCCC2)[nH]n1)c1ccnc(N(C)[C@H]2CC[C@H](N)CC2)n1 ; O=C(O)C1Cc2ccccc2C1 ; CN(c1cc(C2CCCC2)[nH]n1)c1ccnc(N(C)[C@H]2CC[C@H](NC(=O)C3Cc4ccccc4C3)CC2)n1]

### Step-1: Synthesis of N-((1R,4R)-4-((4-((5-cyclopentyl-1H-pyrazol-3-yl)(methyl)amino) pyrimidin-2-yl)(methyl)amino)cyclohexyl)-2,3-dihydro-1H-indene-2-carboxamide

To a solution of 2,3-dihydro-1H-indene-2-carboxylic acid (0.052 g, 0.32 mmol) in dry DMF (5 mL) was added DIPEA (0.35 mL, 2 mmol) followed by HATU (0.235 g, 0.6 mmol). The reaction mixture was stirred for 15 min. Then N2-((1R,4R)-4-aminocyclohexyl)-N4-(5-cyclopentyl-1H-pyrazol-3-yl)-N2,N4-dimethylpyrimidine-2,4-diamine (0.150 g, 0.4 mmol) was added. The reaction was stirred at room temperature for 1 h. The progress of the reaction was monitored by LCMS. After completion of the reaction, the reaction mixture was diluted with DCM and washed with water. The organic layer was dried over anhydrous sodium sulfate, filtered and concentrated under reduced pressure to obtain the crude product. The crude product was purified by reverse phase preparative HPLC (0.1% TFA in water/acetonitrile) to obtain N-((1R,4R)-4-((4-((5-cyclopentyl-1H-pyrazol-3-yl)(methyl) amino)pyrimidin-2-yl)(methyl)amino)cyclohexyl)-2,3-dihydro-1H-indene-2-carboxamide (40 mg, 19.2%). LC purity: 97.66%; m/z: 514.3 [M+H]⁺ (Mol. formula C₃₀H₃₉N₇O calcd. mol. wt. 513.69). ¹H NMR (400 MHz, CD₃OD): δ 7.63 (d, J=7.6 Hz, 1H), 7.19-7.11 (m, 4H), 6.27 (d, J=6.8 Hz, 1H), 6.13 (s, 1H), 4.52-4.49 (m, 1H), 3.77-3.71 (m, 1H), 3.33 (s, 3H), 3.28-3.21 (m, 6H), 3.18-3.11 (s, 3H), 2.14-2.11 (m, 4H), 1.88-1.82 (m, 6H), 1.78-1.65 (m, 4H), 1.65-1.49 (m, 2H).

### Example 142: Synthesis of Compound 144

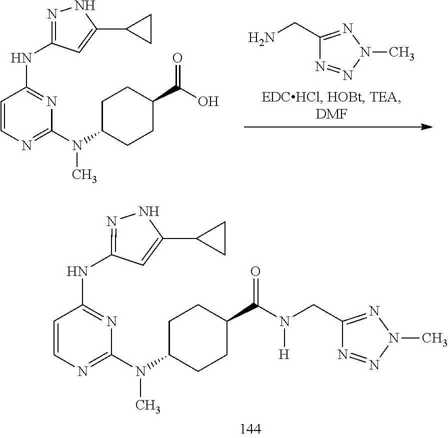

### Step-1: Synthesis of (1R,4R)-4-((4-((5-cyclopropyl-1H-pyrazol-3-yl)amino)pyrimidin-2-yl)(methyl)amino)-N-((2-methyl-2H-tetrazol-5-yl)methyl)cyclohexane-1-carboxamide

To a stirred solution of (1R,4R)-4-((4-((5-cyclopropyl-1H-pyrazol-3-yl)amino)pyrimidin-2-yl)(methyl)amino)cyclohexane-1-carboxylic acid (200 mg, 0.561 mmol) in dry DMF (3 mL) was added triethylamine (0.23 mL, 1.685 mmol) followed by the addition of EDC·HCl (160.9 mg, 0.842 mmol) and HOBt (37.92 mg, 0.280 mmol). The reaction mixture was stirred at room temperature for 15 min. Then (2-methyl-2H-tetrazol-5-yl)methanamine (66.96 mg, 0.449 mmol) was added. The reaction mixture was stirred at room temperature for 16 h. After completion of the reaction (monitored by UPLC), the reaction mixture was diluted with dichloromethane washed with water and brine, dried over anhydrous Na₂SO₄ and concentrated under reduced pressure to yield the crude compound. The crude product was purified by reverse phase preparative HPLC to yield (1R,4R)-4-((4-((5-cyclopropyl-1H-pyrazol-3-yl)amino)pyrimidin-2-yl)(methyl)amino)-N-((2-methyl-2H-tetrazol-5-yl)methyl)cyclohexane-1-carboxamide (30 mg, 12%) as the free base. LC purity: 97.90%; m/z: 452.3 [M+H]⁺ (Mol. formula C₂₁H₂₉N₁₁O, calcd. mol. wt. 451.54). ¹H NMR (400 MHz, CD₃OD): δ 7.85 (d, J=6 Hz, 1H), 6.15-6.21-6.14 (m, 2H), 4.70-4.62 (m, 3H), 4.35 (s, 3H), 2.99 (s, 3H), 2.30-2.25 (m, 1H), 2.04-2.02 (m, 2H), 1.94-1.89 (m, 1H), 1.84-1.82 (m, 2H), 1.77-1.66 (m, 4H), 1.01-1.00 (m, 2H), 0.66-0.44 (m, 2H).

### Example 143: Synthesis of Compound 145

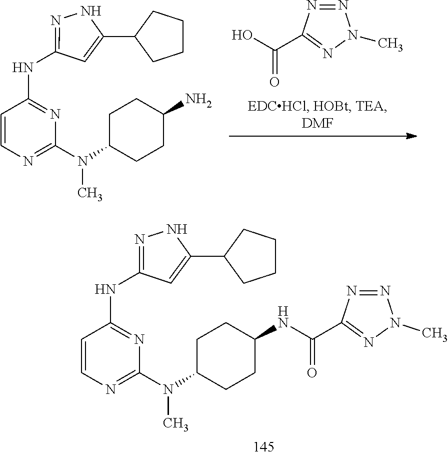

### Step-1: Synthesis of N-((1R,4R)-4-((4-((5-cyclopentyl-1H-pyrazol-3-yl)amino)pyrimidin-2-yl)(methyl)amino)cyclohexyl)-2-methyl-2H-tetrazole-5-carboxamide

To a stirred solution of 2-methyl-2H-tetrazole-5-carboxylic acid (72 mg, 0.563 mmol) in DMF (3 mL) was added DIPEA (0.29 mL, 1.689 mmol) and HATU (321 mg, 0.844 mmol). The reaction was stirred at RT for 10 min. Then N2-((1R,4R)-4-aminocyclohexyl)-N4-(5-cyclopentyl-1H-pyrazol-3-yl)-N2-methylpyrimidine-2,4-diamine (200 mg, 0.563 mmol) was added. The reaction mixture was stirred at RT for 16 h. The progress of the reaction was monitored by TLC. After complete consumption of the starting material, the reaction mixture was quenched with water, extracted with dichloromethane and then concentrated. The crude product was purified by reverse phase prep HPLC to yield N-((1R,4R)-4-((4-((5-cyclopentyl-1H-pyrazol-3-yl)amino)pyrimidin-2-yl)(methyl) amino)cyclohexyl)-2-methyl-2H-tetrazole-5-carboxamide (25 mg, 10%) as the free base. LC purity: 99.26%; m/z: 466.1 [M+H]⁺ (Mol. formula C₂₂H₃₁N₁₁O, calcd. mol. wt. 465.57). ¹H VTNMR (400 MHz, CD₃OD): δ 7.88 (d, J=6.0 Hz, 1H), 6.25-6.15 (m, 2H), 4.64-4.61 (m, 1H), 4.46 (s, 3H), 4.00-3.94 (m, 1H), 3.13-3.09 (m, 1H), 3.02 (s, 3H), 2.16-2.13 (m, 4H), 1.85-1.82 (m, 6H), 1.77-1.66 (m, 6H).

### Example 144: Synthesis of Compound 146

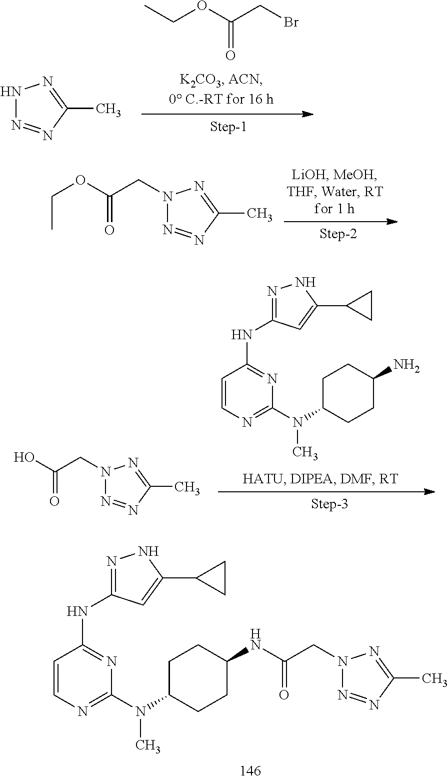

### Step-1: Synthesis of ethyl 2-(5-methyl-2H-tetrazol-2-yl)acetate

To a cooled (0° C.) solution of 5-methyl-2H-tetrazole (500 mg, 5.949 mmol) in acetonitrile (5 mL) was added potassium carbonate (1.64 g, 11.899 mmol) followed by the addition of ethyl 2-bromoacetate (0.99 mL, 8.924 mmol). The reaction mixture was stirred at room temperature for 16 h. After completion of the reaction (monitored by TLC), the reaction mixture was diluted with water and extracted with ethyl acetate. The organic layer was dried over anhydrous Na₂SO₄ and concentrated under reduced pressure to yield ethyl 2-(5-methyl-2H-tetrazol-2-yl)acetate (700 mg, 69.30%). LC purity: 99.30%; m/z: 171.1 [M+H]⁺ (Mol. formula C₆H₁₀N₄O₂ calcd. mol. wt. 170.17).

### Step-2: Synthesis of 2-(5-methyl-2H-tetrazol-2-yl)acetic acid

To a stirred solution of ethyl 2-(5-methyl-2H-tetrazol-2-yl)acetate (700 mg, 4.117 mmol) in THF, methanol, water (1:1:1, 10 mL) was added lithium hydroxide monohydrate (518.3 mg, 12.352 mmol). The reaction mixture was stirred at room temperature for 1 h. After completion of the reaction (monitored by TLC), the reaction mixture was concentrated under reduced pressure to obtain 2-(5-methyl-2H-tetrazol-2-yl)acetic acid (400 mg, 68.49%). LC purity: 88.24%; m/z: 143.2 [M+H]⁺ (Mol. formula C₄H₆N₄O₂ calcd. mol. wt. 142.12).

### Step-3: Synthesis of N-((1R,4R)-4-((4-((5-cyclopropyl-1H-pyrazol-3-yl)amino)pyrimidin-2-yl)(methyl)amino)cyclohexyl)-2-(5-methyl-2H-tetrazol-2-yl)acetamide

To a stirred solution of 2-(5-methyl-2H-tetrazol-2-yl)acetic acid (104 mg, 0.733 mmol) in dry DMF (3 mL) was added triethylamine (0.25 mL, 1.834 mmol) followed by the addition of the EDC·HCl (175.2 mg, 0.917 mmol) and HOBt (41.2 mg, 0.305 mmol). The reaction mixture was stirred at room temperature for 15 min, and then N2-((1R,4R)-4-aminocyclohexyl)-N4-(5-cyclopropyl-1H-pyrazol-3-yl)-N2-methylpyrimidine-2,4-diamine (200 mg, 0.611 mmol) was added. The reaction mixture was stirred at room temperature for 16 h. After completion of the reaction (monitored by UPLC), the reaction mixture was diluted with dichloromethane and washed with water. The aqueous layer was concentrated under reduced pressure to yield the crude compound. The crude product was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-2-(5-methyl-2H-tetrazol-2-yl)acetamide (9 mg, 3.27%) as the free base. LC purity: 99.37%; m/z: 452.0 [M+H]⁺ (Mol. formula C₂₁H₂₉N₁₁O, calcd. mol. wt. 451.54). ¹H NMR (400 MHz, MeOD): δ 7.87 (d, J=5.6 Hz, 1H), 6.20 (s, 1H), 6.13 (d, J=6.8 Hz, 1H), 4.89 (s, 2H), 4.67-4.58 (m, 1H), 3.75-3.69 (m, 1H), 3.02 (s, 3H), 2.53 (s, 3H), 2.12-2.09 (m, 2H), 1.95-1.91 (m, 1H), 1.80-1.74 (m, 4H), 1.57-1.50 (m, 2H), 1.03-0.99 (m, 2H), 0.77-0.73 (m, 2H).

### Example 145: Synthesis of Compound 147

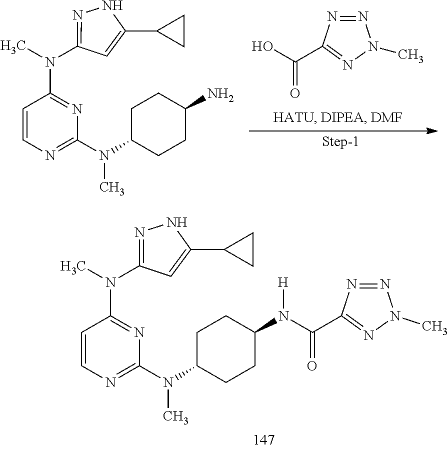

### Step-1: Synthesis of N-((1R,4R)-4-((4-((5-cyclopropyl-1H-pyrazol-3-yl)(methyl)amino) pyrimidin-2-yl)(methyl)amino)cyclohexyl)-2-methyl-2H-tetrazole-5-carboxamide

To a solution of 2-methyl-2H-tetrazole-5-carboxylic acid (75 mg, 0.580 mmol) in dry DMF (2 mL) was added DIPEA (0.3 mL, 1.75 mmol) drop-wise at 0° C. followed by the addition of HATU (445 mg, 1.17 mmol). The reaction mixture was stirred for 5 min and then N2-((1R,4R)-4-aminocyclohexyl)-N4-(5-cyclopropyl-1H-pyrazol-3-yl)-N2,N4-dimethylpyrimidine-2,4-diamine (200 mg, 0.580 mmol) was added. The reaction mixture was stirred at room temperature for 16 h. The progress of the reaction was monitored by TLC. After completion of the reaction, the reaction mixture was diluted with dichloromethane washed with water, and brine, dried over anhydrous Na₂SO₄ and concentrated to remove solvent to provide the crude compound. The crude product was purified by reverse phase preparative HPLC to yield N-((1R,4R)-4-((4-((5-cyclopropyl-1H-pyrazol-3-yl)(methyl)amino)pyrimidin-2-yl)(methyl)amino)cyclohexyl)-2-methyl-2H-tetrazole-5-carboxamide (30 mg, 11.36%) as the free base. LC purity: 99.58%; m/z: 452.3 [M+H]⁺ (Mol. formula C₂₁H₂₉N₁₁O, calcd. mol. wt. 451.54). ¹H NMR (400 MHz, CD₃OD): δ 7.78 (d, J=8.0 Hz, 1H), 5.97 (d, J=6.0 Hz, 1H), 5.92 (s, 1H), 4.61-4.58 (m, 1H), 4.45 (s, 3H), 4.01-3.95 (m, 1H), 3.41 (s, 3H), 3.01 (s, 3H), 2.14-2.05 (m, 2H), 1.98-1.92 (m, 1H), 1.84-1.79 (m, 4H), 1.71-1.61 (m, 2H), 1.03-0.97 (m, 2H), 0.79-0.75 (m, 2H).

### Example 146: Synthesis of Compound 148

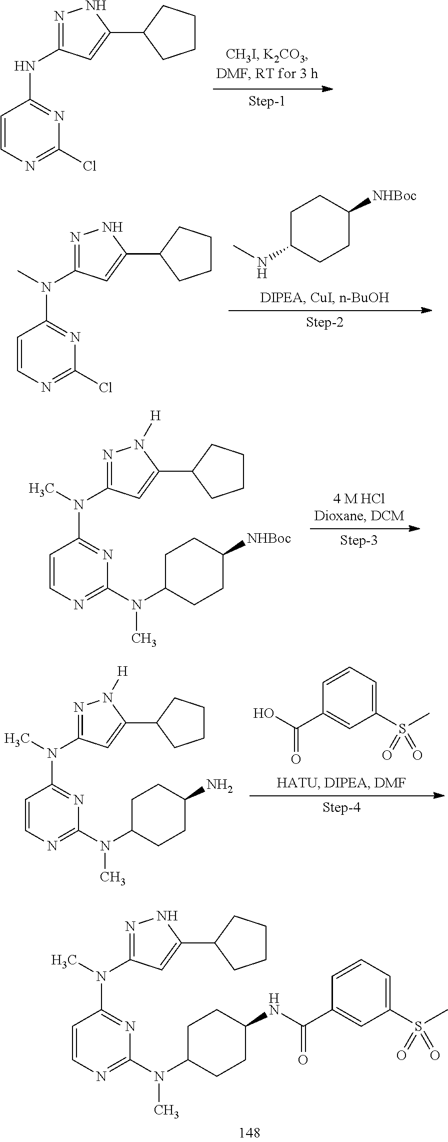

### Step-1: Synthesis of 2-chloro-N-(5-cyclopentyl-1H-pyrazol-3-yl)-N-methylpyrimidin-4-amine

To a solution of 2-chloro-N-(5-cyclopentyl-1H-pyrazol-3-yl)pyrimidin-4-amine (3 g, 11.4 mmol) in dry DMF was added potassium carbonate (3.14 g, 22.8 mmol). The reaction mixture was stirred for 10 min at RT. The reaction mixture was cooled to 0° C. followed by addition of methyl iodide (0.71 mL, 11.4 mmol) dropwise. The reaction mixture was then stirred at RT for 2 h. The progress of the reaction was monitored by TLC (40% SM was remaining). The reaction mixture was diluted with water and the organic layer was extracted with ethyl acetate. The organic layer was dried over anhydrous sodium sulfate, filtered and concentrated under reduced pressure. The crude was purified by Biotage Isolera using silica gel (230-400) with gradient elution of 0-20% ethyl acetate in pet ether to obtain 2-chloro-N-(5-cyclopentyl-1H-pyrazol-3-yl)-N-methylpyrimidin-4-amine (1.1 g, 35.48%). LC purity: 93.2%; m/z: 276.2 [M−H]⁻ (Mol. formula C₁₃H₁₆ClN₅ calcd. mol. wt. 277.76).

### Step-2: Synthesis of tert-butyl (4-((4-((5-cyclopentyl-1H-pyrazol-3-yl)(methyl)amino) pyrimidin-2-yl)(methyl)amino)cyclohexyl)carbamate

To a solution of tert-butyl (4-(methylamino)cyclohexyl)carbamate (1 g, 4.38 mmol) in n-Butanol (10 mL) in a 20 mL microwave vial was added 2-chloro-N-(5-cyclopentyl-1H-pyrazol-3-yl)-N-methylpyrimidin-4-amine (0.607 g, 2.19 mmol), DIPEA (2.29 mL, 25.5 mmol) and copper iodide (100 mg). The reaction mixture was heated in a microwave reactor at 160° C. for 2 h. The progress of the reaction was monitored by TLC. After completion of the reaction, the reaction mixture was cooled to ambient temperature and concentrated to remove solvent. The crude was purified by Biotage Isolera using silica gel (230-400) with gradient elution of 0-60% ethyl acetate in pet ether to obtain tert-butyl (4-((4-((5-cyclopentyl-1H-pyrazol-3-yl)(methyl)amino)pyrimidin-2-yl)(methyl)amino)cyclohexyl)carbamate (614 mg, 30.7%). LC purity: 96.3%; m/z: 470. [M+H]⁺ (Mol. formula C₂₅H₃₉N₇O₂ calcd. mol. wt. 469.3).

### Step-3: Synthesis of N2-((1R,4R)-4-aminocyclohexyl)-N4-(5-cyclopentyl-1H-pyrazol-3-yl)-N2,N4-dimethylpyrimidine-2,4-diamine

A stirred solution of tert-butyl ((1R,4R)-4-((4-((5-cyclopentyl-1H-pyrazol-3-yl)(methyl)amino)pyrimidin-2-yl)(methyl)amino)cyclohexyl)carbamate (0.614 g, 1.39 mmol) in dry DCM (2 mL) was cooled to 0° C. and HCl in dioxane (6 mL, 4M solution) was added. The reaction mixture was allowed to stir at room temperature for 1 h. The progress of the reaction was monitored by TLC. After complete consumption of the starting material, the resulting reaction mixture was concentrated and triturated with pet ether, and then concentrated under high vacuum to yield N2-((1R,4R)-4-aminocyclohexyl)-N4-(5-cyclopentyl-1H-pyrazol-3-yl)-N2,N4-dimethylpyrimidine-2,4-diamine as the HCl salt (600 mg, quantitative yield). LC purity: 95.63%; m/z: 370.3 [M+H]⁺ (Mol. formula C₂₀H₃₁N₇ calcd. mol. wt. 369.2).

### Step-4: Synthesis of N-((1R,4R)-4-((4-((5-cyclopentyl-1H-pyrazol-3-yl)(methyl)amino) pyrimidin-2-yl)(methyl)amino)cyclohexyl)-3-(methylsulfonyl)benzamide

To a solution of 3-(methylsulfonyl)benzoic acid (0.135 g, 0.67 mmol) in dry DMF (5 mL) was added DIPEA (0.35 mL, 2.01 mmol) followed by HATU (0.386 g, 1.01 mmol). The reaction mixture was stirred for 15 min. Then N2-((1R,4R)-4-aminocyclohexyl)-N4-(5-cyclopentyl-1H-pyrazol-3-yl)-N2,N4-dimethylpyrimidine-2,4-diamine (0.250 g, 0.67 mmol) was added. The reaction mixture was stirred at room temperature for 1 h. The progress of the reaction was monitored by UPLC. The reaction mixture was diluted with water and extracted with dichloromethane. The organic layer was dried over anhydrous sodium sulfate, filtered and concentrated under reduced pressure. The crude was purified by reverse phase prep HPLC method to obtain N-((1R,4R)-4-((4-((5-cyclopentyl-1H-pyrazol-3-yl)(methyl)amino)pyrimidin-2-yl)(methyl)amino)cyclohexyl)-3-(methylsulfonyl)benzamide (80 mg, 21.44%) as the free base. LC purity: 96.94%; m/z: 552.3 [M+H]⁺ (Mol. formula C₂₈H₃₇N₇O₃S calcd. mol. wt. 551.71). ¹H NMR (400 MHz, CD₃OD): δ 8.41 (s, 1H), 8.18-8.12 (m, 2H), 7.79-7.74 (m, 2H), 6.06 (s, 1H), 5.98 (d, J=6 Hz, 1H), 4.57-4.49 (m, 1H), 3.98-3.92 (m, 1H), 3.43 (s, 3H), 3.17 (s, 3H), 3.15-3.12 (m, 1H), 3.04 (s, 3H), 2.17-2.11 (m, 4H), 1.86-1.80 (m, 6H), 1.79-1.69 (m, 6H).

### Example 147: Synthesis of Compound 149

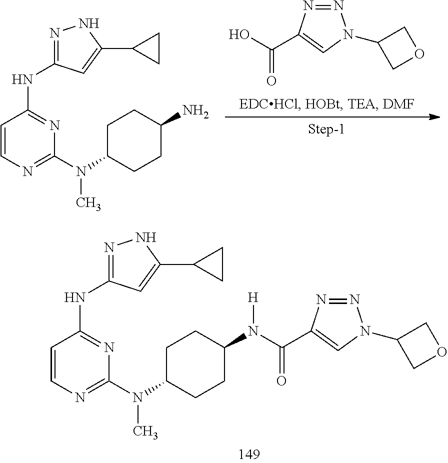 [SMILES: CN(c1nccc(Nc2cc(C3CC3)[nH]n2)n1)[C@H]1CC[C@H](N)CC1 ; O=C(O)c1cn(C2COC2)nn1 ; CN(c1nccc(Nc2cc(C3CC3)[nH]n2)n1)[C@H]1CC[C@H](NC(=O)c2cn(C3COC3)nn2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-1-(oxetan-3-yl)-1H-1,2,3-triazole-4-carboxamide

To a solution of 1-(oxetan-3-yl)-1H-1,2,3-triazole-4-carboxylic acid (103 mg, 0.611 mmol) in DMF (4 mL) was added TEA (0.3 mL, 1.8 mmol) followed by the addition EDC·HCl (232 mg, 1.22 mmol) and HOBt (123 mg, 0.917 mmol). The reaction mixture was stirred for 5 min and then N2-((1R,4R)-4-aminocyclohexyl)-N4-(5-cyclopropyl-1H-pyrazol-3-yl)-N2-methylpyrimidine-2,4-diamine (200 mg, 0.611 mmol) was added. The reaction mixture was stirred at room temperature for 16 h. The progress of the reaction was monitored by TLC. After completion of the reaction, the reaction mixture was diluted with dichloromethane, washed with water and brine and dried over anhydrous Na₂SO₄ and concentrated to remove solvent to provide the residue. The residue was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-1-(oxetan-3-yl)-1H-1,2,3-triazole-4-carboxamide (30 mg, 10.27%) as the free base. LC purity: 99.28%; m/z: 479.1 [M+H]⁺ (Mol. formula C₂₃H₃₀N₁₀O₂, calcd. mol. wt. 478.56). ¹H NMR (400 MHz, CD₃OD): δ 8.57 (s, 1H), 7.88 (d, J=5.2 Hz, 1H), 6.15-6.09 (m, 2H), 5.92-5.86 (m, 1H), 5.23-5.14 (m, 2H), 5.09-5.05 (m, 2H), 4.70-4.60 (m, 1H), 3.95-3.90 (m, 1H), 3.03 (s, 3H), 2.17-2.14 (m, 2H), 1.96-1.92 (m, 1H), 1.85-1.80 (m, 4H), 1.68-1.64 (m, 2H), 1.02-1.00 (m, 2H), 0.77-0.75 (m, 2H).

### Example 148: Synthesis of Compound 150

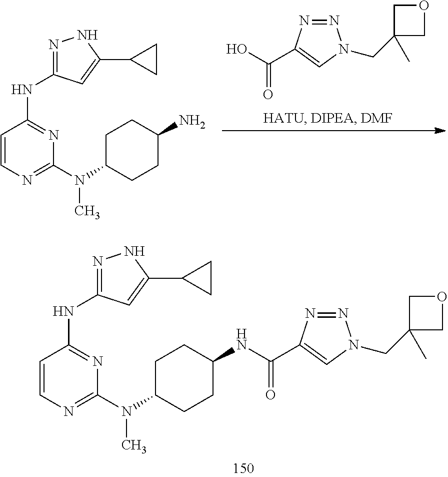 [SMILES: CN(c1nccc(Nc2cc(C3CC3)[nH]n2)n1)[C@H]1CC[C@H](N)CC1 ; CC1(Cn2cc(C(=O)O)nn2)COC1 ; CN(c1nccc(Nc2cc(C3CC3)[nH]n2)n1)[C@H]1CC[C@H](NC(=O)c2cn(CC3(C)COC3)nn2)CC1]

### Synthesis of N-((1R,4R)-4-((4-((5-cyclopropyl-1H-pyrazol-3-yl)amino)pyrimidin-2-yl)(methyl)amino)cyclohexyl)-1-((3-methyloxetan-3-yl)methyl)-1H-1,2,3-triazole-4-carboxamide

To a solution of 1-((3-methyloxetan-3-yl)methyl)-1H-1,2,3-triazole-4-carboxylic acid (84 mg, 0.427 mmol) in dry DMF (2 mL) was added DIPEA (0.32 mL, 1.832 mmol) followed by HATU (464 mg, 1.221 mmol). The reaction mixture was stirred at room temperature for 10 min. Then N2-((1R,4R)-4-aminocyclohexyl)-N4-(5-cyclopropyl-1H-pyrazol-3-yl)-N2-methylpyrimidine-2,4-diamine (200 mg, 0.610 mmol) was added. The reaction mixture was stirred at room temperature for 16 h. After completion of the reaction (monitored by TLC), the reaction mixture was diluted with water and extracted with 10% methanol in dichloromethane. The organic layer was washed with brine, dried over anhydrous Na₂SO₄ and concentrated to yield the crude product. The crude product was purified by reverse phase preparative HPLC to afford N-((1R,4R)-4-((4-((5-cyclopropyl-1H-pyrazol-3-yl)amino)pyrimidin-2-yl)(methyl)amino)cyclohexyl)-1-((3-methyloxetan-3-yl)methyl)-1H-1,2,3-triazole-4-carboxamide (65 mg, 23%) as the free base. LC purity: 97.95%; m/z: 507.2 [M+H]⁺ (Mol. Formula C₂₅H₃₄N₁₀O₂ calcd. mol. wt. 506.62). ¹H NMR (400 MHz, CD₃OD): δ 8.37 (s, 1H), 7.86 (d, J=5.2 Hz, 1H), 6.19-6.17 (d, J=6.4 Hz, 2H), 4.72-4.67 (m, 4H), 4.43-4.41 (d, J=5.2 Hz, 2H), 3.98-3.91 (m, 1H), 3.04 (s, 3H), 2.18-2.15 (m, 2H), 1.97-1.90 (m, 1H), 1.86-1.77 (m, 4H), 1.69-1.62 (m, 2H), 1.31-1.24 (m, 4H), 1.00-0.98 (m, 2H), 0.77-0.73 (m, 2H).

### Example 149: Synthesis of Compound 151

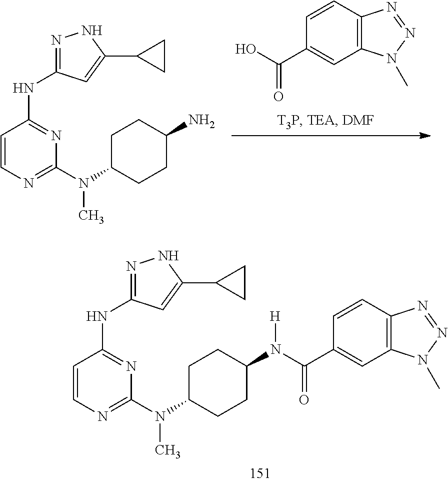 [SMILES: CN(c1nccc(Nc2cc(C3CC3)[nH]n2)n1)[C@H]1CC[C@H](N)CC1 ; Cn1nnc2ccc(C(=O)O)cc21 ; Cn1nnc2ccc(C(=O)N[C@H]3CC[C@H](N(C)c4nccc(Nc5cc(C6CC6)[nH]n5)n4)CC3)cc21]

### Synthesis of N-((1R,4R)-4-((4-((5-cyclopropyl-1H-pyrazol-3-yl)amino)pyrimidin-2-yl)(methyl)amino)cyclohexyl)-1-methyl-1H-benzo[d][1,2,3]triazole-6-carboxamide

To a solution of 1-methyl-1H-benzo[d][1,2,3]triazole-6-carboxylic acid (108 mg, 0.611 mmol) in DMF (4 mL) was added TEA (0.3 mL, 1.83 mmol) followed by the addition of T₃P (0.6 mL, 1.83 mmol, 50% in EtOAc). The reaction mixture was stirred for 5 min and then N2-((1R,4R)-4-aminocyclohexyl)-N4-(5-cyclopropyl-1H-pyrazol-3-yl)-N2-methylpyrimidine-2,4-diamine (200 mg, 0.611 mmol) was added. The reaction mixture was stirred at room temperature for 16 h. The progress of the reaction was monitored by TLC. After completion of the reaction, the reaction mixture was diluted with dichloromethane, washed with water and brine, dried over anhydrous Na₂SO₄ and concentrated to remove solvent to provide the crude product. The crude product was purified by reverse phase preparative HPLC to yield N-((1R,4R)-4-((4-((5-cyclopropyl-1H-pyrazol-3-yl)amino)pyrimidin-2-yl)(methyl)amino)cyclohexyl)-1-methyl-1H-benzo[d][1,2,3] triazole-6-carboxamide (24 mg, 8.08%) as the free base. LC purity: 98.11%; m/z: 487.3 [M+H]⁺ (Mol. formula C₂₅H₃₀N₁₀O, calcd. mol. wt. 486.58). ¹H VTNMR (400 MHz, CD₃OD): δ 8.25 (s, 1H), 8.06-8.04 (m, 1H), 7.90-7.87 (m, 2H), 6.15 (d, J=5.6 Hz, 1H), 6.05 (s, 1H), 4.61-4.57 (m, 1H), 4.40 (s, 3H), 4.03-3.98 (m, 1H), 3.05 (s, 3H), 2.22-2.13 (m, 2H), 1.94-1.83 (m, 5H), 1.72-1.65 (m, 2H), 1.0-0.95 (m, 2H), 0.77-0.73 (m, 2H).

### Example 150: Synthesis of Compound 152

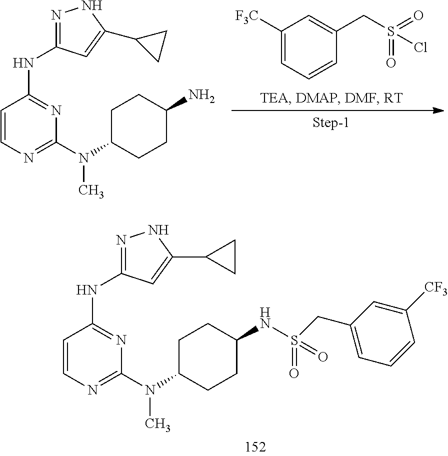 [SMILES: CN(c1nccc(Nc2cc(C3CC3)[nH]n2)n1)[C@H]1CC[C@H](N)CC1 ; O=S(=O)(Cl)Cc1cccc(C(F)(F)F)c1 ; CN(c1nccc(Nc2cc(C3CC3)[nH]n2)n1)[C@H]1CC[C@H](NS(=O)(=O)Cc2cccc(C(F)(F)F)c2)CC1]

### Step-1: Synthesis of N-((1R,4R)-4-((4-((5-cyclopropyl-1H-pyrazol-3-yl)amino)pyrimidin-2-yl)(methyl)amino)cyclohexyl)-1-(3-(trifluoromethyl)phenyl)methanesulfonamide

To a solution of N2-((1R,4R)-4-aminocyclohexyl)-N4-(5-cyclopropyl-1H-pyrazol-3-yl)-N2-methylpyrimidine-2,4-diamine (200 mg, 0.611 mmol) in dry DMF (4.0 mL) was added DMAP (catalytic), triethylamine (0.43 mL, 3.054 mmol) followed by addition of (3-(trifluoromethyl)phenyl)methanesulfonyl chloride (790 mg, 3.054 mmol). The resultant reaction mixture was stirred at room temperature for 16 h. The consumption of starting materials was monitored by TLC and LCMS analysis. The Reaction mixture was concentrated under reduced pressure to afforded the crude product, which was purified by preparative HPLC (0.1% TFA in water/acetonitrile) to afford the title compound N-((1R,4R)-4-((4-((5-cyclopropyl-1H-pyrazol-3-yl)amino)pyrimidin-2-yl)(methyl)amino)cyclohexyl)-1-(3-(trifluoromethyl)phenyl)methanesulfonamide (25 mg, 7%). LC purity: 98.06%; m/z: 550.0 [M+H]⁺ (Mol. formula C₂₅H₃₀N₇O₂S, calcd. mol. wt. 549.62). ¹H NMR (400 MHz, CD₃OD): δ 7.74 (d, J=8 Hz, 1H), 7.71-7.62 (m, 3H), 7.63-7.59 (m, 1H), 6.36-6.33 (m, 2H), 4.89 (s, 2H), 3.16-3.14 (m, 1H), 3.05 (s, 3H), 2.13 (m, 2H), 1.77-1.74 (m, 5H), 1.68-1.45 (m, 3H), 1.07-1.02 (m, 2H), 0.78-0.74 (m, 2H).

### Example 151: Synthesis of Compound 153

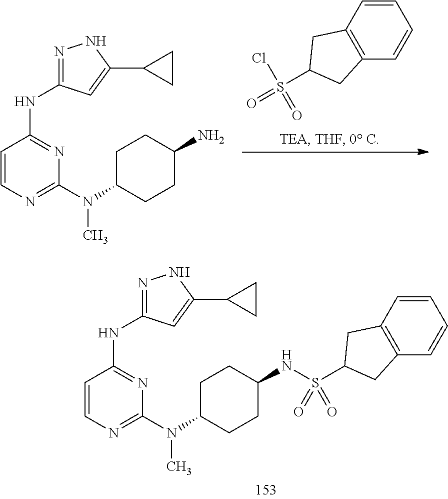 [SMILES: CN(c1nccc(Nc2cc(C3CC3)[nH]n2)n1)[C@H]1CC[C@H](N)CC1 ; O=S(=O)(Cl)C1Cc2ccccc2C1 ; CN(c1nccc(Nc2cc(C3CC3)[nH]n2)n1)[C@H]1CC[C@H](NS(=O)(=O)C2Cc3ccccc3C2)CC1]

### Synthesis of N-((1R,4R)-4-((4-((5-cyclopropyl-1H-pyrazol-3-yl)amino)pyrimidin-2-yl)(methyl)amino)cyclohexyl)-3-methylcyclopent-3-ene-1-sulfonamide

To a solution of N2-((1R,4R)-4-aminocyclohexyl)-N4-(5-cyclopropyl-1H-pyrazol-3-yl)-N2-methylpyrimidine-2,4-diamine (200 mg, 0.611 mmol) in dry THF (4 mL) was added TEA (0.25 mL, 1.83 mmol) followed by the addition of 2,3-dihydro-1H-indene-2-sulfonyl chloride (330 mg, 1.52 mmol) dropwise at 0° C. under nitrogen atmosphere. The reaction mixture was stirred for 4 h at room temperature. The progress of the reaction was monitored by TLC. After completion of the reaction, the solvent was evaporated to obtain the crude product. The crude product was purified by reverse phase preparative HPLC (0.1% TFA in water/acetonitrile) to obtain N-((1R,4R)-4-((4-((5-cyclopropyl-1H-pyrazol-3-yl)amino)pyrimidin-2-yl)(methyl)amino)cyclohexyl)-3-methylcyclopent-3-ene-1-sulfonamide (25 mg, 8.06%). LC purity: 98.96%; m/z: 508.2 [M+H]⁺ (Mol. formula C₂₆H₃₃N₇O₂S, calcd. mol. wt. 507.66). ¹H NMR (400 MHz, CD₃OD): δ 7.71 (d, J=7.2 Hz 1H), 7.26-7.17 (m, 4H), 6.36-6.21 (m, 2H), 4.58 (s, 1H), 4.12-4.08 (m, 1H), 3.42-3.27 (m, 5H), 3.06 (s, 3H), 2.07-1.98 (m, 3H), 1.82-1.76 (m, 4H), 1.49-1.45 (m, 2H), 1.06-1.01 (m, 2H), 0.78-0.74 (m, 2H).

### Example 152: Synthesis of Compound 154

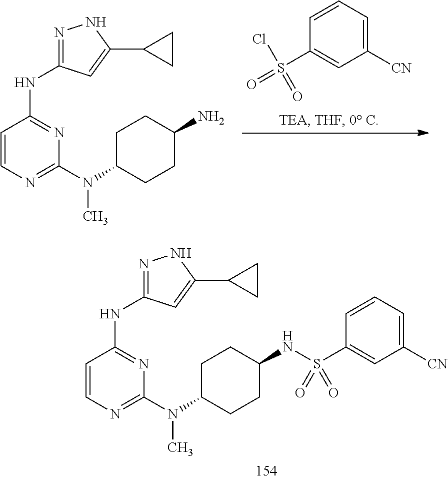 [SMILES: CN(c1nccc(Nc2cc(C3CC3)[nH]n2)n1)[C@H]1CC[C@H](N)CC1 ; N#Cc1cccc(S(=O)(=O)Cl)c1 ; CN(c1nccc(Nc2cc(C3CC3)[nH]n2)n1)[C@H]1CC[C@H](NS(=O)(=O)c2cccc(C#N)c2)CC1]

### Synthesis of 3-cyano-N-((1R,4R)-4-((4-((5-cyclopropyl-1H-pyrazol-3-yl)amino) pyrimidin-2-yl)(methyl)amino)cyclohexyl)benzenesulfonamide

To a solution of N2-((1R,4R)-4-aminocyclohexyl)-N4-(5-cyclopropyl-1H-pyrazol-3-yl)-N2-methylpyrimidine-2,4-diamine (200 mg, 0.611 mmol) in dry THF (4 mL) was added TEA (0.17 mL, 1.22 mmol) followed by the addition of 3-cyanobenzenesulfonyl chloride (123 mg, 0.611 mmol) dropwise at 0° C. under a nitrogen atmosphere. The reaction mixture was stirred for 6 h at room temperature. The reaction was monitored by TLC. After completion of the reaction, the solvent was evaporated to obtain the crude compound, which was purified by reverse phase preparative HPLC (0.1% TFA in water/acetonitrile) to yield 3-cyano-N-((1R,4R)-4-((4-((5-cyclopropyl-1H-pyrazol-3-yl)amino)pyrimidin-2-yl)(methyl)amino)cyclohexyl) benzenesulfonamide (25 mg, 8.33%). LC purity: 99.11%; m/z: 493.2 [M+H]⁺ (Mol. formula C₂₄H₂₈N₈O₂S, calcd. mol. wt. 492.60). (VT) ¹H NMR (400 MHz, CD₃OD): δ 8.24 (s, 1H), 8.23-8.17 (m, 1H), 7.99-7.76 (m, 1H), 7.78 (t, J=8.0 Hz, 1H), 7.70 (d, J=7.2 Hz, 1H), 6.32-6.15 (m, 2H), 4.59-4.54 (m, 1H), 3.21-3.15 (m, 1H), 3.03 (s, 3H), 1.98-1.92 (m, 3H), 1.78-1.69 (m, 4H), 1.50-1.40 (m, 2H), 1.05-1.02 (m, 2H), 0.77-0.73 (m, 2H).

### Example 153: Synthesis of Compound 155

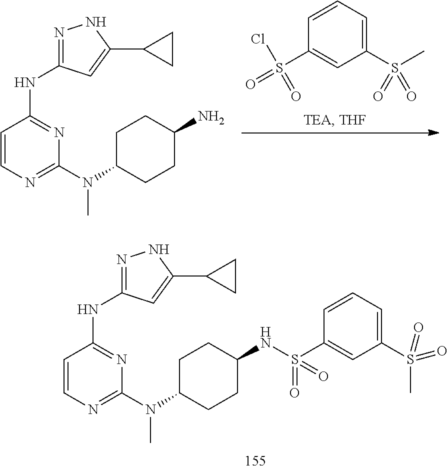 [SMILES: CN(c1nccc(Nc2cc(C3CC3)[nH]n2)n1)[C@H]1CC[C@H](N)CC1 ; CS(=O)(=O)c1cccc(S(=O)(=O)Cl)c1 ; CN(c1nccc(Nc2cc(C3CC3)[nH]n2)n1)[C@H]1CC[C@H](NS(=O)(=O)c2cccc(S(C)(=O)=O)c2)CC1]

### Synthesis of N-((1R,4R)-4-((4-((5-cyclopropyl-1H-pyrazol-3-yl)amino)pyrimidin-2-yl)(methyl)amino)cyclohexyl)-3-(methylsulfonyl)benzenesulfonamide

To a solution of N2-((1R,4R)-4-aminocyclohexyl)-N4-(5-cyclopropyl-1H-pyrazol-3-yl)-N2-methylpyrimidine-2,4-diamine (200 mg, 0.611 mmol) in dry THF (3 mL) was added TEA (0.25 mL, 1.833 mmol). The reaction was cooled to 0° C. and 3-(methylsulfonyl)benzenesulfonyl chloride (310 mg, 1.223 mmol) was added potion wise. The reaction was stirred at 0° C. for 3 h. After completion of the reaction (monitored by UPLC), the reaction mixture was diluted with ethyl acetate, washed with water and brine, dried over anhydrous Na₂SO₄ and concentrated to yield the crude product. The crude product was purified by reverse phase preparative HPLC to provide N-((1R,4R)-4-((4-((5-cyclopropyl-1H-pyrazol-3-yl)amino) pyrimidin-2-yl)(methyl)amino)cyclohexyl)-3-(methylsulfonyl)benzenesulfonamide (30 mg, 9.09%) as the free base. LC purity: 99.44%; m/z: 546.1 [M+H]⁺ (Mol. formula C₂₄H₃₁N₇O₄S₂, calcd. mol. wt. 545.68). ¹H VTNMR (400 MHz, CD₃OD): δ 8.45 (s, 1H), 8.24-8.19 (m, 2H), 7.88-7.84 (m, 2H), 6.15-6.10 (m, 2H), 4.62-4.57 (m, 1H), 3.20 (s, 3H), 3.18-3.14 (m, 1H), 2.94 (s, 3H), 1.93-1.88 (m, 3H), 1.71-1.59 (m, 4H), 1.52-1.44 (m, 2H), 0.99-0.97 (m, 2H), 0.74-0.70 (m, 2H).

### Example 154: Synthesis of Compound 156

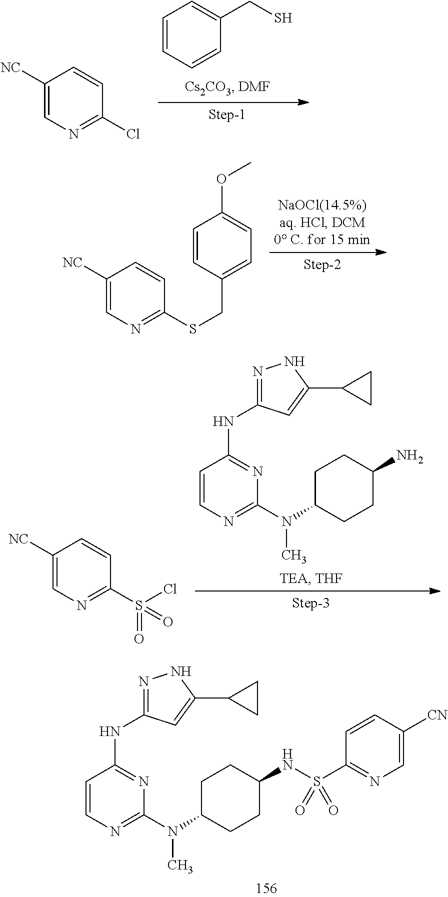

### Step-1: Synthesis of 6-((4-methoxybenzyl)thio)nicotinonitrile

To a stirred solution of 6-chloronicotinonitrile (500 mg, 3.6231 mmol) in dry DMF (8 mL) was added cesium carbonate (1.4 g, 4.3478 mmol) followed by phenylmethanethiol (0.42 mL, 3.6231 mmol). The reaction was stirred at room temperature for 16 h. The resulting solution was stirred for an additional 6 h at 60° C. The progress of the reaction was monitored by UPLC. After completion of the reaction, the reaction mixture was diluted with water and extracted with ethyl acetate. The combined organic layers were washed with water and brine, dried over anhydrous Na₂SO₄, filtered and concentered under reduced pressure to obtain 6-((4-methoxybenzyl)thio)nicotinonitrile (900 mg, crude) as solid. The crude product was used in the next step without further purification. LC purity: 96.21%; m/z: 257.0 [M+H]⁺ (Mol. formula C₁₄H₁₂N₂OS calcd. mol. wt. 256.32).

### Step-2: Synthesis of 5-cyanopyridine-2-sulfonyl chloride

To a stirred solution of 6-((4-methoxybenzyl)thio)nicotinonitrile (300 mg, 2.027 mmol) in DCM (9 mL) was added aqueous HCl (2 mL) and water (4 mL) dropwise at 0° C., followed by addition of sodium hypochlorite (3 mL, 14.5%) dropwise at the same temperature over 15 min. The progress of the reaction was monitored by UPLC, and the reaction mixture was directly evaporated under vacuum to get 5-cyanopyridine-2-sulfonyl chloride (280 mg, crude). Which was taken directly to next step without further purification. LC purity: (Mol. formula C₆H₃ClN₂O₂S calcd. mol. wt. 202.61).

### Step-3: Synthesis of 5-cyano-N-((1R,4R)-4-((4-((5-cyclopropyl-1H-pyrazol-3-yl)amino) pyrimidin-2-yl)(methyl)amino)cyclohexyl)pyridine-2-sulfonamide

To a cooled (0° C.) solution of N2-((1R,4R)-4-aminocyclohexyl)-N4-(3-cyclopropyl-2H-pyrrol-5-yl)-N2-methylpyrimidine-2,4-diamine (150 mg, 0.458 mmol) in dry THF (6 mL) was added TEA (0.2 mL, 1.374 mmol) followed by the dropwise addition of 5-cyanopyridine-2-sulfonyl chloride (278 mg, 1.374 mmol). The reaction was stirred at room temperature for 16 h. The progress of the reaction was monitored by UPLC. After completion of the reaction, the reaction mixture was diluted with water and extracted with 10% methanol in dichloromethane. The combined organic layers were washed with brine, dried over anhydrous Na₂SO₄ and concentrated under reduced pressure to obtain the residue. The residue was purified by reverse phase preparative HPLC to afford 5-cyano-N-((1R,4R)-4-((4-((5-cyclopropyl-1H-pyrazol-3-yl)amino)pyrimidin-2-yl)(methyl)amino)cyclohexyl)pyridine-2-sulfonamide (25 mg, 11%) as the free base. LC purity: 98.81%; m/z: 494 [M+H]⁺ (Mol. formula C₂₃H₂₇N₉O₂S calcd. mol. wt. 493.59). ¹H VTNMR (400 MHz, MeOD): δ 9.04 (s, 1H), 8.42 (d, J=6 Hz, 1H), 8.15 (d, J=7.2 Hz, 1H), 7.85 (d, J=5.6 Hz, 1H), 6.15-6.05 (m, 2H), 4.57-4.51 (m, 1H), 3.32-3.21 (m, 1H), 2.95 (s, 3H), 2.03-1.89 (m, 3H), 1.73-1.64 (m, 4H), 1.52-1.48 (m, 2H), 1.04-0.97 (m, 2H), 0.74-0.70 (m, 2H).

### Example 155: Synthesis of Compound 157

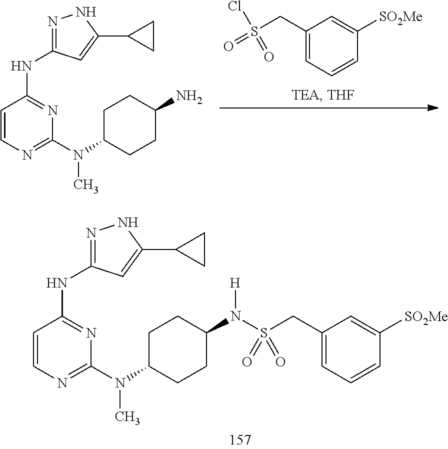 [SMILES: CN(c1nccc(Nc2cc(C3CC3)[nH]n2)n1)[C@H]1CC[C@H](N)CC1 ; CS(=O)(=O)c1cccc(CS(=O)(=O)Cl)c1 ; CN(c1nccc(Nc2cc(C3CC3)[nH]n2)n1)[C@H]1CC[C@H](NS(=O)(=O)Cc2cccc(S(C)(=O)=O)c2)CC1]

### Synthesis of N-((1R,4R)-4-((4-((5-cyclopropyl-1H-pyrazol-3-yl)amino)pyrimidin-2-yl)(methyl)amino)cyclohexyl)-1-(3-(methylsulfonyl)phenyl)methanesulfonamide

To a cooled (0° C.) solution of N2-((1R,4R)-4-aminocyclohexyl)-N4-(5-cyclopropyl-1H-pyrazol-3-yl)-N2-methylpyrimidine-2,4-diamine (150 mg, 0.45 mmol) in dry THF (2 mL) was added TEA (0.2 mL, 1.3 mmol) followed by the addition of (3-(methylsulfonyl)phenyl)methanesulfonyl chloride (260 mg, 0.91 mmol) portion wise under a nitrogen atmosphere. The reaction mixture was stirred at room temperature for 6 h. The progress of the reaction was monitored by TLC. After completion of the reaction, the reaction mixture was evaporated to obtain the crude compound, which was purified by reverse phase preparative HPLC (0.1% TFA in water/acetonitrile) to yield N-((1R,4R)-4-((4-((5-cyclopropyl-1H-pyrazol-3-yl)amino) pyrimidin-2-yl)(methyl)amino)cyclohexyl)-1-(3-(methylsulfonyl)phenyl)methanesulfonamide (12 mg, 3.9% yield). LC purity: 98.69%; m/z: 560.2 [M+H]⁺ (Mol. formula C₂₅H₃₃N₇O₄S₂, calcd. mol. wt. 559.70). ¹H NMR (400 MHz, CD₃OD): δ 8.09 (m, 1H), 8.01-7.98 (m, 1H), 7.83 (d, J=8.0 Hz, 1H), 7.73-7.67 (m, 2H), 6.35-6.21 (s, 2H), 4.51 (s, 2H), 3.33-3.32 (m, 2H), 3.16 (s, 3H), 3.05 (s, 3H), 2.16-2.12 (m, 2H), 1.98-1.91 (m, 1H), 1.80-1.74 (m, 4H), 1.48-1.44 (m, 2H), 1.07-1.02 (m, 2H), 0.78-0.72 (m, 2H).

### Example 156: Synthesis of Compound 158

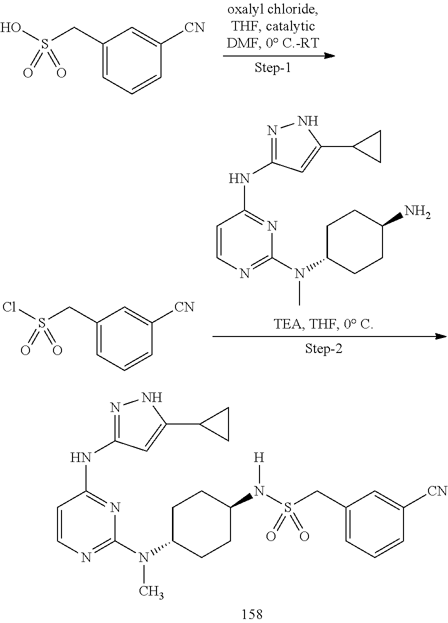

### Step-1: Synthesis of (3-cyanophenyl)methanesulfonyl chloride

To a solution of (3-cyanophenyl)methanesulfonic acid (100 mg, 0.46 mmol) in dry DCM (2 mL) was added oxalyl chloride (0.2 mL, 1.39 mmol) followed by the addition of dry DMF (0.2 mL) at 0° C. under nitrogen atmosphere. The reaction mixture was stirred for 1 h at room temperature. Progress of the reaction was monitored by TLC. After completion of the reaction, the reaction mixture was concentrated to obtain (3-cyanophenyl)methanesulfonyl chloride (120 mg, crude), which was taken directly to the next step. LC purity: Not recorded (Mol. formula C₈H₆ClNO₂S, calcd. mol. wt. 215.65).

### Step-2: Synthesis of 1-(3-cyanophenyl)-N-((1R,4R)-4-((4-((5-cyclopropyl-1H-pyrazol-3-yl)amino)pyrimidin-2-yl)(methyl)amino)cyclohexyl)methanesulfonamide

To a cooled (0° C.) solution of N2-((1R,4R)-4-aminocyclohexyl)-N4-(5-cyclopropyl-1H-pyrazol-3-yl)-N2-methylpyrimidine-2,4-diamine (152 mg, 0.464 mmol) and TEA (0.2 mL, 1.38 mmol) in dry DMF (2 mL) under a nitrogen atmosphere was added (3-cyanophenyl)methanesulfonyl chloride (120 mg, 0.556 mmol). The reaction mixture was stirred at room temperature for 1 h. The progress of the reaction was monitored by TLC. After completion of the reaction, the reaction mixture was evaporated to obtain the residue. The residue obtained was purified by reverse phase preparative HPLC (0.1% TFA in water/acetonitrile) to yield 1-(3-cyanophenyl)-N-((1R,4R)-4-((4-((5-cyclopropyl-1H-pyrazol-3-yl)amino)pyrimidin-2-yl)(methyl)amino) cyclohexyl)methanesulfonamide (25 mg, 10.8%). LC purity: 95.79%; m/z: 507.1 [M+H]⁺ (Mol. formula C₂₅H₃₀N₈O₂S, calcd. mol. wt. 506.63). ¹H NMR (400 MHz, CD₃OD): δ 7.84 (s, 1H), 7.79-7.73 (m, 3H), 7.60 (t, J=7.6 Hz, 1H), 6.37-6.21 (m, 2H), 4.44 (s, 2H), 3.32-3.20 (m, 1H), 3.06 (s, 3H), 2.16-2.13 (m, 2H), 1.99-1.95 (m, 2H), 1.82-1.77 (m, 4H), 1.53-1.44 (m, 2H), 1.07-1.02 (m, 2H), 0.78-0.74 (m, 2H).

### Example 157: Synthesis of Compound 159

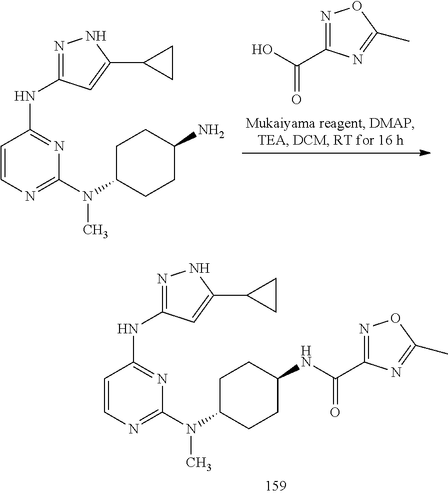

Compound 159 was prepared from N2-((1R,4R)-4-aminocyclohexyl)-N4-(5-cyclopropyl-1H-pyrazol-3-yl)-N2-methylpyrimidine-2,4-diamine. Yield 12 mg. Purity (HPLC) 98.6%, MS (m/e 438).

### Example 158: Synthesis of Compound 160

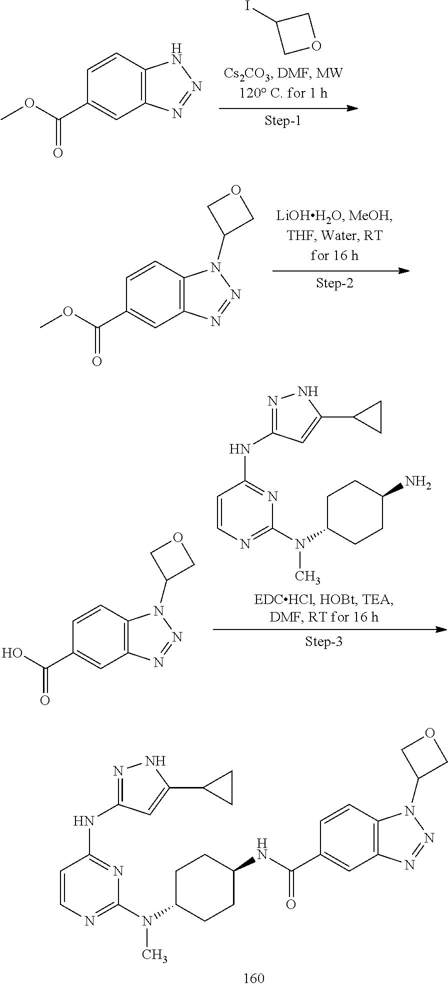

### Example 159: Synthesis of Compound 161

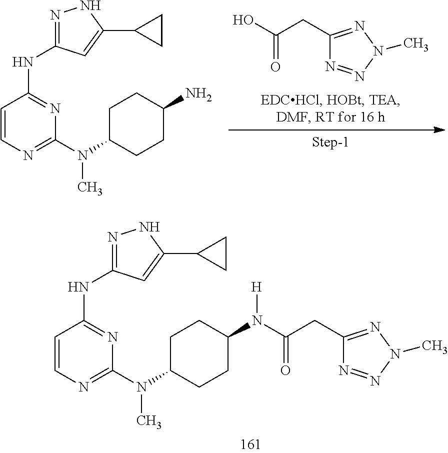

Compound 161 was prepared from N2-((1R,4R)-4-aminocyclohexyl)-N4-(5-cyclopropyl-1H-pyrazol-3-yl)-N2-methylpyrimidine-2,4-diamine. Yield 60 mg. Purity (HPLC) 98.6%, MS (m/e 438).

### Example 160: Degradation of MycN Protein

### Cell Culture

In a T150 flask was plated either 2×10⁶ of L-363 (human leukemia cell line) or SKNBE2 (neuroblastoma cell line) cells in a total of 30 mL containing Roswell Park Memorial Institute (RPMI) 1640 Medium (Thermo Fisher, Cat. #11875-085), 10% Fetal Bovine Serum (FBS, Thermo Fisher: Cat. #10437-028), 1% Penicillin/streptomycin (Thermo Fisher: Cat. #10378016), and 1% Amophotericin B (Thermo Fisher, Cat. #15290026). The cells were split every 72 hours by reseeding the L-363 or SKNBE2 cells, and the passage number was noted. It is noteworthy that cell cultures that were above 40 passages were not used, and most experiments were done with cells of less than 30 passages.

### Western Blotting

In a 6-well cell culture plate, a total of 3 mL of either 1×10⁶ cells/mL of L-363 or SKNBE2 cells were seeded, resulting in a total of 3×10⁶ cells per well. To each well containing 3 mL of either L-363 or SKNBE2 cell line was added 6 uL of MycN modulating compound (1 mM) and the resulting plate was shaken from left to right, and not swirled. After 6 hours, the cells were placed in a 15 mL falcon tube and spun at 500 Gs at 4° C. in a swinging bucket centrifuge. The medium was then carefully removed without disturbing the pellet. The pellet was then washed with 3 mLs of chilled phosphate buffered saline (PBS) and subjected to the spin cycle. PBS was then removed and the pellet was lysed in 200 uL of radioimmunoprecipitation (RIPA) lysis buffer (Thermo Fisher: Cat. #899000) that is supplemented with protease and phosphatase inhibitors (Thermo Fisher: Cat. #A32959). The cell lysate was then subjected to spinning in a centrifuge for 10 minutes at 13000 G at 4° C. The supernatant was then carefully transferred to a fresh eppendorf tube without disturbing the pellet (˜180 uL). The protein concentration of the cell lysate was then determined by using a bicinchoninic acid (BCA assay) according to manufacturer's protocol (Thermo Fisher: Cat. #23227).

### Gel Running and Transfer

Cell lysate, approximately 25 ug-30 ug, was loaded per well in a 4-20% polyacrylamide gel (Biorad. Cat. #5671094). After running the dye front off of the gel, the gel was transferred to a nitrocellulose membrane (Biorad: Cat. #1704159) using the transblot turbo system (Biorad: Cat. #1704150) according to manufacturer's protocol. After transferring for 30 minutes, the membrane was blocked with 5% BSA for 1 hour at room temperature. The BSA was then washed off and the primary antibody of choice (1:500) was added, and the membrane was incubated with the primary antibody at 4° C. for overnight. The next morning, the primary antibody was removed and the membrane was washed with 1×-TBST for 10 minutes and repeated three more times. Following the last wash, a secondary antibody (Molecular Devices. Cat. #R8209 or R8208) was added at 1:5000 dilution and incubated for 1 hour at room temperature. Following the incubation with the secondary antibody, the membrane was washed with 1×-TBST for 10 minutes and repeated three more times. Following the last wash, the membrane was washed with de-ionized water twice and dried for at least two hours. Once the membrane is completely dry, the Molecular Devices Spectra Max western system was used to observe the bands. The western image was saved and the band density was measured with ImageJ software.

TABLE 1
Percent Degradation of MycN/MycC Protein
Table 1. Percent Degradation of MycN Protein by Various Compounds

| | % Degradation | % Degradation |
|---|---|---|
| | of MycN (SKNBE2) | of MycC (L-363) |
| Cmpd No. | 6 h, 2 μM | 6 h, 2 μM |
| 1 | *** | **** |
| 2 | **** | **** |
| 3 | *** | **** |
| 4 | * | ** |
| 5 | | |
| 6 | *** | **** |
| 7 | * | ** |
| 8 | *** | **** |
| 9 | **** | **** |
| 10 | *** | **** |
| 11 | *** | |
| 12 | *** | **** |
| 13 | *** | **** |
| 14 | **** | **** |
| 15 F1 | * | * |
| 15 F2 | **** | **** |
| 101 | *** | |
| 102 | **** | |
| 103 | *** | |
| 104 | ** | |
| 105 | *** | |
| 106 | ** | |
| 107 | *** | |
| 108 | *** | **** |
| 110 | * | ** |
| 109 | * | *** |
| 111 | ** | * |
| 112 | *** | **** |
| 113 F1 | * | *** |
| 113 F2 | *** | *** |
| 114 | *** | |
| 115 | * | *** |
| 116 | * | **** |
| 117 | **** | **** |
| 118 | *** | **** |
| 119 | **** | **** |
| 120 | *** | **** |
| 121 | **** | **** |
| 122 | *** | **** |
| 123 | ** | ** |
| 124 | *** | **** |
| 125 | **** | **** |
| 126 | * | **** |
| 127 | **** | **** |
| 128 | ** | ** |
| 129 | ** | * |
| 130 | * | *** |
| 131 | ** | * |
| 132 | **** | **** |
| 133 | **** | **** |
| 134 | ** | **** |
| 135 | * | **** |
| 136 | *** | **** |
| 137 | * | ** |
| 138 | * | * |
| 139 | **** | **** |
| 140 | **** | **** |
| 141 | **** | **** |
| 142 | **** | **** |
| 143 | * | * |
| 144 | **** | **** |
| 145 | *** | **** |
| 146 | | |
| 147 | * | ** |
| 148 | * | ** |
| 149 | **** | **** |
| 150 | **** | **** |
| 151 | **** | **** |
| 152 | **** | **** |
| 153 | **** | **** |
| 154 | *** | **** |
| 155 | **** | **** |
| 156 | *** | **** |
| 157 | *** | **** |
| 158 | *** | **** |

Key:
**** degradation 80-100%
*** degradation 50-79%
** degradation 20-49%
* degradation <20%

### Example 161: Degradation of MycN Protein

For L363 cells (suspension), 1E6 cells were plated into each well of a 6 well plate. For SK-N-BE(2) cells (adherent), 5E5 cells were plated into each well of a 6 well dish. Cells were cultured for 24 hrs then treated with exemplary compounds at 0.1, 0.5, 1.0, 3.0 and 6.0 mM final assay concentrations plus DMSO control. All compounds were diluted to 10 mM in DMSO. Cells were treated with the compound for 24 hrs, then for both cell types, media containing cells were removed from wells into a 15 ml centrifuge tube, and remaining adherent SK-N-BE(2) cells were also scraped from the wells into the appropriate tubes. Tubes were centrifuged, cells were washed with PBS then re-centrifuged to pellet cells. A RIPA buffer cocktail was added to the cells on ice for 5 mins. Cell lysates were clarified by centrifugation and stored at −80 degrees until required. BCA assay was performed on each lysate to determine the protein concentration.

For validation studies of antibodies, lysates and antibodies were used at a number of concentrations (Lysates: 2, 1, 0.2 mg/ml; Antibodies: 1 in 50, 1 in 200 dilution). Once an appropriate lysate concentration and antibody concentration was determined, samples were screened in the JESS technology (Protein Simple, San Jose, CA—https://www.proteinsimple.com/jess.html).

Target protein antibodies (n-myc and c-myc) were detected in the chemi-luminescence channel and loading controls (tubulin and GAPDH) were detected using a Near Infra Red (NIR) labeled secondary antibody.

Cell Proliferation/Viability Measures

For SK-N-EB2 adherent cells, 5E5 cells in 1.9 mls media were plated into 6 well dishes and incubated for 24 hrs. Exemplary compounds were added (100 ul, 1 in 20 dilution in media) to each well (final assay concentration 6, 3, 1 and 0.5 mM) together with DMSO control wells and incubated for 24 hrs. Cells were scrapped off the plate, centrifuged, washed with PBS, centrifuged, then a RIPA buffer cocktail added to the cells (100 ul), centrifuged and stored at −80 for later analysis. For L363 suspension cells, 1E6 cells were plated into 24 well dishes (950 ul media) and incubated for 24 hrs. Exemplary compounds were added (50 ul, 1 in 20 dilution in media) to each well (final assay concentration 6, 3, 1 and 0.5 mM) together with DMSO control wells and incubated for 24 hrs. Cells were aspirated into tubes, centrifuged, washed with PBS, centrifuged, then a RIPA buffer cocktail added to the cells (100 ul), centrifuged and stored at −80 for later analysis.

For Western blot, a BCA (total protein) assay was run on all samples, these were then run in batches of 5 compounds plus control (DMSO) on each Western blot run—n-myc and c-myc were run in separate experiments in the chemiluminescence channel. Both tubulin and GAPDH were run as loading controls in all lanes in the near infra red channel.

For cytotox, both SK-N-EB2 or L363 cells were run in 384 well format. For SK-N-EB2 cells, 5000 cells per well (in 30 ul media) were incubated for 24 hrs prior to the addition of compounds (10 ul, 1 in 4 dilution, 10 mM top final concentration, 1 in 2 dilutions) for 24 hrs. For L363 cells, 2000 cells per well (30 ul media) were incubated for 24 hrs prior to the addition of compounds (10 ul, 10 mM 1 in 2 dilutions) for 24 hrs. In both cases Promega Cell Titer GLO was added according to the manufactures instructions and the plates read immediately in the luminometer.

TABLE 2
Percent Degradation of MycN Protein by Various Compounds

| | % | % | % | % | % | % | % | % |
|---|---|---|---|---|---|---|---|---|
| | Degradation | Degradation | Degradation | Degradation | Degradation | Degradation | Degradation | Degradation |
| | of MycN | of MycC | of MycN | of MycC | of MycN | of MycC | of MycN | of MycC |
| Cmpd | (SKNBE2) | (L-363) | (SKNBE2) | (L-363) | (SKNBE2) | (L-363) | (SKNBE2) | (L-363) |
| No. | 24 h, 6 μM | 24 h, 6 μM | 24 h, 3 μM | 24 h, 3 μM | 24 h, 1 μM | 24 h, 1 μM | 24 h, 0.5 μM | 24 h, 0.5 μM |
| 7 | **** | **** | ** | *** | * | ** | * | * |
| 28 | ** | **** | ND | **** | ND | ND | ND | ND |
| 16 | **** | **** | **** | **** | ** | **** | ** | *** |
| 17 | **** | **** | **** | **** | ** | **** | * | ND |
| 26 | ** | **** | ND | * | * | * | ND | ND |
| 31 | **** | **** | **** | **** | *** | **** | ** | ND |
| 38 | **** | **** | **** | **** | *** | **** | ** | *** |
| 40 | **** | **** | **** | **** | ** | **** | * | * |
| 39 | **** | **** | **** | **** | ND | *** | ND | ND |
| 22 | ** | ND | ** | ND | ** | ND | * | ND |
| 23 | **** | **** | ND | **** | ND | *** | ND | * |
| 34 | **** | **** | **** | * | * | ND | * | ND |
| 27 | * | **** | ND | *** | ** | ** | ** | * |
| 29 | **** | **** | *** | **** | ** | **** | ** | *** |
| 18 | **** | **** | **** | **** | ** | **** | ND | ND |
| 33 | **** | **** | ND | **** | ND | ND | ND | ND |
| 32 | **** | **** | **** | **** | ND | ND | ND | ND |
| 35 | **** | **** | *** | **** | ** | *** | * | ** |
| 36 | * | ND | * | ND | * | ND | ND | ND |
| 21 | *** | **** | ** | *** | ** | * | * | ND |
| 20 | *** | **** | ** | ND | ND | ND | ND | ND |
| 25 | * | ND | * | ND | ND | ** | ND | ** |
| 30 | ND | * | ND | * | ND | ** | ND | ** |
| 24 | ND | **** | ND | ND | ND | ND | ND | ND |
| 19 | ND | * | ND | * | ND | * | ND | * |
| 37 | *** | **** | ** | * | * | ND | ND | ND |

Key:
**** degradation 80-100%
*** degradation 50-79%
** degradation 20-49%
* degradation <20%
ND—No Degradation

It will be appreciated that compounds reported as a salt form (e.g., a TFA salt) may or may not have a 1:1 stoichiometry, and/or for example, reported potency concentrations or other assay results may be, e.g., slightly higher or lower.

The practice of the present disclosure will employ, unless otherwise indicated, conventional methods of organic chemistry, protein chemistry, biochemistry, recombinant DNA techniques and pharmacology, within the skill of the art. While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

What is claimed is:

1. A compound of Formula I: 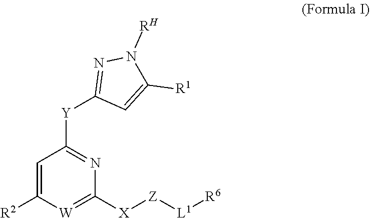
or a pharmaceutically acceptable salt, stereoisomer, and/or N-oxide thereof, wherein:
W is selected from the group consisting of N, C—H, and C—F;
X is selected from the group consisting of NR^{A}, O, S, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —S(O)_{w}—, —NR^{A}—S(O)_{w}—, —CHR^{L}—NR^{A}—S(O)_{w}—, —NR^{A}—S(O)_{w}—CHR^{L}—, —S(O)_{w}—NR^{A}—, —CH₂—S(O)_{w}—NR^{A}—, —S(O)_{w}—NR^{A}—CHR^{L}—, —CHR^{L}—C(O)—, —C(O)—, and bond, where w is 0, 1 or 2;
Z is 4-10 membered heterocyclic having at least one nitrogen, wherein the nitrogen is bound to L1, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-phenyl, C₁₋₃alkylene-heteroaryl, —C(O)-heteroaryl, phenoxy, and H; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of a bond, H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

2. The compound of claim 1, wherein W is N, and having the Formula Ia: 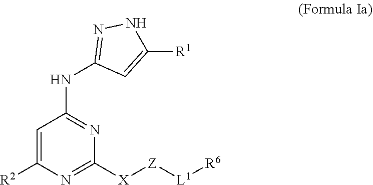
or a pharmaceutically acceptable salt, stereoisomer and/or N-oxide thereof.

3. The compound of claim 1 or 2, wherein R¹ is a 5-6 membered heterocyclyl or C₃₋₆cycloalkyl.

4. The compound of any one of claims 1-3, wherein R¹ is selected from the group consisting of: 2-tetrahydrofuranyl, 3-tetrahydrofuranyl, 2-oxetanyl, cyclohexyl, cyclopropyl, cyclobutyl, and cyclopentyl.

5. The compound of claim 4, wherein R¹ is cyclopropyl.

6. The compound of claim 4, wherein R¹ is cyclopentyl.

7. The compound of claim 1 or 2, wherein R¹ is selected from the group consisting of methyl and ethyl.

8. The compound of any one of claims 1-7, wherein X is NR^{A}.

9. The compound of any one of claims 1-8, wherein Z is selected from the group consisting of 4-6 membered monocyclic heterocycle, a 6-10 membered spiroheterocycle, a 6-10 membered fused bicyclic heterocyclic, and a 6-10 membered bridged cycloheteroalkyl.

10. A compound of Formula Iaa: 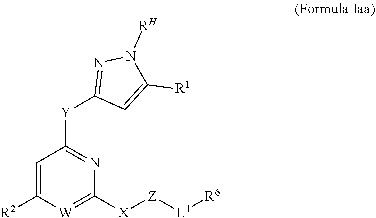
or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂C(CH₂)₂, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
L¹ is selected from the group consisting of —NR^{A}—C(O)—, —CHR^{L}—NR^{A}—C(O)—, —NR^{A}—C(O)—CHR^{L}—, —C(O)—NR^{A}—, —CHR^{L}—C(O)—NR^{A}—, —C(O)—NR^{A}—CH₂—, —S(O)_{w}—, —NR^{A}—S(O)_{w}—, —CHR^{L}—NR^{A}—S(O)_{w}—, —NR^{A}—S(O)_{w}—CHR^{L}—, —S(O)_{w}—NR^{A}—, —CH₂—S(O)—NR^{A}—, —S(O)_{w}—NR^{A}—CHR^{L}—, —CHR^{L}—C(O)—, —C(O)—, and bond, where w is 0, 1 or 2;
Z is selected from a 6-10 membered spiroheterocycle, a 6-10 membered fused bicyclic heterocyclic, and a 6-10 membered bridged cycloheteroalkyl each having at least one nitrogen, wherein the nitrogen is bound to L1, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, heteroaryl, and H; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ is selected from the group consisting of C₁-C₆-alkyl, C₃-C₁₀cycloalkyl, heterocyclyl, benzo-fused heterocyclyl, phenyl, benzyl, heteroaryl, C₁₋₃alkylene-phenyl, C₁₋₃alkylene-heteroaryl, —C(O)-heteroaryl, phenoxy, and H; wherein R⁶ may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{L} is independently selected, for each occurrence, from the group consisting of a bond, H and methyl (optionally substited by one, two or three halogens);
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

11. The compound of any one of claims 1-9, represented by Formula II: 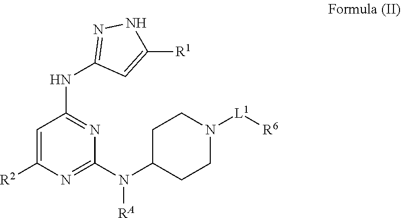
or a pharmaceutically acceptable salt, stereoisomer, and/or N-oxide thereof.

12. The compound of any one of claims 1-9, represented by Formula IIa: 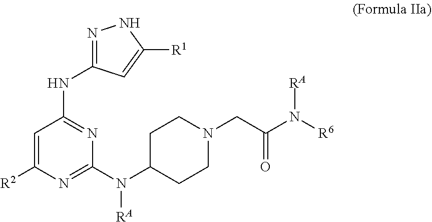
or a pharmaceutically acceptable salt, stereoisomer, and/or N-oxide thereof.

13. The compound of any one of claims 1-9, represented by Formula IIb: 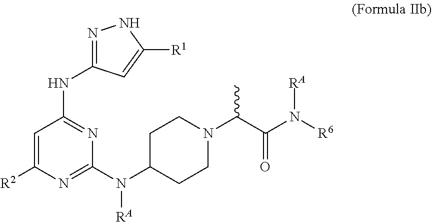
or a pharmaceutically acceptable salt, stereoisomer, and/or N-oxide thereof.

14. The compound of any one of claims 1 and 3-9, represented by Formula IIc: 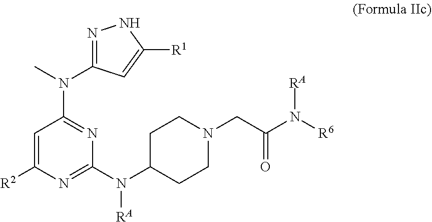
or a pharmaceutically acceptable salt, stereoisomer, and/or N-oxide thereof.

15. The compound of any one of claims 1-10, represented by Formula IId: 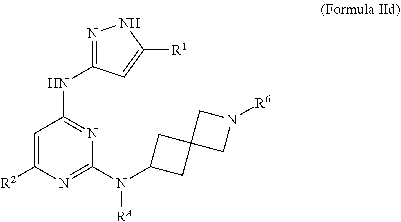
or a pharmaceutically acceptable salt, stereoisomer, and/or N-oxide thereof.

16. The compound of any one of claims 1 and 3-10, represented by Formula IIe: 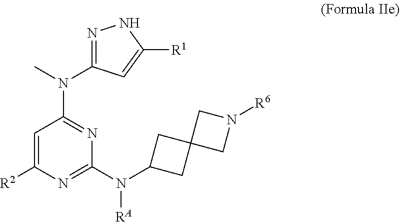
or a pharmaceutically acceptable salt, stereoisomer, and/or N-oxide thereof.

17. The compound of any one of claims 1-9, represented by Formula IIf: 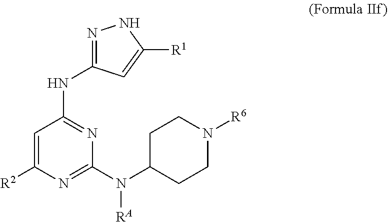
or a pharmaceutically acceptable salt, stereoisomer, and/or N-oxide thereof.

18. The compound of any one of claims 1-17, wherein R^{A} is selected from H and methyl.

19. The compound of any one of claims 1-18, wherein R⁶ is selected from the group consisting of a 8-10 membered bicyclic cycloalkyl and a 8-10 membered bicyclic heterocyclyl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O— heterocyclyl, heterocyclyl and heteroaryl.

20. The compound of any one of claims 1-18, wherein R⁶ is selected from the group consisting of a monocyclic or bridged C₃₋₆cycloalkyl, a monocyclic or bridged heterocyclyl, a bicyclic or fused heterocyclyl, and a heteroaryl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O-heterocyclyl, heterocyclyl and heteroaryl.

21. The compound of any one of claims 1-18, wherein R⁶ is selected from the group consisting of: indanyl, cyclohexyl, cyclobutyl, and cyclopentyl, wherein R⁶ is optionally substituted by one or two substituents each selected from the group consisting of: cyano, halo, phenyl, —C(═N)—NR′R′, C₁₋₄alkyl (optionally substituted by methoxy or by one, two or three fluorine atoms or heterocyclyl), C₁₋₄alkoxy (optionally substituted by one, two or three fluorine atoms), S(O)₂—CH₃, —O-heterocyclyl, heterocyclyl and heteroaryl.

22. The compound of any one of claims 1-18, wherein R⁶ is selected from the group consisting of heterocyclyl, phenyl, and heteroaryl.

23. The compound of claim 21, wherein R⁶ is indanyl.

24. The compound of any one of claims 1-23, wherein R⁶ is represented by: 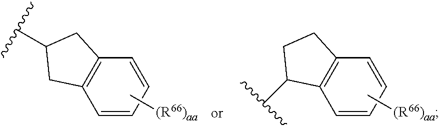
wherein R⁶⁶ is selected from the group consisting of H, halo, and cyano; and aa is 0, 1, or 2.

25. The compound of claim 24, wherein R⁶ is selected from the group consisting of:

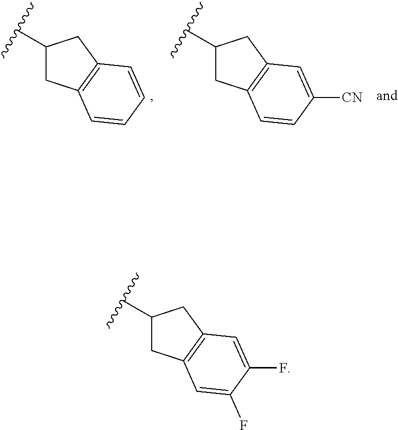 [SMILES: *C1Cc2ccccc2C1 ; *C1Cc2ccc(C#N)cc2C1 ; *C1Cc2cc(F)c(F)cc2C1]

26. The compound of claim of any one of claims 1-18, wherein R⁶ is selected from the group consisting of:

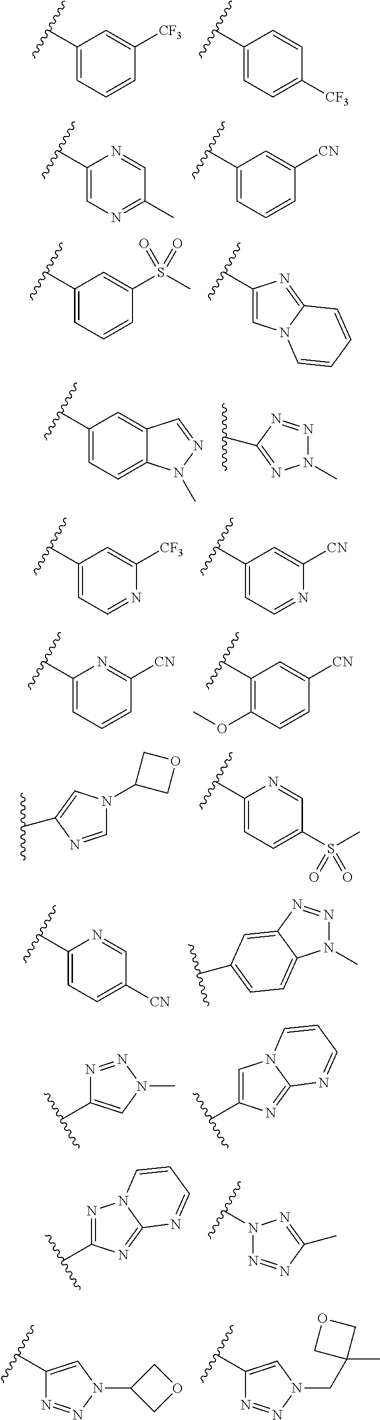

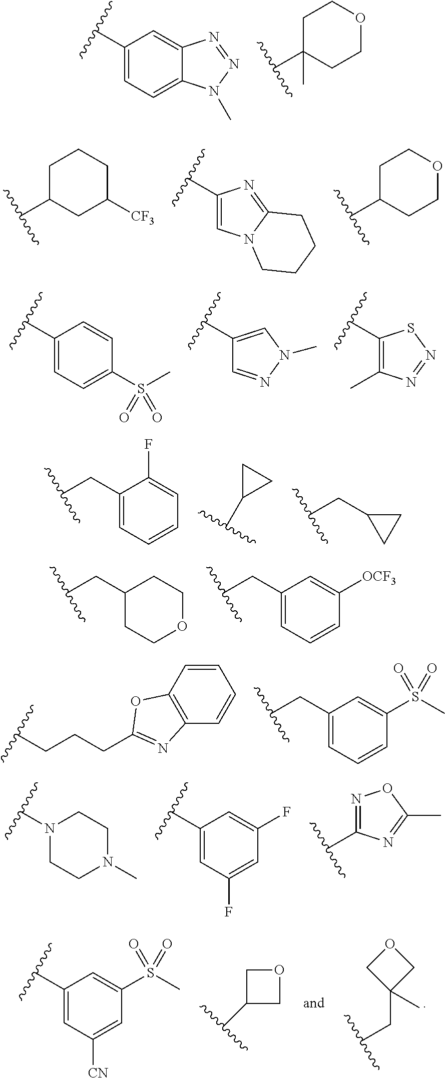

27. The compound of claim 26, wherein R⁶ is selected from the group consisting of:

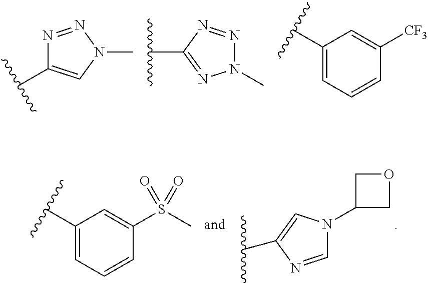

28. The compound of any one of claims 1-18, wherein R⁶ is methyl.

29. The compound of any one of claims 1-18, wherein R⁶ is methyl.

30. The compound of any one of claims 1-29, wherein R² is H.

31. A compound of Formula III: 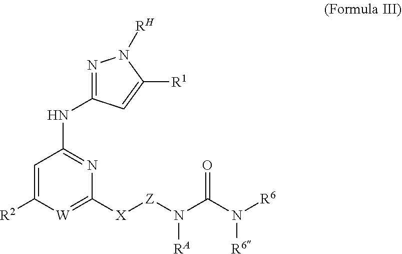
or a pharmaceutically acceptable salt, stereoisomer, and/or N-oxide thereof, wherein:
W is selected from the group consisting of N, C—H, and C—F;
X is selected from the group consisting of NR^{A}, O, S, CH₂, C(CH₃)₂, CF₂, C(CH₂)₂, NR^{A}CH₂, NR^{A}C(O), and C(O);
Y is selected from the group consisting of NH, N—CH₃, O, S, CH₂, CF₂, CH(CH₃), C(CH₃)₂, and C(CH₂CH₂);
R^{H} is selected from the group consisting of H, C₁₋₃alkyl, —C(O)—C₁₋₃alkyl and C(O)—O—C₁₋₃alkyl;
Z is selected from the group consisting of fused bicycloalkyl, C₃-C₇ monocyclic cycloalkyl, C₅-C₉ bridged cycloalkyl and spiro C₅-C₁₀ bicycloalkyl, wherein Z may optionally be substituted by one or two substituents each independently selected from the group consisting of halo, hydroxyl, C₁-C₄ alkyl (optionally substituted by one, two or three halogens), —C(O)OH, —C(O)—O—C₁₋₄alkyl, and oxo;
R¹ is selected from the group consisting of C₁-C₆ alkyl, C₃-C₁₀ cycloalkyl, spiro C₅-C₁₀ bicycloalkyl, heterocyclyl, cyano, halo, and heteroaryl; wherein C₁-C₆ alkyl, C₃-C₇ cycloalkyl, heterocyclyl, or heteroaryl may be substituted by one, two or three substitutents each independently selected from halo and C₁-C₄alkyl (optionally substituted by one, two or three halogens);
R² is selected from the group consisting of H, F, —C(O)—O-methyl, —C(O)OH, —O— methyl, methyl, C₃-C₇ cycloalkyl and heterocyclyl;
R⁶ and R^{6′}, together with the nitrogen attached to R⁶ and R^{6′}, form a 4-8 membered monocyclic heterocyclyl or a 8-10 membered bicyclic heterocyclyl; wherein the monocyclic heterocyclyl or bicyclic heterocyclyl may be optionally substituted by one, two or three substituents each independently selected from the group consisting of R^{P};
R^{A} is selected from the group consisting of H, C₁-C₄ alkyl, —C(O)—C₁₋₄ alkyl, S(O)_{w}—C₁₋₄alkyl, (wherein w is 0, 1 or 2), C₃₋₆cycloalkyl and heterocyclyl; wherein C₁-C₄ alkyl and C₃₋₆ cycloalkyl may be optionally substituted by one, two or three substituents each selected from halo, C₁₋₄ alkoxy, —S(O)_{w}-methyl, —S(O)_{w}-ethyl (wherein w is 0, 1 or 2) and heterocyclyl; and wherein heterocyclyl may be optionally substituted by one or two substituents each selected from methyl, ethyl, and halo;
R^{P} is selected from the group consisting of halo, cyano, C₁₋₆alkyl, C₂₋₆alkenyl, C₂₋₆alkynyl, C₁₋₆alkoxy (optionally substituted by one, two or three substituents each selected from halo, cyano, hydroxyl, and C₁₋₃alkoxy), —C(O)—C₁₋₄ alkyl, C(O)—O—C₁₋₄ alkyl, C(O)—O—C₃₋₆ cycloalkyl, —C(═N)—NR′R′, —C(O)—NR′R′, —S(O)_{w}—NR′R′, —S(O)_{w}—C₁₋₄alkyl, (wherein w is 0, 1 or 2), —NR′R′, oxo, phenyl, phenoxy, C₃₋₆cycloalkyl, heterocyclyl, —O-heterocyclyl and heteroaryl; wherein heterocyclyl, heteroaryl or phenyl may be optionally substituted by hydroxyl, C₁₋₆alkyl, or halo; and wherein C₁₋₆alkyl, C₂₋₆alkenyl, C₂₋₆alkynyl and C₃₋₆cycloalkyl may each be optionally substituted by one, two or three substituents each selected from halo, cyano, hydroxyl, heteroaryl, heterocyclyl, and NR′R′; and
R′ for each occurrence is independently selected from the group consisting of H, methyl, ethyl, heterocyclyl (optionally substituted by C₁₋₃alkyl or halo), phenyl, and C₃₋₆cycloalkyl, or two R's together with the nitrogen to which they are attached form a heterocyclyl which may optionally be subtituted by methyl, halo, cyano, oxo, or hydroxyl.

32. The compound of claim 31, wherein W is N, and having the Formula IIIa: 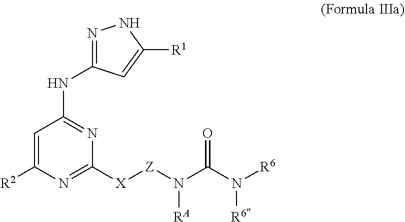
or a pharmaceutically acceptable salt, stereoisomer, and/or N-oxide thereof.

33. The compound of claim 31 or 32, wherein R¹ is a 5-6 membered heterocyclyl or C₃₋₆cycloalkyl.

34. The compound of any one of claims 31-33, wherein R¹ is selected from the group consisting of: 2-tetrahydrofuranyl, 3-tetrahydrofuranyl, 2-oxetanyl, cyclohexyl, cyclopropyl, cyclobutyl, and cyclopentyl.

35. The compound of claim 34, wherein R¹ is cyclopropyl.

36. The compound of claim 31 or 32, wherein R¹ is selected from the group consisting of methyl and ethyl.

37. The compound of any one of claims 31-36, wherein X is NR^{A}.

38. The compound of any one of claims 31-37, wherein Z is selected from the group consisting of cyclohexyl, cyclopentyl, and cyclobutyl.

39. The compound of any one of claims 31-37, wherein Z is a C₅-C₉ bridged cycloalkyl.

40. The compound of any one of claims 31-37, wherein Z is a spiro C₅-C₁₀ bicycloalkyl.

41. The compound of any one of claims 31-37, wherein Z is a fused bicycloalkyl.

42. The compound of any one of claims 31-37, wherein Z is selected from the group consisting of: 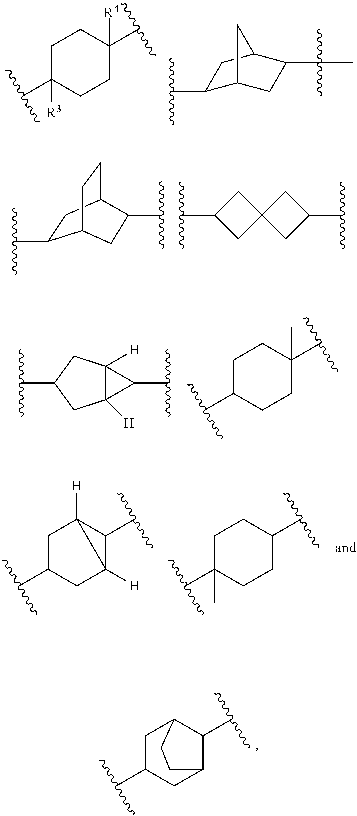
or a pharmaceutically acceptable salt, stereoisomer and/or N-oxide thereof, wherein:
R³ is selected from the group consisting of H, C₁-C₄-alkyl, CO₂H and —C(O)—O—C₁₋₄alkyl;
R⁴ is H or C₁-C₄-alkyl; or
R³ and R⁴ together form —CH₂— or —CH₂CH₂—.

43. The compound of claim 42, represented by Formula IV: 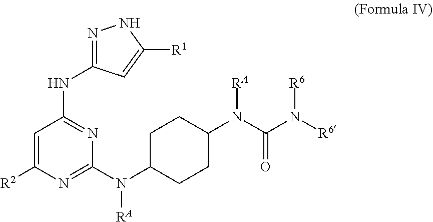
or a pharmaceutically acceptable salt, stereoisomer, and/or N-oxide thereof.

44. The compound of claim 42, represented by Formula IVa: 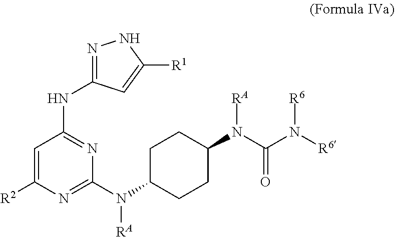
or a pharmaceutically acceptable salt, stereoisomer, and/or N-oxide thereof.

45. The compound of any one of claims 31-44, wherein R⁶ and R^{6′}, together with the nitrogen attached to R⁶ and R^{6′}, form an optionally substituted heterocycyl selected from the group consisting of: 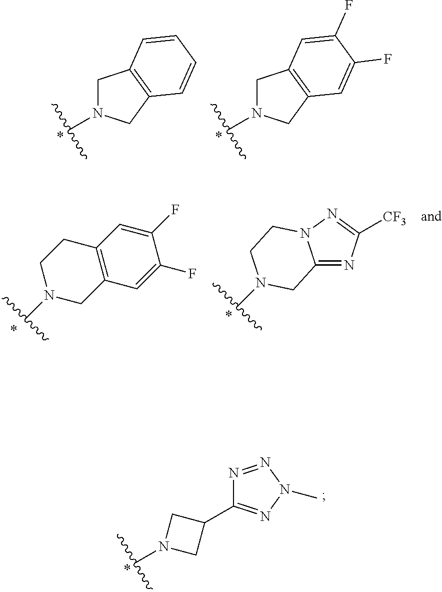 [SMILES: *N1Cc2ccccc2C1 ; *N1Cc2cc(F)c(F)cc2C1 ; *N1CCc2cc(F)c(F)cc2C1 ; *N1CCn2nc(C(F)(F)F)nc2C1 ; *N1CC(c2nnn(C)n2)C1]
wherein * denotes bonding to —C(O)—.

46. The compound of any one of claims 31-45, wherein R² is H.

47. A compound selected from the group consisting of: 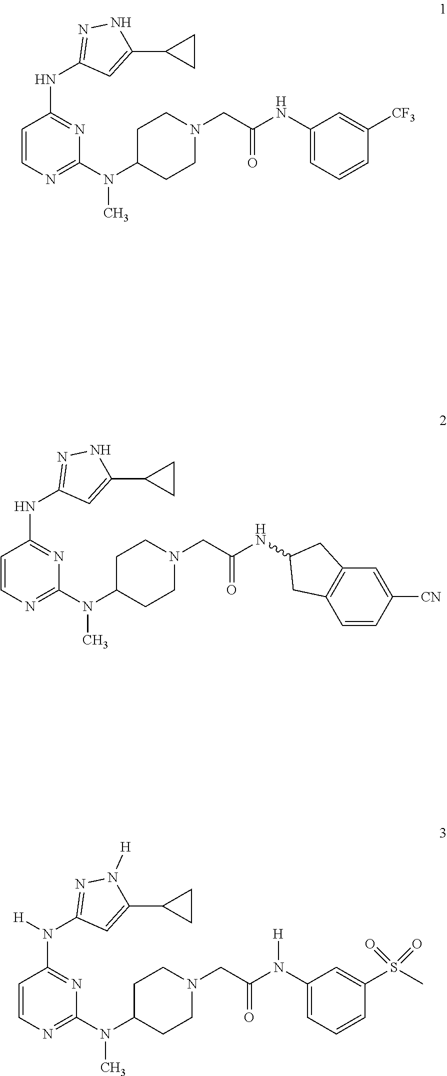 [SMILES: CN(c1nccc(Nc2cc(C3CC3)[nH]n2)n1)C1CCN(CC(=O)Nc2cccc(C(F)(F)F)c2)CC1 ; CN(c1nccc(Nc2cc(C3CC3)[nH]n2)n1)C1CCN(CC(=O)NC2Cc3ccc(C#N)cc3C2)CC1 ; CN(c1nccc(Nc2cc(C3CC3)[nH]n2)n1)C1CCN(CC(=O)Nc2cccc(S(C)(=O)=O)c2)CC1] 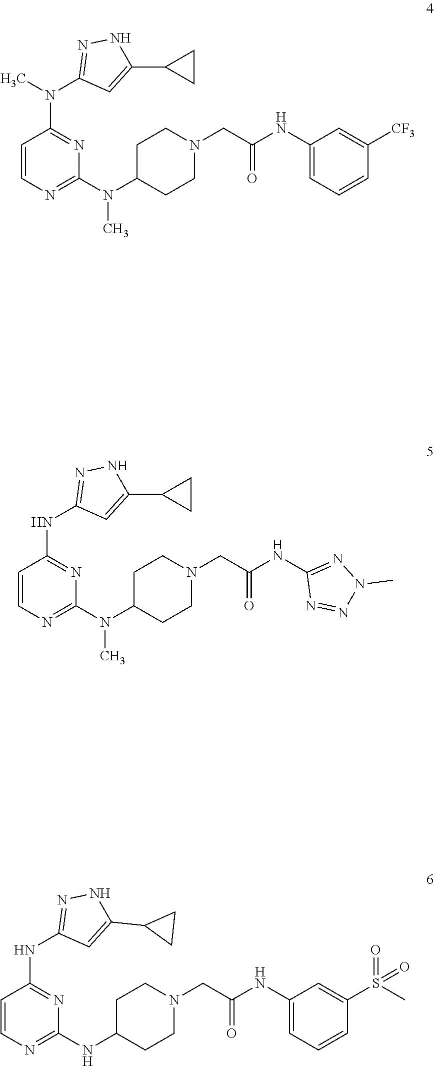 [SMILES: CN(c1cc(C2CC2)[nH]n1)c1ccnc(N(C)C2CCN(CC(=O)Nc3cccc(C(F)(F)F)c3)CC2)n1 ; CN(c1nccc(Nc2cc(C3CC3)[nH]n2)n1)C1CCN(CC(=O)Nc2nnn(C)n2)CC1 ; CS(=O)(=O)c1cccc(NC(=O)CN2CCC(Nc3nccc(Nc4cc(C5CC5)[nH]n4)n3)CC2)c1] 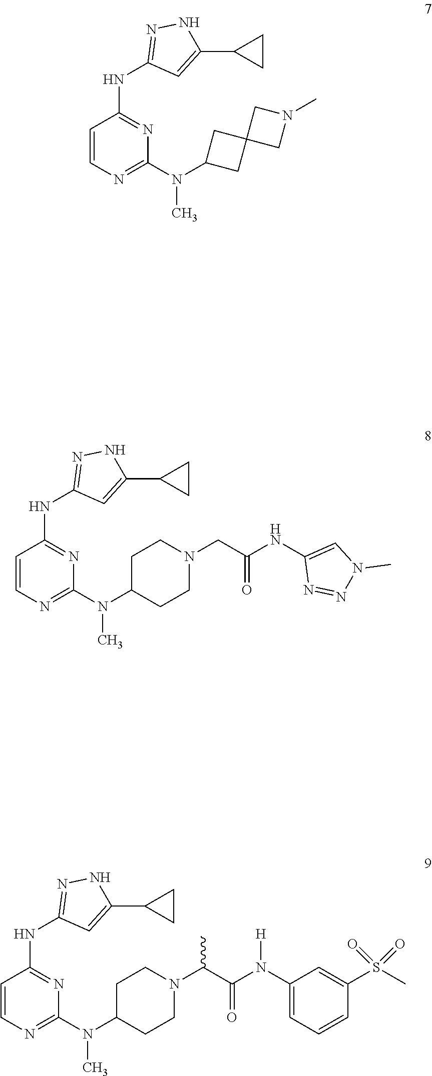 [SMILES: CN1CC2(CC(N(C)c3nccc(Nc4cc(C5CC5)[nH]n4)n3)C2)C1 ; CN(c1nccc(Nc2cc(C3CC3)[nH]n2)n1)C1CCN(CC(=O)Nc2cn(C)nn2)CC1 ; CC(C(=O)Nc1cccc(S(C)(=O)=O)c1)N1CCC(N(C)c2nccc(Nc3cc(C4CC4)[nH]n3)n2)CC1] 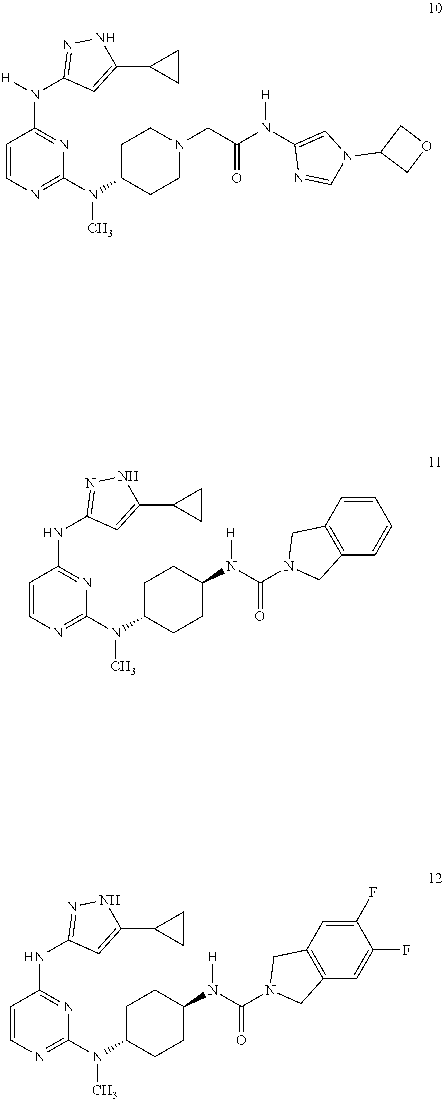 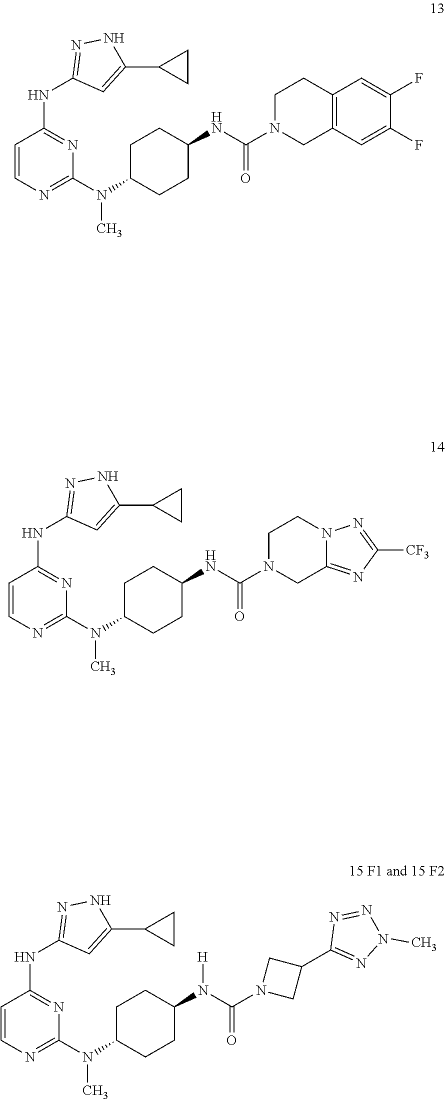 [SMILES: CN(c1nccc(Nc2cc(C3CC3)[nH]n2)n1)[C@H]1CC[C@H](NC(=O)N2CCc3cc(F)c(F)cc3C2)CC1 ; CN(c1nccc(Nc2cc(C3CC3)[nH]n2)n1)[C@H]1CC[C@H](NC(=O)N2CCn3nc(C(F)(F)F)nc3C2)CC1 ; Cn1nnc(C2CN(C(=O)N[C@H]3CC[C@H](N(C)c4nccc(Nc5cc(C6CC6)[nH]n5)n4)CC3)C2)n1] 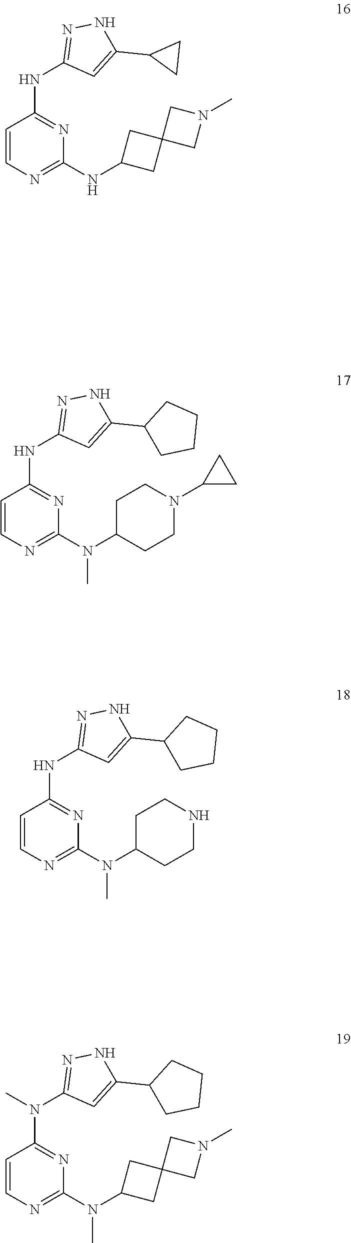 [SMILES: CN1CC2(CC(Nc3nccc(Nc4cc(C5CC5)[nH]n4)n3)C2)C1 ; CN(c1nccc(Nc2cc(C3CCCC3)[nH]n2)n1)C1CCN(C2CC2)CC1 ; CN(c1nccc(Nc2cc(C3CCCC3)[nH]n2)n1)C1CCNCC1 ; CN1CC2(CC(N(C)c3nccc(N(C)c4cc(C5CCCC5)[nH]n4)n3)C2)C1] 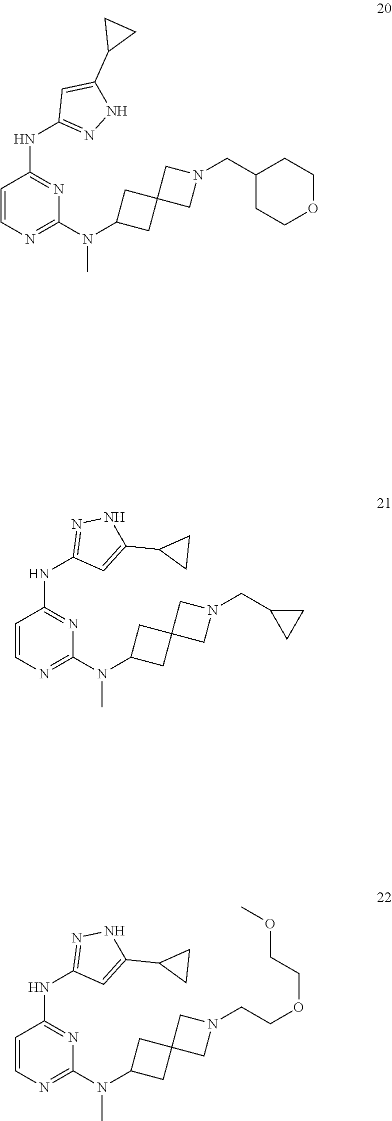 [SMILES: CN(c1nccc(Nc2cc(C3CC3)[nH]n2)n1)C1CC2(C1)CN(CC1CCOCC1)C2 ; CN(c1nccc(Nc2cc(C3CC3)[nH]n2)n1)C1CC2(C1)CN(CC1CC1)C2 ; COCCOCCN1CC2(CC(N(C)c3nccc(Nc4cc(C5CC5)[nH]n4)n3)C2)C1] 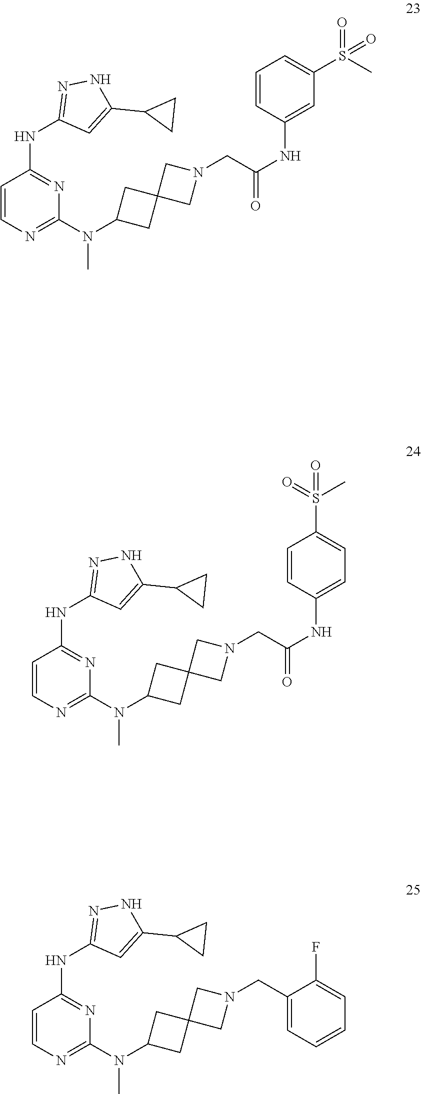 [SMILES: CN(c1nccc(Nc2cc(C3CC3)[nH]n2)n1)C1CC2(C1)CN(CC(=O)Nc1cccc(S(C)(=O)=O)c1)C2 ; CN(c1nccc(Nc2cc(C3CC3)[nH]n2)n1)C1CC2(C1)CN(CC(=O)Nc1ccc(S(C)(=O)=O)cc1)C2 ; CN(c1nccc(Nc2cc(C3CC3)[nH]n2)n1)C1CC2(C1)CN(Cc1ccccc1F)C2] 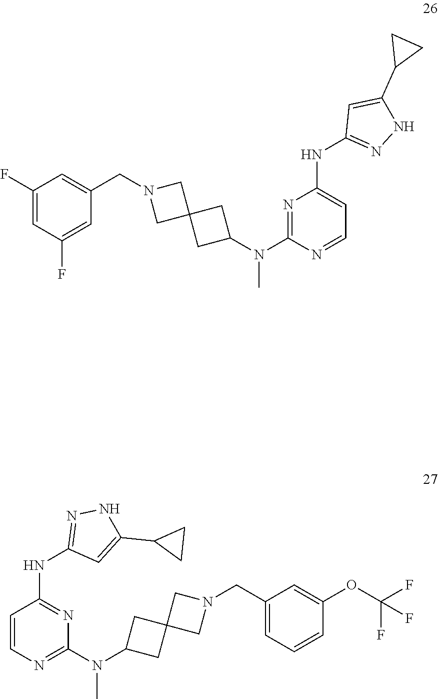 [SMILES: CN(c1nccc(Nc2cc(C3CC3)[nH]n2)n1)C1CC2(C1)CN(Cc1cc(F)cc(F)c1)C2 ; CN(c1nccc(Nc2cc(C3CC3)[nH]n2)n1)C1CC2(C1)CN(Cc1cccc(OC(F)(F)F)c1)C2] 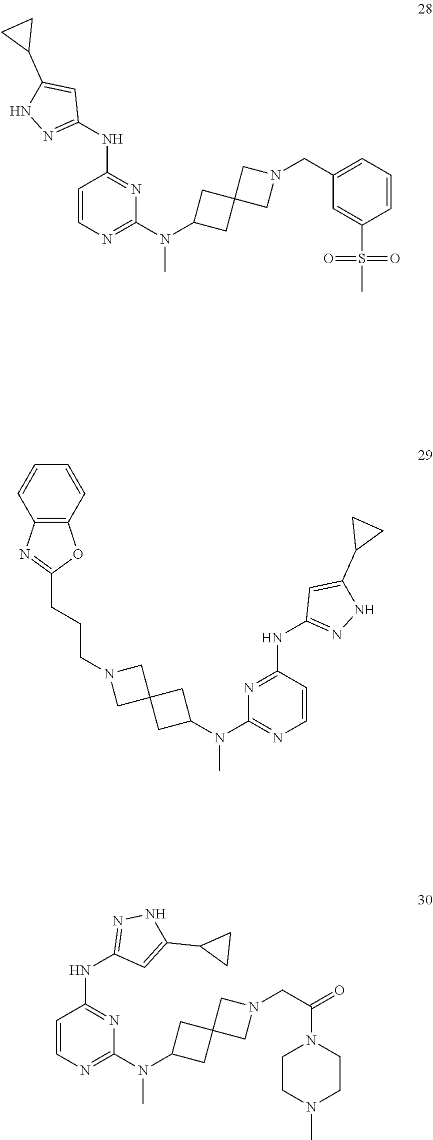 [SMILES: CN(c1nccc(Nc2cc(C3CC3)[nH]n2)n1)C1CC2(C1)CN(Cc1cccc(S(C)(=O)=O)c1)C2 ; CN(c1nccc(Nc2cc(C3CC3)[nH]n2)n1)C1CC2(C1)CN(CCCc1nc3ccccc3o1)C2 ; CN1CCN(C(=O)CN2CC3(CC(N(C)c4nccc(Nc5cc(C6CC6)[nH]n5)n4)C3)C2)CC1] 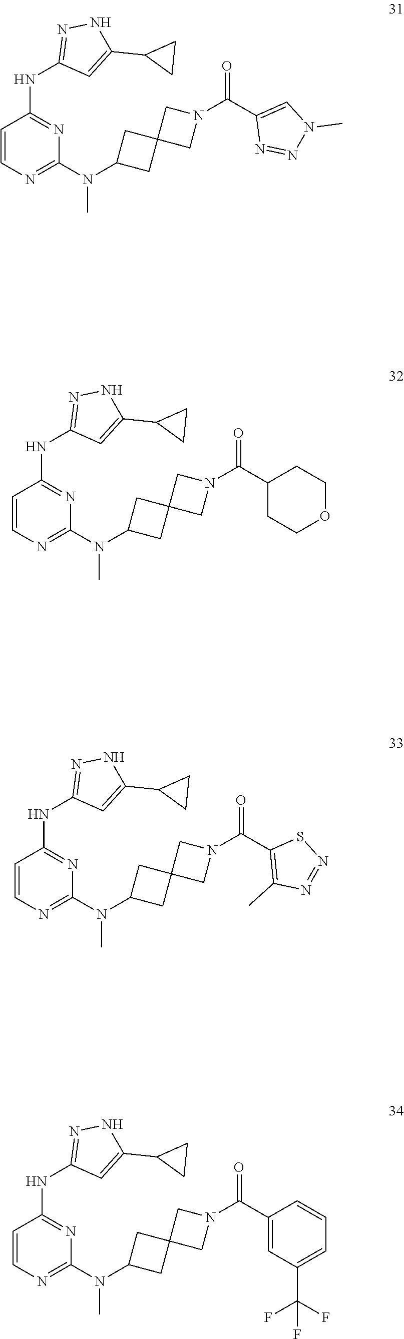 [SMILES: CN(c1nccc(Nc2cc(C3CC3)[nH]n2)n1)C1CC2(C1)CN(C(=O)c1cn(C)nn1)C2 ; CN(c1nccc(Nc2cc(C3CC3)[nH]n2)n1)C1CC2(C1)CN(C(=O)C1CCOCC1)C2 ; Cc1nnsc1C(=O)N1CC2(CC(N(C)c3nccc(Nc4cc(C5CC5)[nH]n4)n3)C2)C1 ; CN(c1nccc(Nc2cc(C3CC3)[nH]n2)n1)C1CC2(C1)CN(C(=O)c1cccc(C(F)(F)F)c1)C2] 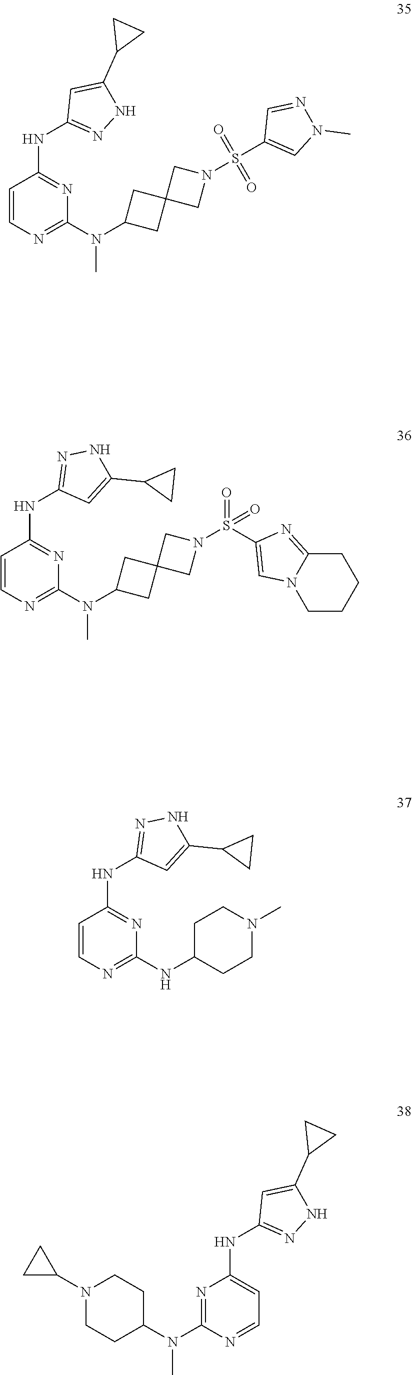 [SMILES: CN(c1nccc(Nc2cc(C3CC3)[nH]n2)n1)C1CC2(C1)CN(S(=O)(=O)c1cnn(C)c1)C2 ; CN(c1nccc(Nc2cc(C3CC3)[nH]n2)n1)C1CC2(C1)CN(S(=O)(=O)c1cn3c(n1)CCCC3)C2 ; CN1CCC(Nc2nccc(Nc3cc(C4CC4)[nH]n3)n2)CC1 ; CN(c1nccc(Nc2cc(C3CC3)[nH]n2)n1)C1CCN(C2CC2)CC1] 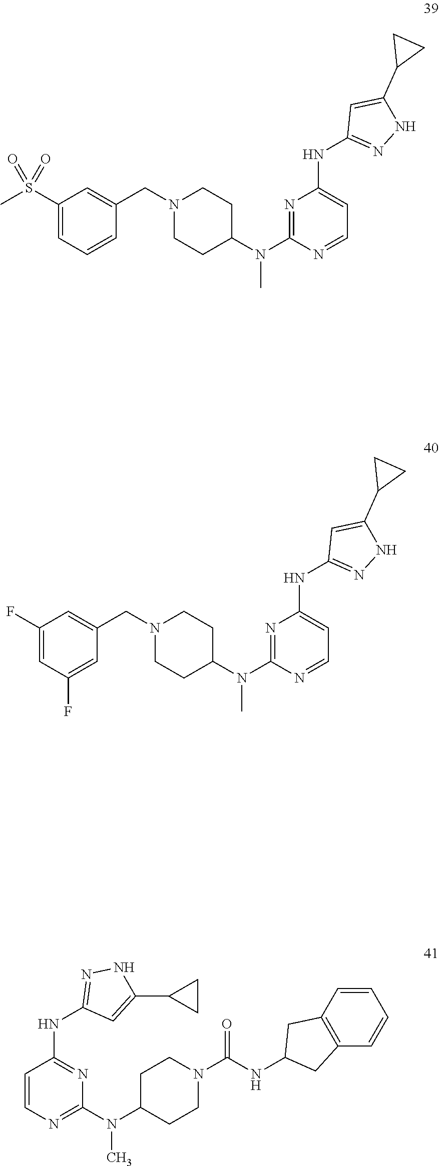 [SMILES: CN(c1nccc(Nc2cc(C3CC3)[nH]n2)n1)C1CCN(Cc2cccc(S(C)(=O)=O)c2)CC1 ; CN(c1nccc(Nc2cc(C3CC3)[nH]n2)n1)C1CCN(Cc2cc(F)cc(F)c2)CC1 ; CN(c1nccc(Nc2cc(C3CC3)[nH]n2)n1)C1CCN(C(=O)NC2Cc3ccccc3C2)CC1] 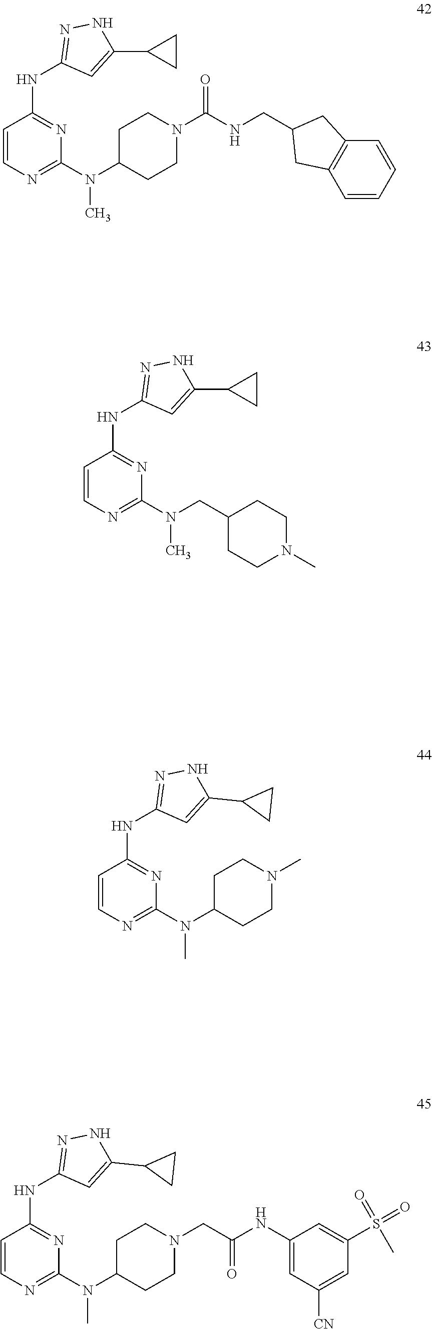 [SMILES: CN(c1nccc(Nc2cc(C3CC3)[nH]n2)n1)C1CCN(C(=O)NCC2Cc3ccccc3C2)CC1 ; CN1CCC(CN(C)c2nccc(Nc3cc(C4CC4)[nH]n3)n2)CC1 ; CN1CCC(N(C)c2nccc(Nc3cc(C4CC4)[nH]n3)n2)CC1 ; CN(c1nccc(Nc2cc(C3CC3)[nH]n2)n1)C1CCN(CC(=O)Nc2cc(C#N)cc(S(C)(=O)=O)c2)CC1] 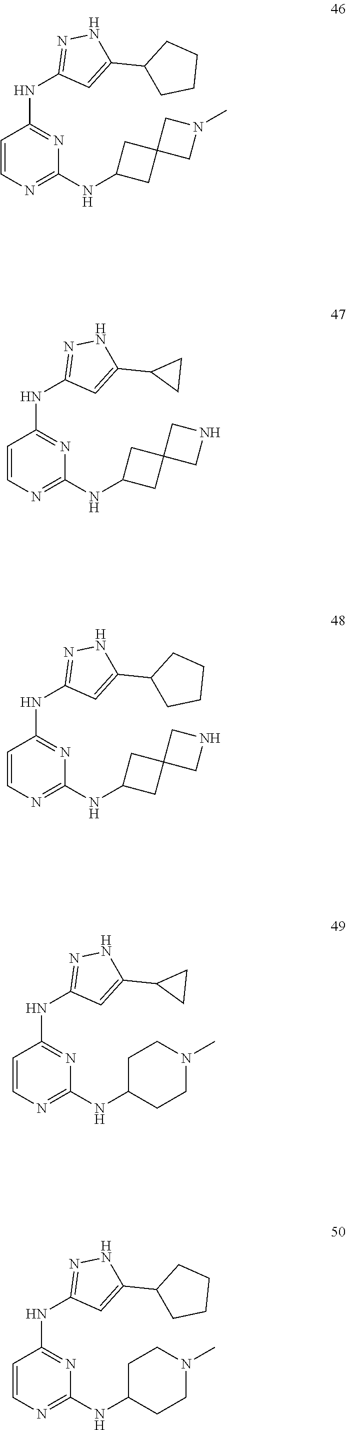 [SMILES: CN1CC2(CC(Nc3nccc(Nc4cc(C5CCCC5)[nH]n4)n3)C2)C1 ; c1cc(Nc2cc(C3CC3)[nH]n2)nc(NC2CC3(CNC3)C2)n1 ; c1cc(Nc2cc(C3CCCC3)[nH]n2)nc(NC2CC3(CNC3)C2)n1 ; CN1CCC(Nc2nccc(Nc3cc(C4CC4)[nH]n3)n2)CC1 ; CN1CCC(Nc2nccc(Nc3cc(C4CCCC4)[nH]n3)n2)CC1] 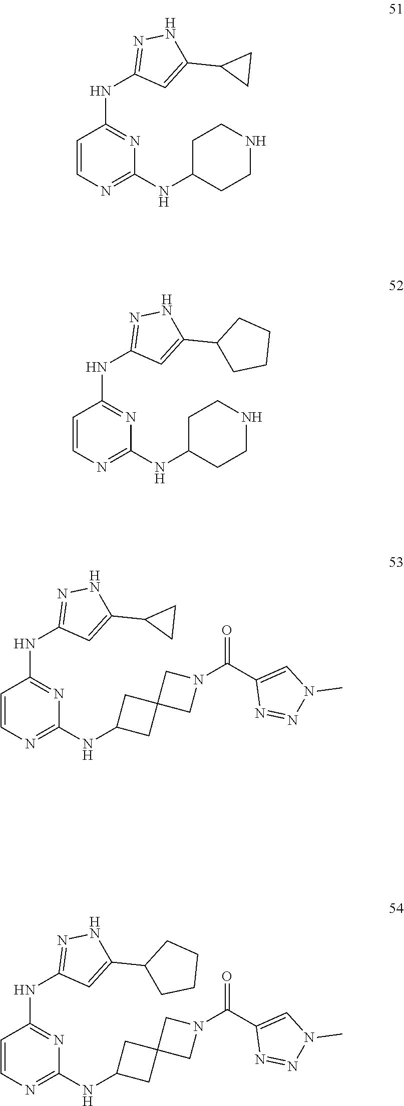 [SMILES: c1cc(Nc2cc(C3CC3)[nH]n2)nc(NC2CCNCC2)n1 ; c1cc(Nc2cc(C3CCCC3)[nH]n2)nc(NC2CCNCC2)n1 ; Cn1cc(C(=O)N2CC3(CC(Nc4nccc(Nc5cc(C6CC6)[nH]n5)n4)C3)C2)nn1 ; Cn1cc(C(=O)N2CC3(CC(Nc4nccc(Nc5cc(C6CCCC6)[nH]n5)n4)C3)C2)nn1] 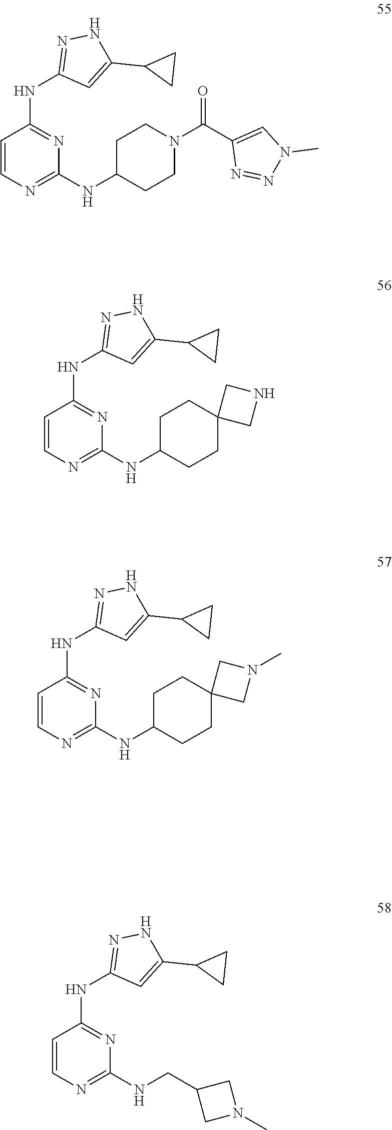 [SMILES: Cn1cc(C(=O)N2CCC(Nc3nccc(Nc4cc(C5CC5)[nH]n4)n3)CC2)nn1 ; c1cc(Nc2cc(C3CC3)[nH]n2)nc(NC2CCC3(CC2)CNC3)n1 ; CN1CC2(CCC(Nc3nccc(Nc4cc(C5CC5)[nH]n4)n3)CC2)C1 ; CN1CC(CNc2nccc(Nc3cc(C4CC4)[nH]n3)n2)C1] 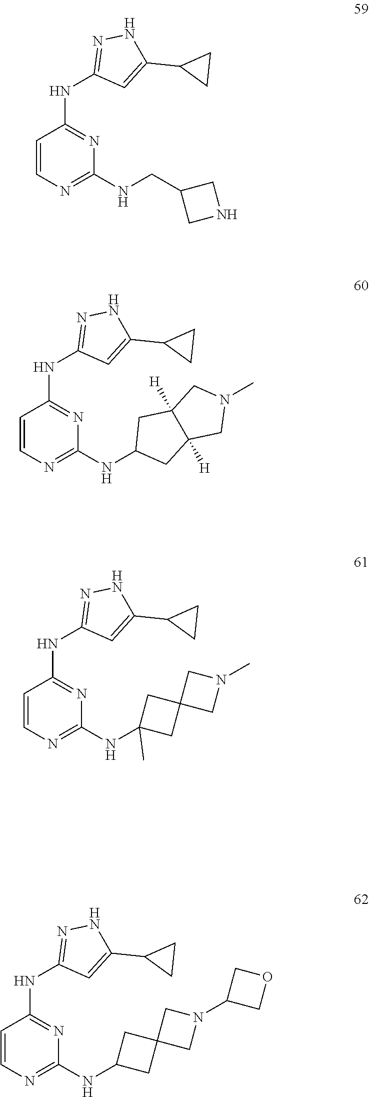 [SMILES: c1cc(Nc2cc(C3CC3)[nH]n2)nc(NCC2CNC2)n1 ; CN1C[C@H]2CC(Nc3nccc(Nc4cc(C5CC5)[nH]n4)n3)C[C@H]2C1 ; CN1CC2(C1)CC(C)(Nc1nccc(Nc3cc(C4CC4)[nH]n3)n1)C2 ; c1cc(Nc2cc(C3CC3)[nH]n2)nc(NC2CC3(C2)CN(C2COC2)C3)n1] 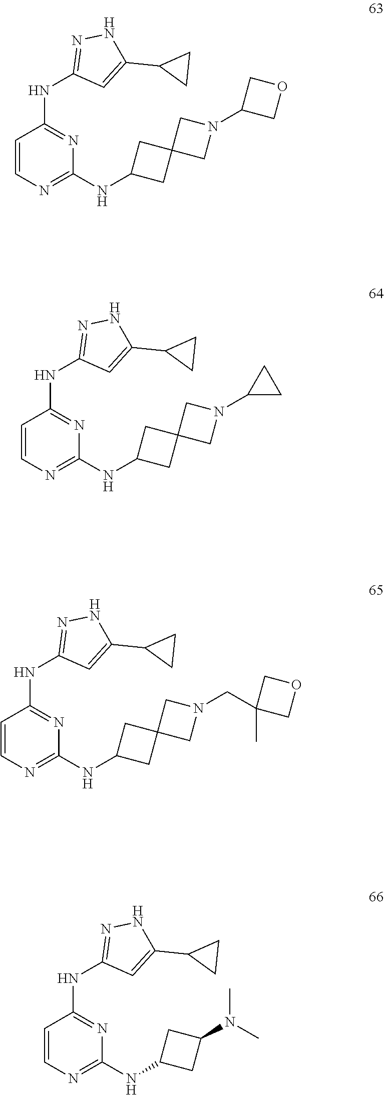 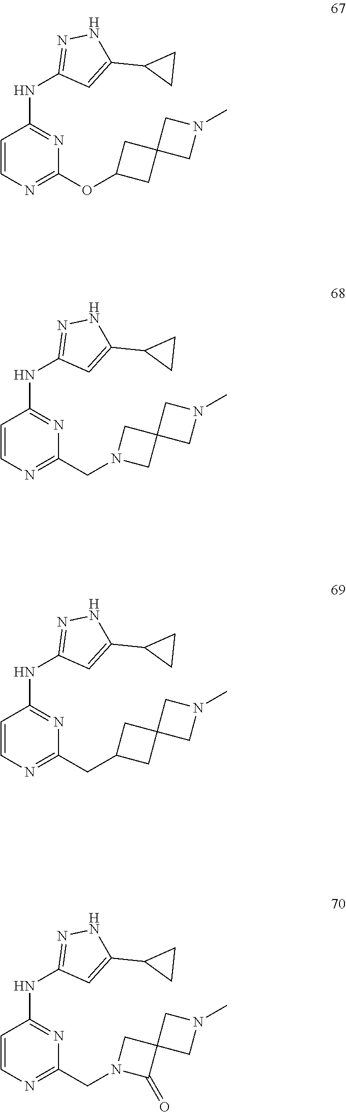 [SMILES: CN1CC2(CC(Oc3nccc(Nc4cc(C5CC5)[nH]n4)n3)C2)C1 ; CN1CC2(C1)CN(Cc1nccc(Nc3cc(C4CC4)[nH]n3)n1)C2 ; CN1CC2(CC(Cc3nccc(Nc4cc(C5CC5)[nH]n4)n3)C2)C1 ; CN1CC2(C1)CN(Cc1nccc(Nc3cc(C4CC4)[nH]n3)n1)C2=O] 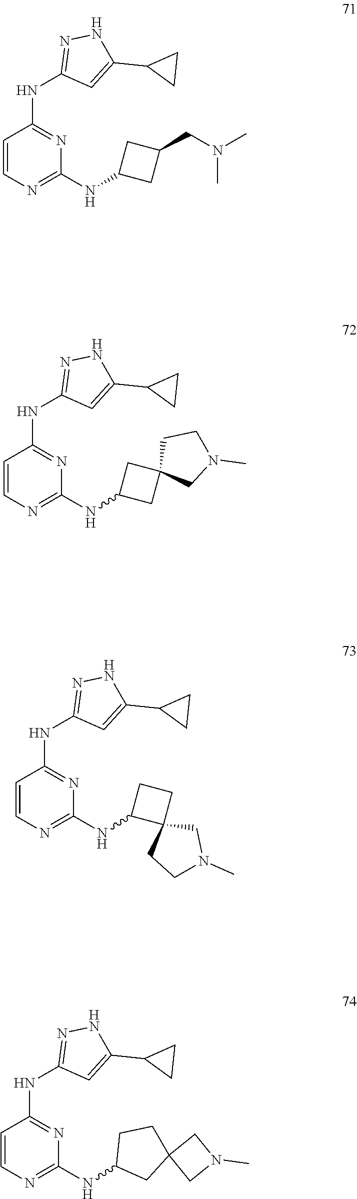 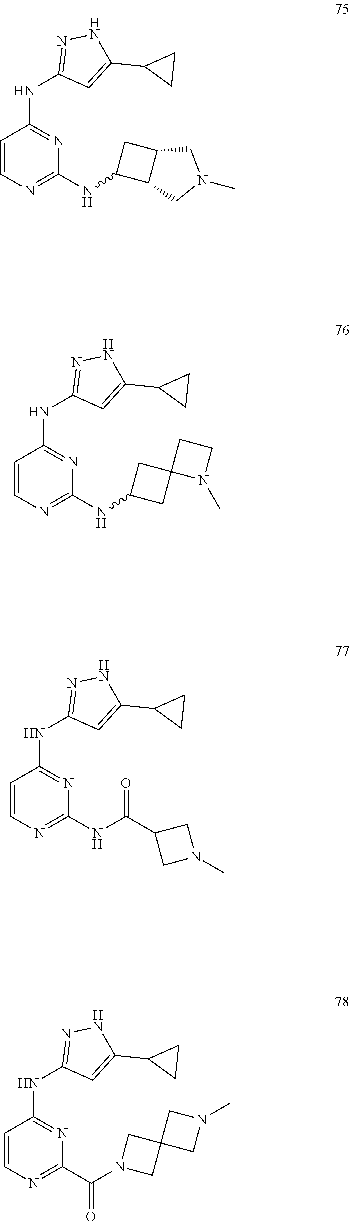 [SMILES: CN1C[C@H]2CC(Nc3nccc(Nc4cc(C5CC5)[nH]n4)n3)[C@H]2C1 ; CN1CCC12CC(Nc1nccc(Nc3cc(C4CC4)[nH]n3)n1)C2 ; CN1CC(C(=O)Nc2nccc(Nc3cc(C4CC4)[nH]n3)n2)C1 ; CN1CC2(C1)CN(C(=O)c1nccc(Nc3cc(C4CC4)[nH]n3)n1)C2] 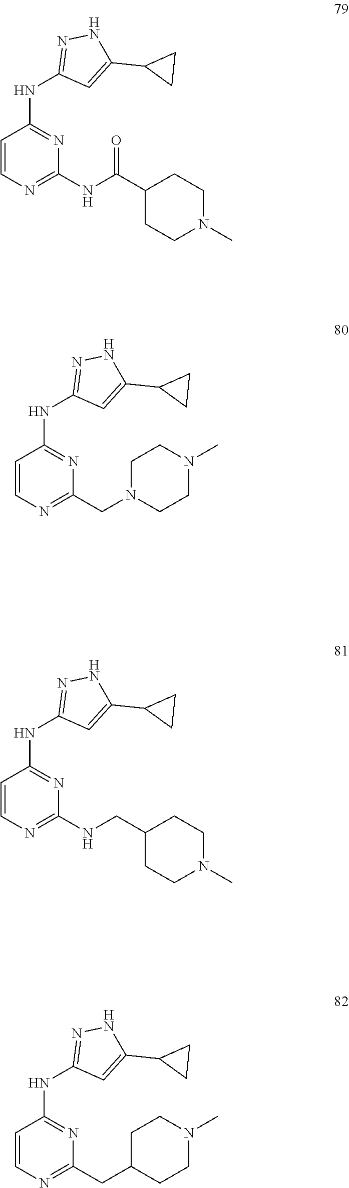 [SMILES: CN1CCC(C(=O)Nc2nccc(Nc3cc(C4CC4)[nH]n3)n2)CC1 ; CN1CCN(Cc2nccc(Nc3cc(C4CC4)[nH]n3)n2)CC1 ; CN1CCC(CNc2nccc(Nc3cc(C4CC4)[nH]n3)n2)CC1 ; CN1CCC(Cc2nccc(Nc3cc(C4CC4)[nH]n3)n2)CC1] 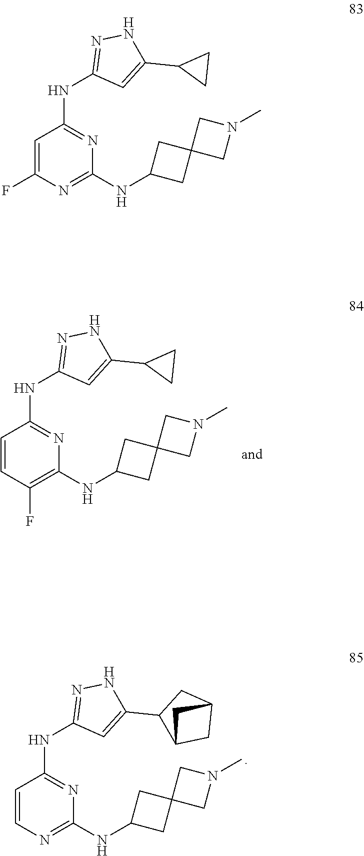 [SMILES: CN1CC2(CC(Nc3nc(F)cc(Nc4cc(C5CC5)[nH]n4)n3)C2)C1 ; CN1CC2(CC(Nc3nc(Nc4cc(C5CC5)[nH]n4)ccc3F)C2)C1 ; CN1CC2(CC(Nc3nccc(Nc4cc(C5C[C@H]6C[C@@H]5C6)[nH]n4)n3)C2)C1]
and a pharmaceutically acceptable salt, stereoisomer, and/or N-oxide thereof.

48. A pharmaceutical composition comprising a compound according to any one of claims 1-47 or a pharmaceutically acceptable salt, stereoisomer, and/or N-oxide thereof, and at least one pharmaceutically acceptable carrier or diluent.

49. The pharmaceutical composition of claim 48, wherein the composition is formulated for parenteral administration.

50. The pharmaceutical composition of claim 48, wherein the composition is formulated for intravenous administration.

51. The pharmaceutical composition of claim 48, wherein the composition is formulated for subcutaneous administration.

52. A method of treating a proliferative disease, comprising:
administering to a subject with a proliferative disease a therapeutically effective amount of a compound according to any one of claims 1-47, or a pharmaceutically acceptable salt, stereoisomer and/or N-oxide thereof, or a therapeutically effective amount of the pharmaceutical composition of any one of claims 48-51.

53. The method of claim 52, wherein the proliferative disease is cancer.

54. The method of claim 53, wherein the cancer is selected from the group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

55. A method of modulating MycN in cells of a subject in need thereof, comprising:
administering to a subject in need thereof an amount of a compound according to any one of claims 1-47, or a pharmaceutically acceptable salt, stereoisomer and/or N-oxide thereof, or a pharmaceutical composition according to any one of claims 48-51, that is effective to cause MycN modulation in cells of the subject.

56. The method of any one of claims 52-55, further comprising administering to the subject a second therapy.

57. The method of claim 56, wherein the second therapy is an antineoplastic therapy.

58. The method of claim 57, wherein the antineoplastic therapy is administration of one or more agents selected from a DNA topoisomerase I or II inhibitor, a DNA damaging agent, an immunotherapeutic agent, an antimetabolite or a thymidylate synthase (TS) inhibitor, a microtubule targeted agent, ionising radiation, an inhibitor of a mitosis regulator or a mitotic checkpoint regulator, an inhibitor of a DNA damage signal transducer, and an inhibitor of a DNA damage repair enzyme.

59. The method of claim 57, wherein the antineoplastic therapy is selected from the group consisting of immunotherapy, radiation therapy, photodynamic therapy, gene-directed enzyme prodrug therapy (GDEPT), antibody-directed enzyme prodrug therapy (ADEPT), gene therapy, and controlled diets.
